SEQUENCE LISTING (284 sequences)

SEQ ID NO: 1 - protein, 453 aa, Homo sapiens [sequence omitted]

SEQ ID NO: 2 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 3 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 4 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 5 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 6 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 9 - protein, 5 aa, synthetic construct
GGGGS

SEQ ID NO: 10 - protein, 10 aa, synthetic construct
GGGGSGGGGS

SEQ ID NO: 11 - protein, 15 aa, synthetic construct
GGGGSGGGGSGGGGS

SEQ ID NO: 12 - protein, 20 aa, synthetic construct
GGGGSGGGGSGGGGSGGGGS

SEQ ID NO: 13 - protein, 25 aa, synthetic construct
GGGGSGGGGSGGGGSGGGGSGGGGS

SEQ ID NO: 14 - protein, 20 aa, synthetic construct
GKPGSGKPGSGKPGSGKPGS

SEQ ID NO: 15 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 16 - protein, 120 aa, synthetic construct [sequence omitted]

SEQ ID NO: 17 - protein, 5 aa, synthetic construct
SYTMH

SEQ ID NO: 18 - protein, 17 aa, synthetic construct
YINPRSGYTHYNQKLKD

SEQ ID NO: 19 - protein, 11 aa, synthetic construct
SAYYDYDGFAY

SEQ ID NO: 20 - protein, 106 aa, synthetic construct [sequence omitted]

SEQ ID NO: 21 - protein, 10 aa, synthetic construct
SASSSVSYMN

SEQ ID NO: 22 - protein, 7 aa, synthetic construct
DTSKLAS

SEQ ID NO: 23 - protein, 9 aa, synthetic construct
QQWSSNPPT

SEQ ID NO: 24 - protein, 125 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSS

SEQ ID NO: 25 - protein, 5 aa, synthetic construct
TYAMN

SEQ ID NO: 26 - protein, 19 aa, synthetic construct
RIRSKYNNYATYYADSVKD

SEQ ID NO: 27 - protein, 14 aa, synthetic construct
HANFGAGYVSWFAH

SEQ ID NO: 28 - protein, 109 aa, synthetic construct
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 29 - protein, 14 aa, synthetic construct
GSSTGAVTTSNYAN

SEQ ID NO: 30 - protein, 7 aa, synthetic construct
GTDKRAP

SEQ ID NO: 31 - protein, 9 aa, synthetic construct
ALWYSNHWV

SEQ ID NO: 32 - protein, 109 aa, synthetic construct
QTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 33 - protein, 9 aa, synthetic construct
ALWYSDLWV

SEQ ID NO: 34 - protein, 119 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 35 - protein, 5 aa, synthetic construct
DYYMH

SEQ ID NO: 36 - protein, 17 aa, synthetic construct
WIDLENANTIYDAKFQG

SEQ ID NO: 37 - protein, 10 aa, synthetic construct
DAYGRYFYDV

SEQ ID NO: 38 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 39 - protein, 17 aa, synthetic construct
KSSQSLLNARTGKNYLA

SEQ ID NO: 40 - protein, 7 aa, synthetic construct
WASTRES

SEQ ID NO: 41 - protein, 8 aa, synthetic construct
KQSYSRRT

SEQ ID NO: 42 - protein, 119 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSS

SEQ ID NO: 43 - protein, 17 aa, synthetic construct
WIDLENANTVYDAKFQG

SEQ ID NO: 44 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 45 - protein, 8 aa, synthetic construct
KQSYFRRT

SEQ ID NO: 46 - protein, 119 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSS

SEQ ID NO: 47 - protein, 10 aa, synthetic construct
DAYGQYFYDV

SEQ ID NO: 48 - protein, 112 aa, synthetic construct
DIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 49 - protein, 8 aa, synthetic construct
TQSYFRRT

SEQ ID NO: 50 - protein, 125 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 51 - protein, 14 aa, synthetic construct
HGNFGNSYVSWFAY

SEQ ID NO: 52 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 53 - protein, 14 aa, synthetic construct
RSSTGAVTTSNYAN

SEQ ID NO: 54 - protein, 7 aa, synthetic construct
GTNKRAP

SEQ ID NO: 55 - protein, 9 aa, synthetic construct
ALWYSNLWV

SEQ ID NO: 56 - protein, 112 aa, synthetic construct
DIVLTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKR

SEQ ID NO: 57 - protein, 118 aa, synthetic construct
EVQLLESGGGLGQPGGSLRLSCAASEFTFTSYAMSWVRQAPGKGLEWVSAISGSGGRTYYADSVKGRFTISRDNSKNTLYLQMNSLRAEDTAVYYCAKGAGYWYFDLWGRGTLVTVSS

SEQ ID NO: 58 - protein, 121 aa, synthetic construct
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSS

SEQ ID NO: 59 - protein, 121 aa, synthetic construct
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSS

SEQ ID NO: 60 - protein, 118 aa, synthetic construct
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSS

SEQ ID NO: 61 - protein, 121 aa, synthetic construct
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSS

SEQ ID NO: 62 - protein, 122 aa, synthetic construct
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSS

SEQ ID NO: 63 - protein, 120 aa, synthetic construct
QVQLQESGPGLVKPSQTLSLTCTVSGFSLSDYGVHWVRQPPGKGLEWLGVIWAGGGTNYNPSLKSRKTISKDTSKNQVSLKLSSVTAADTAVYYCARDKGYSYYYSMDYWGQGTTVTVSS

SEQ ID NO: 64 - protein, 121 aa, synthetic construct
QVQLQQPGSELVRPGASVKLSCKASGYTFTSYWMHWVKQRHGQGLEWIGNIYPGSGRTNYDEKFKSKGTLTVDTSSRTAYMHLSSLTSEDSAVYYCTREHYGSNSHPMDYWGQGTSVTVSS

SEQ ID NO: 65 - protein, 121 aa, synthetic construct
QVQLQQSGTELVKPASSVKISCKASGYTSTSNYMHWIRQQPGNGLEWIGRIYPGNGNTKYNQKFDGKATPTADKSSSTAYMQLSRLTFEDSAVYFCASAPLDYGGHIMDAWGQGTTVTVSS

SEQ ID NO: 66 - protein, 115 aa, synthetic construct
QVQLQQWGAGLLKPSETLSLICAVYGGSFSGYYWSWIRQPPGKGLEWIGDINHSGNTNFNPSLKSRVTISVDTSKNQFSLKLSSVTAADTAVYYCAKGEAWFDPWGQGIMVTVSS

SEQ ID NO: 67 - protein, 115 aa, synthetic construct
QVQLQQWGAGLLKPSETLSLTCAVYGGSFSGYYWNWIRQPPGKGLQWIGEINHSGTTNYNPSLKSRVTISVDTSKNQFSLKLSSLTAADTAVYYCARGEAVALYWGQGTLVTVSS

SEQ ID NO: 68 - protein, 124 aa, synthetic construct
QVQLVESGGGVVQPGRSLRLSCAASGFTFSSYGMHWVRQAPGKGLEWVAVISYAGNNKYYADSVKGRFTVSRDNSKKTLYLQMNSLRSEDTAVYYCAKDSYYDFLTDPDVLDIWGQGTMVTVSS

SEQ ID NO: 69 - protein, 120 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 70 - protein, 120 aa, synthetic construct
QVQLVQSGAEVKKPGASVKVSCKASGYTFTSYYIHWVRQAPGQGLEWIGSIYPGNVNTNYNEKFKDRATLTVDTSISTAYMELSRLRSDDTAVYFCTRSHYGLDWNFDVWGQGTTVTVSS

SEQ ID NO: 71 - protein, 120 aa, synthetic construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSS

SEQ ID NO: 72 - protein, 120 aa, synthetic construct
QVQLVQSGGGVVQPGQSLRLSCAASGFSLSDYGVHWVRQAPGKGLEWLGVIWAGGGTNYNSALQSRFTISKDNSKNTVYLQMNSLRAEDTAVYYCARDKGYSYYYSMDYWGQGTTVTVSS

SEQ ID NO: 73 - protein, 120 aa, synthetic construct
VKLQQSGAELVKPGASVRLSCKASGYTFTEYIIHWIKLRSGQGLEWIGWFYPGSNDIQYNAKFKGKATLTADKSSSTVYMELTGLTSEDSAVYFCARRDDFSGYDALPYWGQGTMVTVSS

SEQ ID NO: 74 - protein, 120 aa, synthetic construct
VQLQQSGAELKKPGASVKVSCKASGYTFTEYIIHWIKLRSGQGLEWIGWFYPGSNDIQYNAQFKGKATLTADKSSSTVYMELTGLTPEDSAVYFCARRDDFSGYDALPYWGQGTLVTVSA

SEQ ID NO: 75 - protein, 108 aa, synthetic construct
DIQLTQSPAFLSASLGETVSIECLGSEDIYGYLAWYQQKPGKSPQLLIYVANRLQDGVPSRFSGSGSGTQYSLKISGMQPEDEGDYYCLQGSKFPLTFGSGTKLEIKR

SEQ ID NO: 76 - protein, 107 aa, synthetic construct
DIQMTQSPASLSASVGETVTITCGASENIYGSLNWYQRKQGKSPQLLIYGATNLADGMSSRFSGSGSGRQFSLKISSLHPDDVATYYCQNLLSTPYTFGGGTKLELK

SEQ ID NO: 77 - protein, 109 aa, synthetic construct
DIQMTQSPASLSVSVGETVTITCRTNENIYSNLAWYQQKQGKSPQLLIYAATHLVEGVPSRFSGSGSGTQYSLKITSLQSEDFGNYYCQHFWGTPCTFGGGTKLEIKRT

SEQ ID NO: 78 - protein, 107 aa, synthetic construct
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 79 - protein, 108 aa, synthetic construct
  VARIANT 96: note = Any amino acid
DIQMTQSPSSLSASVGDRVTITCKTNENIYSNLAWYQQKDGKSPQLLIYAATHLVEGVPSRFSGSGSGTQYSLTISSLQPEDFGNYYCQHFWGTPXTFGGGTKLEIKR

SEQ ID NO: 80 - protein, 107 aa, synthetic construct
DIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIK

SEQ ID NO: 81 - protein, 111 aa, synthetic construct
DIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 82 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 83 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 84 - protein, 111 aa, synthetic construct
DIQMTQSPSTLSASVGDRVTITCRASESVEYYVTSLMQWYQQKPGKAPKLLIFAASNVESGVPSRFSGSGSGTEFTLTISSLQPDDFATYYCQQSRKVPYTFGQGTKLEIK

SEQ ID NO: 85 - protein, 111 aa, synthetic construct
DIVLTQSPASLAVSPGQRATITCRASESVEYYVTSLMQWYQQKPGQPPKLLIFAASNVESGVPARFSGSGSGTDFTLTINPVEANDVANYYCQQSRKVPYTFGQGTKLEIK

SEQ ID NO: 86 - protein, 107 aa, synthetic construct
EIVLTQSPATLSLSPGERAILSCRASQSVSSYLAWYQQKPGQAPRLLIYDASNRATGIPARFSGSGSGTDFTLTISSLEPEDFAVYYCQQRSNWPLTFGGGTKVEIK

SEQ ID NO: 87 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 88 - protein, 107 aa, synthetic construct
EIVMTQSPATLSVSPGERATLSCRASQSVNSNLAWYQQKPGQAPRLLIYGASARATGIPARFSGSGSGTEFTLTISSLQSEDFAVYYCQQYYNWPLTFGGGTKVEIK

SEQ ID NO: 89 - protein, 107 aa, synthetic construct
EIVMTQSPATLSVSPGERATLSCRASQSVRSNLDWYQQKPGQAPRLLIYGASTRATGIPARFSGSGSGTEFTLTISSLQSEDFAVYYCQQYNNWPLTFGGGTKVEIK

SEQ ID NO: 90 - protein, 120 aa, synthetic construct
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSS

SEQ ID NO: 91 - protein, 5 aa, synthetic construct
DYGVH

SEQ ID NO: 92 - protein, 5 aa, synthetic construct
EYIIH

SEQ ID NO: 93 - protein, 5 aa, synthetic construct
GYYWN

SEQ ID NO: 94 - protein, 5 aa, synthetic construct
GYYWS

SEQ ID NO: 95 - protein, 15 aa, synthetic construct
RASESVEYYVTSLMQ

SEQ ID NO: 96 - protein, 5 aa, synthetic construct
RNNMH

SEQ ID NO: 97 - protein, 5 aa, synthetic construct
SNYMH

SEQ ID NO: 98 - protein, 5 aa, synthetic construct
SYAMS

SEQ ID NO: 99 - protein, 5 aa, synthetic construct
SYGMH

SEQ ID NO: 100 - protein, 5 aa, synthetic construct
SYGVH

SEQ ID NO: 101 - protein, 5 aa, synthetic construct
SYWMH

SEQ ID NO: 102 - protein, 5 aa, synthetic construct
SYYIH

SEQ ID NO: 103 - protein, 5 aa, synthetic construct
SYYMS

SEQ ID NO: 104 - protein, 5 aa, synthetic construct
SYYWS

SEQ ID NO: 105 - protein, 7 aa, synthetic construct
AASNVES

SEQ ID NO: 106 - protein, 17 aa, synthetic construct
AISGSGGRTYYADSVKG

SEQ ID NO: 107 - protein, 17 aa, synthetic construct
CIYPGNVNTNYNEKFKD

SEQ ID NO: 108 - protein, 16 aa, synthetic construct
DINHSGNTNFNPSLKS

SEQ ID NO: 109 - protein, 16 aa, synthetic construct
EINHSGTTNYNPSLKS

SEQ ID NO: 110 - protein, 17 aa, synthetic construct
GISSNGGRTYYADSVKG

SEQ ID NO: 111 - protein, 17 aa, synthetic construct
NIYPGSGRTNYDEKFKS

SEQ ID NO: 112 - protein, 17 aa, synthetic construct
RIYPGNGNTKYNQKFDG

SEQ ID NO: 113 - protein, 17 aa, synthetic construct
SIYPGNVNTNYAQKFQG

SEQ ID NO: 114 - protein, 17 aa, synthetic construct
SIYPGNVNTNYNEKFKD

SEQ ID NO: 115 - protein, 17 aa, synthetic construct
TISESGDSTYYADSVKG

SEQ ID NO: 116 - protein, 17 aa, synthetic construct
TISGSGDSTYYADSVKG

SEQ ID NO: 117 - protein, 17 aa, synthetic construct
VISYAGNNKYYADSVKG

SEQ ID NO: 118 - protein, 16 aa, synthetic construct
VIWAGGGTNYNPSLKS

SEQ ID NO: 119 - protein, 16 aa, synthetic construct
VIWAGGGTNYNSALQS

SEQ ID NO: 120 - protein, 16 aa, synthetic construct
VIWPGGGTNFNSALMS

SEQ ID NO: 121 - protein, 17 aa, synthetic construct
WFYPGSNDIQYNAKFKG

SEQ ID NO: 122 - protein, 17 aa, synthetic construct
WFYPGSNDIQYNAQFKG

SEQ ID NO: 123 - protein, 16 aa, synthetic construct
YIYYSGITHYNPSLKS

SEQ ID NO: 124 - protein, 12 aa, synthetic construct
APLDYGGHIMDA

SEQ ID NO: 125 - protein, 9 aa, synthetic construct
DDELLSFDY

SEQ ID NO: 126 - protein, 12 aa, synthetic construct
DKGYSYYYSMDY

SEQ ID NO: 127 - protein, 13 aa, synthetic construct
DRAYGNYLYAMDY

SEQ ID NO: 128 - protein, 15 aa, synthetic construct
DSYYDFLTDPDVLDI

SEQ ID NO: 129 - protein, 12 aa, synthetic construct
EHYGSNSHPMDY

SEQ ID NO: 130 - protein, 9 aa, synthetic construct
GAGYWYFDL

SEQ ID NO: 131 - protein, 7 aa, synthetic construct
GEAVALY

SEQ ID NO: 132 - protein, 7 aa, synthetic construct
GEAWFDP

SEQ ID NO: 133 - protein, 9 aa, synthetic construct
QQSRKVPYT

SEQ ID NO: 134 - protein, 12 aa, synthetic construct
RDDFSGYDALPY

SEQ ID NO: 135 - protein, 12 aa, synthetic construct
SGPGLRQVGFDY

SEQ ID NO: 136 - protein, 11 aa, synthetic construct
SHYGLDWNFDV

SEQ ID NO: 137 - protein, 14 aa, synthetic construct
WGVRRDYYYYGMDV

SEQ ID NO: 138 - protein, 5 aa, synthetic construct
DYGVH

SEQ ID NO: 139 - protein, 11 aa, synthetic construct
GASENIYGSLN

SEQ ID NO: 140 - protein, 11 aa, synthetic construct
HASQNIYVWLN

SEQ ID NO: 141 - protein, 11 aa, synthetic construct
KTNENIYSNLA

SEQ ID NO: 142 - protein, 11 aa, synthetic construct
LGSEDIYGYLA

SEQ ID NO: 143 - protein, 11 aa, synthetic construct
QASQNIYVWLN

SEQ ID NO: 144 - protein, 122 aa, synthetic construct
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 145 - protein, 112 aa, synthetic construct
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 146 - protein, 15 aa, synthetic construct
RASESVEYYVTSLMQ

SEQ ID NO: 147 - protein, 11 aa, synthetic construct
RASQSISSYLN

SEQ ID NO: 148 - protein, 11 aa, synthetic construct
RASQSVNSNLA

SEQ ID NO: 149 - protein, 11 aa, synthetic construct
RASQSVRSNLD

SEQ ID NO: 150 - protein, 12 aa, synthetic construct
RASQSVSSSYLA

SEQ ID NO: 151 - protein, 11 aa, synthetic construct
RASQSVSSYLA

SEQ ID NO: 152 - protein, 11 aa, synthetic construct
RTNENIYSNLA

SEQ ID NO: 153 - protein, 7 aa, synthetic construct
AASNVDS

SEQ ID NO: 154 - protein, 7 aa, synthetic construct
AASNVES

SEQ ID NO: 155 - protein, 7 aa, synthetic construct
AASSLQS

SEQ ID NO: 156 - protein, 7 aa, synthetic construct
AATHLVE

SEQ ID NO: 157 - protein, 7 aa, synthetic construct
DASNRAT

SEQ ID NO: 158 - protein, 7 aa, synthetic construct
GASARAT

SEQ ID NO: 159 - protein, 7 aa, synthetic construct
GASSRAT

SEQ ID NO: 160 - protein, 7 aa, synthetic construct
GASTRAT

SEQ ID NO: 161 - protein, 7 aa, synthetic construct
GATNLAD

SEQ ID NO: 162 - protein, 7 aa, synthetic construct
KASNLHT

SEQ ID NO: 163 - protein, 7 aa, synthetic construct
VANRLQD

SEQ ID NO: 164 - protein, 16 aa, synthetic construct
VIWAGGGTNYNSALMS

SEQ ID NO: 165 - protein, 12 aa, synthetic construct
DKGYSYYYSMDY

SEQ ID NO: 166 - protein, 9 aa, synthetic construct
LQGSKFPLT

SEQ ID NO: 167 - protein, 9 aa, synthetic construct
QHFWGTPCT

SEQ ID NO: 168 - protein, 9 aa, synthetic construct
  VARIANT 8: note = Any amino acid
QHFWGTPXT

SEQ ID NO: 169 - protein, 9 aa, synthetic construct
QNLLSTPYT

SEQ ID NO: 170 - protein, 9 aa, synthetic construct
QQGQTYPYT

SEQ ID NO: 171 - protein, 9 aa, synthetic construct
QQRSNWPLT

SEQ ID NO: 172 - protein, 9 aa, synthetic construct
QQSRKVPFT

SEQ ID NO: 173 - protein, 9 aa, synthetic construct
QQSRKVPYT

SEQ ID NO: 174 - protein, 10 aa, synthetic construct
QQSYSTPPIT

SEQ ID NO: 175 - protein, 9 aa, synthetic construct
QQVYSTPFT

SEQ ID NO: 176 - protein, 9 aa, synthetic construct
QQYGSSPWT

SEQ ID NO: 177 - protein, 9 aa, synthetic construct
QQYNNWPLT

SEQ ID NO: 178 - protein, 9 aa, synthetic construct
QQYYNWPLT

SEQ ID NO: 179 - protein, 120 aa, synthetic construct
EVQLVQSGGALVQAGGSLRLSCVAPRTIGLISLMGWYRQAPGKQRELVATIIASGRTNYAESVKDRFTISRDNAENKVYLQVNSLKPEDTAVYYCRVNAETAPWSRRGFWGQGTQVTVKP

SEQ ID NO: 180 - protein, 117 aa, synthetic construct
QVQLQQSGGGLVQAGGSLRLSCVASGISFNVVDMDWHRQAPGKERELVAGITRGGATYYADSVKGRFTISRDNAKNSAYLQLANLKPEDTAVYYCNARWPSRYQNYWGQGTQVTVKP

SEQ ID NO: 181 - protein, 124 aa, synthetic construct
EVQLVQSGGGLVQTGGSLRLSCAASGRMFSNYAMGWFRQAPGKEREFVAAINYRRDSADYADSVKGRFTISRDNAKNTVYLEMNSLKPEDTAIYYCGFTYAGWASSRRDDYNYWGQGTQVTVKP

SEQ ID NO: 182 - protein, 108 aa, synthetic construct
DIQMTQSPSSLSASVGDRVTITCRASRPIWPFLEWYQQKPGKAPKLLIYFTSRLRHGVPSRFSGSGSGTCFTLTISSLQPEDFATYYCLQNVANPATFSQGTKVEIKR

SEQ ID NO: 183 - protein, 5 aa, synthetic construct
ISLMG

SEQ ID NO: 184 - protein, 5 aa, synthetic construct
VVDMD

SEQ ID NO: 185 - protein, 5 aa, synthetic construct
NYAMG

SEQ ID NO: 186 - protein, 11 aa, synthetic construct
RASRPIWPFLE

SEQ ID NO: 187 - protein, 16 aa, synthetic construct
TIIASGRTNYAESVKD

SEQ ID NO: 188 - protein, 16 aa, synthetic construct
GITRGGATYYADSVKG

SEQ ID NO: 189 - protein, 17 aa, synthetic construct
AINYRRDSADYADSVKG

SEQ ID NO: 190 - protein, 7 aa, synthetic construct
FTSRLRH

SEQ ID NO: 191 - protein, 12 aa, synthetic construct
NAETAPWSRRGF

SEQ ID NO: 192 - protein, 9 aa, synthetic construct
RWPSRYQNY

SEQ ID NO: 193 - protein, 15 aa, synthetic construct
TYAGWASSRRDDYNY

SEQ ID NO: 194 - protein, 9 aa, synthetic construct
LQNVANPAT

SEQ ID NO: 195 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 196 - protein, 197 aa, synthetic construct
ARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 197 - protein, 184 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 198 - protein, 621 aa, synthetic construct
ARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 199 - protein, 582 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 200 - protein, 611 aa, synthetic construct
ARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 201 - protein, 572 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 202 - protein, 773 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 203 - protein, 773 aa, synthetic construct
ARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 204 - protein, 734 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 205 - protein, 734 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 206 - protein, 719 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 207 - protein, 719 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 208 - protein, 724 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 209 - protein, 724 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 210 - protein, 1409 aa, synthetic construct
ARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 211 - protein, 1331 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 212 - protein, 401 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 213 - protein, 998 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 214 - protein, 998 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSEVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 215 - protein, 241 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIK

SEQ ID NO: 216 - protein, 393 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 217 - protein, 393 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 218 - protein, 249 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 219 - protein, 249 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVL

SEQ ID NO: 220 - protein, 401 aa, synthetic construct; identical to SEQ ID NO: 212

SEQ ID NO: 221 - protein, 249 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSDLWVFGGGTKLTVL

SEQ ID NO: 222 - protein, 246 aa, synthetic construct
QVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIK

SEQ ID NO: 223 - protein, 246 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGRYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYFRRTFGGGTKVEIK

SEQ ID NO: 224 - protein, 246 aa, synthetic construct
QVQLVQSGAEVKKPGASVKVSCKASGFNIKDYYMHWVRQAPGQRLEWIGWIDLENANTVYDAKFQGRVTITRDTSASTAYMELSSLRSEDTAVYYCARDAYGQYFYDVWGQGTLVTVSSGGGGSGGGGSGGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCTQSYFRRTFGGGTKVEIK

SEQ ID NO: 225 - protein, 248 aa, synthetic construct
EVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 226 - protein, 248 aa, synthetic construct
EVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 227 - protein, 252 aa, synthetic construct
QVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSGKPGSGKPGSGKPGSGKPGSDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 228 - protein, 400 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 229 - protein, 400 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 230 - protein, 404 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSGKPGSGKPGSGKPGSGKPGSDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 231 - protein, 664 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSEVQLLESGGGLVQPGGSLRLSCAASGFTFSSYAMSWVRQAPGKGLEWVSTISGSGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 232 - protein, 664 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSEVQLLESGGGLVQPGGSLRLSCAASGFTFSSYYMSWVRQAPGKGLEWVSTISESGDSTYYADSVKGRFTISRDNSKNTLYLQMNSLRAEDTAVYYCAKSGPGLRQVGFDYWGQGTLVTVSSGKPGSGKPGSGKPGSGKPGSDIQMTQSPSSLSASVGDRVTITCRASQSISSYLNWYQQKPGKAPKLLIYAASSLQSGVPSRFSGSGSGTDFTLTISSLQPEDFATYYCQQVYSTPFTFGQGTKLEIK

SEQ ID NO: 233 - protein, 667 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSGKPGSGKPGSGKPGSGKPGSDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 234 - protein, 668 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSQVQLQESGPGLVKPSETLSLTCAVSGFSLTSYGVHWIRQPPGKGLEWLGVIWPGGGTNFNSALMSRLTISEDTSKNQVSLKLSSVTAADTAVYYCARDRAYGNYLYAMDYWGQGTLVTVSSGKPGSGKPGSGKPGSGKPGSDIQMTQSPSSLSASVGDRVTITCRASESVEYYVTSLMQWYQQKPGKAPKLLIYAASNVDSGVPSRFSGSGSGTDFTLTISSLQPEDIATYYCQQSRKVPFTFGGGTKVEIK

SEQ ID NO: 235 - protein, 14 aa, synthetic construct
GGGGSGGGGSGGGG

SEQ ID NO: 236 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 237 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 239 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 453 aa, synthetic construct
EVQLVESGGGLVQPGRSLRLSCVASGFTFNDYAMHWVRQAPGKGLEWVSVISWNSDSIGYADSVKGRFTISRDNAKNSLYLQMHSLRAEDTALLYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLEPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 241 - protein, 449 aa, synthetic construct
EVQLVESGGGLVQPGRSLRLSCAASGFTFDDYTMHWVRQAPGKGLEWVSGISWNSGSIGYADSVKGRFTISRDNAKKSLYLQMNSLRAEDTALLYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 242 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 993 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSGGGGSGGGGSGGGGSDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 244 - protein, 119 aa, synthetic construct
QVQLQESGAELVMPGASVKLSCKASGYTFINYWMHWVKQRPGQGLEWIGEISPSDSYTDYNQKFKGKATLTVDTSSNAAYMQLSGLTFEDSAVYYCTRGGKSLGFFDYWGQGTTLTVSS

SEQ ID NO: 245 - protein, 109 aa, synthetic construct
EFMDILMTQSPSSLSASLGGKVTISCKASQDINNYIAWYQHKPGKGPRLLIHFTSTLQPGIPSRFSGSGSGRDYSFSISNLEPEDIGTYFCLQYNYLWTFGGGTKLEIK

SEQ ID NO: 246 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 247 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 248 - protein, 116 aa, synthetic construct
QVQLVQSGPEVKKPGASVKVSCKASGYTFTIYWLGWVRQAPGQGLEWMGNIFPGSAYINYNEKFKGKVTITADTSTSTAYMELSSLRSEDTAVYFCAREGSNSGYWGQGTLVTVSS

SEQ ID NO: 249 - protein, 113 aa, synthetic construct
DIVMTQSPDSLAVSLGERATINCKSSQSLLNSGNQQNYLAWYQQKPGQPPKLLIYGASTRESGVPDRFSGSGSGTDFTLTISSLQAEDVAVYYCQSDHIYPYTFGQGTKLEIK

SEQ ID NO: 250 - protein, 121 aa, synthetic construct
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 251 - protein, 109 aa, synthetic construct
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 252 - protein, 121 aa, synthetic construct
QIQLVQSGHEVKKPGASVKVSCKASGYTFTNYGMNWVRQAPGQGLEWMGWINTKTGEPTYAEEFKGRFVFSLETSASTAYLQISSLKAEDMAMYFCGRGGYGSSYWYFDVWGQGTTVTVSS

SEQ ID NO: 253 - protein, 107 aa, synthetic construct
DIQMTQSPSSLSASVGDRVTITCKASQDVSIAVAWYQQKPGKAPKVLIYSASYRYTGVPDRFSGSGSGTDFTFTISSLQPEDIAVYYCQQHYITPLTFGGGTKVEIK

SEQ ID NO: 254 - protein, 119 aa, synthetic construct
QVQLQESGPGLVKPSETLSLTCTVSGGSISSYGVHWIRQPPGKGLEWIGVIWTSGVTDYNSALMGRVTISVDTSKNQFSLKLSSVTAADTAVYYCARDGDYDRYTMDYWGQGTLVTVSS

SEQ ID NO: 255 - protein, 111 aa, synthetic construct
DIVMTQSPDSLAVSLGERATINCRASKSVSTSGYSYMHWYQQKPGQPPKLLIYLASNLESGVPDRFSGSGSGTDFTLTISSLQAEDVAVYYCQHSRELPYTFGQGTKLEIK

SEQ ID NO: 256 - protein, 124 aa, synthetic construct
QVKLKQSGGGLVQPGGSLKVSCAASGFDFSRYWMSWVRQAPGKGLEWIGEINPDSSTIVYTPSLKDKFIMSRDNAKNTLYLQTSKVRSADTALYYCARRGSHYYGYRTGYFDVWGAGTTVTVSS

SEQ ID NO: 257 - protein, 107 aa, synthetic construct
DVVLTQSPAIMSASPGEKVTMTCSASSSVSYMYWHQQKPGSSPRLLIYDTSNLASGVPVRFSGSGSGTSYSLTISRMEAEDAATYYCQQWSSYPPTFGGGTKLEIKR

SEQ ID NO: 258 - protein, 119 aa, synthetic construct
QVQLQESGPGLVKPSETLSLTCTVSGGSISSSSYYWGWIRQPPGKGLEWIGSIYYSGITNYNPSLKSRVTISVDTSKNQFSLKLSSVTAADTAVYYCARPSNYDAFDIWGQGTMVTVSS

SEQ ID NO: 259 - protein, 111 aa, synthetic construct
QSVLTQPPSVSGAPGQRVTISCTGSSSNIGAGYDVHWYQQVPGTAPKLLIYGNSKRPSGVPDRFSGSKSGTSASLAITGLQAEDEADYYCQSYDSSLGGWVFGGGTKLTVL

SEQ ID NO: 260 - protein, 117 aa, synthetic construct
QVQLVESGGGLVKPGGSLRLSCAASGFTFSDYYMTWIRQAPGKGLEWISYISSSGSTIYYADSVKGRFTISRDNAKNSLYLQMNSLRAEDTAVYYCARDRNSHFDYWGQGTLVTVSS

SEQ ID NO: 261 - protein, 107 aa, synthetic construct
DIQMTQSPSSVSASVGDRVTITCRASQGITRWLAWYQQKPGKAPKLLIYAASVLQSGVPSRFSGSGSGTDFTLTISSLQPEDFATYYCQQSNSFPRTFGQGTKVEIK

SEQ ID NO: 262 - protein, 117 aa, synthetic construct
QVQLVESGGGLVKPGGSLRLSCAASGFTFSDHYMSWIRQAPGKGLEWFSYISSSGGIIYYADSVKGRFTISRDNAKNSLYLQMNSLRAEDTAVYYCARDVGSHFDYWGQGTLVTVSS

SEQ ID NO: 263 - protein, 107 aa, synthetic construct
DIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGQGTKVEIK

SEQ ID NO: 264 - protein, 118 aa, synthetic construct
EVQLVESGGGLIQPGGSLRLSCAVSGFTVSSKFMTWVRQAPGKGLEWVSVIYSGGKTYYADSVKGRFTISRDNSKNTLYLQMNSLRAEDTAVYYCARDSGGWGYFDYWGQGTLVTVSS

SEQ ID NO: 265 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPPTFGQGTKVEIK

SEQ ID NO: 266 - protein, 120 aa, synthetic construct
QVTLRESGPALVKPTQTLTLTCTFSGFSLSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 267 - protein, 106 aa, synthetic construct
DIQMTQSPSTLSASVGDRVTITCKCQLSVGYMHWYQQKPGKAPKLLIYDTSKLASGVPSRFSGSGSGTEFTLTISSLQPDDFATYYCFQGSGYPFTFGGGTKLEIK

SEQ ID NO: 268 - skipped

SEQ ID NO: 269 - protein, 401 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 270 - protein, 126 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDSPFLLDDYYYYYYMDVWGKGTTVTVSS

SEQ ID NO: 271 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGHLFPITFGGGTKVEIK

SEQ ID NO: 272 - protein, 122 aa, synthetic construct
QAYLQQSGAEVVRSGASVKMSCKAAGYTFTSYNMHWVKQTPGQGLEWIGYIYPGNGGTNYNQKFKGKATLTADTSSTTAYMQISSLTSEDSAVYFCARGASMITTGAWFAYWGQGTLVTVSA

SEQ ID NO: 273 - protein, 111 aa, synthetic construct
NIVLTQSPASLAVSLGQRATISCRASESLDTYGNSFMYWYQQKPGQPPKLLIYLASSLESGVPARFSGSGSRTDFTLTIDPVEADDAATYYCHQNHEDPWTFGGGTKLEIK

SEQ ID NO: 274 - protein, 445 aa, synthetic construct
QVQLVQSGPEVKKPGASVKVSCKASGYTFTIYWLGWVRQAPGQGLEWMGNIFPGSAYINYNEKFKGKVTITADTSTSTAYMELSSLRSEDTAVYFCAREGSNSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 275 - protein, 220 aa, synthetic construct
DIVMTQSPDSLAVSLGERATINCKSSQSLLNSGNQQNYLAWYQQKPGQPPKLLIYGASTRESGVPDRFSGSGSGTDFTLTISSLQAEDVAVYYCQSDHIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 480 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLEPKSSDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 446 aa, synthetic construct
QVQLVESGGGLVKPGGSLRLSCAASGFTFSDHYMSWIRQAPGKGLEWFSYISSSGGIIYYADSVKGRFTISRDNAKNSLYLQMNSLRAEDTAVYYCARDVGSHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSDTKVDKKVEPKSCDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 278 - protein, 214 aa, synthetic construct
DIQMTQSPSSVSASVGDRVTITCRASQDISRWLAWYQQKPGKAPKLLISAASRLQSGVPSRFSGSGSGTDFTLTISSLQPEDFAIYYCQQAKSFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 25 aa, synthetic construct
  VARIANT 1..25: note = This sequence may encompass 2-5 GGGGS repeating units
GGGGSGGGGSGGGGSGGGGSGGGGS

SEQ ID NO: 280 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 281 - protein, 15 aa, synthetic construct
  VARIANT 1..15: note = This sequence may encompass 2-3 GGGGS repeating units
GGGGSGGGGSGGGGS

SEQ ID NO: 282 - protein, 399 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGKPGSGKPGSGKPGSGKPGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 283 - protein, 662 aa, synthetic construct
EVQLLESGGGLVQPGGSLRLSCAASGFTFDTYAMNWVRQAPGKGLEWVARIRSKYNNYATYYADSVKDRFTISRDDSKSTLYLQMESLRAEDTAVYYCVRHANFGAGYVSWFAHWGQGTLVTVSSGGGGSGGGGSGGGGSQTVVTQEPSLSVSPGGTVTLTCGSSTGAVTTSNYANWVQQTPGQAPRGLIGGTDKRAPGVPDRFSGSLLGDKAALTITGAQAEDEADYYCALWYSNHWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGKPGSGKPGSGKPGSGKPGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 284 - protein, 247 aa, synthetic construct
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGKPGSGKPGSGKPGSGKPGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

(19) United States
(12) Patent Application Publication
(10) Pub. No.: US 2026/0347469 A1
(43) Pub. Date: Nov. 5, 2026

(54) T CELL COSTIMULATORY MULTIMERIC BINDING MOLECULES AND USES THEREOF

(71) Applicant: IGM Biosciences, Inc., Mountain View, CA (US)

(72) Inventors: Liqin LIU, Woodland Hills, CA (US); Deepal PANDYA, Fremont, CA (US); Beatrice Tien-Yi WANG, Sunnyvale, CA (US); Hyewon PHEE, San Francisco, CA (US); Keyu LI, San Carlos, CA (US); Umesh S. MUCHHAL, Monrovia, CA (US); Angus SINCLAIR, San Francisco, CA (US)

(21) Appl. No.: 19/106,186

(22) PCT Filed: Jan. 5, 2024
(86) PCT No.: PCT/US2024/010591

§ 371 Date: Feb. 24, 2025

Related U.S. Application Data
(60) Provisional application No. 63/478,886, filed on Jan. 6, 2023.
(60) Provisional application No. 63/498,948, filed on Apr. 28, 2023.
(60) Provisional application No. 63/510,092, filed on Jun. 23, 2023.
(60) Provisional application No. 63/590,779, filed on Oct. 16, 2023.

(51) Int. Cl. C07K 16/28; A61K 39/00; A61P 35/00

(52) U.S. Cl. CPC C07K 16/2809; A61P 35/00; C07K 16/2818; A61K 2039/505; C07K 2317/31; C07K 2317/622; C07K 2317/73; C07K 2317/75; C07K 2317/92

(57) ABSTRACT

This disclosure provides multimeric T cell engaging binding molecules comprising five, four, or two bivalent binding units that bind to a target antigen and a modified J chain, where the modified J chain includes a J chain or a functional fragment or variant thereof, an anti-CD3 antigen-binding domain, and a heterologous polypeptide comprising a polypeptide agonist of a T cell costimulatory molecule. The T cell costimulatory molecule can be, e.g., CD28 or 4-1BB. This disclosure further provides multimeric binding molecules comprising five, four, or two bivalent binding units that bind to a target antigen and a modified J chain, where the modified J chain includes a J chain or a functional fragment or variant thereof and a 4-1BB ligand (4-1BBL) trimer that can engage and activate 4-1BB on a target cell.

### CROSS-REFERENCE TO RELATED APPLICATIONS

This application claims the benefit of U.S. Provisional Patent Application Ser. Nos. 63/478,886, filed 6 Jan. 2023; 63/498,948, filed 28 Apr. 2023; 63/510,092, filed 23 Jun. 2023; and 63/590,779, filed 16 Oct. 2023, each of which is incorporated herein by reference in its entirety.

### SEQUENCE LISTING

The instant application contains a Sequence Listing which has been submitted electronically in XML format and is hereby incorporated by reference in its entirety. The XML copy, created on 5 Jan. 2024, is named 043WO1-Sequence-Listing.xml and is 281 kilobytes in size.

### BACKGROUND

T cell-engaging bispecific antibodies that can bind to a tumor antigen and simultaneously target cytotoxic T cells, e.g., by binding to CD3 to promote killing of tumor cells are a valuable and rapidly expanding tool in the treatment of various malignancies. See, e.g., Einsele, H., et al., Cancer 126:3192-3201 (2020). While the advancements made in T cell engager (TCE) therapeutics has been significant, a need remains for improved therapeutic results, especially in solid tumor indications, where the tumor microenvironment can pose a significant barrier to T cell infiltration. See, e.g., Lim, A. R., et al., eLife 9: doi: 10.7554/eLife.55185 (2020).

T cell activation typically involves two signals-activation through MHC-antigen engagement with a T cell receptor (TCR) (“signal 1”) and coactivation through a costimulatory molecule (“signal 2”). Costimulatory molecules are receptors expressed on T cells that upon coactivation can, e.g., induce signaling to activate T cells more fully upon T cell receptor engagement, thereby enhancing signaling and cytokine stimulation. This co-signaling can further modulate T cell differentiation, effector function, and survival. See, e.g., Baeuerle, P. A., and H. Wesche, Curr. Opin. Oncol. 34:552-558 (2022). Exemplary T cell costimulatory molecules include, without limitation, CD28 and 4-1BB. See, e.g., Jeong, S., and S-H. Park, Immune Netw. doi: 10.4110/in.2020.20.e3 (2020).

CD3 engagement by a TCE can replace the conventional signal 1 but lacks the signal 2 coactivation. Combining a bispecific TCE that engages CD3 with a tumor-targeted bispecific antibody that engages and coactivates a T cell costimulatory molecule has shown promise in preclinical studies. See, e.g., Skokos, D., et al., Sci. Transl. Med. 12(525):eaaw7888. doi: 10.1126/scitranslmed.aaw7888 (2020).

Binding molecules, e.g., antibodies that can multimerize, such as IgA and IgM antibodies, have emerged as promising drug candidates in the fields of, e.g., immuno-oncology and infectious diseases, allowing for improved specificity, improved avidity, and the ability to bind to multiple targets. See, e.g., U.S. Pat. Nos. 9,951,134, 9,938,347, 10,351,631, 10,400,038, 10,570,191, 1,0604,559, 10,689,449, 10,787,520, 10,899,835, 11,555,075, 10,618,978, and 11,639,389, U.S. Patent Application Publication Nos. US 2022/0106399, US 2020/0392239, and US 2022/0340676, the contents of which are incorporated herein by reference in their entireties. Bispecific IgM antibodies have demonstrated safety and efficacy as TCEs in preclinical studies. See, e.g., U.S. Pat. No. 10,787,520 (describing, among other things, bispecific IgM antibodies targeting CD20 and CD3 proteins), U.S. Patent Application Publication No. US 2022/0289856 (describing, among other things, bispecific IgM antibodies targeting CD123 and CD3 proteins), and PCT Publication No. WO 2023/150677 A2 (describing, among other things, bispecific IgM antibodies targeting CD38 and CD3 proteins). In addition, imvotamab (IGM-2323), a CD20×CD3 IgM bispecific antibody, has shown promise in early clinical studies. See, e.g., Budde, E., et al., Blood 138 (Suppl. 1): 132 doi: 10.1182/blood-2021-153355 (2021).

A need remains for safe TCEs with improved potency through both signal 1 and signal 2 T cell engagement.

### SUMMARY

Provided herein is a multimeric binding molecule comprising five, four, or two bivalent binding units and a modified J chain, wherein each binding unit comprises two antibody heavy chains, each comprising an IgM or IgA heavy chain constant region or multimerizing variant or fragment thereof each associated with an antigen-binding domain that specifically binds to a target antigen (a “target antigen-binding domain”), and wherein the modified J chain comprises (a) a J chain or a functional fragment or variant thereof (“J”), (b) an anti-CD3 scFv (“C”), and (c) a heterologous polypeptide (“H”), wherein H comprises a polypeptide agonist of a T cell costimulatory molecule, wherein the J, C, and H are associated as a fusion protein. In some embodiments, the T cell costimulatory molecule comprises CD28 or 4-1BB. In some embodiments, H comprises an anti-CD28 antibody or antigen-binding fragment thereof or a 4-1BB ligand (“4-1BBL”) trimer.

In some embodiments, the modified J chain comprises, in the N-terminal to C-terminal direction, C-J-H or H-J-C. In some embodiments, C, J, and H are fused via amino acid linkers which can be the same or different. In some embodiments, each linker consists, independently, of 5 to 25 amino acids. In some embodiments, each linker consists, independently, of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235). In some embodiments, the linker consists of GGGGGGGGS (SEQ ID NO: 10). In some embodiments, the linker consists of GGGGSGGGGSGGGGS (SEQ ID NO: 11). In some embodiments, the linker consists of GKPGSGKPGSGKPGSGKPGS (SEQ ID NO: 14).

In some embodiments, H comprises an antigen-binding fragment of an anti-CD28 antibody. In some embodiments, the antigen-binding fragment of the anti-CD28 antibody comprises a single-chain Fv (scFv) fragment.

In some embodiments, the anti-CD28 scFv fragment comprises a heavy chain variable region (VH) and a light chain variable region (VL), wherein the VH comprises VH complementarity-determining regions VHCDR1, VHCDR2, and VHCDR3 and the VL comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 95, SEQ ID NO: 105, SEQ ID NO: 133, SEQ ID NO: 138, SEQ ID NO: 164, and SEQ ID NO: 165; SEQ ID NO: 98, SEQ ID NO: 106, SEQ ID NO: 130, SEQ ID NO: 151, SEQ ID NO: 157, and SEQ ID NO: 171; SEQ ID NO: 98, SEQ ID NO: 116, SEQ ID NO: 135, SEQ ID NO: 147, SEQ ID NO: 155, and SEQ ID NO: 175; SEQ ID NO: 103, SEQ ID NO: 115, SEQ ID NO: 135, SEQ ID NO: 147, SEQ ID NO: 155, and SEQ ID NO: 175; SEQ ID NO: 96, SEQ ID NO: 110, SEQ ID NO: 125, SEQ ID NO: 147, SEQ ID NO: 155, and SEQ ID NO: 174; SEQ ID NO: 96, SEQ ID NO: 110, SEQ ID NO: 125, SEQ ID NO: 150, SEQ ID NO: 159, and SEQ ID NO: 176; SEQ ID NO: 100, SEQ ID NO: 120, SEQ ID NO: 127, SEQ ID NO: 146, SEQ ID NO: 153, and SEQ ID NO: 172; SEQ ID NO: 104, SEQ ID NO: 123, SEQ ID NO: 137, SEQ ID NO: 150, SEQ ID NO: 159, and SEQ ID NO: 176; SEQ ID NO: 91, SEQ ID NO: 118, SEQ ID NO: 126, SEQ ID NO: 146, SEQ ID NO: 154, and SEQ ID NO: 173; SEQ ID NO: 101, SEQ ID NO: 111, SEQ ID NO: 129, SEQ ID NO: 139, SEQ ID NO: 161, and SEQ ID NO: 169; SEQ ID NO: 97, SEQ ID NO: 112, SEQ ID NO: 124, SEQ ID NO: 142, SEQ ID NO: 163, and SEQ ID NO: 166; SEQ ID NO: 94, SEQ ID NO: 108, SEQ ID NO: 132, SEQ ID NO: 149, SEQ ID NO: 160, and SEQ ID NO: 177; SEQ ID NO: 93, SEQ ID NO: 109, SEQ ID NO: 131, SEQ ID NO: 148, SEQ ID NO: 158, and SEQ ID NO: 178; SEQ ID NO: 99, SEQ ID NO: 117, SEQ ID NO: 128, SEQ ID NO: 147, SEQ ID NO: 155, and SEQ ID NO: 174; SEQ ID NO: 99, SEQ ID NO: 117, SEQ ID NO: 128, SEQ ID NO: 150, SEQ ID NO: 159, and SEQ ID NO: 176; SEQ ID NO: 102, SEQ ID NO: 107, SEQ ID NO: 136, SEQ ID NO: 140, SEQ ID NO: 162, and SEQ ID NO: 170; SEQ ID NO: 102, SEQ ID NO: 114, SEQ ID NO: 136, SEQ ID NO: 140, SEQ ID NO: 162, and SEQ ID NO: 170; SEQ ID NO: 102, SEQ ID NO: 113, SEQ ID NO: 136, SEQ ID NO: 143, SEQ ID NO: 162, and SEQ ID NO: 170; SEQ ID NO: 91, SEQ ID NO: 119, SEQ ID NO: 126, SEQ ID NO: 146, SEQ ID NO: 154, and SEQ ID NO: 173; SEQ ID NO: 92, SEQ ID NO: 121, SEQ ID NO: 134, SEQ ID NO: 152, SEQ ID NO: 156, and SEQ ID NO: 167; or SEQ ID NO: 92, SEQ ID NO: 122, SEQ ID NO: 134, SEQ ID NO: 141, SEQ ID NO: 156, and SEQ ID NO: 168.

In some embodiments, the VH and VL of the anti-CD28 scFv comprise amino acid sequences at least 80%, 85%, 90%, 95%, or 100% identical to the amino acid sequences SEQ ID NO: 56 and SEQ ID NO: 90, SEQ ID NO: 57 and SEQ ID NO: 86, SEQ ID NO: 58 and SEQ ID NO: 83, SEQ ID NO: 59 and SEQ ID NO: 83, SEQ ID NO: 60 and SEQ ID NO: 82, SEQ ID NO: 60 and SEQ ID NO: 87, SEQ ID NO: 61 and SEQ ID NO: 81, SEQ ID NO: 62 and SEQ ID NO: 87, SEQ ID NO: 63 and SEQ ID NO: 85, SEQ ID NO: 64 and SEQ ID NO: 76, SEQ ID NO: 65 and SEQ ID NO: 75, SEQ ID NO: 66 and SEQ ID NO: 89, SEQ ID NO: 67 and SEQ ID NO: 88, SEQ ID NO: 68 and SEQ ID NO: 82, SEQ ID NO: 68 and SEQ ID NO: 87, SEQ ID NO: 69 and SEQ ID NO: 78, SEQ ID NO: 70 and SEQ ID NO: 78, SEQ ID NO: 71 and SEQ ID NO: 80, SEQ ID NO: 72 and SEQ ID NO: 84, SEQ ID NO: 73 and SEQ ID NO: 77, or SEQ ID NO: 74 and SEQ ID NO: 79, respectively.

In some embodiments, the VH and VL are fused via an amino acid linker. In some embodiments, the linker consists of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235). In some embodiments, the linker consists of GGGGSGGGGS (SEQ ID NO: 10). In some embodiments, the linker consists of GGGGSGGGGSGGGGS (SEQ ID NO: 11). In some embodiments, the linker consists of GKPGSGKPGSGKPGSGKPGS (SEQ ID NO: 14).

In some embodiments, the antigen-binding fragment of an antibody is an anti-CD28 single domain heavy chain variable region (sdVH) or an anti-CD28 single domain light chain variable region (sdVL). In some embodiments, the anti-CD28 sdVH comprises complementarity-determining regions CDR1, CDR2, and CDR3, wherein the CDR1, CDR2, and CDR3 comprise, respectively, the amino acid sequences SEQ ID NO: 183, SEQ ID NO: 187, and SEQ ID NO: 191; SEQ ID NO: 184, SEQ ID NO: 188, and SEQ ID NO: 192; or SEQ ID NO: 185, SEQ ID NO: 189, and SEQ ID NO: 193; or wherein the anti-CD28 sdVL comprises complementarity-determining regions CDR1, CDR2, and CDR3, wherein the CDR1, CDR2, and CDR3 comprise, respectively, the amino acid sequences SEQ ID NO: 186, SEQ ID NO: 190, and SEQ ID NO: 194. In some embodiments, the anti-CD28 sdVH comprises an amino acid sequence at least 80%, 85%, 90%, 95%, or 100% identical to the amino acid sequence SEQ ID NO: 179, SEQ ID NO: 180, or SEQ ID NO: 181, or wherein the anti-CD28 sdVL comprises an amino acid sequence at least 80%, 85%, 90%, 95%, or 100% identical to the amino acid sequence SEQ ID NO: 182.

In some embodiments, H comprises a 4-1BBL trimer. In some embodiments, the 4-1BBL trimer comprises three 4-1BBL monomers. In some embodiments, each 4-1BBL monomer is a soluble fragment of human 4-1BBL comprising amino acids X to 254 of SEQ ID NO: 15, wherein X is an integer from 50 to 99. In some embodiments, the soluble fragments of 4-1BBL each comprise amino acids 71 to 254 of SEQ ID NO: 15 (SEQ ID NO: 197). In some embodiments, the soluble fragments of 4-1BBL each comprise amino acids 58 to 254 of SEQ ID NO: 15 (SEQ ID NO: 196).

In some embodiments, the 4-1BBL monomers are fused via amino acid linkers which can the same or different. In some embodiments, each linker consists, independently, of 5 to 25 amino acids. In some embodiments, each linker consists, independently, of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235). In some embodiments, the linker consists of GGGGSGGGGS (SEQ ID NO: 10). In some embodiments, the linker consists of GGGGSGGGGSGGGGS (SEQ ID NO: 11). In some embodiments, the linker consists of GKPGSGKPGSGKPGSGKPGS (SEQ ID NO: 14). In some embodiments, H comprises the amino acid sequence SEQ ID NO: 198, SEQ ID NO: 199, SEQ ID NO: 200, or SEQ ID NO: 201.

In some embodiments, C comprises a heavy chain variable region (VH) and a light chain variable region (VL), wherein the VH of C comprises VH complementarity-determining regions VHCDR1, VHCDR2, and VHCDR3 and the VL of C comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; SEQ ID NO: 35, SEQ ID NO: 43, SEQ ID NO: 47, SEQ ID NO: 39, SEQ ID NO: 40, and SEQ ID NO: 49, or SEQ ID NO: 25, SEQ ID NO: 26, SEQ ID NO: 51, SEQ ID NO: 53, SEQ ID NO: 54, and SEQ ID NO: 55. In some embodiments, the VHCDR1, VHCDR2, VHCDR3, VLCDR1, VLCDR2, and VLCDR3 of C comprise, respectively, SEQ ID NO: 25, SEQ ID NO: 26, SEQ ID NO: 27, SEQ ID NO: 29, SEQ ID NO: 30, and SEQ ID NO: 31.

In some embodiments, the VH and VL of C comprise an amino acid sequence at least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; or SEQ ID NO: 46 and SEQ ID NO: 48, respectively. In some embodiments, the C comprises the VH and VL amino acid sequences SEQ ID NO: 24 and SEQ ID NO: 28, respectively.

In some embodiments, the VH and VL are fused via an amino acid linker. In some embodiments, the linker consists of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235). In some embodiments, the linker consists of GGGGSGGGGS (SEQ ID NO: 10). In some embodiments, the linker consists of GGGGSGGGGSGGGGS (SEQ ID NO: 11). In some embodiments, the linker consists of GKPGSGKPGSGKPGSGKPGS (SEQ ID NO: 14).

In some embodiments, J comprises SEQ ID NO: 7 or a functional fragment or variant thereof. In some embodiments, J comprises an alanine substitution at the amino acid position corresponding to amino acid Y102 of the mature wild-type human J chain (SEQ ID NO: 7). In some embodiments, J comprises the amino acid sequence SEQ ID NO: 8 (“J*”). In some embodiments, the modified J chain comprises the amino acid sequence SEQ ID NO: 231, SEQ ID NO: 232, SEQ ID NO: 233, or SEQ ID NO: 234. In some embodiments, the modified J chain comprises the amino acid sequence SEQ ID NO: 213 or SEQ ID NO: 214.

Also provided herein is a multimeric binding molecule comprising five, four, or two bivalent binding units and a modified J chain, wherein each binding unit comprises two antibody heavy chains, each comprising an IgM or IgA heavy chain constant region or multimerizing variant or fragment thereof, and wherein the modified J chain comprises a J chain or a functional fragment or variant thereof and a 4-1BBL trimer associated as a fusion protein, wherein the 4-1BBL trimer comprises three 4-1BBL monomers each comprising a soluble fragment of 4-1BBL.

In some embodiments, each 4-1BBL monomer is a soluble fragment of human 4-1BBL comprising amino acids X to 254 of SEQ ID NO: 15, wherein X is an integer from 50 to 99. In some embodiments, the soluble fragments of 4-1BBL each comprise amino acids 71 to 254 of SEQ ID NO: 15 (SEQ ID NO: 197). In some embodiments, the soluble fragments of 4-1BBL each comprise amino acids 58 to 254 of SEQ ID NO: 15 (SEQ ID NO: 196). In some embodiments, the 4-1BBL monomers are fused via amino acid linkers which can the same or different. In some embodiments, each linker consists, independently, of 5 to 25 amino acids. In some embodiments, each linker consists, independently, of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235). In some embodiments, the linker consists of GGGGSGGGGS (SEQ ID NO: 10). In some embodiments, the linker consists of GGGGSGGGGSGGGGS (SEQ ID NO: 11). In some embodiments, the linker consists of GKPGSGKPGSGKPGSGKPGS (SEQ ID NO: 14). In some embodiments, the 4-1BB trimer comprises the amino acid sequence SEQ ID NO: 198, SEQ ID NO: 199, SEQ ID NO: 200, or SEQ ID NO: 201.

In some embodiments, the 4-1BBL trimer is fused to the N terminus of the J chain or fragment or variant thereof or is fused to the C terminus of the J chain or fragment or variant thereof, or a copy of the 4-1BB trimer is fused to both the N terminus and the C terminus of the J chain or fragment or variant thereof. In some embodiments, the J chain or a functional fragment or variant thereof comprises the mature human J chain SEQ ID NO: 7 or a functional fragment or variant thereof. In some embodiments, the J chain comprises an alanine substitution at the amino acid position corresponding to amino acid Y102 of the mature wild-type human J chain (SEQ ID NO: 7). In some embodiments, the J chain comprises the amino acid sequence SEQ ID NO: 8 (“J*”). In some embodiments, the modified J chain comprises the amino acid sequence SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211.

In some embodiments, the multimeric binding molecule is a pentameric IgM antibody or IgM-like antibody comprising five bivalent binding units, wherein each binding unit comprises two IgM heavy chain constant regions or multimerizing fragments or variants thereof, each comprising an IgM Cμ4 domain and an IgM tailpiece (μtp) domain. In some embodiments, each IgM heavy chain constant region or multimerizing fragment or variant thereof further comprises a Cμ1 domain, a Cμ2 domain, a Cμ3 domain, or any combination thereof. In some embodiments, each IgM heavy chain constant region or multimerizing fragment or variant thereof is a human IgM constant region. In some embodiments, each IgM heavy chain constant region comprises the amino acid sequence SEQ ID NO: 1, SEQ ID NO: 2, or a multimerizing variant or fragment thereof. In some embodiments, each IgM heavy chain constant region is a variant human IgM constant region comprising one or more single amino acid substitutions, deletions, or insertions relative to SEQ ID NO: 1 or SEQ ID NO: 2, wherein the multimeric binding molecule has reduced CDC activity relative to a multimeric binding molecule where each IgM heavy chain constant region comprises the amino acid sequence SEQ ID NO: 1 or SEQ ID NO: 2. In some embodiments, each variant human IgM constant region comprises an amino acid substitution corresponding to position L310 of SEQ ID NO: 1 or SEQ ID NO: 2, an amino acid substitution corresponding to position P311 of SEQ ID NO: 1 or SEQ ID NO: 2, an amino acid substitution corresponding to position P313 of SEQ ID NO: 1 or SEQ ID NO: 2, an amino acid substitution corresponding to position K315 of SEQ ID NO: 1 or SEQ ID NO: 2, or any combination thereof. In some embodiments, each IgM heavy chain constant region is a variant human IgM constant region comprising one or more single amino acid substitutions, deletions, or insertions relative to SEQ ID NO: 1 or SEQ ID NO: 2, wherein the multimeric binding molecule exhibits increased serum half-life upon administration to a subject animal relative to a multimeric binding molecule in which each IgM heavy chain constant region comprises the amino acid sequence SEQ ID NO: 1 or SEQ ID NO: 2, which is administered in the same way to the same animal species. In some embodiments, each variant IgM heavy chain constant region comprises half-life altering amino acid substitutions at one or more amino acid positions corresponding to amino acid E345, S401, E402, or E403 of SEQ ID NO: 1 or SEQ ID NO: 2.

In some embodiments, the multimeric binding molecule is a dimeric or tetrameric IgA antibody or IgA-like antibody comprising two or four bivalent IgA or IgA-like binding units, respectively, wherein each binding unit comprises two IgA heavy chain constant regions or multimerizing fragments or variants thereof, each of which comprises a Cα3 domain and an IgA tailpiece (αtp) domain. In some embodiments, each IgA heavy chain constant region or multimerizing fragment or variant thereof further comprises a Cal domain, a Cα2 domain, an IgA hinge region, or any combination thereof. In some embodiments, each IgA heavy chain constant region or multimerizing fragment or variant thereof is a human IgA constant region.

In some embodiments, each target antigen-binding domain comprises a heavy chain variable region (VH) and a light chain variable region (VL), or a single-domain heavy chain variable region (sdVH). In some embodiments, each heavy chain comprises, from N-terminus to C-terminus, a sdVH fused to the heavy chain constant region or multimerizing fragment or variant thereof. In some embodiments, each heavy chain comprises, from N-terminus to C-terminus, a VH fused to the heavy chain constant region or multimerizing fragment or variant thereof. In some embodiments, each binding unit further comprises two antibody light chains, each comprising, from N-terminus to C-terminus, a VL fused to a light chain constant region. In some embodiments, at least three, four, five, six, seven, eight, nine, or ten of the target antigen-binding domains are identical. In some embodiments, the at least three, four, five, six, seven, eight, nine, or ten identical target antigen-binding domains specifically bind to a tumor-associated target antigen. In some embodiments, the tumor-associated target antigen is CD38, CD20, CD19, CD22, CD28, B cell maturation antigen (BCMA), CD123, PD-L1, tumor-associated calcium signal transducer 2 (TROP-2), mesothelin, Muc16, prostate-specific membrane antigen (PSMA), or six-transmembrane epithelial antigen of prostate-1·(STEAP-1). In some embodiments, the tumor-associated target antigen is CD20. In some embodiments, the tumor-associated target antigen is mesothelin (MSLN). In some embodiments, the tumor-associated target antigen is CD38. In some embodiments the tumor-associated target antigen is tumor-associated calcium signal transducer 2 (TROP-2). In some embodiments, the multimeric binding molecule conditionally activates T cells in the presence of the tumor-associated target antigen.

In some embodiments, the at least three, four, five, six, seven, eight, nine, or ten identical target antigen-binding domains specifically bind to a tumor-associated target antigen. In some embodiments, the target antigen is CD38, CD20, CD19, CD22, CD28, B cell maturation antigen (BCMA), CD123, PD-L1, tumor-associated calcium signal transducer 2 (TROP-2), mesothelin, Muc16, prostate-specific membrane antigen (PSMA), or six-transmembrane epithelial antigen of prostate-1·(STEAP-1). In some embodiments, the target antigen is CD20. In some embodiments, the tumor-associated target antigen is mesothelin (MSLN). In some embodiments, the tumor-associated target antigen is CD38. In some embodiments the tumor-associated target antigen is tumor-associated calcium signal transducer 2 (TROP-2).

In some embodiments, the multimeric binding molecule conditionally activates T cells in the presence of the target antigen. In some embodiments, the multimeric binding molecule activates T cells after 7 days in the presence of the target antigen. In some embodiments, the multimeric binding molecule activates T cells after 10 days in the presence of the target antigen. In some embodiments, the multimeric binding molecule activates T cells after 14 days in the presence of the target antigen. In some embodiments, the multimeric binding molecule directs T cell-mediated killing of cells expressing the target antigen after 7 days in the presence of the target antigen. In some embodiments, the multimeric binding molecule directs T cell-mediated killing of cells expressing the target antigen after 10 days in the presence of the target antigen. In some embodiments, the multimeric binding molecule directs T cell-mediated killing of cells expressing the target antigen after 14 days in the presence of the target antigen. In some embodiments, the T cells are CD4-positive T cells. In some embodiments, the T cells are CD8-positive T cells.

Also provided herein is a composition comprising a multimeric binding molecule as disclosed herein and a pharmaceutically acceptable carrier.

Also provided herein is a polynucleotide comprising a nucleic acid sequence that encodes a modified J chain of a multimeric binding molecule disclosed herein.

Also provided herein is a vector comprising a polynucleotide disclosed herein.

Also provided herein is a composition comprising a polynucleotide comprising a nucleic acid sequence that encodes a modified J chain of a multimeric binding molecule disclosed herein, and a polynucleotide encoding a heavy chain of the multimeric binding molecule disclosed herein. In some embodiments, the composition further comprises a polynucleotide encoding a light chain of a multimeric binding molecule described herein.

Also provided herein is a vector comprising a composition disclosed herein.

Also provided herein is a host cell comprising a composition disclosed herein, or a vector disclosed herein, wherein the host cell can express a multimeric binding molecule disclosed herein.

Also provided herein is a method of producing a multimeric binding molecule disclosed herein, comprising culturing the host cell disclosed herein, and recovering the multimeric binding molecule.

Also provided herein is a method of treating cancer, comprising administering to a subject in need of treatment a multimeric binding molecule disclosed herein.

Also provided herein is a multimeric binding molecule as disclosed herein for use in treating cancer.

Also provided herein is a use of a multimeric binding molecule disclosed herein in the preparation of a medicament for the treatment of cancer.

### BRIEF DESCRIPTION OF THE DRAWINGS/FIGURES

FIG. 1 shows a schematic of a co-stimulatory IgM T cell engager and a schematic of how the co-stimulatory IgM T cell engager would act in the immune synapse.

FIGS. 2A-2G show exemplary IgM pentamers with a modified human J chain fused to various moieties. FIG. 2A shows a modified human J chain fused to an anti-CD28 scFv on the N-terminus and an anti-CD3 scFv on the C-terminus. FIG. 2B shows a modified human J chain fused to an anti-CD28 scFv on the C-terminus and an anti-CD3 scFv on the N-terminus. FIG. 2C shows a modified human J chain fused to a 4-1BBL trimer on the N-terminus. FIG. 2D shows a modified human J chain fused to a 4-1BBL trimer on the C-terminus. FIG. 2E shows a modified human J chain fused to a 4-1BBL trimer on the C-terminus and an anti-CD3 scFv on the N-terminus. FIG. 2F shows a modified human J chain fused to a 4-1BBL trimer on the N-terminus and an anti-CD3 scFv on the C-terminus. FIG. 2G shows a modified human J chain fused to a 4-1BBL trimer on the N-terminus and a 4-1BBL trimer on the C-terminus.

FIG. 3 shows the amount of binding of IgM antibodies having a 4-1BBL-modified J chain in various formats at various concentrations to 4-1BB+ cells.

FIGS. 4A-4D show the binding over time of various concentrations of A5 (FIG. 4A), A6 (FIG. 4B), A9 (FIG. 4C), and D2 (FIG. 4D) as measured by biolayer interferometry (BLI).

FIGS. 5A-5B show the luminescence measured at each concentration for A5, A6, A8, and A9 with CD20 coating (FIG. 5A) or without CD20 coating (FIG. 5B). FIG. 5C shows the luminescence (in RLU) measured at the highest concentration tested for A5, A6, A8, A9, D1, D2 (with and without crosslinking), D3, and D4 (with and without crosslinking) in the presence of CD20 or without.

FIG. 6A shows the luminescence measured at each concentration for A1, A2, A3, A4, D1, and D2 with CD20+ (Ramos) cells. FIG. 6B shows the luminescence measured at each concentration for A5, A6, A8, A9, D1, and D2 with CD20+ (Ramos) cells. FIG. 6C shows the luminescence (RLU) measured at the highest concentration tested for A1, A2, A3, A4, A5, A6, A8, A9, D1, and D2 in the presence of CD20+ cells (Ramos) or CD20-cells (OPM-2).

FIGS. 7A-7B show the luminescence measured at each concentration for A5, A6, A7, A8, A9, A10, A11, A12, D1, and D2 with low levels of CD20 (CA46) cells (FIG. 7A) and high levels of CD20 (DOHH2) cells (FIG. 7B). FIG. 7C shows the luminescence measured at the highest concentration tested for A5, A6, A8, A9, A10, A11, A12, and D1 in the presence of cells expressing high levels of CD20 cells (DOHH2), cells expressing low levels of CD20 (CA46), or CD20-cells (OPM-2).

FIG. 8 shows the amount of binding of B1, B2, B3, B4, D5, or D6 to 4-1BB as determined by ELISA.

FIGS. 9A-9B show the luminescence measured at each concentration for B1, B2, B3, B4, D5, or D6 with mesothelin-negative cells (FIG. 9A) and mesothelin-positive cells (FIG. 9B). FIG. 9C shows the luminescence measured at the highest concentration tested for B1, B2, B3, B4, D5, and D6 in the presence of mesothelin+ cells or mesothelin-cells.

FIGS. 10A-10D show the activation of CD4+ T cells (FIGS. 10A-10B) or CD8+ T cells (FIGS. 10C-10D) by C4, C5, C6, and D7 at various concentrations after 24 hours (FIGS. 10A, 10C) and 48 hours (FIGS. 10B, 10D).

FIGS. 11A-11B show the amount of T cell dependent cytotoxicity (TDCC) on Ramos cells after 24 hours (FIG. 11A) or 48 hours (FIG. 11B) of co-culture with PBMCs and C4, C5, C6, and D7.

FIGS. 12A-12J show IL-2 (FIGS. 12A-12B), IL-6 (FIGS. 12C-12D), IL-10 (FIGS. 12E-12F), TNFα (FIGS. 12G-12H), and IFNγ (FIGS. 12I-12J) concentrations in supernatants from Ramos and PBMC cells treated with various concentrations of C4. C5, C6, and D7 after 24 hours (FIGS. 12A, 12C, 12E, 12G, 12I) and 48 hours (FIGS. 12B, 12D, 12F, 12H, 12J).

FIGS. 13A-13D show the activation of CD4+ T cells by D18 or E1 in a co-culture of PBMC cells and H929_Luc/GFP (FIG. 13A), OPM2_Luc/GFP (FIG. 13B), MMIS_Luc/GFP (FIG. 13C), and RPMI8226_Luc/GFP (FIG. 13D) cells after 48 hours.

FIGS. 14A-14D show the activation of CD8+ T cells by D18 or E1 in a co-culture of PBMC cells and H929_Luc/GFP (FIG. 14A), OPM2_Luc/GFP (FIG. 14B), MMIS_Luc/GFP (FIG. 14C), and RPMI8226_Luc/GFP (FIG. 14D) cells after 48 hours.

FIGS. 15A-15D show the amount of TDCC on H929_Luc/GFP (FIG. 15A), OPM2_Luc/GFP (FIG. 15B), MM1S_Luc/GFP (FIG. 15C), and RPMI8226_Luc/GFP (FIG. 15D) cells after 72 hours of co-culture with PBMC cells and D18 or E1.

FIGS. 16A-16D show IL-2 concentrations in supernatants from a co-culture of PBMC cells and H929_Luc/GFP (FIG. 16A), OPM2_Luc/GFP (FIG. 16B), MMIS_Luc/GFP (FIG. 16C), and RPMI8226_Luc/GFP (FIG. 16D) cells after 48 hours treated with D18 or E1.

FIGS. 17A-17D show IL-4 concentrations in supernatants from a co-culture of PBMC cells and H929_Luc/GFP (FIG. 17A), OPM2_Luc/GFP (FIG. 17B), MMIS_Luc/GFP (FIG. 17C), and RPMI8226_Luc/GFP (FIG. 17D) cells after 48 hours treated with D18 or E1.

FIGS. 18A-18D show IL-6 concentrations in supernatants from a co-culture of PBMC cells and H929_Luc/GFP (FIG. 18A), OPM2_Luc/GFP (FIG. 18B), MMIS_Luc/GFP (FIG. 18C), and RPMI8226_Luc/GFP (FIG. 18D) cells after 48 hours treated with D18 or E1.

FIGS. 19A-19D show IL-10 concentrations in supernatants from a co-culture of PBMC cells and H929_Luc/GFP (FIG. 19A), OPM2_Luc/GFP (FIG. 19B), MMIS_Luc/GFP (FIG. 19C), and RPMI8226_Luc/GFP (FIG. 19D) cells after 48 hours treated with D18 or E1.

FIGS. 20A-20D show TNFα concentrations in supernatants from a co-culture of PBMC cells and H929_Luc/GFP (FIG. 20A), OPM2_Luc/GFP (FIG. 20B), MM1S_Luc/GFP (FIG. 20C), and RPMI8226_Luc/GFP (FIG. 20D) cells after 48 hours treated with D18 or E1.

FIGS. 21A-21D show IFN-gamma concentrations in supernatants from a co-culture of PBMC cells and H929_Luc/GFP (FIG. 21A), OPM2_Luc/GFP (FIG. 21B), MMIS_Luc/GFP (FIG. 21C), and RPMI8226_Luc/GFP (FIG. 21D) cells after 48 hours treated with D18 or E1.

FIGS. 22A-22B show the activation of CD4+ T cells (FIG. 22A) or CD8+ T cells (FIG. 22B) by C6, C3, or D7 in a co-culture of PBMC cells and Raji-luc-GFP cells after 96 hours. FIG. 22C shows the amount of TDCC on Ramos cells after 72 hours of co-culture with PBMC cells and C6, C3, or D7.

FIGS. 23A-23B show IL-2 (FIG. 23A) or IFNγ (FIG. 23B) concentrations in supernatants from a co-culture of PBMC cells and Raji-luc-GFP after 96 hours treated with C6, C3, or D7.

FIGS. 24A-24D show tumor volume over time for human PBMC-engrafted NSG-MHC I/II DKO mice treated with vehicle, D7 dosed at 10 mg/kg, 1 mg/kg, or 0.1 mg/kg; C7 dosed at 10 mg/kg, 1 mg/kg, or 0.1 mg/kg; and C3 dosed at 10 mg/kg (FIG. 24A), vehicle, D7 dosed at 10 mg/kg, C7 dosed at 10 mg/kg, or C3 dosed at 10 mg/kg (FIG. 24B), vehicle, D7 dosed at 1 mg/kg, C7 dosed at 1 mg/kg, or C3 dosed at 10 mg/kg (FIG. 24C), and vehicle, D7 dosed at 0.1 mg/kg, C7 dosed at 0.1 mg/kg, or C3 dosed at 10 mg/kg (FIG. 24D).

FIGS. 25A-25J show IL-2 (FIG. 25A), IL-10 (FIG. 25B), IFN-γ (FIG. 25C), TNF-α (FIG. 25D), IL-4 (FIG. 25E), IL-6 (FIG. 25F), IL-8 (FIG. 25G), IL-1β (FIG. 25H), IL-12p70 (FIG. 25I), and IL-13 (FIG. 25J) concentrations in serum collected from tumor-bearing human PBMC-engrafted NSG-MHC I/II DKO mice treated with vehicle, D7 dosed at 10 mg/kg, 1 mg/kg, or 0.1 mg/kg; C7 dosed at 10 mg/kg, 1 mg/kg, or 0.1 mg/kg; and C3 dosed at 10 mg/kg as described in Example 19.

FIGS. 26A-26B show total infiltrated CD4+ T cells (FIG. 26A) or CD8+ T cells (FIG. 26B) normalized to tumor volume (mm³) detected in tumors harvested from human PBMC-engrafted NSG-MHC I/II DKO mice treated with vehicle, D7 dosed at 0.1 mg/kg; C7 dosed at 0.1 mg/kg.

FIGS. 27A-27C show T cell dependent cellular cytotoxicity mediated by vehicle or by incubation with antibodies D8, D9, D10, D7, or C6, each at 10 nM concentration, in a PBMC (effector)/Ramos cell (target) coculture assay at effector to target ratios 3:1 (FIG. 27A), 1:1 (FIG. 27B) or 1:3 (FIG. 27C) at 3-day, 7-day, 10-day, and 14-day timepoints.

FIGS. 28A-28C show CD4+ T cell levels mediated by vehicle or by incubation with antibodies D8, D9, D10, D7, or C6, each at 10 nM concentration, in a PBMC (effector)/Ramos cell (target) coculture assay at effector to target ratios 3:1 (FIG. 28A), 1:1 (FIG. 28B) or 1:3 (FIG. 28C) at 3-day, 7-day, 10-day, and 14-day timepoints.

FIGS. 29A-29C show CD8+ T cell levels mediated by vehicle or by incubation with antibodies D8, D9, D10, D7, or C6, each at 10 nM concentration, in a PBMC (effector)/Ramos cell (target) coculture assay at effector to target ratios 3:1 (FIG. 29A), 1:1 (FIG. 29B) or 1:3 (FIG. 29C) at 3-day, 7-day, 10-day, and 14-day timepoints.

FIGS. 30A-30F show IL-2 (FIG. 30A), IL-4 (FIG. 30B), IL-6 (FIG. 30C), IL-10 (FIG. 30D), TNFα (“TNFa,” FIG. 30E), and interferon-gamma (“IFN-gamma,” FIG. 30F) levels mediated by vehicle or by incubation with antibodies D8, D9, D10, D7, or C6, each at 10 nM concentration, in a PBMC (effector)/Ramos cell (target) coculture assay at effector to target ratios 3:1, 1:1, or 1:3, at the 72-hour (3-day) timepoint.

FIG. 31 is a survival plot showing survival probabilities for tumor-bearing human PBMC-engrafted NSG-MHC I/II DKO mice treated with: vehicle, D7 dosed at 10 mg/kg and 1 mg/kg; C7 dosed at 10 mg/kg and 1 mg/kg; and C3 dosed at 10 mg/kg as described in Example 21.

FIGS. 32A-C show tumor volume over time for human PBMC-engrafted NSG-MHC I/II DKO mice treated with vehicle, control (dosed at 10 mg/kg), C7 dosed at 10 mg/kg and 1 mg/kg, and D7 dosed at 10 mg/kg and 1 mg/kg as described in Example 22. More particularly, tumor volumes over time for each group are provided in FIG. 32A (vehicle, control dosed at 10 mg/kg), C7 dosed at 10 mg/kg and 1 mg/kg, and D7 dosed at 10 mg/kg and 1 mg/kg. Tumor volumes over time for the 10 mg/kg groups (control, C7 and D7) are shown in FIG. 32B; tumor volumes for the 1 mg/kg C7 and D7 groups, and for the 10 mg/kg control group are shown in FIG. 32C.

FIG. 33 is a plot showing survival probabilities for tumor-bearing human PBMC-engrafted NSG-MHC I/II DKO mice treated with vehicle, control, C7 dosed at 10 mg/kg and at 1 mg/kg, and D7 dosed at 10 mg/kg and 1 mg/kg, over time as evaluated in a Ramos human B lymphocyte cancer xenograft model as described in Example 22.

FIGS. 34A-34E show IL-2 (FIG. 34A), IL-4 (FIG. 34B), IL-10 (FIG. 34C), IFN-γ (FIG. 34D) and TNF-α (FIG. 34E) concentrations in serum collected from tumor-bearing human PBMC-engrafted NSG-MHC I/II DKO mice treated with vehicle, control, C7 dosed at 10 mg/kg and 1 mg/kg and D7 dosed at 10 mg/kg and 1 mg/kg as described in Example 22.

FIG. 35 is a graph showing tumor volume over time for human PBMC-engrafted NSG-MHC I/II DKO mice treated with vehicle, control antibody, C7 and D7 dosed at 10 mg/kg as described in Example 23.

FIGS. 36A-36E show absolute cell counts of CD4 T cell subsets in peripheral blood measured at the end of the study and normalized to blood volume for the various treatment groups (Vehicle, Control, C7, and D7) described in Example 23.

FIGS. 37A-37E show absolute cell counts of CD8 T cell subsets in peripheral blood measured at the end of the study and normalized to blood volume for the various treatment groups (Vehicle, Control, C7, and D7) described in Example 23.

FIG. 38 shows T cell-dependent cytotoxicity (TDCC) over time as measured by Real Time Cell Analysis (RTCA) for anti-mesothelin antibody constructs M2, M3, and M1, as compared to no antibody and isotype control D15 (each at 5 nM) as tested with 50,000 peripheral blood mononuclear cells (PBMC) and 15,000 OVCAR3 ovarian cancer cell line cells.

FIGS. 39A-39F show IL-2 (39A), IL-4 (39B), IL-6 (39C), IL-10 (39D), IFN-γ (39E), and TNFα (39F) expression at hour 69 of the RTCA assay shown in FIG. 38.

FIGS. 40A-40D show T cell counts and T cell activation at hour 96 of the RTCA assay shown in FIG. 38. FIGS. 40A and 40B show CD4+ T cell counts and T cell activation, respectively, and FIGS. 40C and 40D show CD8+ T cell counts and T cell activation, respectively.

FIG. 41 shows T cell-dependent cytotoxicity over time as measured by Real Time Cell Analysis (RTCA) for anti-TROP-2 antibody constructs T2, T3, and T1, as compared to no antibody and isotype control D15 (each at 5 nM) as tested with 50,000 peripheral blood mononuclear cells (PBMC) and 15,000 OVCAR3 ovarian cancer cell line cells.

FIGS. 42A-42F show IL-2 (42A), IL-4 (42B), IL-6 (42C), IL-10 (42D), IFN-γ (42E), and TNFα (42F) expression at hour 69 of the RTCA assay shown in FIG. 41.

FIGS. 43A-43D show T cell counts and T cell activation at hour 96 of the RTCA assay shown in FIG. 41. FIGS. 43A and 43B show CD4+ T cell counts and T cell activation, respectively, and FIGS. 43C and 43D show CD8+ T cell counts and T cell activation, respectively.

FIG. 44 is a graph of percent of cytotoxicity versus antibody concentration as measured in a TDCC assay in which human PBMCs as effector cells (E) and TROP2 transduced HEK293 cells as target cells (T) were incubated with serial dilutions of T1 or T3 as described in Example 24.

FIG. 45 is a graph of percent cell viability versus antibody concentration as measured in a TDCC assay in which human PBMCs as effector cells (E) and A431-Luc cells as target cells (T) were incubated with serial dilutions of antibodies T3, T1, or D16 as determined by a luminescence-based viability assay as described in Example 24.

FIG. 46 is a graph of percent cell viability versus antibody concentration as measured in a TDCC assay in which human PBMCs as effector cells (E) and SKOV-3 cells as target cells (T) were incubated with serial dilutions of antibodies T3, T1, or D16 as determined by a luminescence-based viability assay as described in Example 24.

FIG. 47 is a graph of percent target cell killing (i.e., antibody-mediated redirected T cell killing of OVCAR3-Luc cells) versus antibody concentration for exemplary antibodies: D16, T3, T1, D17, M3 and M1, as determined in a TDCC assay and measured using a luciferase assay as described in Example 24.

FIGS. 48A-F show cytokine release profiles mediated by test articles D16, T3, T1, D17, M3 and M1 for cytokines IL-2 (FIG. 48A), IL-4 (FIG. 48B), IL-6 (FIG. 48C), IL-10 (FIG. 48D), TNFα (FIG. 48E), and IFN-γ (FIG. 48F) at hour 72 of the assay as described in Example 24.

FIG. 49 is a graph of percent target cell killing (i.e., antibody-mediated redirected T cell killing of RERF-LC-KJ/CMV-Luc cells) versus antibody concentration for exemplary antibodies: D16, T3, and T1, as determined in a TDCC assay and measured using a luciferase assay as described in Example 24.

FIGS. 50A-F show cytokine release profiles mediated by test articles D16, T3 and T1 for cytokines IL-2 (FIG. 50A), IL-4 (FIG. 50B), IL-6 (FIG. 50C), IL-10 (FIG. 50D), TNFα (FIG. 50E), and IFN-γ (FIG. 50F) at hour 72 of the assay as described in Example 24.

FIG. 51 is a plot showing tumor-infiltrating lymphocyte (TIL) data for CD4+ and CD8+TILs (cells/gram of tumor) that had invaded tumor tissue as measured in a Ramos human B lymphocyte cancer xenograft model study for indicated treatment groups.

FIGS. 52A-C are plots of percent cell viability versus antibody concentration (log, μg/mL) from a TDCC assay as described in Example 25 in which the cytotoxicity of exemplary antibodies with or without CD28 co-stimulation, T1 (anti-TROP-2 x anti-CD3 x anti-CD28 IgM antibody, half-shaded diamonds) and T3 (anti-TROP-2 x anti-CD3 IgM, filled circles), respectively, was evaluated on high, medium, and low TROP-2 expressing cell lines (TROP-2 transduced HEK293T cells with TROP-2 high expression (FIG. 52A), A431 melanoma cells with TROP-2 high expression (FIG. 52B), and SKOV-3 ovarian cancer cells with TROP-2 medium/low expression (FIG. 52C)).

FIGS. 53A-53C are graphs of tumor volume (mm³) over time (days) for different antibody treatment groups (T1: TROP-2×CD3 x CD28 IgM at doses of 0.3 mg/kg, 1 mg/kg, and 3 mg/kg; T3: TROP-2×CD3 IgM at doses of 0.3 mg/kg, 1 mg/kg, and 3 mg/kg) and vehicle, evaluated in a humanized HEK293/TROP-2 xenograft model as described in Example 26. FIG. 53A provides data for the 0.3 mg/kg treatment groups;

FIG. 53B provides data for the 1 mg/kg treatment groups, and FIG. 53C provides data for the 3 mg/kg treatment groups

FIGS. 54A-D are bar graphs showing serum concentrations of IFN-γ (FIG. 54A), IL-2 (FIG. 54B), TNF-α (FIG. 54C), and IL-10 (FIG. 54D), respectively, for antibody treatment groups (T1: TROP-2×CD3 x CD28 IgM at doses of 0.3 mg/kg, 1 mg/kg, and 3 mg/kg; T3: TROP-2×CD3 IgM at doses of 0.3 mg/kg, 1 mg/kg, and 3 mg/kg) and vehicle, as evaluated using a humanized HEK293/TROP-2 xenograft model as described in Example 26.

FIG. 55 is a bar graph showing absolute counts of anti-apoptotic T-cells, CD8+Bcl_xL T cells, in mouse peripheral blood samples stained with an antibody cocktail containing variability dye and analyzed by flow cytometry. The blood samples were taken from different antibody treatment groups (T1: TROP-2×CD3 x CD28 IgM at doses of 0.3 mg/kg, 1 mg/kg, and 3 mg/kg; T3: TROP-2×CD3 IgM at doses of 0.3 mg/kg, 1 mg/kg, and 3 mg/kg) and vehicle, evaluated in a humanized HEK293/TROP-2 xenograft model as described in Example 26.

FIG. 56 illustrates the results of a TDCC assay in which the T cell dependent cytotoxicity activity of exemplary IgM antibodies, both with and without costimulatory activity, M1 (anti-mesothelin (MSLN) x anti-CD3 x anti-CD28 IgM antibody) and M3 (anti-mesothelin (MSLN) x anti-CD3 IgM antibody), respectively, on the gastric cancer cell line, MKN45, was assessed by luminescence, as described in Example 27.

FIGS. 57A, 57B are graphs of tumor volume (mm³) over time (days post MKN45 cell implant) for different antibody treatment groups: M1: anti-mesothelin (MSLN) x anti-CD3 x anti-CD28 IgM antibody, at doses of 3 mg/kg, and 10 mg/kg (n=7); M3: anti-mesothelin (MSLN) x anti-CD3 IgM antibody at doses of 3 mg/kg and 10 mg/kg (n=7), and vehicle (n=7), evaluated in a humanized MKN45 xenograft model as described in Example 28. FIG. 57A provides data for the 3 mg/kg treatment groups; FIG. 57B provides data for the 10 mg/kg treatment groups.

FIGS. 58A-D are bar graphs showing serum concentrations of IFN-γ (FIG. 58A), IL-2 (FIG. 58B), TNF-α (FIG. 58C), and IL-10 (FIG. 58D), respectively, for antibody treatment groups: M1: anti-mesothelin (MSLN) x anti-CD3 x anti-CD28 IgM antibody, at doses of 3 mg/kg, and 10 mg/kg (n=7); M3: anti-mesothelin (MSLN) x anti-CD3 IgM antibody at doses of 3 mg/kg and 10 mg/kg (n=7), and vehicle (n=7), evaluated in a humanized MKN45 xenograft model as described in Example 28.

FIGS. 59A-C are bar graphs showing the ability of various combinations of anti-CD28 and/or anti CD3 IgM or IgG4 antibodies to activate T cells and induce cytokine release in a plate-based assay as described in Example 29. The results of one of two exemplary PMBC donors (PBMC Donor 1) is shown. The various antibody and antibody combinations are detailed in Table 27. FIG. 59A shows CD4+ T cell activation. FIG. 59B shows CD8+ T cell activation. FIG. 59C shows IL-2 expression.

FIG. 60A-B are bar graphs showing absolute intra-tumoral CD4+ T cell counts (FIG. 60A) and absolute intra-tumoral CD8+ T cell counts (FIG. 60B) at day 43 for antibody treatment groups: M1: anti-mesothelin (MSLN) x anti-CD3 x anti-CD28 IgM antibody, at doses of 3 mg/kg, and 10 mg/kg (n=7); M3: anti-mesothelin (MSLN) x anti-CD3 IgM antibody at doses of 3 mg/kg and 10 mg/kg (n=7), and vehicle (n=7), evaluated in a humanized MKN45 xenograft model as described in Example 28.

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein means specific disclosure of each of the two specified features or components with or without the other. Thus, the term “and/or” as used in a phrase such as “A and/or B” herein includes “A and B,” “A or B,” “A” (alone), and “B” (alone). Likewise, the term “and/or” as used in a phrase such as “A, B, and/or C” encompasses each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, P-S(ed.), 2nd ed., 2002, CRC Press; The Dictionary of Cell and Molecular Biology, Lacki, J. M. (ed.) 5th ed., 2013, Academic Press; and the Oxford Dictionary of Biochemistry and Molecular Biology, Cammack, R., et al., (eds.) 2d ed., 2006, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” encompasses a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of any of these terms. The term “polypeptide” also refers to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety attached to the protein via an oxygen-containing or a nitrogen-containing side chain of an amino acid, e.g., a serine or an asparagine. Asparagine (N)-linked glycans are described in more detail elsewhere in this disclosure.

An “isolated” polypeptide or a fragment, variant, or derivative thereof is a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. As used herein a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is a multimeric binding molecule, e.g., an antibody, e.g., an IgM antibody, an IgM-like antibody, an IgA antibody, or an IgA-like antibody as described in detail herein that retains antigen-specific binding. In certain embodiments a “binding molecule” comprises a multimeric binding molecule, e.g., an IgM antibody, an IgM-like antibody, an IgA antibody, or an IgA-like antibody as described in detail herein.

As used herein, the terms “binding domain” or “antigen-binding domain” (can be used interchangeably) refer to a region of a binding molecule, e.g., an antibody, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is a “binding domain.” Other antigen-binding domains include, without limitation, a single domain heavy chain variable region (sdVH) of an antibody derived from, e.g., a camelid species or a shark species, and single domain light chain variable regions (sdVL). Single-domain antigen-binding domains typically include three complementarity-determining regions (CDRs). Antigen-binding domains can also be engineered, e.g., six immunoglobulin complementarity determining regions (CDRs) expressed in a fibronectin scaffold. A “binding molecule,” e.g., an “antibody” as described herein can include one, two, three, four, five, six, seven, eight, nine, ten, eleven, twelve, or more “antigen-binding domains.” An antigen-binding domain that specifically binds to a target antigen, e.g., a tumor-associated target antigen, is referred to herein as a “target antigen-binding domain.”

The term “antibody” includes at least the variable domain of a heavy chain (e.g., an sdVH from a camelid species), at least the variable domain of a light chain (e.g., an sdVL), or at least the variable domains of a heavy chain and a light chain. Basic immunoglobulin structures in vertebrate systems are relatively well understood. See, e.g., Greenfield, E. A. (ed.), Antibodies: A Laboratory Manual, Second Edition, Cold Spring Harbor Laboratory Press, 2014). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody or IgA-like antibody that can include four or eight complete heavy chains and four or eight complete light chains and a J chain and/or a secretory component, or an IgM antibody or IgM-like antibody, that can include ten complete heavy chains and ten complete light chains and a J chain or functional fragment or variant thereof.

The term “immunoglobulin” comprises various broad classes of polypeptides that can be distinguished biochemically. Immunoglobulin heavy chains are classified as gamma, mu, alpha, delta, or epsilon, (γ, μ, α, δ, ε) with some subclasses among them (e.g., γ1-γ4 or α1-α2). It is the nature of this chain that determines the “isotype” of an antibody as IgG, IgM, IgA IgD, or IgE, respectively. The immunoglobulin subclasses (subtypes) e.g., IgG1, IgG2, IgG3, IgG4, IgA1, IgA2, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, which corresponds to a standard immunoglobulin structure that includes at least two heavy chains and can further include two light chains. In certain embodiments, e.g., where the binding molecule is a bivalent IgG antibody or antigen-binding fragment thereof, the terms “binding molecule” and “binding unit” are equivalent. Such binding molecules are also referred to herein as “monomeric.” In other embodiments, e.g., where the binding molecule is a “multimeric binding molecule,” e.g., a dimeric or tetrameric IgA antibody or a pentameric IgM antibody, a dimeric, tetrameric, or pentameric IgA- or IgM-like antibody, or a dimeric, tetrameric, or pentameric IgA- or IgM-derived binding molecule, the binding molecule comprises two or more “binding units.” Two in the case of an IgA dimer, four in the case of an IgA tetramer, or five in the case of an IgM pentamer, respectively. A binding unit need not include full-length antibody heavy and light chains, but will typically be bivalent, i.e., will include two “antigen-binding domains,” e.g., “target antigen-binding domains,” as defined above. As used herein, certain binding molecules provided in this disclosure are “pentameric” and include five bivalent binding units that include IgM constant regions or multimerizing fragments or variants thereof and a J chain or functional fragment or variant thereof. A binding molecule, e.g., an antibody, comprising two or more, e.g., two, four, or five binding units, is referred to herein as a “multimeric binding molecule.”

The term “J chain” as used herein refers to the J chain of IgM or IgA antibodies of any animal species, any functional fragment thereof, derivative thereof, and/or variant thereof, including a mature human J chain, the amino acid sequence of which is presented as SEQ ID NO: 7. Various J chain variants and modified J chain derivatives are available, e.g., in U.S. Pat. No. 10,899,835. As persons of ordinary skill in the art will recognize, “a functional fragment” or “a functional variant” includes those fragments and variants that can associate with IgM heavy chain constant regions to form a pentameric IgM antibody.

The term “modified J chain” is used herein to refer to a derivative of a J chain polypeptide comprising a heterologous moiety, e.g., a heterologous polypeptide, e.g., an extraneous antigen-binding domain or functional domain (e.g., a ligand that binds to an effector cell, e.g., a T cell receptor or an immunostimulatory molecule) introduced into or attached to the J chain sequence. The introduction can be achieved by any means, including direct or indirect fusion of the heterologous polypeptide or other moiety or by attachment through a peptide or chemical linker. The term “modified human J chain” encompasses, without limitation, a native sequence human J chain comprising the amino acid sequence of SEQ ID NO: 7 or functional fragment thereof, or functional variant thereof, modified by the introduction of a heterologous moiety, e.g., a heterologous polypeptide, e.g., an extraneous binding domain. In certain embodiments, the heterologous moiety does not interfere with efficient polymerization of IgM into a pentamer or IgA into a multimer, e.g., a dimer or tetramer, and binding of such polymers to a target. Exemplary modified J chains can be found, e.g., in U.S. Pat. Nos. 9,951,134, 10,400,038, and 10,618,978, in U.S. Patent Application Publication No. US-2019-0185570, and in PCT Publication No. WO2021030688A1, each of which is incorporated herein by reference in its entirety.

The terms “valency,” “bivalent,” “multivalent” and grammatical equivalents, refer to the number of binding domains, e.g., antigen-binding domains, e.g., target antigen-binding domains, in a given antibody or in a given binding unit. As such, the terms “bivalent,” “tetravalent,” and “hexavalent” in reference to a given antibody, denote the presence of two antigen-binding domains, four antigen-binding domains, and six antigen-binding domains, respectively. A typical pentameric IgM antibody, where each binding unit is bivalent, has 10 or more valencies, e.g., target antigen-binding domains. A bivalent or multivalent antibody can be monospecific, i.e., all of the antigen-binding domains are the same, or can be bispecific or multispecific, e.g., where two or more antigen-binding domains are different, e.g., bind to different epitopes on the same antigen, bind to entirely different antigens, and/or bind to an effector cell, e.g., a T cell.

The term “epitope” includes any molecular determinant capable of specific binding to an antigen-binding domain of a binding molecule, e.g., an antibody. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., a multimeric binding molecule as provided herein. A target can be, e.g., a polypeptide, a nucleic acid, a carbohydrate, a lipid, or other molecule. Moreover, a “target” can, for example, be a cell, an organ, or an organism that comprises an epitope that can be bound by a binding molecule, e.g., a multimeric binding molecule as provided herein. As used herein, a “target antigen” is a target molecule, e.g., a polypeptide, a nucleic acid, a carbohydrate, a lipid, or other molecule that can be bound by a multimeric binding molecule as provided herein. In certain embodiments, a target antigen can appear on the surface of a cell, e.g., a tumor cell. A “tumor-specific antigen” as used herein is a protein or other cell surface target antigen that is unique to tumor cells, at least at later stages of development of the organism. As used herein, a “tumor-associated target antigen” is a protein or other cell surface target antigen that is not necessarily unique to tumor cells but is typically expressed much more abundantly and/or at higher density on tumor cells than on normal, healthy cells.

Both the light and heavy chain of a binding molecule, e.g., a multimeric binding molecule as provided herein, are divided into regions of structural and functional homology. The terms “constant” and “variable” are used functionally. In a standard antibody, the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding domains or amino-terminus of the antibody. The N-terminal portion is a variable region and at the C-terminal portion is a constant region; the CH3 (or CH4-μtp, e.g., in the case of IgM) and CL domains comprise the carboxy-terminus of the heavy and light chain, respectively.

A “full length IgM antibody heavy chain” is a polypeptide that includes, in N-terminal to C terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a u tailpiece.

As used herein, “IgM-like antibody” refers to a binding molecule that includes at least a multimerizing fragment or variant of an IgM heavy chain constant region that retains the ability to form hexamers or pentamers, e.g., in association with a J chain. An IgM-like antibody typically includes at least the Cμ4 and IgM tailpiece (μtp) domains of an IgM constant region but can include heavy chain constant region domains from other antibody isotypes, e.g., IgG, from the same species or from a different species. An IgM-like antibody can likewise be an antibody fragment in which one or more constant region domains are deleted, as long as the IgM-like antibody is capable of multimerizing into a hexamer and/or a pentamer. Thus, an IgM-like antibody can be, e.g., a hybrid IgM/IgG antibody or can be a “multimerizing fragment” of an IgM antibody.

A “full length IgA antibody heavy chain” is a polypeptide that includes, in N-terminal to C terminal direction, an antibody heavy chain variable domain (VH), an antibody constant heavy chain constant domain 1 (CA1 or Cal), an antibody heavy chain constant domain 2 (CA2 or Cα2), and an antibody heavy chain constant domain 3 (CA3 or Cα3) that can include an a tailpiece.

As used herein, “IgA-like antibody” refers to a binding molecule that includes at least a multimerizing fragment or variant of an IgA heavy chain constant region that retains the ability to form dimers or tetramers, e.g., in association with a J chain. An IgA-like antibody typically includes at least the Cα3 and IgA tailpiece (αtp) domains of an IgA constant region but can include heavy chain constant region domains from other antibody isotypes, e.g., IgG, from the same species or from a different species. An IgA-like antibody can likewise be an antibody fragment in which one or more constant region domains are deleted, as long as the IgA-like antibody is capable of multimerizing into a dimer and/or a tetramer. Thus, an IgA-like antibody can be, e.g., a hybrid IgA/IgG antibody or can be a “multimerizing fragment” of an IgA antibody.

As indicated above, variable region(s) allow a binding molecule, e.g., a multimeric binding molecule as provided herein, to selectively recognize and specifically bind epitopes on target antigens. For example, the VL domain and VH domain, an sdVH domain, an sdVL domain, or a subset of the complementarity determining regions (CDRs), of an antibody combine to form the antigen-binding domain. More specifically, an antigen-binding domain can be defined by the six CDRs on the VH and VL chains, or the three CDRs on an sdVH or sdVL chain, respectively. Certain antibodies form larger structures. For example, IgA can form a molecule that includes two or four H2L2 binding units and a J chain covalently connected via disulfide bonds, which can be further associated with a secretory component, and IgM can form a pentameric molecule that includes five H2L2 binding units and a J chain covalently connected via disulfide bonds.

The six (or in the case of single domain antibodies, three) “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the target antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein includes all such meanings unless explicitly stated to the contrary. A specific example is the use of the term “complementarity determining region” (“CDR”) to describe the non-contiguous antigen combining sites found within the variable region of both heavy and light chain polypeptides. These CDR regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Other overlapping CDR definitions can be found, e.g., in A1-Lazikani B. et al., J. Mol. Biol. 273:927-948 (1997); MacCallum et al., J. Mol. Biol. 262:732-745 (1996); Abhinandan and Martin, Mol. Immunol. 45:3832-3839 (2008); Lefranc M. P. et al., Dev. Comp. Immunol. 27:55-77 (2003); and Honegger and Plückthun, J. Mol. Biol. 309:657-670 (2001), which are incorporated herein by reference in their entireties. Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res. 36: W503-508, 2008). Application of any particular definition (or other definitions known to those of ordinary skill in the art) to refer to a CDR of an antibody or variant thereof is intended to be within the scope of the term as defined and used herein, unless otherwise indicated. The appropriate amino acids which encompass the “Kabat” and “Chothia” CDRs as defined by the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody. Computer software packages that can identify CDRs in antibody variable regions, according to the various definitions are readily available including ab Ysis (abysis.org, last visited Jan. 5, 2023).

TABLE 1
CDR Definitions

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

**Numbering of all CDR definitions in Table 1 is according to the numbering conventions set forth by Kabat et al. (1983).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J chain, T cell Receptors for Antigen, T cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 1 (allele IGHM*03) and SEQ ID NO: 2 (allele IGHM*04)) and by the Kabat system is set out below. The underlined amino acid residues are not accounted for in the Kabat system (“X,” double underlined below, can be serine(S) (SEQ ID NO: 1) or glycine (G) (SEQ ID NO: 2)):

| Sequential (SEQ ID NO: 1 or SEQ ID NO: 2)/KABAT numbering key for | |
|---|---|
| IgM heavy chain | |
| 1/127 | GSASAPTLFP LVSCENSPSD TSSVAVGCLA QDFLPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGFS PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VFVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

Antibodies include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is meant that a binding molecule, e.g., an antibody, binds to an epitope via its antigen-binding domain, and that the binding entails some complementarity between the antigen-binding domain and the epitope. According to this definition, a binding molecule, e.g., antibody, “specifically binds” to an epitope when it binds to that epitope via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

As used herein, the term “affinity” refers to a measure of the strength of the binding of an individual epitope with one or more antigen-binding domains, e.g., of an immunoglobulin molecule. See, e.g., Greenfield, E. A. (ed.), Antibodies: A Laboratory Manual, Second Edition, Cold Spring Harbor Laboratory Press, 2014. As used herein, the term “avidity” refers to the overall stability of the complex between a population of antigen-binding domains and an antigen. Avidity is related to both the affinity of individual antigen-binding domains in the population with specific epitopes, and the valencies of the immunoglobulins and the antigen. For example, the interaction between a bivalent monoclonal antibody and a target antigen with a highly repeating epitope structure, such as a polymer, would be one of high avidity. An interaction between a bivalent monoclonal antibody with a receptor present at a high density on a cell surface would also be of high avidity.

Binding molecules, such as multimeric binding molecules as provided herein can also be described or specified in terms of their cross-reactivity. As used herein, the term “cross-reactivity” refers to the ability of a binding molecule, e.g., an antibody specific for one antigen, to react with a second antigen, a measure of relatedness between two different antigenic substances. Thus, an antibody is cross reactive if it binds to an epitope other than the one that induced its formation. The cross-reactive epitope contains many of the same complementary structural features as the inducing epitope, and in some cases, can fit better than the original.

“Antigen-binding antibody fragments” including single-chain antibodies or other antigen-binding domains can exist alone or in combination with one or more of the following: hinge region, CH1, CH2, CH3, or CH4 domains, J chain, or secretory component. Also included are antigen-binding fragments that can include any combination of variable region(s) with one or more of a hinge region, CH1, CH2, CH3, or CH4 domains, a J chain, or a secretory component. Antibodies can be from any animal origin including fish, birds, and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, multimeric binding molecule, e.g., an IgM antibody as described herein can include an antigen-binding fragment of an antibody, e.g., a scFv fragment, so long as the IgM antibody is able to form a multimer, e.g., a hexamer or a pentamer, and an IgA antibody as provided herein can include an antigen-binding fragment of an antibody, e.g., a scFv fragment, so long as the IgA antibody is able to form a multimer, e.g., a dimer or a tetramer.

As used herein, the term “heavy chain subunit” includes amino acid sequences derived from an immunoglobulin heavy chain. A binding molecule, e.g., an antibody comprising a heavy chain subunit can include: a VH domain, a CH1 domain, a hinge (e.g., upper, middle, and/or lower hinge region) domain, a CH2 domain, a CH3 domain, a CH4 domain, a tailpiece, e.g., an IgM tailpiece, or any variant or fragment thereof. For example, a binding molecule, e.g., an antibody, can include without limitation, in addition to a VH domain: a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody can include, in addition to a VH domain, a CH3 domain and a CH4 domain; or a CH3 domain, a CH4 domain, an IgM tailpiece, and a J chain. Further, a binding molecule, e.g., an antibody can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, a multimeric binding molecule, e.g., a pentameric IgM antibody, IgM-like antibody, or other IgM-derived binding molecule or a dimeric or tetrameric IgA antibody, IgA-like antibody, or other IgA-derived binding molecule as provided herein includes sufficient portions of an IgM or IgA heavy chain constant region to allow the pentameric IgM antibody, IgM-like antibody, or other IgM-derived binding molecule, or dimeric or tetrameric IgA antibody, IgA-like antibody, or other IgA-derived binding molecule to form a multimer, e.g., a pentamer, a tetramer, or a dimer. As used herein such a heavy chain constant region fragment comprises a “multimerizing fragment.”

As used herein, the term “light chain subunit” includes amino acid sequences derived from an immunoglobulin light chain. The light chain subunit includes at least a VL, and can further include a CL (e.g., Ck or CA) domain.

Binding molecules, e.g., antibodies, can be described or specified in terms of the epitope(s) or portion(s) of a target, e.g., a target antigen that they recognize or specifically bind. The portion of a target antigen that specifically interacts with the target antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody, which has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J chain is altered by at least partial deletion, addition, or replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it is well within the competence of those skilled in the art, by carrying out routine experimentation, to obtain a functional engineered or humanized antibody.

As used herein, the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, and in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “cancer” and “cancerous” refer to or describe the physiological condition in mammals in which a population of cells are characterized by unregulated cell growth. Cancers can be categorized, e.g., as solid tumors or malignancies, or hematological cancers or malignancies. Both types can migrate to remote sites as metastases. A solid tumor can be categorized, e.g., as a sarcoma, a carcinoma, a melanoma, or a metastasis thereof.

The terms “proliferative disorder” and “proliferative disease” refer to disorders associated with abnormal cell proliferation such as cancer. “Tumor” and “neoplasm” as used herein refer to any mass of tissue that result from excessive cell growth or proliferation, either benign (noncancerous) or malignant (cancerous) including pre-cancerous lesions.

The terms “metastasis,” “metastases,” “metastatic,” and other grammatical equivalents as used herein refer to cancer cells which spread or transfer from the site of origin (e.g., a primary tumor) to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures. The terms also refer to the process of metastasis, which includes, but is not limited to detachment of cancer cells from a primary tumor, intravasation of the tumor cells to circulation, their survival and migration to a distant site, attachment and extravasation into a new site from the circulation, and microcolonization at the distant site, and tumor growth and development at the distant site.

Examples of such solid tumors can include, e.g., squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer (NSCLC), adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, prostate cancer, vulval cancer, thyroid cancer, head and neck cancer, any metastases thereof, or any combination thereof.

Examples of hematologic cancers or malignancies include without limitation leukemia, lymphoma, myeloma, acute myeloid leukemia (AML), chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

In certain embodiments, cancers that are amenable to treatment via the methods provided herein include, but are not limited to sarcomas, breast carcinomas, ovarian cancer, cervical cancer, head and neck cancer, NSCLC, esophageal cancer, gastric cancer, kidney cancer, liver cancer, bladder cancer, colorectal cancer, and pancreatic cancer.

The term “therapeutically effective amount” refers to an amount of an antibody, polypeptide, polynucleotide, small organic molecule, or other drug effective to “treat” a disease or disorder in a subject, e.g., a human. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality; improve quality of life; or a combination of such effects.

Terms such as “treating” or “treatment” or “to treat” or “alleviating” or “to alleviate” refer to therapeutic measures that cure, slow down, lessen symptoms of, lessen the severity of symptoms of, and/or halt or slow the progression of an existing diagnosed pathologic condition or disorder. Terms such as “prevent,” “prevention,” “avoid,” “deterrence,” “prophylactic,” and the like refer to prophylactic or preventative measures that prevent the development of an undiagnosed targeted pathologic condition or disorder. Thus, “those in need of treatment” can include those already with the disorder.

A subject is successfully “treated” according to the methods of the present disclosure if the patient shows one or more of the following: a reduction in the number of or complete absence of cancer cells; a reduction in the tumor size; or retardation or reversal of tumor growth, inhibition, e.g., suppression, prevention, retardation, shrinkage, delay, or reversal of metastases, e.g., of cancer cell infiltration into peripheral organs including, for example, the spread of cancer into soft tissue and bone; inhibition of, e.g., suppression of, retardation of, prevention of, shrinkage of, reversal of, delay of, or an absence of tumor metastases; inhibition of, e.g., suppression of, retardation of, prevention of, shrinkage of, reversal of, delay of, or an absence of tumor growth; relief of one or more symptoms associated with the specific cancer; reduced morbidity and mortality; improvement in quality of life; or some combination of effects. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder.

By “subject” or “individual” or “animal” or “patient” or “mammal,” is meant any mammalian subject. In certain embodiments, the subject is a subject for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

As used herein the terms “serum half-life” or “plasma half-life” refer to the time it takes (e.g., in minutes, hours, or days) following administration for the serum or plasma concentration of a drug, e.g., a binding molecule such as an antibody as described herein, to be reduced by 50%. Two half-lives can be described: the alpha half-life, a half-life, or t1/2α, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t1/2β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve can be measured, e.g., from time 0 (t0) to infinity (o) and is dependent on the rate of elimination of the drug from the body and the dose administered.

As used herein, the term “mean residence time” or “MRT” refers to the average length of time the drug remains in the body.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases which can be used to prepared salts include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound in its free acid or base form with a suitable organic or inorganic base or acid, respectively, and isolating the salt thus formed during subsequent purification.

The term “excipient” as used herein means an inert or inactive substance that may be used in the production of a drug or pharmaceutical product as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethyl cellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenan, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### IgM Antibodies and IgM-Like Antibodies

IgM is the first immunoglobulin produced by B cells in response to stimulation by antigen. Naturally occurring IgM is naturally present at around 1.5 mg/ml in serum with a half-life of about 5 days. IgM is a typically pentameric or hexameric molecule and thus can include five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal IgM tailpiece The human IgM constant region typically comprises the amino acid sequence SEQ ID NO: 1 (identical to, e.g., GenBank Accession Nos. pir∥S37768, CAA47708.1, and CAA47714.1, allele IGHM*03) or SEQ ID NO: 2 (identical to, e.g., GenBank Accession No. sp|P01871.4, allele IGHM*04). The human Cμ1 region ranges from about amino acid 5 to about amino acid 102 of SEQ ID NO: 1 or SEQ ID NO: 2; the human Cμ2 region ranges from about amino acid 114 to about amino acid 205 of SEQ ID NO: 1 or SEQ ID NO: 2, the human Cμ3 region ranges from about amino acid 224 to about amino acid 319 of SEQ ID NO: 1 or SEQ ID NO: 2, the Cμ4 region ranges from about amino acid 329 to about amino acid 430 of SEQ ID NO: 1 or SEQ ID NO: 2, and the μtp ranges from about amino acid 431 to about amino acid 453 of SEQ ID NO: 1 or SEQ ID NO: 2.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 1 or SEQ ID NO: 2 described and claimed elsewhere in this disclosure can likewise be incorporated into alternate human IgM sequences, as well as into IgM constant region amino acid sequences of other species.

Five IgM binding units can form a complex with an additional small polypeptide chain (the J chain), or a functional fragment or variant thereof, to form a pentameric IgM antibody, as discussed elsewhere herein. The precursor form of the human J chain is presented as SEQ ID NO: 6. The signal peptide extends from amino acid 1 to about amino acid 22 of SEQ ID NO: 6, and the mature human J chain extends from about amino acid 23 to amino acid 159 of SEQ ID NO: 6. The mature human J chain includes the amino acid sequence SEQ ID NO: 7.

Exemplary variant and modified J chains are provided elsewhere herein. Without the J chain, an IgM antibody or IgM-like antibody typically assembles into a hexamer, comprising up to twelve target antigen-binding domains. With a J chain, an IgM antibody or IgM-like antibody typically assembles into a pentamer, comprising up to ten target antigen-binding domains, or more, if the J chain is a modified J chain comprising one or more heterologous polypeptides comprising additional antigen-binding domain(s) that can bind to, e.g., an effector cell such as a T cell. The assembly of five or six IgM binding units into a pentameric or hexameric IgM antibody or IgM-like antibody is thought to involve the Cμ4 and IgM tailpiece domains. See, e.g., Braathen, R., et al., J. Biol. Chem. 277:42755-42762 (2002). Accordingly, a pentameric or hexameric IgM antibody typically includes at least the Cμ4 and μtp domains. A “multimerizing fragment” of an IgM heavy chain constant region thus includes at least the Cμ4 and μtp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM antibody as provided herein can include a complete IgM heavy (u) chain constant domain, e.g., SEQ ID NO: 1 or SEQ ID NO: 2, or a variant, derivative, or analog thereof, e.g., as provided herein.

In certain embodiments, the disclosure provides a pentameric binding molecule, e.g., an IgM antibody, that includes ten IgM-derived heavy chains, and a modified J chain and where the IgM-derived heavy chains comprise IgM heavy chain constant regions each associated with a target antigen-binding domain, i.e., an antigen-binding domain that specifically binds to a target antigen, such as tumor-associated target antigen. Moreover, an IgM-based binding molecule, e.g., an IgM antibody, can consistently form pentameric oligomers resulting in a more homogeneous expression product. Superior complement fixation can also be an advantageous effector function of IgM-based antibodies.

In certain embodiments, the two IgM heavy chain constant regions included in each binding unit are human heavy chain constant regions. In some embodiments, the heavy chains are glycosylated. In some embodiments, the heavy chains can be mutated to affect glycosylation. See, e.g., U.S. Patent Application Publication No. US2022/0306760 A1, which is incorporated herein by reference in its entirety.

Where the IgM antibody provided in this disclosure is pentameric, the IgM antibody further includes a modified J chain or variant thereof that further comprises one or more heterologous moieties attached to a J chain, as described elsewhere herein. In certain embodiments, the J chain can be mutated to affect, e.g., enhance, the serum half-life of the IgM antibody provided herein, as discussed elsewhere in this disclosure. In certain embodiments the J chain can be mutated to affect glycosylation, as discussed elsewhere in this disclosure.

An IgM heavy chain constant region can include one or more of a Cμ1 domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the IgM antibody, e.g., associate with second IgM constant region to form a binding unit with one, two, or more target antigen-binding domain(s), and/or associate with other binding units (and in the case of a pentamer, a J chain) to form a hexamer or a pentamer. In certain embodiments, the two IgM heavy chain constant regions or fragments or variants thereof within an individual binding unit each comprise a Cμ4 domain or fragment or variant thereof, an IgM tailpiece (μtp) or fragment or variant thereof, or a combination of a Cμ4 domain and a μtp or fragment or variant thereof. In certain embodiments, the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In certain embodiments, each of the two IgM heavy chain constant regions in a given binding unit is associated with a target antigen-binding domain, for example an Fv portion of an antibody, e.g., a VH and a VL of a human or murine antibody, where the VL can be associated with a light chain constant region. In certain embodiments, a multimeric binding molecule as provided herein comprises at least three, e.g., three, four, five, six, seven, eight, nine, or ten target antigen-binding domains that specifically bind to a tumor-specific target antigen or a tumor-associated target antigen.

In some embodiments, the binding units of the IgM antibody, IgM-like antibody, or other IgM-derived binding molecule comprise two light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

### IgA Antibodies and IgA-Like Antibodies

IgA plays a critical role in mucosal immunity and comprises about 15% of total immunoglobulin produced. IgA antibodies can be monomeric, dimeric, or tetrameric, including one, two, or four IgA binding units, respectively. An IgA binding unit includes two IgA heavy chains and can further include two light chains. An IgA heavy chain constant region contains three heavy chain constant domains (Cα1, Cα2, and Cα3), and includes a hinge region between Cα1 and Cα2, and a C-terminal IgA tailpiece. Human IgA has two subtypes, IgA1 and IgA2. The human IgA1 constant region typically includes the amino acid sequence SEQ ID NO: 3. The human Cα1 domain extends from about amino acid 6 to about amino acid 98 of SEQ ID NO: 3; the human IgA1 hinge region extends from about amino acid 102 to about amino acid 124 of SEQ ID NO: 3, the human Cα3 domain extends from about amino acid 228 to about amino acid 330 of SEQ ID NO: 3, and the αtp extends from about amino acid 331 to about amino acid 352 of SEQ ID NO: 3. The human IgA2 constant region typically includes the amino acid sequence SEQ ID NO: 4. The human Cα1 domain extends from about amino acid 6 to about amino acid 98 of SEQ ID NO: 4; the human IgA2 hinge region extends from about amino acid 102 to about amino acid 111 of SEQ ID NO: 4, the human Cα2 domain extends from about amino acid 113 to about amino acid 206 of SEQ ID NO: 4, the human Cα3 domain extends from about amino acid 215 to about amino acid 317 of SEQ ID NO: 4, and the αtp extends from about amino acid 318 to about amino acid 340 of SEQ ID NO: 4.

Two or four IgA binding units can form a complex with two additional polypeptide chains, the J chain (e.g., SEQ ID NO: 7 or SEQ ID NO: 8) and the secretory component (precursor, SEQ ID NO: 5, mature: amino acids 19 to 603 of SEQ ID NO: 5) to form a secretory IgA (sIgA) antibody. The assembly of IgA binding units into a dimeric or tetrameric sIgA antibody is thought to involve the Cα3 and αtp domains (also referred to herein collectively as the Cα3-tp domain). Accordingly, a dimeric or tetrameric sIgA antibody provided in this disclosure typically includes IgA constant regions that include at least the Cα3 and αtp domains.

An IgA heavy chain constant region can additionally include a Cα2 domain or a fragment thereof, an IgA hinge region, a Cα1 domain or a fragment thereof, and/or other IgA heavy chain domains. In certain embodiments, an IgA antibody or IgA-like binding molecule as provided herein can include a complete IgA heavy (α) chain constant domain (e.g., SEQ ID NO: 3 or SEQ ID NO: 4), or a variant thereof. In some embodiments, each IgA heavy chain constant region or multimerizing fragment thereof is a human IgA constant region.

In some embodiments, the binding units of the IgA antibody, IgA-like antibody, or other IgA-derived binding molecule comprise two light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

In some embodiments, this disclosure provides a dimeric or tetrameric binding molecule, e.g., an IgA antibody or an IgA-like antibody with two or four IgA “binding units,” that can specifically bind to a target antigen, e.g., a tumor-specific or tumor-associated target antigen. Use of an IgA-based binding molecule can allow, for example, greater tissue distribution of a binding molecule provided herein. Mucosal distribution could be beneficial for certain cancers, e.g., lung cancer, gastric cancer, ovarian cancer, colorectal cancer, or squamous cell carcinoma. Likewise, a tetrameric or dimeric IgA antibody or IgA-like antibody as provided herein can possess binding characteristics or biological activity that can be distinguished from a binding molecule comprising five binding units, e.g., a pentameric IgM antibody. For example, a dimeric binding molecule would be smaller, and could, for example, achieve better tissue penetration in certain solid tumors.

In certain embodiments, the disclosure provides a dimeric or tetrameric IgA antibody or IgA-like antibody comprising two or four bivalent binding units and a modified J chain, where each binding unit includes two IgA heavy chain constant regions or multimerizing fragments thereof. In certain embodiments, the two IgA heavy chain constant regions are human heavy chain constant regions.

An IgA heavy chain constant region can include one or more of a Cα1 domain, an IgA hinge region, a Cα2 domain, a Cα3 domain, and/or an αtp, provided that the constant region can serve a desired function in the binding molecule, e.g., associate with a light chain constant region to facilitate formation of a target antigen-binding domain or associate with another IgA binding unit to form a dimeric binding molecule. In certain embodiments the two IgA heavy chain constant regions or fragments thereof within an individual binding unit each comprise a Cα3 domain or multimerizing fragment thereof, a tailpiece (αtp) or multimerizing fragment thereof, or any combination of a Cα3 domain, a tp, or multimerizing fragment thereof. In certain embodiments the two IgA heavy chain constant regions or fragments thereof within an individual binding unit each further comprise a Cα2 domain or fragment thereof, a Cα1 domain or fragment thereof, an IgA hinge region, or a Cα1 domain or fragment thereof, an IgA hinge region, and a Cα2 domain or fragment thereof.

In certain embodiments, each of the two IgA heavy chain constant regions in a given binding unit is associated with a target antigen-binding domain, for example an Fv portion of an antibody, e.g., a VH and a VL of a human or murine antibody, where the VL can be associated with a light chain constant region. In certain embodiments, a multimeric binding molecule as provided herein comprises at least three target antigen-binding domains that can specifically bind to a target antigen, e.g., a tumor-associated or tumor-specific target antigen.

### J Chains and Functional Fragments or Variants Thereof

Certain multimeric binding molecules provided herein include a modified J chain or functional fragment or variant thereof. In certain embodiments, the multimeric binding molecule provided herein is pentameric and comprises a modified J chain or functional fragment or variant thereof. In certain embodiments, the binding molecule provided herein is dimeric or tetrameric and comprises a modified J chain or functional fragment or variant thereof. In some embodiments, the dimeric, tetrameric, or pentameric binding molecule includes a modified J chain comprising a naturally occurring J chain sequence, such as a mature human J chain sequence (e.g., SEQ ID NO: 7). Alternatively, in some embodiments, the dimeric, tetrameric, or pentameric binding molecule includes a modified J chain comprising a variant J chain sequence, such as a variant sequence described herein with reduced glycosylation and/or reduced binding to a polymeric Ig receptor (e.g., plgR, Fc alpha-mu receptor (FcauR), or Fc mu receptor (FcuR)). See, e.g., U.S. Pat. No. 10,899,835, which is incorporated herein by reference in its entirety). In some embodiments, the modified J chain of the dimeric, tetrameric, or pentameric binding molecule can comprise a functional fragment of a naturally occurring or variant J chain. As persons of ordinary skill in the art will recognize, “a functional fragment” or a “functional variant” in this context includes those fragments and variants that can associate with binding units, e.g., IgM or IgA heavy chain constant regions, to form a pentameric IgM antibody, IgM-like antibody, or IgM-derived binding molecule or a dimeric or tetrameric IgA antibody, IgA-like antibody, or IgA-derived binding molecule, and/or can associate with certain immunoglobulin receptors, e.g., pIgR.

As provided herein, the J chain is modified, e.g., by introduction of a heterologous moiety, or two or more heterologous moieties, e.g., polypeptides, without interfering with the ability of binding molecule to assemble and bind to its binding target(s). See U.S. Pat. Nos. 9,951,134, 10,400,038, 10,618,978, and 11,639,389, each of which is incorporated herein by reference in its entirety.

Accordingly, a multimeric binding molecule provided by this disclosure, including IgA, IgA-like, IgM, or IgM-like antibodies as described elsewhere herein, comprises a modified J chain or functional fragment or variant thereof comprising a heterologous moiety, e.g., a heterologous polypeptide, attached, e.g., fused or chemically conjugated, into the J chain or fragment or variant thereof. In certain embodiments, the heterologous polypeptide can be fused to the N-terminus of the J chain or functional fragment or variant thereof, or the C-terminus of the J chain or functional fragment or variant thereof. In certain embodiments a heterologous moiety, e.g., a heterologous polypeptide, is attached to both the N-terminus and C-terminus of the J chain or functional fragment or variant thereof. In certain embodiments, the heterologous polypeptide can be fused internally within the J chain or functional fragment or variant thereof. In some embodiments, the heterologous polypeptide can be introduced into the J chain at or near a glycosylation site. In some embodiments, the heterologous polypeptide can be introduced into the J chain within about 10 amino acid residues from the C-terminus, or within about 10 amino acids from the N-terminus. In certain embodiments, the heterologous polypeptide can be introduced into the mature human J chain of SEQ ID NO: 7 between cysteine residues 92 and 101 of SEQ ID NO: 7, or an equivalent location in a J chain sequence, e.g., a J chain variant or functional fragment of a J chain. In a further embodiment, the heterologous polypeptide can be introduced into the mature human J chain of SEQ ID NO: 7 at or near a glycosylation site. In a further embodiment, the heterologous polypeptide can be introduced into the mature human J chain of SEQ ID NO: 7 within about 10 amino acid residues from the C-terminus, or within about 10 amino acids from the N-terminus.

In certain embodiments, the heterologous moiety can be a peptide or polypeptide sequence fused in frame to the J chain or chemically conjugated to the J chain or fragment or variant thereof. In certain embodiments, the heterologous polypeptide is fused to the J chain or functional fragment thereof via an amino acid linker. Any suitable linker can be used, for example the amino acid linker can include at least 5 amino acids, at least ten amino acids, and least 20 amino acids, at least 30 amino acids or more, and so on. In certain embodiments, the amino acid linker includes least 5 amino acids, but no more than 25 amino acids. In certain embodiments the amino acid linker can consist of 5 amino acids, 10 amino acids, 15 amino acids, 20 amino acids, or 25 amino acids. In certain embodiments, the amino acid linker consists of the amino acid sequence (GGGGS)n (SEQ ID NO: 279), where n is an integer between 2 and 5. In certain embodiments the amino acid linker consists of GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), GGGGSGGGGSGGGGSGGGGSGGGGS (SEQ ID NO: 13), GKPGSGKPGSGKPGSGKPGS (SEQ ID NO: 14), or GGGGSGGGGSGGGG (SEQ ID NO: 235).

In certain embodiments, the heterologous moiety can be a chemical moiety conjugated to the J chain. Heterologous moieties to be attached to a J chain can include, without limitation, a binding moiety, e.g., an antibody or antigen-binding fragment thereof, e.g., a single chain Fv (scFv) molecule, a cytokine, e.g., IL-2 or IL-15 (see, e.g., PCT Publication No. WO2021030688A1, which is incorporated herein by reference in its entirety), a stabilizing peptide that can increase the half-life of the binding molecule, e.g., human serum albumin (HSA) or an HSA binding molecule, or a heterologous chemical moiety such as a polymer or a cytotoxin. In certain embodiments described in more detail elsewhere herein, the heterologous moiety includes a polypeptide agonist of a T cell costimulatory molecule. In certain embodiments, a modified J chain of a multimeric binding molecule as provided herein comprises two heterologous moieties, an anti-CD3 scFv and a polypeptide agonist of a T cell costimulatory molecule. In certain embodiments, the T cell costimulatory molecule is CD28. In certain embodiments, the T cell costimulatory molecule is 4-1BB. In certain embodiments, the polypeptide agonist of the T cell costimulatory molecule is an anti-CD28 antibody or antigen-binding fragment thereof. In certain embodiments, the polypeptide agonist of a T cell costimulatory molecule is a 4-1BB ligand (4-1BBL) trimer.

In some embodiments, the modified J chain comprises an antigen-binding domain. In certain embodiments, an antigen-binding domain associated with a modified J chain is an antibody or an antigen-binding fragment thereof. In certain embodiments, the antigen-binding domain can be a scFv antigen-binding domain or a single-chain antigen-binding domain derived, e.g., from a camelid or condricthoid antibody (sdVH) or from an antibody light chain (sdVL). In certain embodiments, an antigen-binding domain binds to a T cell, e.g., a cytotoxic T cell.

An antigen-binding domain can be introduced into the J chain at any location that allows the binding of the antigen-binding domain to its antigen without interfering with J chain function or the function of an associated multimeric binding molecule, e.g., a pentameric IgM or dimeric IgA antibody. Insertion locations include but are not limited to at or near the C-terminus, at or near the N-terminus or at an internal location that, based on the three-dimensional structure of the J chain, is accessible.

Variant J Chains that Confer Increased Serum Half-Life

In certain embodiments, the modified J chain of a multimeric binding molecule as provided herein comprises a functional variant J chain that includes one or more single amino acid substitutions, deletions, or insertions relative to a reference J chain identical to the variant J chain except for the one or more single amino acid substitutions, deletions, or insertions. For example, certain amino acid substitutions, deletions, or insertions can result in the IgM-derived multimeric binding molecule exhibiting an increased serum half-life upon administration to a subject animal relative to a reference IgM-derived binding molecule that is identical except for the one or more single amino acid substitutions, deletions, or insertions in the variant modified J chain, and is administered using the same method to the same animal species. In certain embodiments, the variant modified J chain can include one, two, three, or four single amino acid substitutions, deletions, or insertions relative to the reference J chain.

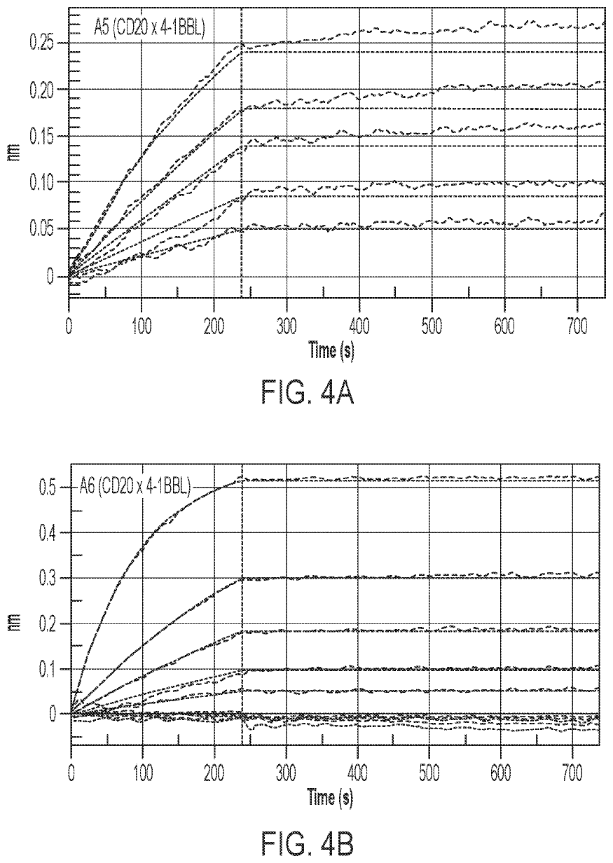

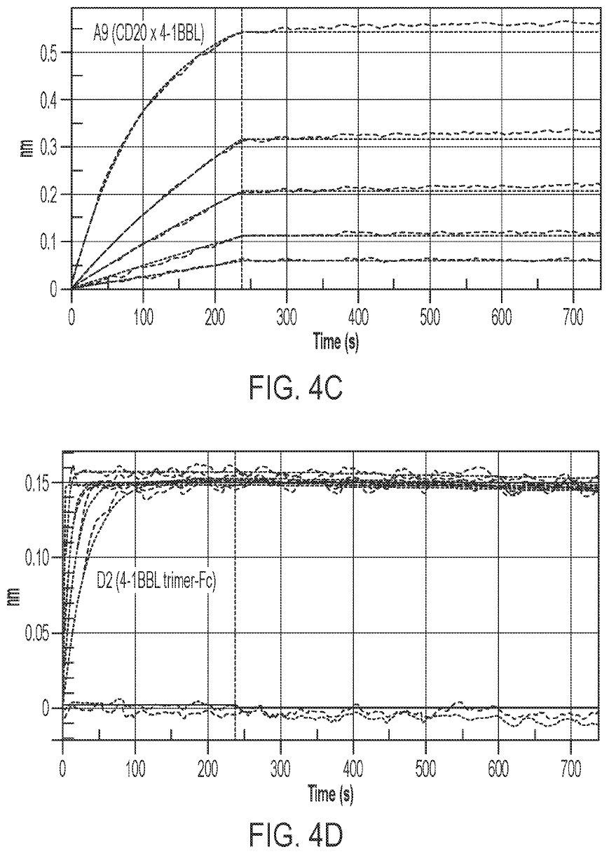

In certain embodiments, the modified J chain comprises an amino acid substitution at the amino acid position corresponding to amino acid Y102 of the mature wild-type human J chain (SEQ ID NO: 7). By “an amino acid corresponding to amino acid Y102 of the mature wild-type human J chain” is meant the amino acid in the sequence of the J chain, which is homologous to Y102 in the human J chain. For example, see U.S. Pat. No. 10,899,835, which is incorporated herein by reference in its entirety. The position corresponding to Y102 in SEQ ID NO: 7 is conserved in the J chain amino acid sequences of at least 43 other species. See FIG. 4 of U.S. Pat. No. 9,951,134, which is incorporated by reference. Certain mutations at the position corresponding to Y102 of SEQ ID NO: 7 can inhibit the binding of certain immunoglobulin receptors, e.g., the human or murine Fcau receptor, the murine Fcu receptor, and/or the human or murine polymeric Ig receptor (pIgR) to an IgM pentamer comprising the variant J chain.

A multimeric binding molecule comprising a modified J chain with a mutation at the amino acid corresponding to Y102 of SEQ ID NO: 7 has an improved serum half-life when administered to an animal than a corresponding multimeric binding molecule that is identical except for the substitution, and which is administered to the same species in the same manner. In certain embodiments, the amino acid corresponding to Y102 of SEQ ID NO: 7 can be substituted with any amino acid. In certain embodiments, the amino acid corresponding to Y102 of SEQ ID NO: 7 can be substituted with alanine (A), serine(S) or arginine (R). In a particular embodiment, the amino acid corresponding to Y102 of SEQ ID NO: 7 can be substituted with alanine. In a particular embodiment the modified J chain or functional fragment or variant thereof comprises a variant human J chain referred to herein as “J*,” comprising the amino acid sequence SEQ ID NO: 8.

Wild-type J chains typically include one N-linked glycosylation site. In certain embodiments, a modified J chain comprising a variant J chain or functional fragment thereof of a multimeric binding molecule as provided herein includes a mutation within the asparagine (N)-linked glycosylation motif N-X1-S/T, e.g., starting at the amino acid position corresponding to amino acid 49 (motif N6) of the mature human J chain (SEQ ID NO: 7) or J* (SEQ ID NO: 8), where Nis asparagine, X1 is any amino acid except proline, and S/T is serine or threonine, and where the mutation prevents glycosylation at that motif. As demonstrated in U.S. Pat. No. 10,899,835, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J chain, and is administered in the same way to the same animal species.

For example, in certain embodiments a modified J chain comprising a variant J chain or functional fragment thereof of a pentameric IgM-derived or dimeric IgA-derived binding molecule as provided herein can include an amino acid substitution at the amino acid position corresponding to amino acid N49 or amino acid S51 of SEQ ID NO: 7 or SEQ ID NO: 8, provided that the amino acid corresponding to S51 is not substituted with threonine (T), or where the variant J chain comprises amino acid substitutions at the amino acid positions corresponding to both amino acids N49 and S51 of SEQ ID NO: 7 or SEQ ID NO: 8. In certain embodiments, the position corresponding to N49 of SEQ ID NO: 7 or SEQ ID NO: 8 is substituted with any amino acid, e.g., alanine (A), glycine (G), threonine (T), serine(S) or aspartic acid (D). In a particular embodiment, the position corresponding to N49 of SEQ ID NO: 7 or SEQ ID NO: 8 can be substituted with alanine (A). In another embodiment, the position corresponding to N49 of SEQ ID NO: 7 or SEQ ID NO: 8 can be substituted with aspartic acid (D).

### Variant IgM Constant Regions

IgM heavy chain constant regions of a multimeric binding molecule as provided herein can be engineered to confer certain desirable properties to the multimeric binding molecules provided herein. For example, in certain embodiments, IgM heavy chain constant regions can be engineered to confer enhanced serum half-life to multimeric binding molecules as provided herein. Exemplary IgM heavy chain constant region mutations that can enhance serum half-life of an IgM-derived binding molecule are disclosed in U.S. Pat. No. 10,899,835, which is incorporated by reference herein in its entirety. For example, a variant IgM heavy chain constant region of the IgM antibody, IgM-like antibody, or IgM-derived binding molecule as provided herein can include an amino acid substitution at a position corresponding to amino acid S401, E402, E403, R344, and/or E345 of a wild-type human IgM constant region (e.g., SEQ ID NO: 1 or SEQ ID NO: 2). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 1 or SEQ ID NO: 2 can be substituted with any amino acid, e.g., alanine.

In certain embodiments, an IgM antibody, IgM-like antibody, or other IgM-derived binding molecule as provided herein, can be engineered to exhibit reduced complement-dependent cytotoxic (CDC) activity to cells in the presence of complement, relative to a reference IgM antibody, IgM-like antibody, or other IgM-derived binding molecule with corresponding reference human IgM constant regions identical, except for the mutations conferring reduced CDC activity. These CDC mutations can be combined with any of the mutations to confer increased serum half-life as provided herein. By “corresponding reference human IgM constant region” is meant a human IgM constant region that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in U.S. Pat. No. 11,401,337, which is incorporated herein by reference in its entirety. Assays for measuring CDC are well known to those of ordinary skill in the art, and exemplary assays are described e.g., in U.S. Pat. No. 11,401,337.

In certain embodiments, a variant human IgM constant region conferring reduced CDC activity includes an amino acid substitution corresponding to the wild-type human IgM constant region at position L310, P311, P313, and/or K315 of SEQ ID NO: 1 (human IgM constant region allele IGHM*03) or SEQ ID NO: 2 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 1 or SEQ ID NO: 2. In other embodiments the variant IgM constant region as provided herein contains an amino acid substitution corresponding to the wild-type human IgM constant region at position P313 of SEQ ID NO: 1 or SEQ ID NO: 2. In other embodiments the variant IgM constant region as provided herein contains a combination of substitutions corresponding to the wild-type human IgM constant region at positions P311 of SEQ ID NO: 1 or SEQ ID NO: 2 and P313 of SEQ ID NO: 1 or SEQ ID NO: 2. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 1 or SEQ ID NO: 2. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 1 or SEQ ID NO: 2, which can be substituted, e.g., with aspartic acid.

Human and certain non-human primate IgM constant regions typically include five (5) naturally occurring asparagine (N)-linked glycosylation motifs or sites. As used herein “an N-linked glycosylation motif” comprises or consists of the amino acid sequence N-X1-S/T, where N is asparagine, X1 is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 1 or SEQ ID NO: 2 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan. In certain embodiments, at least one, at least two, at least three, or at least four of the N-X1-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiments, the IgM constant region comprises two or more substitutions relative to a wild-type human IgM constant region at positions 46, 209, 272, or 440 of SEQ ID NO: 1 (human IgM constant region allele IGHM*03) or SEQ ID NO: 2 (human IgM constant region allele IGHM*04). See, e.g., US2022/036760, which is incorporated herein by reference in its entirety.

Multimeric Binding Molecules that Induce T Cell Costimulation

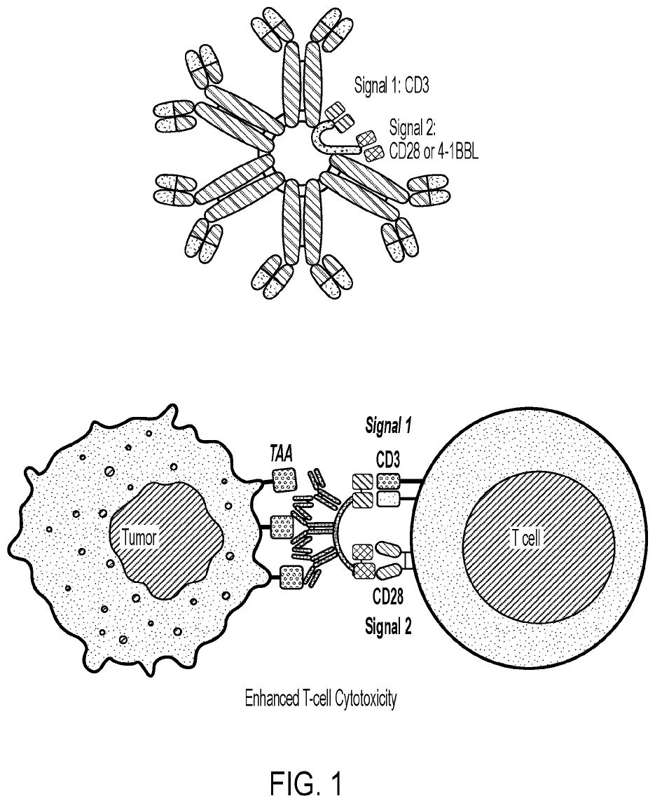

This disclosure provides a T cell-engaging multimeric binding molecule, e.g., an IgM, IgM-like, IgA, and IgA-like antibody comprising two, four, or ten binding units collectively comprising three, four, five, six, seven, eight, nine, or ten target antigen-binding domains, i.e., antigen-binding domains that specifically bind to a target antigen, e.g., a tumor-associated target antigen or a tumor-specific target antigen, and a modified J chain, where the binding molecule targets and activates T cells through two signals, “signal 1,” targeting CD3, and “signal 2,” targeting a T cell costimulatory molecule. The signal 1 and signal 2 targeting is accomplished, e.g., through dual T cell engagement via a “bidentate” modified J chain that comprises a J chain or a functional fragment or variant thereof, an scFv that specifically binds to CD3, and a polypeptide agonist of a T cell costimulatory molecule. The T cell costimulatory molecule can be, e.g., CD28 or 4-1BB, and the polypeptide agonist can be, e.g., an scFv of an anti-CD28 agonist antibody, or a trimer of the receptor-binding portion of the 4-1BB ligand (4-1BBL). A schematic of an exemplary IgM antibody that includes a bidentate modified J chain, and how it would function to enhance T cell activation, is shown in FIG. 1. A multimeric binding molecule as provided herein allows for dual engagement of T cells, while maintaining a full avidity advantage for targeting a desired target antigen, e.g., a tumor-associated antigen. This enhanced T cell activation can increase the ability of the multimeric binding molecule to facilitate T cell killing of tumor cells, can increase the number and variety of T cells available to kill tumor cells, and can, in some embodiments, increase T cell-mediated killing in settings with typically low T cell counts, e.g., in solid tumors.

Costimulatory molecules are receptors expressed on T cells that upon coactivation can, e.g., induce signaling to more fully activate T cells upon T cell receptor (TCR) engagement, thereby enhancing signaling and cytokine stimulation. This co-signaling can further modulate T cell differentiation, effector function, and survival. See, e.g., Baeuerle, P. A., and H. Wesche, Curr. Opin. Oncol. 34:552-558 (2022). Exemplary T cell costimulatory molecules include, without limitation, CD28 and 4-1BB. See, e.g., Jeong, S., and S-H. Park, Immune Netw. doi: 10.4110/in.2020.20.e3 (2020).

CD28 is expressed on approximately 80% of human CD4+ T cells and 50% CD8+ T cells, and interacts with various ligands, including CD80 and CD86. Engagement of CD28 with its ligands promotes T cell stimulation and cytokine expression. See Esensten, J. H., et al., Immunity 44:973-988 (2016). Human CD28, a type 1 membrane protein, is presented as SEQ ID NO: 236. The signal peptide of human CD28 extends from amino acid 1 to about amino acid 18 of SEQ ID NO: 236. The extracellular domain of human CD28 extends from about amino acid 19 to about amino acid 152 of SEQ ID NO: 236. The transmembrane domain of human CD28 extends from about amino acid 153 to about amino acid 179 of SEQ ID NO: 236. The cytoplasmic domain of human CD28 extends from about amino acid 180 to amino acid 220 of SEQ ID NO: 236.

4-1BB, also known as CD137 or TNFRSF9, is a tumor necrosis factor superfamily receptor (TNFSFR) expressed, e.g., on activated T cells. 4-1BB is expressed on both activated CD4+ and CD8+ T cells (Bartkowiak, T, and M A Curran, Front Oncol. 5: doi: 10.3389/fonc.2015.00117 (2015); Vinay, D S, and B S Kwon, Mol Cancer Ther. 11: DOI: 10.1158/1535-7163.MCT-11-0677 (2012)). Upon activation, CD8+ effector T cells upregulate 4-1BB expression, and 4-1BB signaling promotes survival, T cell proliferation, and enhanced effector function (Bartkowiak, T, and M A Curran, Front Oncol. 5: doi: 10.3389/fonc.2015.00117 (2015)). Interaction with its trimeric ligand (4-1BBL, TNFSF9) expressed on activated antigen-presenting cells (APCs), e.g., macrophages and dendritic cells (DC), provides enhanced costimulatory proliferation, survival, and effector functions in CD8+ effector T cells (Moran, A E, et al., Curr Opin Immunol. 25:10.1016/j.coi.2013.01.004 (2013)). Human 4-1BB, a type 1 membrane protein, is presented as SEQ ID NO: 195. The signal peptide of human 4-1BB extends from amino acid 1 to about amino acid 23 of SEQ ID NO: 195. The extracellular domain of human 4-1BB extends from about amino acid 24 to about amino acid 186 of SEQ ID NO: 195. The transmembrane domain of human 4-1BB extends from about amino acid 187 to about amino acid 213 of SEQ ID NO: 195. The cytoplasmic domain of human 4-1BB extends from about amino acid 214 to amino acid 255 of SEQ ID NO: 195.

The ligand for 4-1BB (4-1BBL) is a type 2 transmembrane glycoprotein receptor that is found on antigen presenting cells (APCs). Gramaglia et al., Eur. J. Immunol. 30(2):392-402, 2000. 4-1BBL is the high affinity ligand of 4-1BB. Chin et al., Nature Comm. 9:4679, 2018. 4-1BBL induces the proliferation of activated peripheral blood T cells and may have a role in activation-induced cell death (AICD). Vinay et al., BMB Rep. 47(3):122-129, 2014. Furthermore, 4-1BBL provides T cells co-stimulatory signals for survival, proliferation, and differentiation and may play a role in cognate interactions between T cells and B cells/macrophages. Eun et al., J. Immunol. 194(1):134-141, 2015. Accordingly, 4-1BBL is believed to be involved in cancers, infectious diseases, and autoimmune diseases. Vinay et al., Mol. Cancer Therap. 11(5):1062-1070, 2012. Human 4-1BBL, a type 2 membrane protein, is presented as SEQ ID NO: 15. The cytoplasmic domain of human 4-1BBL extends from amino acid 1 to about amino acid 28 of SEQ ID NO: 15. The transmembrane domain of human 4-1BBL extends from about amino acid 29 to about amino acid 49 of SEQ ID NO: 15. The extracellular domain of human 4-1BBL extends from about amino acid 50 to amino acid 254 of SEQ ID NO: 15.

The 4-1BB/4-1BBL complex typically consists of three monomeric 4-1BBs bound to a trimeric 4-1BBL. Won et al., J. Biol. Chem. 285:9202-9210 (2010); Rabu et al., J. Biol. Chem. 280(50):41472-41481 (2005). Each 4-1BB monomer binds to two 4-1BBLs via cysteine-rich domains (CRDs). Bitra et al., J. Biol. Chem. 293(4):1317-1329 (2018).

An exemplary T cell-engaging multimeric binding molecule provided by the disclosure includes a modified J chain, wherein the modified J chain comprises (a) a J chain or a functional fragment or variant thereof (“J”), (b) an anti-CD3 scFv (“C”), and (c) a heterologous polypeptide (“H”), wherein H comprises a polypeptide agonist of a T cell costimulatory molecule, where the J, C, and H are associated as a fusion protein, such as in the N-terminal to C-terminal direction, C-J-H or H-J-C. In some embodiments, the T cell costimulatory molecule comprises CD28, and the agonist is, for example, an anti-CD28 antibody or antigen-binding fragment thereof. In some embodiments, the T cell costimulatory molecule comprises 4-1BB, and the agonist is, for example, a 4-1BB ligand (4-1BBL) trimer as described elsewhere herein.

In some embodiments, C and J are fused via an amino acid linker. In some embodiments, H and J are fused via an amino acid linker. When a linker is present between C and J and between H and J, the linkers can be the same or different. In some embodiments, one or both individual linkers consist of 5 to 25 amino acids, such as the amino acid sequence (GGGGS)n, wherein n is an integer between 1 and 5 (SEQ ID NO: 280), 2 and 5 (SEQ ID NO: 279), or 2 and 3 (SEQ ID NO: 281). Exemplary linkers include GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), GGGGSGGGGSGGGGSGGGGSGGGGS (SEQ ID NO 13), GKPGSGKPGSGKPGSGKPGS (SEQ ID NO: 14), or GGGGSGGGGSGGGG (SEQ ID NO: 235). In some embodiments, one or both individual linkers consist of GGGGSGGGGS (SEQ ID NO: 10). In some embodiments, one or both individual linkers consist of GGGGSGGGGSGGGGS (SEQ ID NO: 11). In some embodiments, one or both individual linkers consist of GKPGSGKPGSGKPGSGKPGS (SEQ ID NO: 14).

In some embodiments, the T cell costimulatory molecule comprises CD28, and the costimulatory molecule agonist comprises an anti-CD28 antibody or antigen-binding fragment thereof, such as a single-chain Fv (scFv) fragment. In some embodiments, the anti-CD28 antibody or antigen-binding fragment includes a heavy chain variable region (VH) and a light chain variable region (VL). In some embodiments, the VH comprises VH complementarity-determining regions VHCDR1, VHCDR2, and VHCDR3, such as VHCDR1, VHCDR2, and VHCDR3 sequences listed in Table 3 and the VL comprises VL complementarity-determining regions VLCDR1, VLCDR2, and VLCDR3, such as VLCDR1, VLCDR2, and VLCDR3 sequences listed in Table 4.

In some embodiments, the VHCDR1, VHCDR2, VHCDR3, VLCDR1, VLCDR2, and VLCDR3 comprise, respectively, the amino acid sequences SEQ ID NO: 95, SEQ ID NO: 105, SEQ ID NO: 133, SEQ ID NO: 138, SEQ ID NO: 164, and SEQ ID NO: 165; SEQ ID NO: 98, SEQ ID NO: 106, SEQ ID NO: 130, SEQ ID NO: 151, SEQ ID NO: 157, and SEQ ID NO: 171; SEQ ID NO: 98, SEQ ID NO: 116, SEQ ID NO: 135, SEQ ID NO: 147, SEQ ID NO: 155, and SEQ ID NO: 175; SEQ ID NO: 103, SEQ ID NO: 115, SEQ ID NO: 135, SEQ ID NO: 147, SEQ ID NO: 155, and SEQ ID NO: 175; SEQ ID NO: 96, SEQ ID NO: 110, SEQ ID NO: 125, SEQ ID NO: 147, SEQ ID NO: 155, and SEQ ID NO: 174; SEQ ID NO: 96, SEQ ID NO: 110, SEQ ID NO: 125, SEQ ID NO: 150, SEQ ID NO: 159, and SEQ ID NO: 176; SEQ ID NO: 100, SEQ ID NO: 120, SEQ ID NO: 127, SEQ ID NO: 146, SEQ ID NO: 153, and SEQ ID NO: 172; SEQ ID NO: 104, SEQ ID NO: 123, SEQ ID NO: 137, SEQ ID NO: 150, SEQ ID NO: 159, and SEQ ID NO: 176; SEQ ID NO: 91, SEQ ID NO: 118, SEQ ID NO: 126, SEQ ID NO: 146, SEQ ID NO: 154, and SEQ ID NO: 173; SEQ ID NO: 101, SEQ ID NO: 111, SEQ ID NO: 129, SEQ ID NO: 139, SEQ ID NO: 161, and SEQ ID NO: 169; SEQ ID NO: 97, SEQ ID NO: 112, SEQ ID NO: 124, SEQ ID NO: 142, SEQ ID NO: 163, and SEQ ID NO: 166; SEQ ID NO: 94, SEQ ID NO: 108, SEQ ID NO: 132, SEQ ID NO: 149, SEQ ID NO: 160, and SEQ ID NO: 177; SEQ ID NO: 93, SEQ ID NO: 109, SEQ ID NO: 131, SEQ ID NO: 148, SEQ ID NO: 158, and SEQ ID NO: 178; SEQ ID NO: 99, SEQ ID NO: 117, SEQ ID NO: 128, SEQ ID NO: 147, SEQ ID NO: 155, and SEQ ID NO: 174; SEQ ID NO: 99, SEQ ID NO: 117, SEQ ID NO: 128, SEQ ID NO: 150, SEQ ID NO: 159, and SEQ ID NO: 176; SEQ ID NO: 102, SEQ ID NO: 107, SEQ ID NO: 136, SEQ ID NO: 140, SEQ ID NO: 162, and SEQ ID NO: 170; SEQ ID NO: 102, SEQ ID NO: 114, SEQ ID NO: 136, SEQ ID NO: 140, SEQ ID NO: 162, and SEQ ID NO: 170; SEQ ID NO: 102, SEQ ID NO: 113, SEQ ID NO: 136, SEQ ID NO: 143, SEQ ID NO: 162, and SEQ ID NO: 170; SEQ ID NO: 91, SEQ ID NO: 119, SEQ ID NO: 126, SEQ ID NO: 146, SEQ ID NO: 154, and SEQ ID NO: 173; SEQ ID NO: 92, SEQ ID NO: 121, SEQ ID NO: 134, SEQ ID NO: 152, SEQ ID NO: 156, and SEQ ID NO: 167; or SEQ ID NO: 92, SEQ ID NO: 122, SEQ ID NO: 134, SEQ ID NO: 141, SEQ ID NO: 156, and SEQ ID NO: 168.

In some embodiments, the VH that includes the three VHCDRs as noted above comprises an amino acid sequence at least 80%, 85%, 90%, 95%, or 100% identical to a VH amino acid sequence listed in Table 2. In some embodiments the VL that includes the three VLCDRs as noted above comprises an amino acid sequence at least 80%, 85%, 90%, 95%, or 100% identical to a VL amino acid sequence listed in Table 2. In some embodiments, the VH comprises a VH amino acid sequence listed in Table 2 and the VL comprises a VL amino acid sequence listed in Table 2. In some embodiments, the VH and VL that include the VHCDRs and VLCDRs as noted above comprise amino acid sequences at least 80%, 85%, 90%, 95%, or 100% identical to the amino acid sequences SEQ ID NO: 56 and SEQ ID NO: 90, SEQ ID NO: 57 and SEQ ID NO: 86, SEQ ID NO: 58 and SEQ ID NO: 83, SEQ ID NO: 59 and SEQ ID NO: 83, SEQ ID NO: 60 and SEQ ID NO: 82, SEQ ID NO: 60 and SEQ ID NO: 87, SEQ ID NO: 61 and SEQ ID NO: 81, SEQ ID NO: 62 and SEQ ID NO: 87, SEQ ID NO: 63 and SEQ ID NO: 85, SEQ ID NO: 64 and SEQ ID NO: 76, SEQ ID NO: 65 and SEQ ID NO: 75, SEQ ID NO: 66 and SEQ ID NO: 89, SEQ ID NO: 67 and SEQ ID NO: 88, SEQ ID NO: 68 and SEQ ID NO: 82, SEQ ID NO: 68 and SEQ ID NO: 87, SEQ ID NO: 69 and SEQ ID NO: 78, SEQ ID NO: 70 and SEQ ID NO: 78, SEQ ID NO: 71 and SEQ ID NO: 80, SEQ ID NO: 72 and SEQ ID NO: 84, SEQ ID NO: 73 and SEQ ID NO: 77, or SEQ ID NO: 74 and SEQ ID NO: 79, respectively.

In some embodiments, the anti-CD28 antibody or antigen-binding fragment of an antibody is an anti-CD28 single domain heavy chain variable region (sdVH) or an anti-CD28 single domain light chain variable region (sdVL). In some embodiments, the sdVH or sdVL comprise complementarity-determining regions CDR1, CDR2, and CDR3 listed in Table 6. In some embodiments, the anti-CD28 sdVH comprises complementarity-determining regions CDR1, CDR2, and CDR3, wherein the CDR1, CDR2, and CDR3 comprise, respectively, the amino acid sequences SEQ ID NO: 183, SEQ ID NO: 187, and SEQ ID NO: 191; SEQ ID NO: 184, SEQ ID NO: 188, and SEQ ID NO: 192; or SEQ ID NO: 185, SEQ ID NO: 189, and SEQ ID NO: 193. In some embodiments, the anti-CD28 sdVL comprises complementarity-determining regions CDR1, CDR2, and CDR3, wherein the CDR1, CDR2, and CDR3 comprise, respectively, the amino acid sequences SEQ ID NO: 186, SEQ ID NO: 190, and SEQ ID NO: 194.

In some embodiments, the anti-CD28 sdVH or sdVL that includes the CDRs noted above comprises an amino acid sequence at least 80%, 85%, 90%, 95%, or 100% identical to an amino acid sequence listed in Table 5. In some embodiments, the anti-CD28 sdVH or sdVL that includes the CDRs noted above comprises an amino acid sequence listed in Table 5. In some embodiments, the anti-CD28 sdVH that includes the CDRs noted above comprises an amino acid sequence at least 80%, 85%, 90%, 95%, or 100% identical to the amino acid sequence SEQ ID NO: 179, SEQ ID NO: 180, or SEQ ID NO: 181, or the anti-CD28 sdVL that includes the CDRs noted above comprises an amino acid sequence at least 80%, 85%, 90%, 95%, or 100% identical to the amino acid sequence SEQ ID NO: 182. In some embodiments, the anti-CD28 sdVH comprises SEQ ID NO: 179, SEQ ID NO: 180, or SEQ ID NO: 181, or the anti-CD28 sdVL comprises SEQ ID NO: 182.

In some embodiments, the anti-CD3 scFv (“C”) comprises a heavy chain variable region (VH) and a light chain variable region (VL), wherein the VH of C comprises VH complementarity-determining regions VHCDR1, VHCDR2, and VHCDR3, such as VHCDR1, VHCDR2, and VHCDR3 sequences listed in Table 9 and the VL of C comprises VL complementarity-determining regions VLCDR1, VLCDR2, and VLCDR3, such as VLCDR1, VLCDR2, and VLCDR3 sequences listed in Table 10.

In some embodiments, the VHCDR1, VHCDR2, VHCDR3, VLCDR1, VLCDR2, and VLCDR3 of C comprise, respectively, the amino acid sequences SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 31; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; SEQ ID NO: 35, SEQ ID NO: 43, SEQ ID NO: 47, SEQ ID NO: 39, SEQ ID NO: 40, and SEQ ID NO: 49, or SEQ ID NO: 25, SEQ ID NO: 26, SEQ ID NO: 51, SEQ ID NO: 53, SEQ ID NO: 54, and SEQ ID NO: 55.

In some embodiments, the VH of C comprises an amino acid sequence at least 80%, 85%, 90%, 95%, or 100% identical to a VH sequence listed in Table 8 and the VL of C comprises an amino acid sequence at least 80%, 85%, 90%, 95%, or 100% identical to a VL sequence listed in Table 8. In some embodiments, the VH and VL of C comprise an amino acid sequence at least 80%, 85%, 90%, 95%, or 100% identical to the amino acid sequence of SEQ ID NO: 16 and SEQ ID NO: 20; SEQ ID NO: 24 and SEQ ID NO: 28; SEQ ID NO: 24 and SEQ ID NO: 32; SEQ ID NO: 34 and SEQ ID NO: 38; SEQ ID NO: 42 and SEQ ID NO: 44; or SEQ ID NO: 46 and SEQ ID NO: 48, respectively.

In some embodiments, the VH and VL of the anti-CD28 antibody or antigen-binding fragment thereof are fused via an amino acid linker. In some embodiments, the VH and VL of the anti-CD3 antibody or antigen-binding fragment thereof are fused via an amino acid linker. In some embodiments, the linker consists of 5 to 25 amino acids, such as the amino acid sequence (GGGGS)n, wherein n is an integer between 1 and 5 (SEQ ID NO: 280), 2 and 5 (SEQ ID NO: 279), or 2 and 3 (SEQ ID NO: 281). Exemplary linkers include GGGGS (SEQ ID NO: 9), GGGGSGGGGS (SEQ ID NO: 10), GGGGSGGGGGGGGS (SEQ ID NO: 11), GGGGSGGGGSGGGGSGGGGS (SEQ ID NO: 12), GGGGSGGGGSGGGGSGGGGSGGGGS ID NO: (SEQ 13), GKPGSGKPGSGKPGSGKPGS (SEQ ID NO: 14), or GGGGSGGGGSGGGG (SEQ ID NO: 235). In some embodiments, the linker consists of GGGGSGGGGS (SEQ ID NO: 10). In some embodiments, the linker consists of GGGGSGGGGSGGGGS (SEQ ID NO: 11). In some embodiments, the linker consists of GKPGSGKPGSGKPGSGKPGS (SEQ ID NO: 14).

In some embodiments, the modified J chain comprises SEQ ID NO: 225, SEQ ID NO: 226, SEQ ID NO: 227, SEQ ID NO: 228, SEQ ID NO: 229, SEQ ID NO: 230, SEQ ID NO: 231, SEQ ID NO: 232, SEQ ID NO: 233, or SEQ ID NO: 234.

### Multimeric Binding Molecules Expressing a 4-1BBL Trimer

This disclosure further provides a multimeric binding molecule, e.g., an IgM, IgM-like, IgA, and IgA-like binding molecule comprising two, four, or ten binding units collectively comprising three, four, five, six, seven, eight, nine, or ten target antigen-binding domains, i.e., antigen-binding domains that specifically bind to a target antigen, e.g., a tumor-associated target antigen or a tumor-specific target antigen, and a modified J chain, where the binding molecule activates T cells through engagement of the T cell costimulatory molecule 4-1BB via expression of a trimer of the receptor-binding portion of the 4-1BB ligand (4-1BBL) on the modified J chain. Human 4-1BB and B-1BBL are described above. A multimeric binding molecule as provided herein allows for engagement and stimulation of T cells via activation of the 4-1BB, while maintaining a full avidity advantage for targeting a desired target antigen, e.g., a tumor-associated antigen.

In some embodiments, the modified J chain comprises a 4-1BBL trimer comprising three 4-1BBL monomers. In some embodiments, each 4-1BBL monomer is a soluble fragment of human 4-1BBL comprising amino acids X to 254 of SEQ ID NO: 15, wherein X is an integer from 50 to 99, such as 58 or 71. In some embodiments, the 4-1BBL trimer comprises SEQ ID NO: 196 or SEQ ID NO: 197.

In some embodiments, the C-terminus of a first 4-1BBL monomer is fused to the N-terminus of a second 4-1BBL monomer via an amino acid linker. In some embodiments, the C-terminus of a second 4-1BBL monomer is fused to the N-terminus of a third 4-1BBL monomer via an amino acid linker. When a linker is present between the first and second 4-1BBL monomer and the second and third 4-1BBL monomer, the linkers can be the same or different. In some embodiments, one or both individual linkers consist of 5 to 25 amino acids, such as the amino acid sequence (GGGGS)n, wherein n is an integer between 1 and 5 (SEQ ID NO: 280), 2 and 5 (SEQ ID NO: 279), or 2 and 3 (SEQ ID NO: 281). Exemplary linkers include GGGGS (SEQ ID NO: 9), GGGGSGGGGS (SEQ ID NO: 10), GGGGSGGGGSGGGGS (SEQ ID NO: 11), GGGGSGGGGSGGGGSGGGGS (SEQ ID NO: 12), GGGGSGGGGSGGGGSGGGGSGGGGS (SEQ ID NO: 13), GKPGSGKPGSGKPGSGKPGS (SEQ ID NO: 14), or GGGGSGGGGSGGGG (SEQ ID NO: 235). In some embodiments, one or both individual linkers consist of GGGGSGGGGS (SEQ ID NO: 10). In some embodiments, one or both individual linkers consist of GGGGGGGGSGGGGS (SEQ ID NO: 11). In some embodiments, one or both individual linkers consist of GKPGSGKPGSGKPGSGKPGS (SEQ ID NO: 14).

In some embodiments, the 4-1BBL trimer comprises SEQ ID NO: 198, SEQ ID NO: 199, SEQ ID NO: 200, or SEQ ID NO: 201.

In some embodiments, the modified J chain comprises a sequence listed in Table 7. In some embodiments, the modified J chain comprises SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211, SEQ ID NO: 212, SEQ ID NO: 213, or SEQ ID NO: 214.

TABLE 2
Anti-CD28 Antibody VH (or Heavy Chain) and VL (or Light Chain)
Sequences

| | VH | | VL | |
|---|---|---|---|---|
| | SEQ | | SEQ | |
| Source | ID NO | VH | ID NO | VL |
| US7538196B2 | 56 | DIVITQSPASLAVSLGQRAT | 90 | QVKLQQSGPGLVTPSQSLSITCT |
| | | ISCRASESVEYYVTSLMQWY | | VSGFSLSDYGVHWVRQSPGQGLE |
| | | QQKPGQPPKLLIFAASNVES | | WLGVIWAGGGTNYNSALMSRKSI |
| | | GVPARFSGSGSGTNFSLNIH | | SKDNSKSQVELKMNSLQADDTAV |
| | | PVDEDDVAMYFCQQSRKVPY | | YYCARDKGYSYYYSMDYWGQGTT |
| | | TFGGGTKLEIKR | | VTVSS |
| US20220089736 | 57 | EVQLLESGGGLGQPGGSLRL | 86 | EIVLTQSPATLSLSPGERAILSC |
| | | SCAASEFTFTSYAMSWVRQA | | RASQSVSSYLAWYQQKPGQAPRL |
| | | PGKGLEWVSAISGSGGRTYY | | LIYDASNRATGIPARFSGSGSGT |
| | | ADSVKGRFTISRDNSKNTLY | | DFTLTISSLEPEDFAVYYCQQRS |
| | | LQMNSLRAEDTAVYYCAKGA | | NWPLTFGGGTKVEIK |
| | | GYWYFDLWGRGTLVTVSS | | |
| US20220119530A1 | 58 | EVQLLESGGGLVQPGGSLRL | 83 | DIQMTQSPSSLSASVGDRVTITC |
| | | SCAASGFTFSSYAMSWVRQA | | RASQSISSYLNWYQQKPGKAPKL |
| | | PGKGLEWVSTISGSGDSTYY | | LIYAASSLQSGVPSRFSGSGSGT |
| | | ADSVKGRFTISRDNSKNTLY | | DETLTISSLQPEDFATYYCQQVY |
| | | LQMNSLRAEDTAVYYCAKSG | | STPFTFGQGTKLEIK |
| | | PGLRQVGFDYWGQGTLVTVS | | |
| | | S | | |
| US20220119530A1 | 59 | EVQLLESGGGLVQPGGSLRL | 83 | DIQMTQSPSSISASVGDRVTITC |
| | | SCAASGFTESSYYMSWVRQA | | RASQSISSYLNWYQQKPGKAPKL |
| | | PGKGLEWVSTISESGDSTYY | | LIYAASSLQSGVPSRFSGSGSGT |
| | | ADSVKGRFTISRDNSKNTLY | | DETLTISSLQPEDFATYYCQQVY |
| | | LQMNSLRAEDTAVYYCAKSG | | STPFTFGQGTKLEIK |
| | | PGLRQVGFDYWGQGTLVTVS | | |
| | | S | | |
| WO | 60 | EVQLVESGGGLVQPGGSLRL | 82 | DIQMTQSPSSISASVGDRVTITC |
| 2022/061098 | | SCAASGFTFSRNNMHWVRQA | | RASQSISSYLNWYQQKPGKAPKL |
| | | PGKGLEYVSGISSNGGRTYY | | LIYAASSLQSGVPSRFSGSGSGT |
| | | ADSVKGRFTISRDNSKNTLY | | DETLTISSLQPEDFATYYCQQSY |
| | | LQMGGLRAADMAVYFCTRDD | | STPPITFGQGTRLEIK |
| | | ELLSFDYWGQGTLVTVSS | | |
| WO | 60 | EVQLVESGGGLVQPGGSLRL | 87 | EIVLTQSPGTESLSPGERATLSC |
| 2022/061098 | | SCAASGFTFSRNNMHWVRQA | | RASQSVSSSYLAWYQQKPGQAPR |
| | | PGKGLEYVSGISSNGGRTYY | | LLIYGASSRATGIPDRFSGSGSG |
| | | ADSVKGRFTISRDNSKNTLY | | TQFTLTISRLEPEDFAVYYCQQY |
| | | LQMGGLRAADMAVYFCTRDD | | GSSPWTFGQGTKVEIK |
| | | ELLSFDYWGQGTLVTVSS | | |
| US | 61 | QVQLQESGPGLVKPSETLSL | 81 | DIQMTQSPSSLSASVGDRVTITC |
| 2004/0116675 | | TCAVSGFSLTSYGVHWIRQP | | RASESVEYYVTSLMQWYQQKPGK |
| | | PGKGLEWLGVIWPGGGTNEN | | APKLLIYAASNVDSGVPSRFSGS |
| | | SALMSRLTISEDTSKNQVSL | | GSGTDFTLTISSLQPEDIATYYC |
| | | KLSSVTAADTAVYYCARDRA | | QQSRKVPFTEGGGTKVEIK |
| | | YGNYLYAMDYWGQGTLVTVS | | |
| | | S | | |
| WO | 62 | QVQLQESGPGLVKPSETLSL | 87 | EIVLTQSPGTLSLSPGERATLSC |
| 2022/061098 | | TCTVSGGSISSYYWSWIRQP | | RASQSVSSSYLAWYQQKPGQAPR |
| | | PGKGLEWIGYIYYSGITHYN | | LLIYGASSRATGIPDRFSGSGSG |
| | | PSLKSRVTISVDTSKIQFSL | | TDFTLTISRLEPEDFAVYYCQQY |
| | | KLSSVTAADTAVYYCARWGV | | GSSPWTFGQGTKVEIK |
| | | RRDYYYYGMDVWGQGTTVTV | | |
| | | SS | | |
| WO2017/180913 | 63 | QVQLQESGPGLVKPSQTLSL | 85 | DIVLTQSPASLAVSPGQRATITC |
| | | TCTVSGFSLSDYGVHWVRQP | | RASESVEYYVTSLMQWYQQKPGQ |
| | | PGKGLEWLGVIWAGGGTNYN | | PPKLLIFAASNVESGVPARFSGS |
| | | PSLKSRKTISKDTSKNQVSL | | GSGTDFTLTINPVEANDVANYYC |
| | | KLSSVTAADTAVYYCARDKG | | QQSRKVPYTFGQGTKLEIK |
| | | YSYYYSMDYWGQGTTVTVSS | | |
| US20220089736 | 64 | QVQLQQPGSELVRPGASVKL | 76 | DIQMTQSPASLSASVGETVTITC |
| | | SCKASGYTETSYWMHWVKQR | | GASENIYGSLNWYQRKQGKSPQL |
| | | HGQGLEWIGNIYPGSGRTNY | | LIYGATNLADGMSSRFSGSGSGR |
| | | DEKFKSKGTLTVDTSSRTAY | | QFSLKISSLHPDDVATYYCQNLL |
| | | MHLSSLTSEDSAVYYCTREH | | STPYTFGGGTKLELK |
| | | YGSNSHPMDYWGQGTSVTVS | | |
| | | S | | |
| US20200181260 | 65 | QVQLQQSGTELVKPASSVKI | 75 | DIQLTQSPAFLSASLGETVSIEC |
| | | SCKASGYTSTSNYMHWIRQQ | | LGSEDIYGYLAWYQQKPGKSPQL |
| | | PGNGLEWIGRIYPGNGNTKY | | LIYVANRLQDGVPSRFSGSGSGT |
| | | NQKEDGKATPTADKSSSTAY | | QYSLKISGMQPEDEGDYYCLQGS |
| | | MQLSRLTFEDSAVYFCASAP | | KFPLTFGSGTKLEIKR |
| | | LDYGGHIMDAWGQGTTVTVS | | |
| | | S | | |
| US20220089736 | 66 | QVQLQQWGAGLLKPSETLSL | 89 | EIVMTQSPATLSVSPGERATLSC |
| | | ICAVYGGSFSGYYWSWIRQP | | RASQSVRSNLDWYQQKPGQAPRE |
| | | PGKGLEWIGDINHSGNTNEN | | LIYGASTRATGIPARFSGSGSGT |
| | | PSLKSRVTISVDTSKNQFSL | | EFTLTISSLQSEDFAVYYCQQYN |
| | | KLSSVTAADTAVYYCAKGEA | | NWPLTFGGGTKVEIK |
| | | WEDPWGQGIMVTVSS | | |
| US20220089736 | 67 | QVQLQQWGAGLLKPSETLSL | 88 | EIVMTQSPATLSVSPGERATLSC |
| | | TCAVYGGSFSGYYWNWIRQP | | RASQSVNSNLAWYQQKPGQAPRL |
| | | PGKGLQWIGEINHSGTTNYN | | LIYGASARATGIPARFSGSGSGT |
| | | PSLKSRVTISVDTSKNQFSL | | EFTLTISSLQSEDFAVYYCQQYY |
| | | KLSSLTAADTAVYYCARGEA | | NWPLTFGGGTKVEIK |
| | | VALYWGQGTLVTVSS | | |
| WO | 68 | QVQLVESGGGVVQPGRSLRL | 82 | DIQMTQSPSSISASVGDRVTITC |
| 2022/061098 | | SCAASGFTFSSYGMHWVRQA | | RASQSISSYLNWYQQKPGKAPKL |
| | | PGKGLEWVAVISYAGNNKYY | | LIYAASSLQSGVPSRFSGSGSGT |
| | | ADSVKGRFTVSRDNSKKTLY | | DFTLTISSLQPEDFATYYCQQSY |
| | | LQMNSLRSEDTAVYYCAKDS | | STPPITFGQGTRLEIK |
| | | YYDELTDPDVLDIWGQGTMV | | |
| | | TVSS | | |
| WO | 68 | QVQLVESGGGVVQPGRSLRL | 87 | EIVLTQSPGTLSLSPGERATLSC |
| 2022/061098 | | SCAASGFTESSYGMHWVRQA | | RASQSVSSSYLAWYQQKPGQAPR |
| | | PGKGLEWVAVISYAGNNKYY | | LLIYGASSRATGIPDRFSGSGSG |
| | | ADSVKGRFTVSRDNSKKTLY | | TQFTLTISRLEPEDFAVYYCQQY |
| | | LQMNSLRSEDTAVYYCAKDS | | GSSPWTFGQGTKVEIK |
| | | YYDFLTDPDVLDIWGQGTMV | | |
| | | TVSS | | |
| US | 69 | QVQLVQSGAEVKKPGASVKV | 78 | DIQMTQSPSSLSASVGDRVTITC |
| 8,586,386 | | SCKASGYTETSYYIHWVRQA | | HASQNIYVWLNWYQQKPGKAPKL |
| | | PGQGLEWIGCIYPGNVNTNY | | LIYKASNLHTGVPSRFSGSGSGT |
| | | NEKFKDRATLTVDTSISTAY | | DETLTISSLQPEDFATYYCQQGQ |
| | | MELSRLRSDDTAVYFCTRSH | | TYPYTFGGGTKVEIK |
| | | YGLDWNFDVWGQGTTVTVSS | | |
| US | 70 | QVQLVQSGAEVKKPGASVKV | 78 | DIQMTQSPSSLSASVGDRVTITC |
| 7,939,638 | | SCKASGYTFTSYYIHWVRQA | | HASQNIYVWLNWYQQKPGKAPKL |
| | | PGQGLEWIGSIYPGNVNTNY | | LIYKASNLHTGVPSRFSGSGSGT |
| | | NEKEKDRATLTVDTSISTAY | | DFTLTISSLQPEDEATYYCQQGQ |
| | | MELSRLRSDDTAVYFCTRSH | | TYPYTFGGGTKVEIK |
| | | YGLDWNFDVWGQGTTVTVSS | | |
| WO2017/180913 | 71 | QVQLVQSGAEVVKPGASVKV | 80 | DIQMTQSPSSLSASVGDRVTITC |
| | | SCKASGYTFTSYYIHWVRQA | | QASQNIYVWLNWYQQKPGKAPKL |
| | | PGQGLEWIGSIYPGNVNTNY | | LIYKASNLHTGVPSRFSGSGSGT |
| | | AQKFQGRATLTVDTSISTAY | | DETLTISSLQPEDIATYYCQQGQ |
| | | MELSRLRSDDTAVYYCTRSH | | TYPYTFGQGTKLEIK |
| | | YGLDWNFDVWGKGTTVTVSS | | |
| WO | 72 | QVQLVQSGGGVVQPGQSLRL | 84 | DIQMTQSPSTLSASVGDRVTITC |
| 2022/096536 | | SCAASGFSISDYGVHWVRQA | | RASESVEYYVTSLMQWYQQKPGK |
| | | PGKGLEWLGVIWAGGGTNYN | | APKLLIFAASNVESGVPSRFSGS |
| | | SALQSRFTISKDNSKNTVYL | | GSGTEFTLTISSLQPDDEATYYC |
| | | QMNSLRAEDTAVYYCARDKG | | QQSRKVPYTFGQGTKLEIK |
| | | YSYYYSMDYWGQGTTVTVSS | | |
| US7723482 | 73 | VKLQQSGAELVKPGASVRLS | 77 | DIQMTQSPASLSVSVGETVTITC |
| | | CKASGYTFTEYIIHWIKLRS | | RTNENIYSNLAWYQQKQGKSPQL |
| | | GQGLEWIGWFYPGSNDIQYN | | LIYAATHLVEGVPSRESGSGSGT |
| | | AKFKGKATLTADKSSSTVYM | | QYSLKITSLQSEDEGNYYCQHEW |
| | | ELTGLTSEDSAVYFCARRDD | | GTPCTEGGGTKLEIKRT |
| | | FSGYDALPYWGQGTMVTVSS | | |
| US | 74 | VQLQQSGAELKKPGASVKVS | 79 | DIQMTQSPSSLSASVGDRVTITC |
| 8,785,604 | | CKASGYTFTEYIIHWIKLRS | | KTNENIYSNLAWYQQKDGKSPQL |
| | | GQGLEWIGWFYPGSNDIQYN | | LIYAATHLVEGVPSRFSGSGSGT |
| | | AQFKGKATLTADKSSSTVYM | | QYSLTISSLQPEDEGNYYCQHFW |
| | | ELTGLTPEDSAVYFCARRDD | | GTPXTFGGGTKLEIKR |
| | | FSGYDALPYWGQGTLVTVSA | | |

TABLE 3
Anti-CD28 Antibody VHCDR Sequences According to Kabat

| | SEQ | | SEQ | | SEQ | |
|---|---|---|---|---|---|---|
| Source | ID NO | VHCDR1 | ID NO | VHCDR2 | ID NO | VHCDR3 |
| U.S. Pat. No. 7,538,196B2 | 95 | RASESVEYY | 105 | AASNVES | 133 | QQSRKVPYT |
| | | VTSIMQ | | | | |
| US20220089736 | 98 | SYAMS | 106 | AISGSGGRTY | 130 | GAGYWYFDL |
| | | | | YADSVKG | | |
| US20220119530 | 98 | SYAMS | 116 | TISGSGDSTY | 135 | SGPGLRQVG |
| | | | | YADSVKG | | EDY |
| US20220119530 | 103 | SYYMS | 115 | TISESGDSTY | 135 | SGPGLRQVG |
| | | | | YADSVKG | | EDY |
| WO 2022/061098 | 96 | RNNMH | 110 | GISSNGGRTY | 125 | DDELLSFDY |
| | | | | YADSVKG | | |
| WO 2022/061098 | 96 | RNNMH | 110 | GISSNGGRTY | 125 | DDELLSFDY |
| | | | | YADSVKG | | |
| US 2004/0116675 | 100 | SYGVH | 120 | VIWPGGGTNF | 127 | DRAYGNYLY |
| | | | | NSALMS | | AMDY |
| WO 2022/061098 | 104 | SYYWS | 123 | YIYYSGITHY | 137 | WGVRRDYYY |
| | | | | NPSLKS | | YGMDV |
| WO2017/180913 | 91 | DYGVH | 118 | VIWAGGGTNY | 126 | DKGYSYYYS |
| | | | | NPSLKS | | MDY |
| US20220089736 | 101 | SYWMH | 111 | NIYPGSGRTN | 129 | EHYGSNSHP |
| | | | | YDEKEKS | | MDY |
| US20200181260 | 97 | SNYMH | 112 | RIYPGNGNTK | 124 | APLDYGGHI |
| | | | | YNQKFDG | | MDA |
| US20220089736 | 94 | GYYWS | 108 | DINHSGNTNF | 132 | GEAWFDP |
| | | | | NPSLKS | | |
| US20220089736 | 93 | GYYWN | 109 | EINHSGTTNY | 131 | GEAVALY |
| | | | | NPSLKS | | |
| WO 2022/061098 | 99 | SYGMH | 117 | VISYAGNNKY | 128 | DSYYDELTD |
| | | | | YADSVKG | | PDVLDI |
| WO 2022/061098 | 99 | SYGMH | 117 | VISYAGNNKY | 128 | DSYYDELTD |
| | | | | YADSVKG | | PDVLDI |
| U.S. Pat. No. 8,586,386 | 102 | SYYIH | 107 | CIYPGNVNTN | 136 | SHYGLDWNF |
| | | | | YNEKFKD | | DV |
| U.S. Pat. No. 7,939,638 | 102 | SYYIH | 114 | SIYPGNVNTN | 136 | SHYGLDWNF |
| | | | | YNEKFKD | | DV |
| WO2017/180913 | 102 | SYYIH | 113 | SIYPGNVNTN | 136 | SHYGLDWNF |
| | | | | YAQKFQG | | DV |
| WO 2022/096536 | 91 | DYGVH | 119 | VIWAGGGTNY | 126 | DKGYSYYYS |
| | | | | NSALQS | | MDY |
| U.S. Pat. No. 7,723,482 | 92 | EYIIH | 121 | WFYPGSNDIQ | 134 | RDDFSGYDA |
| | | | | YNAKFKG | | LPY |
| U.S. Pat. No. 8,785,604 | 92 | EYIIH | 122 | WFYPGSNDIQ | 134 | RDDESGYDA |
| | | | | YNAQFKG | | LPY |

TABLE 4
Anti-CD28 Antibody VLCDR Sequences According to Kabat

| | SEQ | | SEQ | | SEQ | |
|---|---|---|---|---|---|---|
| Source | ID NO | VLCDR1 | ID NO | VLCDR2 | ID NO | VLCDR3 |
| U.S. Pat. No. 7,538,196B2 | 138 | DYGVH | 164 | VIWAGGGTNY | 165 | DKGYSYYYS |
| | | | | NSALMS | | MDY |
| US20220089736 | 151 | RASQSVSSY | 157 | DASNRAT | 171 | QQRSNWPLT |
| | | LA | | | | |
| US20220119530 | 147 | RASQSISSY | 155 | AASSLOS | 175 | QQVYSTPFT |
| | | LN | | | | |
| US20220119530 | 147 | RASQSISSY | 155 | AASSLOS | 175 | QQVYSTPFT |
| | | LN | | | | |
| WO 2022/061098 | 147 | RASQSISSY | 155 | AASSLOS | 174 | QQSYSTPPI |
| | | LN | | | | T |
| WO 2022/061098 | 150 | RASQSVSSS | 159 | GASSRAT | 176 | QQYGSSPWT |
| | | YLA | | | | |
| US 2004/0116675 | 146 | RASESVEYY | 153 | AASNVDS | 172 | QQSRKVPFT |
| | | VTSLMQ | | | | |
| WO 2022/061098 | 150 | RASQSVSSS | 159 | GASSRAT | 176 | QQYGSSPWT |
| | | YLA | | | | |
| WO2017/180913 | 146 | RASESVEYY | 154 | AASNVES | 173 | QQSRKVPYT |
| | | VTSLMQ | | | | |
| US20220089736 | 139 | GASENIYGS | 161 | GATNLAD | 169 | QNLLSTPYT |
| | | LN | | | | |
| US20200181260 | 142 | LGSEDIYGY | 163 | VANRLQD | 166 | LQGSKFPLT |
| | | LA | | | | |
| US20220089736 | 149 | RASQSVRSN | 160 | GASTRAT | 177 | QQYNNWPLT |
| | | LD | | | | |
| US20220089736 | 148 | RASQSVNSN | 158 | GASARAT | 178 | QQYYNWPLT |
| | | LA | | | | |
| WO 2022/061098 | 147 | RASQSISSY | 155 | AASSLOS | 174 | QQSYSTPPI |
| | | LN | | | | T |
| WO 2022/061098 | 150 | RASQSVSSS | 159 | GASSRAT | 176 | QQYGSSPWT |
| | | YLA | | | | |
| U.S. Pat. No. 8,586,386 | 140 | HASQNIYVW | 162 | KASNLHT | 170 | QQGQTYPYT |
| | | LN | | | | |
| U.S. Pat. No. 7,939,638 | 140 | HASQNIYVW | 162 | KASNLHT | 170 | QQGQTYPYT |
| | | LN | | | | |
| WO2017/180913 | 143 | QASQNIYVW | 162 | KASNLHT | 170 | QQGQTYPYT |
| | | LN | | | | |
| WO 2022/096536 | 146 | RASESVEYY | 154 | AASNVES | 173 | QQSRKVPYT |
| | | VTSLMQ | | | | |
| U.S. Pat. No. 7,723,482 | 152 | RTNENIYSN | 156 | AATHLVE | 167 | QHFWGTPCT |
| | | LA | | | | |
| U.S. Pat. No. 8,785,604 | 141 | KTNENIYSN | 156 | AATHLVE | 168 | QHFWGTPXT |
| | | LA | | | | |

TABLE 5
Anti-CD28 Single Domain Antibody Sequences

| | SEQ ID | |
|---|---|---|
| Source | NO | Variable Domain |
| WO2021/155071 | 179 | EVQLVQSGGALVQAGGSLRLSCVAPRTIGLISLMGWYRQAPGKQREL |
| (sdVH) | | VATIIASGRINYAESVKDRFTISRDNAENKVYLQVNSLKPEDTAVYY |
| | | CRVNAETAPWSRRGFWGQGTQVTVKP |
| WO2021/155071 | 180 | QVQLQQSGGGLVQAGGSLRLSCVASGISENVVDMDWHRQAPGKEREL |
| (sdVH) | | VAGITRGGATYYADSVKGRFTISRDNAKNSAYLQLANLKPEDTAVYY |
| | | CNARWPSRYQNYWGQGTQVTVKP |
| WO2021/155071 | 181 | EVQLVQSGGGLVQTGGSIRLSCAASGRMESNYAMGWFRQAPGKEREF |
| (sdVH) | | VAAINYRRDSADYADSVKGRETISRDNAKNTVYLEMNSLKPEDTATY |
| | | YCGFTYAGWASSRRDDYNYWGQGTQVTVKP |
| U.S. Pat. No. | 182 | DIQMTQSPSSLSASVGDRVTITCRASRPIWPFLEWYQQKPGKAPKLL |
| 8,168,759 | | IYFTSRLRHGVPSRESGSGSGTCFTITISSLQPEDEATYYCLQNVAN |
| (sdVL) | | PATFSQGTKVEIKR |

TABLE 6
Anti-CD28 Single Domain Antibody CDR Sequences According to
Kabat

| | SEQ | | SEQ | | SEQ | |
|---|---|---|---|---|---|---|
| Source | ID NO | CDR1 | ID NO | CDR2 | ID NO | CDR3 |
| WO2021/155071 | 183 | ISLMG | 187 | TIIASGRT | 191 | NAETAPWSR |
| (sd VH) | | | | NYAESVKD | | RGF |
| WO2021/155071 | 184 | VVDMD | 188 | GITRGGATYY | 192 | RWPSRYQNY |
| (sd VH) | | | | ADSVKG | | |
| WO2021/155071 | 185 | NYAMG | 189 | AINYRRDSAD | 193 | TYAGWASSR |
| (sd VH) | | | | YADSVKG | | RDDYNY |
| U.S. Pat. No. | 186 | RASRPI | 190 | FTSRLRH | 194 | LQNVANPAT |
| 8,168,759 | | | | | | |
| (sd VL) | | WPELE | | | | |

TABLE 7
4-1BBL Sequences

| SEQ | | |
|---|---|---|
| ID NO | Description | Sequence |
| 196 | 4-1BBL “58” | ARASPGSAASPRLREGPELSPDDPAGLLDLRQGMFAQLVAQNVLLIDGP |
| | | LSWYSDPGLAGVSLTGGLSYKEDTKELVVAKAGVYYVFFQLELRRVVAG |
| | | EGSGSVSLALHLQPLRSAAGAAALALTVDLPPASSEARNSAFGEQGRLL |
| | | HLSAGQRLGVHLHTEARARHAWQLTQGATVLGLFRVTPEIPAGLPSPRS |
| | | E |
| 197 | 4-1BBL “71” | REGPELSPDDPAGLLDLRQGMFAQLVAQNVLLIDGPLSWYSDPGLAGVS |
| | | LTGGLSYKEDTKELVVAKAGVYYVFFQLELRRVVAGEGSGSVSLALHLQ |
| | | PLRSAAGAAALALTVDLPPASSEARNSAFGFQGRLLHLSAGQRLGVHLH |
| | | TEARARHAWQLTQGATVLGLFRVTPEIPAGLPSPRSE |
| 198 | 4-1BBL “58” | ARASPGSAASPRLREGPELSPDDPAGLLDLRQGMFAQLVAQNVLLIDGP |
| | trimer with | LSWYSDPGLAGVSLTGGLSYKEDTKELVVAKAGVYYVFFQLELRRVVAG |
| | GS15 linker | EGSGSVSLALHLQPLRSAAGAAALALTVDLPPASSEARNSAFGFQGRIL |
| | | HLSAGQRLGVHLHTEARARHAWQLTQGATVLGLFRVTPEIPAGLPSPRS |
| | | EGGGGSGGGGSGGGGSARASPGSAASPRLREGPELSPDDPAGLLDLRQG |
| | | MFAQLVAQNVLLIDGPLSWYSDPGLAGVSLTGGLSYKEDTKELVVAKAG |
| | | VYYVFFQLELRRVVAGEGSGSVSLALHLQPLRSAAGAAALALTVDLPPA |
| | | SSEARNSAFGFQGRLLHLSAGQRLGVHLHTEARARHAWQLTQGATVLGL |
| | | FRVTPEIPAGLPSPRSEGGGGSGGGGSGGGGSARASPGSAASPRIREGP |
| | | ELSPDDPAGLLDLRQGMFAQLVAQNVLLIDGPLSWYSDPGLAGVSLTGG |
| | | LSYKEDTKELVVAKAGVYYVFFQLELRRVVAGEGSGSVSLALHLQPLRS |
| | | AAGAAALALTVDLPPASSEARNSAFGFQGRLLHLSAGQRLGVHLHTEAR |
| | | ARHAWQLTQGATVLGLFRVTPEIPAGLPSPRSE |
| 199 | 4-1BBL “71” | REGPELSPDDPAGLLDLRQGMFAQLVAQNVLLIDGPLSWYSDPGLAGVS |
| | trimer with | LTGGLSYKEDTKELVVAKAGVYYVFFQLELRRVVAGEGSGSVSLALHLQ |
| | GS15 linker | PLRSAAGAAALALTVDLPPASSEARNSAFGFQGRLLHLSAGQRLGVHLH |
| | | TEARARHAWQLTQGATVLGLFRVTPEIPAGLPSPRSEGGGGSGGGGSGG |
| | | GGSREGPELSPDDPAGLLDERQGMFAQLVAQNVLLIDGPLSWYSDPGLA |
| | | GVSLTGGLSYKEDTKELVVAKAGVYYVFFQLELRRVVAGEGSGSVSLAL |
| | | HLQPLRSAAGAAALALTVDLPPASSEARNSAFGFQGRLLHLSAGQRLGV |
| | | HLHTEARARHAWQLTQGATVLGLFRVTPEIPAGLPSPRSEGGGGSGGGG |
| | | SGGGGSREGPELSPDDPAGLLDLRQGMFAQLVAQNVLLIDGPLSWYSDP |
| | | GLAGVSLTGGLSYKEDTKELVVAKAGVYYVFFQLELRRVVAGEGSGSVS |
| | | LALHLQPLRSAAGAAALALTVDLPPASSEARNSAFGFQGRLLHLSAGQR |
| | | LGVHLHTEARARHAWQLTQGATVLGLFRVTPEIPAGLPSPRSE |
| 200 | 4-1BBL “58” | ARASPGSAASPRIREGPELSPDDPAGLIDLRQGMFAQLVAQNVILIDGP |
| | trimer with | LSWYSDPGLAGVSLTGGLSYKEDTKELVVAKAGVYYVFFQLELRRVVAG |
| | GS10 linker | EGSGSVSLALHLQPLRSAAGAAALALTVDLPPASSEARNSAFGFQGRLL |
| | | HLSAGQRLGVHLHTEARARHAWQLTQGATVLGLFRVTPEIPAGLPSPRS |
| | | EGGGGSGGGGSARASPGSAASPRLREGPELSPDDPAGLLDLRQGMFAQL |
| | | VAQNVLLIDGPLSWYSDPGLAGVSITGGLSYKEDTKELVVAKAGVYYVE |
| | | FQLELRRVVAGEGSGSVSLALHLQPLRSAAGAAALALTVDLPPASSEAR |
| | | NSAFGFQGRLLHLSAGQRLGVHLHTEARARHAWQLTQGATVLGLFRVTP |
| | | EIPAGLPSPRSEGGGGSGGGGSARASPGSAASPRLREGPELSPDDPAGL |
| | | LDLRQGMFAQLVAQNVLLIDGPLSWYSDPGLAGVSITGGLSYKEDTKEL |
| | | VVAKAGVYYVFFQLELRRVVAGEGSGSVSLALHLQPLRSAAGAAALALT |
| | | VDLPPASSEARNSAFGFQGRLLHLSAGQRLGVHLHTEARARHAWQLTQG |
| | | ATVLGLFRVTPEIPAGLPSPRSE |
| 201 | 4-1BBL “71” | REGPELSPDDPAGLLDLRQGMFAQLVAQNVLLIDGPLSWYSDPGLAGVS |
| | trimer with | LTGGLSYKEDTKELVVAKAGVYYVFFQLELRRVVAGEGSGSVSLALHLQ |
| | GS10 linker | PLRSAAGAAALALTVDLPPASSEARNSAFGFQGRLLHLSAGQRLGVHLH |
| | | TEARARHAWQLTQGATVLGLFRVTPEIPAGLPSPRSEGGGGSGGGGSRE |
| | | GPELSPDDPAGLLDLRQGMFAQLVAQNVLLIDGPLSWYSDPGLAGVSLT |
| | | GGLSYKEDTKELVVAKAGVYYVFFQLELRRVVAGEGSGSVSLALHLQPL |
| | | RSAAGAAALALTVDLPPASSEARNSAFGFQGRLLHLSAGQRLGVHLHTE |
| | | ARARHAWQLTQGATVLGLFRVTPEIPAGLPSPRSEGGGGSGGGGSREGP |
| | | ELSPDDPAGLLDLRQGMFAQLVAQNVLLIDGPLSWYSDPGLAGVSLTGG |
| | | LSYKEDTKELVVAKAGVYYVFFQLELRRVVAGEGSGSVSLALHLQPLRS |
| | | AAGAAALALTVDLPPASSEARNSAFGFQGRLLHLSAGQRLGVHLHTEAR |
| | | ARHAWQLTQGATVLGLFRVTPEIPAGLPSPRSE |
| 202 | J*15 + 4- | QEDERIVEVDNKCKCARITSRIIRSSEDPNEDIVERNIRIIVPLNNREN |
| | 1BBL “58” | ISDPTSPLRTRFVYHLSDLCKKCDPTEVELDNQIVTATQSNICDEDSAT |
| | trimer with | ETCATYDRNKCYTAVVPLVYGGETKMVETALTPDACYPDGGGGSGGGGS |
| | GS15 linker | GGGGSARASPGSAASPRLREGPELSPDDPAGLLDLRQGMFAQLVAQNVL |
| | | LIDGPLSWYSDPGLAGVSLTGGLSYKEDTKELVVAKAGVYYVFFQLELR |
| | | RVVAGEGSGSVSLALHLQPERSAAGAAALALTVDLPPASSEARNSAFGE |
| | | QGRLLHLSAGQRLGVHLHTEARARHAWQLTQGATVLGLFRVTPEIPAGL |
| | | PSPRSEGGGGSGGGGSGGGGSARASPGSAASPRLREGPELSPDDPAGLL |
| | | DLRQGMFAQLVAQNVLLIDGPLSWYSDPGLAGVSLTGGLSYKEDTKELV |
| | | VAKAGVYYVFFQLELRRVVAGEGSGSVSLALHLQPERSAAGAAALALTV |
| | | DLPPASSEARNSAFGFQGRLLHLSAGQRLGVHLHTEARARHAWQLTQGA |
| | | TVLGLFRVTPEIPAGLPSPRSEGGGGSGGGGSGGGGSARASPGSAASPR |
| | | LREGPELSPDDPAGLLDLRQGMFAQLVAQNVLLIDGPLSWYSDPGLAGV |
| | | SLTGGLSYKEDTKELVVAKAGVYYVFFQLELRRVVAGEGSGSVSLALHL |
| | | QPLRSAAGAAALALTVDLPPASSEARNSAFGFQGRLLHLSAGQRLGVHL |
| | | HTEARARHAWQLTQGATVLGLFRVTPEIPAGLPSPRSE |
| 203 | 4-1BBL “58” | ARASPGSAASPRLREGPELSPDDPAGLLDLRQGMFAQLVAQNVLLIDGP |
| | trimer with | LSWYSDPGLAGVSLTGGLSYKEDTKELVVAKAGVYYVFFQLELRRVVAG |
| | GS15 linker | EGSGSVSIALHLQPLRSAAGAAALALTVDLPPASSEARNSAFGFQGRIL |
| | + J*15 | HLSAGQRLGVHLHTEARARHAWQLTQGATVLGLFRVTPEIPAGLPSPRS |
| | | EGGGGSGGGGSGGGGSARASPGSAASPRLREGPELSPDDPAGLLDLRQG |
| | | MFAQLVAQNVLLIDGPLSWYSDPGLAGVSLTGGLSYKEDTKELVVAKAG |
| | | VYYVFFQLELRRVVAGEGSGSVSLALHLQPLRSAAGAAALALTVDLPPA |
| | | SSEARNSAFGEQGRLLHLSAGQRLGVHLHTEARARHAWQLTQGATVLGL |
| | | FRVTPEIPAGLPSPRSEGGGGSGGGGSGGGGSARASPGSAASPRIREGP |
| | | ELSPDDPAGLLDLRQGMFAQLVAQNVLLIDGPLSWYSDPGLAGVSLTGG |
| | | LSYKEDTKELVVAKAGVYYVFFQLELRRVVAGEGSGSVSLALHLQPLRS |
| | | AAGAAALALTVDLPPASSEARNSAFGFQGRLLHLSAGQRLGVHLHTEAR |
| | | ARHAWQLTQGATVLGLFRVTPEIPAGLPSPRSEGGGGSGGGGSGGGGSQ |
| | | EDERIVLVDNKCKCARITSRIIRSSEDPNEDIVERNIRIIVPLNNRENI |
| | | SDPTSPLRTRFVYHLSDLCKKCDPTEVELDNQIVTATQSNICDEDSATE |
| | | TCATYDRNKCYTAVVPLVYGGETKMVETALTPDACYPD |
| 204 | J*15+ 4-1BBL | QEDERIVEVDNKCKCARITSRIIRSSEDPNEDIVERNIRIIVPLNNREN |
| | “71” trimer | ISDPTSPLRTRFVYHLSDLCKKCDPTEVELDNQIVTATQSNICDEDSAT |
| | with GS15 | ETCATYDRNKCYTAVVPLVYGGETKMVETALTPDACYPDGGGGSGGGGS |
| | linker | GGGGSREGPELSPDDPAGLLDLRQGMFAQLVAQNVLLIDGPLSWYSDPG |
| | | LAGVSLTGGLSYKEDTKELVVAKAGVYYVFFQLELRRVVAGEGSGSVSL |
| | | ALHLQPLRSAAGAAALALTVDLPPASSEARNSAFGFQGRLLHLSAGQRL |
| | | GVHLHTEARARHAWQLTQGATVLGLFRVTPEIPAGLPSPRSEGGGGSGG |
| | | GGSGGGGSREGPELSPDDPAGLLDLRQGMFAQLVAQNVLLIDGPLSWYS |
| | | DPGLAGVSLTGGLSYKEDTKELVVAKAGVYYVFFQLELRRVVAGEGSGS |
| | | VSLALHLQPLRSAAGAAALALTVDLPPASSEARNSAFGFQGRLLHLSAG |
| | | QRLGVHLHTEARARHAWQLTQGATVLGLFRVTPEIPAGLPSPRSEGGGG |
| | | SGGGGSGGGGSREGPELSPDDPAGLLDLRQGMFAQLVAQNVLLIDGPLS |
| | | WYSDPGLAGVSITGGLSYKEDTKELVVAKAGVYYVEFQLELRRVVAGEG |
| | | SGSVSLALHLQPLRSAAGAAALALTVDIPPASSEARNSAFGFQGRLLHL |
| | | SAGQRLGVHLHTEARARHAWQLTQGATVLGLFRVTPEIPAGLPSPRSE |
| 205 | 4-1BBL “71” | REGPELSPDDPAGLLDLRQGMFAQLVAQNVLLIDGPLSWYSDPGLAGVS |
| | trimer with | LTGGLSYKEDTKELVVAKAGVYYVFFQLELRRVVAGEGSGSVSLALHLQ |
| | GS15 linker | PLRSAAGAAALALTVDLPPASSEARNSAFGFQGRLLHLSAGQRLGVHLH |
| | + J*15 | TEARARHAWQLTQGATVLGLFRVTPEIPAGLPSPRSEGGGGSGGGGSGG |
| | | GGSREGPELSPDDPAGLLDLRQGMFAQLVAQNVLLIDGPLSWYSDPGLA |
| | | GVSLIGGISYKEDTKELVVAKAGVYYVFFQLELRRVVAGEGSGSVSLAL |
| | | HLQPLRSAAGAAALALTVDLPPASSEARNSAFGFQGRLLHLSAGQRLGV |
| | | HLHTEARARHAWQLTQGATVLGLERVTPEIPAGLPSPRSEGGGGSGGGG |
| | | SGGGGSREGPELSPDDPAGLLDLRQGMFAQLVAQNVLLIDGPLSWYSDP |
| | | GLAGVSLTGGLSYKEDTKELVVAKAGVYYVFFQLELRRVVAGEGSGSVS |
| | | LALHLQPLRSAAGAAALALTVDLPPASSEARNSAFGEQGRLLHLSAGQR |
| | | LGVHLHTEARARHAWQLTQGATVLGLFRVTPEIPAGLPSPRSEGGGGSG |
| | | GGGSGGGGSQEDERIVLVDNKCKCARITSRIIRSSEDPNEDIVERNIRI |
| | | IVPLNNRENISDPTSPLRTRFVYHLSDLCKKCDPTEVELDNQIVTATQS |
| | | NICDEDSATETCATYDRNKCYTAVVPLVYGGETKMVETALTPDACYPD |
| 206 | 4-1BBL “71” | REGPELSPDDPAGLLDLRQGMFAQLVAQNVLLIDGPLSWYSDPGLAGVS |
| | trimer with | LTGGLSYKEDTKELVVAKAGVYYVFFQLELRRVVAGEGSGSVSLALHLQ |
| | GS10 linker | PLRSAAGAAALALTVDLPPASSEARNSAFGFQGRLLHLSAGQRLGVHLH |
| | + J*10 | TEARARHAWQLTQGATVLGLFRVTPEIPAGLPSPRSEGGGGSGGGGSRE |
| | | GPELSPDDPAGLLDLRQGMFAQLVAQNVLLIDGPLSWYSDPGLAGVSLT |
| | | GGLSYKEDTKELVVAKAGVYYVFFQLELRRVVAGEGSGSVSLALHLQPL |
| | | RSAAGAAALALTVDLPPASSEARNSAFGFQGRLLHLSAGQRLGVHLHTE |
| | | ARARHAWQLTQGATVLGLFRVTPEIPAGLPSPRSEGGGGSGGGGSREGP |
| | | ELSPDDPAGLLDLRQGMFAQLVAQNVLLIDGPLSWYSDPGLAGVSLTGG |
| | | LSYKEDTKELVVAKAGVYYVFFQLELRRVVAGEGSGSVSLALHLQPLRS |
| | | AAGAAALALTVDLPPASSEARNSAFGFQGRLLHLSAGQRLGVHLHTEAR |
| | | ARHAWQLTQGATVLGLERVTPEIPAGLPSPRSEGGGGSGGGGSQEDERI |
| | | VLVDNKCKCARITSRIIRSSEDPNEDIVERNIRIIVPLNNRENISDPTS |
| | | PLRTRFVYHLSDLCKKCDPTEVELDNQIVTATQSNICDEDSATETCATY |
| | | DRNKCYTAVVPLVYGGETKMVETALTPDACYPD |
| 207 | J*10 + 4- | QEDERIVIVDNKCKCARITSRIIRSSEDPNEDIVERNIRIIVPLNNREN |
| | 1BBL “71” | ISDPTSPIRTRFVYHISDLCKKCDPTEVELDNQIVTATQSNICDEDSAT |
| | trimer with | ETCATYDRNKCYTAVVPLVYGGETKMVETALTPDACYPDGGGGSGGGGS |
| | GS10 linker | REGPELSPDDPAGLLDLRQGMFAQLVAQNVLLIDGPLSWYSDPGLAGVS |
| | | LTGGLSYKEDTKELVVAKAGVYYVFFQLELRRVVAGEGSGSVSLALHLQ |
| | | PLRSAAGAAALALTVDLPPASSEARNSAFGFQGRLLHLSAGQRLGVHLH |
| | | TEARARHAWQLTQGATVLGLFRVTPEIPAGLPSPRSEGGGGSGGGGSRE |
| | | GPELSPDDPAGLLDLRQGMFAQLVAQNVLLIDGPLSWYSDPGLAGVSLT |
| | | GGLSYKEDTKELVVAKAGVYYVFFQLELRRVVAGEGSGSVSLALHLQPL |
| | | RSAAGAAALALTVDLPPASSEARNSAFGFQGRLLHLSAGQRLGVHLHTE |
| | | ARARHAWQLTQGATVLGLFRVTPEIPAGLPSPRSEGGGGSGGGGSREGP |
| | | ELSPDDPAGLLDLRQGMFAQLVAQNVLLIDGPLSWYSDPGLAGVSLTGG |
| | | LSYKEDTKELVVAKAGVYYVFFQLELRRVVAGEGSGSVSLALHLQPLRS |
| | | AAGAAALALTVDLPPASSEARNSAFGFQGRLLHLSAGQRLGVHLHTEAR |
| | | ARHAWQLTQGATVLGLERVTPEIPAGLPSPRSE |
| 208 | J*15 + 4- | QEDERIVEVDNKCKCARITSRIIRSSEDPNEDIVERNIRIIVPINNREN |
| | 1BBL “71” | ISDPTSPLRTREVYHLSDLCKKCDPTEVELDNQIVTATQSNICDEDSAT |
| | trimer with | ETCATYDRNKCYTAVVPLVYGGETKMVETALTPDACYPDGGGGSGGGGS |
| | GS10 linker | GGGGSREGPELSPDDPAGLLDLRQGMFAQLVAQNVLLIDGPLSWYSDPG |
| | | LAGVSLTGGLSYKEDTKELVVAKAGVYYVFFQLELRRVVAGEGSGSVSL |
| | | ALHLQPLRSAAGAAALALTVDLPPASSEARNSAFGFQGRILHLSAGQRL |
| | | GVHLHTEARARHAWQLTQGATVLGLFRVTPEIPAGLPSPRSEGGGGSGG |
| | | GGSREGPELSPDDPAGLLDLRQGMFAQLVAQNVLLIDGPLSWYSDPGLA |
| | | GVSLTGGLSYKEDTKELVVAKAGVYYVFFQLELRRVVAGEGSGSVSLAL |
| | | HLQPLRSAAGAAALALTVDLPPASSEARNSAFGFQGRLLHLSAGQRLGV |
| | | HLHTEARARHAWQLTQGATVLGLERVTPEIPAGLPSPRSEGGGGSGGGG |
| | | SREGPELSPDDPAGLLDLRQGMFAQLVAQNVLLIDGPLSWYSDPGLAGV |
| | | SLTGGLSYKEDTKELVVAKAGVYYVFFQLELRRVVAGEGSGSVSLALHL |
| | | QPLRSAAGAAALALTVDLPPASSEARNSAFGFQGRLLHLSAGQRLGVHL |
| | | HTEARARHAWQLTQGATVLGLFRVTPEIPAGLPSPRSE |
| 209 | 4-1BBL “71” | REGPELSPDDPAGLLDLRQGMFAQLVAQNVLLIDGPLSWYSDPGLAGVS |
| | trimer with | LTGGLSYKEDTKELVVAKAGVYYVFFQLELRRVVAGEGSGSVSLALHLQ |
| | GS10 linker | PLRSAAGAAALALTVDLPPASSEARNSAFGFQGRLLHLSAGQRLGVHLH |
| | + J*15 | TEARARHAWQLTQGATVLGLFRVTPEIPAGLPSPRSEGGGGSGGGGSRE |
| | | GPELSPDDPAGLLDLRQGMFAQLVAQNVLLIDGPLSWYSDPGLAGVSIT |
| | | GGLSYKEDTKELVVAKAGVYYVFFQLELRRVVAGEGSGSVSLALHLQPL |
| | | RSAAGAAALALTVDLPPASSEARNSAFGFQGRLLHLSAGQRLGVHLHTE |
| | | ARARHAWQLTQGATVLGLFRVTPEIPAGLPSPRSEGGGGSGGGGSREGP |
| | | ELSPDDPAGLLDLRQGMFAQLVAQNVLLIDGPLSWYSDPGLAGVSLTGG |
| | | LSYKEDTKELVVAKAGVYYVFFQLELRRVVAGEGSGSVSLALHLQPLRS |
| | | AAGAAALALTVDLPPASSEARNSAFGFQGRLLHLSAGQRLGVHLHTEAR |
| | | ARHAWQLTQGATVLGLFRVTPEIPAGLPSPRSEGGGGSGGGGSGGGGSQ |
| | | EDERIVLVDNKCKCARITSRIIRSSEDPNEDIVERNIRIIVPLNNRENI |
| | | SDPTSPLRTREVYHLSDLCKKCDPTEVELDNQIVTATQSNICDEDSATE |
| | | TCATYDRNKCYTAVVPLVYGGETKMVETALTPDACYPD |
| 210 | 4-1BBL “58” | ARASPGSAASPRLREGPELSPDDPAGLLDLRQGMFAQLVAQNVLLIDGP |
| | with GS15 + | LSWYSDPGLAGVSLTGGLSYKEDTKELVVAKAGVYYVFFQLELRRVVAG |
| | J*15 + 4- | EGSGSVSIALHLQPLRSAAGAAALALTVDLPPASSEARNSAFGFQGRIL |
| | 1BBL “58” | HLSAGQRLGVHLHTEARARHAWQLTQGATVLGLFRVTPEIPAGLPSPRS |
| | with GS15 | EGGGGSGGGGSGGGGSARASPGSAASPRLREGPELSPDDPAGLLDLRQG |
| | | MFAQLVAQNVLLIDGPLSWYSDPGLAGVSLTGGLSYKEDTKELVVAKAG |
| | | VYYVFFQLELRRVVAGEGSGSVSLALHLQPLRSAAGAAALALTVDLPPA |
| | | SSEARNSAFGEQGRLLHLSAGQRLGVHLHTEARARHAWQLTQGATVLGL |
| | | FRVTPEIPAGLPSPRSEGGGGSGGGGSGGGGSARASPGSAASPRLREGP |
| | | ELSPDDPAGLLDLRQGMFAQLVAQNVLLIDGPLSWYSDPGLAGVSLTGG |
| | | LSYKEDTKELVVAKAGVYYVFFQLELRRVVAGEGSGSVSLALHLQPLRS |
| | | AAGAAALALTVDLPPASSEARNSAFGFQGRLLHLSAGQRLGVHLHTEAR |
| | | ARHAWQLTQGATVLGLFRVTPEIPAGLPSPRSEGGGGSGGGGSGGGGSQ |
| | | EDERIVLVDNKCKCARITSRIIRSSEDPNEDIVERNIRIIVPLNNRENI |
| | | SDPTSPLRTRFVYHLSDLCKKCDPTEVELDNQIVTATQSNICDEDSATE |
| | | TCATYDRNKCYTAVVPLVYGGETKMVETALTPDACYPDGGGGSGGGGSG |
| | | GGGSARASPGSAASPRLREGPELSPDDPAGLLDLRQGMFAQLVAQNVLL |
| | | IDGPLSWYSDPGLAGVSLTGGLSYKEDTKELVVAKAGVYYVFFQLELRR |
| | | VVAGEGSGSVSLALHLQPLRSAAGAAALALTVDLPPASSEARNSAFGEQ |
| | | GRLLHLSAGQRLGVHLHTEARARHAWQLTQGATVLGLFRVTPEIPAGLP |
| | | SPRSEGGGGSGGGGSGGGGSARASPGSAASPRLREGPELSPDDPAGLLD |
| | | LRQGMFAQLVAQNVLLIDGPLSWYSDPGLAGVSLTGGLSYKEDTKELVV |
| | | AKAGVYYVFFQLELRRVVAGEGSGSVSLALHLQPLRSAAGAAALALTVD |
| | | LPPASSEARNSAFGFQGRLLHLSAGQRLGVHLHTEARARHAWQLTQGAT |
| | | VLGLFRVTPEIPAGLPSPRSEGGGGSGGGGSGGGGSARASPGSAASPRL |
| | | REGPELSPDDPAGLLDLRQGMFAQLVAQNVLLIDGPLSWYSDPGLAGVS |
| | | LTGGLSYKEDTKELVVAKAGVYYVFFQLELRRVVAGEGSGSVSLALHLQ |
| | | PLRSAAGAAALALTVDLPPASSEARNSAFGFQGRLLHLSAGQRLGVHLH |
| | | TEARARHAWQLTQGATVLGLFRVTPEIPAGLPSPRSE |
| 211 | 4-1BBL “71” | REGPELSPDDPAGLLDLRQGMFAQLVAQNVLLIDGPLSWYSDPGLAGVS |
| | with GS15 + | LTGGLSYKEDTKELVVAKAGVYYVFFQLELRRVVAGEGSGSVSLALHLQ |
| | J*15 + 4- | PLRSAAGAAALALTVDLPPASSEARNSAFGFQGRLLHLSAGQRLGVHLH |
| | 1BBL “71” | TEARARHAWQLTQGATVLGLFRVTPEIPAGLPSPRSEGGGGSGGGGSGG |
| | with GS15 | GGSREGPELSPDDPAGLLDLRQGMFAQLVAQNVLLIDGPLSWYSDPGLA |
| | | GVSLTGGLSYKEDTKELVVAKAGVYYVFFQLELRRVVAGEGSGSVSLAL |
| | | HLQPLRSAAGAAALALTVDLPPASSEARNSAFGFQGRLLHLSAGQRLGV |
| | | HLHTEARARHAWQLTQGATVLGLFRVTPEIPAGLPSPRSEGGGGSGGGG |
| | | SGGGGSREGPELSPDDPAGLLDLRQGMFAQLVAQNVLLIDGPLSWYSDP |
| | | GLAGVSLTGGLSYKEDTKELVVAKAGVYYVFFQLELRRVVAGEGSGSVS |
| | | LALHLQPLRSAAGAAALALTVDLPPASSEARNSAFGFQGRLLHLSAGQR |
| | | LGVHLHTEARARHAWQLTQGATVLGLFRVTPEIPAGLPSPRSEGGGGSG |
| | | GGGSGGGGSQEDERIVLVDNKCKCARITSRIIRSSEDPNEDIVERNIRI |
| | | IVPLNNRENISDPTSPLRTRFVYHLSDLCKKCDPTEVELDNQIVTATQS |
| | | NICDEDSATETCATYDRNKCYTAVVPLVYGGETKMVETALTPDACYPDG |
| | | GGGSGGGGSGGGGSREGPELSPDDPAGLLDLRQGMFAQLVAQNVLLIDG |
| | | PLSWYSDPGLAGVSLTGGLSYKEDTKELVVAKAGVYYVFFQLELRRVVA |
| | | GEGSGSVSLALHLQPIRSAAGAAALALTVDLPPASSEARNSAFGFQGRL |
| | | LHLSAGQRLGVHLHTEARARHAWQLTQGATVLGLFRVTPEIPAGLPSPR |
| | | SEGGGGSGGGGSGGGGSREGPELSPDDPAGLLDLRQGMFAQLVAQNVLL |
| | | IDGPLSWYSDPGLAGVSLTGGLSYKEDTKELVVAKAGVYYVFFQLELRR |
| | | VVAGEGSGSVSIALHLQPLRSAAGAAALALTVDLPPASSEARNSAFGFQ |
| | | GRLLHLSAGQRIGVHLHTEARARHAWQLTQGATVLGLFRVTPEIPAGLP |
| | | SPRSEGGGGSGGGGSGGGGSREGPELSPDDPAGLLDLRQGMFAQLVAQN |
| | | VLLIDGPLSWYSDPGLAGVSLTGGLSYKEDTKELVVAKAGVYYVFFQLE |
| | | LRRVVAGEGSGSVSLALHLQPLRSAAGAAALALTVDLPPASSEARNSAF |
| | | GFQGRLLHLSAGQRLGVALHTEARARHAWQLTQGATVLGLFRVTPEIPA |
| | | GLPSPRSE |
| 212 | antiCD3 AA + | EVQLLESGGGLVQPGGSLRLSCAASGFTFDTYAMNWVRQAPGKGLEWVA |
| | J*15 | RIRSKYNNYATYYADSVKDRFTISRDDSKSTLYLQMESLRAEDTAVYYC |
| | | VRHANFGAGYVSWFAHWGQGTLVTVSSGGGGSGGGGSGGGGSQTVVTQE |
| | | PSLSVSPGGTVTLTCGSSTGAVTTSNYANWVQQTPGQAPRGLIGGTDKR |
| | | APGVPDRESGSLLGDKAALTITGAQAEDEADYYCALWYSNHWVEGGGTK |
| | | LTVLGGGGSGGGGSGGGGSQEDERIVLVDNKCKCARITSRIIRSSEDPN |
| | | EDIVERNIRIIVPLNNRENISDPTSPLRTRFVYHLSDLCKKCDPTEVEL |
| | | DNQIVTATQSNICDEDSATETCATYDRNKCYTAVVPLVYGGETKMVETA |
| | | LTPDACYPD |
| 213 | antiCD3 AA + | EVQLLESGGGLVQPGGSLRISCAASGETFDTYAMNWVRQAPGKGLEWVA |
| | J*15 +4-1BLL | RIRSKYNNYATYYADSVKDRFTISRDDSKSTLYLQMESLRAEDTAVYYC |
| | “71” with | VRHANFGAGYVSWFAHWGQGTLVTVSSGGGGSGGGGSGGGGSQTVVTQE |
| | GS15 | PSLSVSPGGTVTLTCGSSTGAVTTSNYANWVQQTPGQAPRGLIGGTDKR |
| | | APGVPDRESGSLLGDKAALTITGAQAEDEADYYCALWYSNHWVFGGGTK |
| | | LTVLGGGGSGGGGSGGGGSQEDERIVLVDNKCKCARITSRIIRSSEDPN |
| | | EDIVERNIRIIVPLNNRENISDPTSPLRTRFVYHLSDLCKKCDPTEVEL |
| | | DNQIVTATQSNICDEDSATETCATYDRNKCYTAVVPLVYGGETKMVETA |
| | | LTPDACYPDGGGGSGGGGSGGGGSREGPELSPDDPAGLLDLRQGMFAQL |
| | | VAQNVLLIDGPLSWYSDPGLAGVSLTGGLSYKEDTKELVVAKAGVYYVF |
| | | FQLELRRVVAGEGSGSVSLALHLQPLRSAAGAAALALTVDLPPASSEAR |
| | | NSAFGFQGRLLHLSAGQRLGVHLHTEARARHAWQLTQGATVLGLFRVTP |
| | | EIPAGLPSPRSEGGGGSGGGGSGGGGSREGPELSPDDPAGLLDLRQGMF |
| | | AQLVAQNVLLIDGPLSWYSDPGLAGVSLTGGLSYKEDTKELVVAKAGVY |
| | | YVFFQLELRRVVAGEGSGSVSLALHLQPLRSAAGAAALALTVDLPPASS |
| | | EARNSAFGFQGRLLHLSAGQRLGVHLHTEARARHAWQLTQGATVLGLER |
| | | VTPEIPAGIPSPRSEGGGGSGGGGSGGGGSREGPELSPDDPAGLLDLRQ |
| | | GMFAQLVAQNVLLIDGPLSWYSDPGLAGVSLTGGLSYKEDTKELVVAKA |
| | | GVYYVFFQLELRRVVAGEGSGSVSLALHLQPLRSAAGAAALALTVDLPP |
| | | ASSEARNSAFGFQGRLLHLSAGQRLGVHLHTEARARHAWQLTQGATVLG |
| | | LFRVTPEIPAGLPSPRSE |
| 214 | 4-1BLL “71” | REGPELSPDDPAGLLDLRQGMFAQLVAQNVLLIDGPLSWYSDPGLAGVS |
| | with GS15 + | LTGGLSYKEDTKELVVAKAGVYYVFFQLELRRVVAGEGSGSVSLALHLQ |
| | J*15 + | PLRSAAGAAALALTVDLPPASSEARNSAFGFQGRLLHLSAGQRLGVHLH |
| | antiCD3 AA | TEARARHAWQLTQGATVLGLFRVTPEIPAGLPSPRSEGGGGSGGGGSGG |
| | | GGSREGPELSPDDPAGLLDLRQGMFAQLVAQNVLLIDGPLSWYSDPGLA |
| | | GVSLTGGLSYKEDTKELVVAKAGVYYVFFQLELRRVVAGEGSGSVSLAL |
| | | HLQPLRSAAGAAALALTVDLPPASSEARNSAFGFQGRLLHLSAGQRLGV |
| | | HLHTEARARHAWQLTQGATVLGLERVTPEIPAGLPSPRSEGGGGSGGGG |
| | | SGGGGSREGPELSPDDPAGLLDLRQGMFAQLVAQNVLLIDGPLSWYSDP |
| | | GLAGVSLTGGLSYKEDTKELVVAKAGVYYVFFQLELRRVVAGEGSGSVS |
| | | LALHLQPERSAAGAAALALTVDLPPASSEARNSAFGFQGRLLHLSAGQR |
| | | LGVHLHTEARARHAWQLTQGATVLGLFRVTPEIPAGLPSPRSEGGGGSG |
| | | GGGSGGGGSQEDERIVLVDNKCKCARITSRIIRSSEDPNEDIVERNIRI |
| | | IVPINNRENISDPTSPLRTREVYHLSDICKKCDPTEVELDNQIVTATQS |
| | | NICDEDSATETCATYDRNKCYTAVVPLVYGGETKMVETALTPDACYPDG |
| | | GGGSGGGGSGGGGSEVQLLESGGGLVQPGGSLRLSCAASGFTEDTYAMN |
| | | WVRQAPGKGLEWVARIRSKYNNYATYYADSVKDRFTISRDDSKSTLYLQ |
| | | MESLRAEDTAVYYCVRHANFGAGYVSWFAHWGQGTLVTVSSGGGGSGGG |
| | | GSGGGGSQTVVTQEPSLSVSPGGTVTLTCGSSTGAVTTSNYANWVQQTP |
| | | GQAPRGLIGGTDKRAPGVPDRESGSLLGDKAALTITGAQAEDEADYYCA |
| | | LWYSNHWVFGGGTKLTVL |

TABLE 8
Anti-CD3 Antibody VH and VL Sequences

| | SEQ | | SEQ | |
|---|---|---|---|---|
| | ID | | ID | |
| Citation | NO | VH | NC | VL |
| Visilizumab | 16 | QVQLVQSGAEVKKPGASVKVSCK | 20 | DIQMTQSPSSISASVGDRVTIT |
| U.S. Pat. No. | | ASGYTFISYTMHWVRQAPGQGLE | | CSASSSVSYMNWYQQKPGKAPK |
| 5,834,597A | | WMGYINPRSGYTHYNQKLKDKAT | | RLIYDTSKLASGVPSRESGSGS |
| | | LTADKSASTAYMELSSLRSEDTA | | GTDFTLTISSLQPEDFATYYCQ |
| | | VYYCARSAYYDYDGFAYWGQGTL | | QWSSNPPTFGGGTKVEIK |
| | | VTVSS | | |
| WO2018208864 | 24 | EVQLLESGGGLVQPGGSLRISCA | 28 | QTVVTQEPSLSVSPGGTVTLTC |
| | | ASGETEDTYAMNWVRQAPGKGLE | | GSSTGAVTTSNYANWVQQTPGQ |
| | | WVARIRSKYNNYATYYADSVKDR | | APRGLIGGTDKRAPGVPDRESG |
| | | FTISRDDSKSTLYLQMESLRAED | | SILGDKAALTITGAQAEDEADY |
| | | TAVYYCVRHANFGAGYVSWEAHW | | YCALWYSNHWVFGGGTKLTVL |
| | | GQGTLVTVSS | | |
| WO2018208864 | 24 | EVQLLESGGGLVQPGGSLRISCA | 32 | QTVVTQEPSLSVSPGGTVTLTC |
| | | ASGFTFDTYAMNWVRQAPGKGLE | | GSSTGAVTTSNYANWVQQTPGQ |
| | | WVARIRSKYNNYATYYADSVKDR | | APRGLIGGTDKRAPGVPDRESG |
| | | FTISRDDSKSTLYLQMESLRAED | | SLLGDKAALTITGAQAEDEADY |
| | | TAVYYCVRHANFGAGYVSWFAHW | | YCALWYSDLWVEGGGTKLTVL |
| | | GQGTLVTVSS | | |
| WO2018208864 | 34 | QVQLVQSGAEVKKPGASVKVSCK | 38 | DIVMTQSPDSLAVSLGERATIN |
| | | ASGFNIKDYYMHWVRQAPGQRLE | | CKSSQSLLNARTGKNYLAWYQQ |
| | | WMGWIDLENANTIYDAKFQGRVT | | KPGQPPKLLIYWASTRESGVPD |
| | | ITRDTSASTAYMELSSLRSEDTA | | RESGSGSGTDFTLTISSLQAED |
| | | VYYCARDAYGRYFYDVWGQGTLV | | VAVYYCKQSYSRRTEGGGTKVE |
| | | TVSS | | IK |
| WO2018208864 | 42 | QVQLVQSGAEVKKPGASVKVSCK | 44 | DIVMTQSPDSLAVSIGERATIN |
| | | ASGENIKDYYMHWVRQAPGQRLE | | CKSSQSLLNARTGKNYLAWYQQ |
| | | WIGWIDLENANTVYDAKFQGRVT | | KPGQPPKLLIYWASTRESGVPD |
| | | ITRDTSASTAYMELSSLRSEDTA | | RFSGSGSGTDFTLTISSLQAED |
| | | VYYCARDAYGRYFYDVWGQGTLV | | VAVYYCKQSYFRRTFGGGTKVE |
| | | TVSS | | IK |
| WO2018208864 | 46 | QVQLVQSGAEVKKPGASVKVSCK | 48 | DIVMTQSPDSLAVSLGERATIN |
| | | ASGFNIKDYYMHWVRQAPGQRLE | | CKSSQSLLNARTGKNYLAWYQQ |
| | | WIGWIDLENANTVYDAKFQGRVT | | KPGQPPKLLIYWASTRESGVPD |
| | | ITRDTSASTAYMELSSLRSEDTA | | RESGSGSGTDFTLTISSLQAED |
| | | VYYCARDAYGQYFYDVWGQGTLV | | VAVYYCTQSYFRRTFGGGTKVE |
| | | TVSS | | IK |
| WO2015095392 | 50 | EVQLVESGGGLVQPKGSLKLSCA | 52 | QAVVTQESALTTSPGETVTLTC |
| | | ASGFTENTYAMNWVRQAPGKGLE | | RSSTGAVTTSNYANWVQEKPDH |
| | | WVARIRSKYNNYATYYADSVKDR | | LETGLIGGINKRAPGVPARFSG |
| | | FTISRDDSQSILYLQMNNLKTED | | SLIGDKAALTITGAQTEDEAIY |
| | | TAMYYCVRHGNEGNSYVSWFAYW | | FCALWYSNLWVFGGGTKLTVL |
| | | GQGTLVTVSS | | |

TABLE 9
Anti-CD3 Antibody VHCDR Sequences According to Kabat

| | SEQ | | SEQ | | SEQ | |
|---|---|---|---|---|---|---|
| | ID | | ID | | ID | |
| Citation | NO | VHCDR1 | NO | VHCDR2 | NO | VHCDR3 |
| Visilizumab | 17 | SYTMH | 18 | YINPRSGYTHYNQKLKD | 19 | SAYYDYDGFAY |
| U.S. Pat. No. | | | | | | |
| 5,834,597A | | | | | | |
| WO2018208864 | 25 | TYAMN | 26 | RIRSKYNNYATYYADSVKD | 27 | HANFGAGYVSWFAH |
| WO2018208864 | 25 | TYAMN | 26 | RIRSKYNNYATYYADSVKD | 27 | HANFGAGYVSWFAH |
| WO2018208864 | 35 | DYYMH | 36 | WIDLENANTIYDAKFQG | 37 | DAYGRYFYDV |
| WO2018208864 | 35 | DYYMH | 43 | WIDLENANTVYDAKFQG | 37 | DAYGRYFYDV |
| WO2018208864 | 35 | DYYMH | 43 | WIDLENANTVYDAKFQG | 47 | DAYGQYFYDV |
| WO2015095392 | 25 | TYAMN | 26 | RIRSKYNNYATYYADSVKD | 51 | HGNFGNSYVSWFAY |

TABLE 10
Anti-CD3 Antibody VLCDR Sequences According to Kabat

| | SEQ | | SEQ | | SEQ | |
|---|---|---|---|---|---|---|
| | ID | | ID | | ID | |
| Citation | NO | VLCDR1 | NO | VLCDR2 | NO | VLCDR3 |
| Visilizumab | 21 | SASSSVSYMN | 22 | DTSKLAS | 23 | QQWSSNPPT |
| U.S. Pat. No. | | | | | | |
| 5,834,597A | | | | | | |
| WO2018208864 | 29 | GSSTGAVTTSNYAN | 30 | GTDKRAP | 31 | ALWYSNHWV |
| WO2018208864 | 29 | GSSTGAVTTSNYAN | 30 | GTDKRAP | 33 | ALWYSDLWV |
| WO2018208864 | 39 | KSSQSLLNARTGKNYLA | 40 | WASTRES | 41 | KQSYSRRT |
| WO2018208864 | 39 | KSSQSLLNARTGKNYLA | 40 | WASTRES | 45 | KQSYFRRT |
| WO2018208864 | 39 | KSSQSLINARTGKNYLA | 40 | WASTRES | 49 | TQSYFRRT |
| WO2015095392 | 53 | RSSTGAVTTSNYAN | 54 | GTNKRAP | 55 | ALWYSNLWV |

While a variety of different multimeric binding molecules can be contemplated by a person of ordinary skill in the art based on this disclosure, and as such are included in this disclosure, in certain embodiments, a binding molecule as described above is provided in which each binding unit comprises two IgA or IgM heavy chains each comprising a VH situated amino terminal to the IgA or IgM constant region or multimerizing fragment thereof, and two immunoglobulin light chains each comprising a VL situated amino terminal to an immunoglobulin light chain constant region.

Moreover, in certain embodiments, at least one binding unit of the binding molecule, or at least two, at least three, at least four, at least five, or at least six binding units of the binding molecule, comprises or comprise two of the target antigen-binding domains as described above. In certain embodiments the two-target antigen-binding domains in the at least one binding unit of the binding molecule, or at least two, at least three, at least four, at least five, or at least six binding units of the binding molecule, can be different from each other, or they can be identical.

In certain embodiments, the two IgA or IgM heavy chains within the at least one binding unit of the binding molecule, or at least two, at least three, at least four, at least five, or at least six binding units of the binding molecule, are identical.

In certain embodiments, the two light chains within the at least one binding unit of the binding molecule, or at least two, at least three, at least four, at least five, or at least six binding units of the binding molecule, are identical. In certain embodiments, two identical light chains within at least one binding unit, or within at least two, at least three, at least four, at least five, or at least six binding units of the binding molecule are kappa light chains, e.g., human kappa light chains, or lambda light chains, e.g., human lambda light chains.

### Tumor-Associated Target Antigens

In certain embodiments, this disclosure provides a multimeric binding molecule, e.g., an IgM, IgM-like, IgA, and IgA-like binding molecule comprising two, four, or ten binding units collectively comprising three, four, five, six, seven, eight, nine, or ten “target-antigen binding domains,” i.e., antigen-binding domains that specifically bind to a target antigen, e.g., a tumor-associated or tumor-specific target antigen. In certain embodiments the target antigen is a tumor-specific antigen, i.e., a target antigen that is largely expressed only on tumor or cancer cells, or that may be expressed only at undetectable levels in normal healthy cells of an adult. In certain embodiments the target is a tumor-associated antigen, i.e., a target antigen that is expressed on both healthy and cancerous cells but is expressed at much higher density on cancerous cells than on normal healthy cells. Exemplary tumor-specific and tumor-associated antigens include, without limitation, CD38, CD20, CD19, CD22, CD28, B cell maturation antigen (BCMA), CD123, PD-L1, tumor-associated calcium signal transducer 2 (TROP-2), mesothelin, Muc16, prostate-specific membrane antigen (PSMA), or Six-Transmembrane Epithelial Antigen of Prostate-1·(STEAP-1).

In some embodiments, the tumor-associated target antigen is CD20. CD20 (UniProt P11836) is a 36 kDa non-glycosylated; tetra-spanning membrane protein (MS4A1 gene product) expressed exclusively on B-lymphocytes and on more than 90% of B-lymphocytic lymphomas. CD20 is expressed at the late pre-B cell stage and is upregulated on most normal and malignant B lineage cells before it is downregulated in terminally differentiated plasma cells. This B-lymphocyte surface molecule is involved with development and differentiation of B cells into plasma cells. Exemplary binding domains that bind to CD20 are provided, e.g., in U.S. Pat. No. 10,787,520, which is incorporated herein by reference in its entirety.

In some embodiments, the tumor-associated target antigen is mesothelin (MSLN). Mesothelin is a 71 kDa cell surface GPI-linked glycoprotein (UniProt Q13421), with restricted normal tissue distribution (mesothelial cells in peritoneum, pericardium, pleura), but which is overexpressed in a wide variety of solid tumors. See, e.g., Ordonez, N. G. Am. J. Surg. Pathol. 27:1418-1428 (2003). A variety of mesothelin-targeting therapies are in preclinical and clinical development. Target antigen-binding domains that specifically bind to MSLN have been described, for example, in U.S. Pat. No. 5,489,525, U.S. Patent Application Publication Nos. US2017/0029502A1, and U.S. Pat. Nos. 10,851,175 B2; and 8,435,494 B2, which are incorporated herein by reference in their entireties. In certain embodiments, the target antigen-binding domain that specifically binds MSLN comprises a heavy chain variable region (VH) and a light chain variable region (VL), wherein the VH and VL comprise, respectively, the amino acid sequences SEQ ID NO: 256 and SEQ ID NO: 257, SEQ ID NO: 258 and SEQ ID NO: 259, SEQ ID NO: 260 and SEQ ID NO: 261, SEQ ID NO: 262 and SEQ ID NO: 263, or SEQ ID NO: 264 and SEQ ID NO: 265 (Table 25). In certain embodiments, the VH and VL comprise, respectively, the amino acid sequences SEQ ID NO: 262 and SEQ ID NO: 263.

In some embodiments, the tumor-associated target antigen is CD38. CD38 (UniProt P28907) is a type II transmembrane glycoprotein expressed at relatively low levels on normal myeloid and lymphoid cells, but overexpressed in various hematologic malignancies including chronic lymphocytic leukemia (CLL), multiple myeloma (MM), Hodgkin's lymphoma (HL), diffuse large B cell lymphoma (DLBCL), and peripheral T cell lymphoma (PTCL), and various solid tumors, including prostate cancer, non-small cell lung cancer (NSCLC), squamous cell carcinoma of the head and neck (SCCHN), ovarian cancer, and liver cancer and is therefore considered a potential target for directed therapeutics (Martin et al., Cells 8:1522 (2019)). Monoclonal antibodies that specifically bind to CD38, daratumumab, and isatuximab, have been approved for the treatment of multiple myeloma. Exemplary binding domains and multimeric binding molecules that specifically bind to CD38 are described, e.g., in International Publication No. WO 2023/150677 A2, which is incorporated herein by reference in its entirety.

In some embodiments the tumor-associated target antigen is tumor-associated calcium signal transducer 2 (TROP-2). TROP-2 (UniProt P09758) is a glycoprotein that that is highly expressed in a variety of epithelial cancers, including thyroid, lung, head and neck, stomach, kidney, uterine, colorectal, oral, pancreatic, urothelial, prostate, breast, cervical, endometrial and ovarian cancer. Lombardi, P. et al., Cancers 15:1744 (2023). As such, agents that target TROP-2, including monoclonal antibodies, are commercially available or are in preclinical and clinical development. Id. For example, an anti-TROP-2 antibody-drug conjugate, sacituzumab govitecan (TRODELVY®), was recently approved for the treatment of some patients with triple-negative breast cancer or metastatic urothelial cancer. Target antigen-binding domains that specifically bind to TROP-2 have been described, for example, in International Publication No. WO 1998/012227 A1, U.S. Pat. No. 9,849,176 B2, U.S. Patent Application Publication No. US2012/0237518 A1, U.S. Pat. No. 9,850,312 B2, U.S. Patent Application Publication No. US2008/0213267 A1, and U.S. Pat. No. 9,399,074 B2, which are incorporated herein by reference in their entireties. In certain embodiments the target antigen-binding domain that specifically binds TROP-2 comprises a heavy chain variable region (VH) and a light chain variable region (VL), wherein the VH and VL comprise, respectively, the amino acid sequences SEQ ID NO: 244 and SEQ ID NO: 245, SEQ ID NO: 246 and SEQ ID NO: 247, SEQ ID NO: 248 and SEQ ID NO: 249, SEQ ID NO: 250 and SEQ ID NO: 251, SEQ ID NO: 252 and SEQ ID NO: 253, or SEQ ID NO: 254 and SEQ ID NO: 255 (Table 24). In certain embodiments, the VH and VL comprise, respectively, the amino acid sequences SEQ ID NO: 248 and SEQ ID NO: 249.

In some embodiments, a multimeric binding molecule as provided herein conditionally activates T cells in the presence of the tumor-associated or tumor-specific target antigen. More specifically in certain embodiments, a multimeric binding molecule as provided herein only activates T cells in the presence of the tumor-associated or tumor-specific target antigen. In certain embodiments, a multimeric binding molecule as provided herein provides efficient T cell activation with moderate cytokine release, even when the T cell costimulatory molecule is CD28, and anti-CD28 binding domain on the J chain has superagonist activity. In certain embodiments, a multimeric binding molecule as provided herein can improve the ability of cytotoxic T lymphocytes (CTLs) to infiltrate the tumor microenvironment of solid tumors and effect tumor cell killing. The skilled person will understand that tumor-specific and tumor-associated target antigens appear in the literature by a number of different names, but that these well-known therapeutic targets can be easily identified using databases available online, e.g., EXPASY.org.

### Methods of Use

The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include osteosarcoma, chondrosarcoma, fibrosarcoma, squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al. Br. J. Cancer 94:1057-1065 (2006)) and triple negative breast cancer (TNBC), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer including, but not limited to, squamous cell cancers, and cancers of mucinous origins, such as, mucinous ovarian cancer, cholangiocarcinoma (liver) and renal papillary carcinoma. Mucosal distribution, for example as provided by an IgA-based binding molecule as provided herein, could be beneficial for certain cancers, e.g., lung cancer, ovarian cancer, colorectal cancer, or squamous cell carcinoma.

Effective doses of compositions for treatment of cancer vary depending upon many different factors, including means of administration, target site, physiological state of the patient, whether the patient is human or an animal, other medications administered, and whether treatment is prophylactic or therapeutic. In certain embodiments the treatment methods provided herein can provide increased safety, in that the composition exhibits greater cytotoxicity (e.g., induces apoptosis to a greater extent) on cancer cells than on non-cancer cells, e.g., normal human hepatocytes. Usually, the patient is a human, but non-human mammals including transgenic mammals can also be treated. Treatment dosages can be titrated using routine methods known to those of skill in the art to optimize safety and efficacy.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

The subject to be treated can be any animal, e.g., mammal, in need of treatment, in certain embodiments, subject is a human subject.

In its simplest form, a preparation to be administered to a subject comprises a multimeric binding molecule, such as a dimeric, tetrameric, or pentameric antibody as provided herein, administered in a conventional dosage form.

A multimeric binding molecule as provided herein can be administered by any suitable method as described elsewhere herein, e.g., via IV infusion. In certain embodiments, a multimeric binding molecule as provided herein is introduced into a tumor, or in the vicinity of a tumor cell.

All types of tumors are potentially amenable to treatment by this approach including, without limitation, carcinoma of the breast, lung, pancreas, ovary, kidney, colon, and bladder, as well as melanomas, sarcomas, and lymphomas. Mucosal distribution could be beneficial for certain cancers, e.g., lung cancer, ovarian cancer, colorectal cancer, or squamous cell carcinoma.

Accordingly, in some embodiments, the method provided herein is a method for inhibiting, delaying, or reducing malignant cell growth in a subject with cancer, where the cancer is a hematologic cancer or a solid tumor. In some embodiments, the cancer is a hematologic cancer, such as acute myeloid leukemia (AML), chronic myeloid leukemia (CML), acute lymphocytic leukemia (ALL), chronic lymphocytic leukemia, hairy cell leukemia, Hodgkin lymphoma, non-Hodgkin lymphoma, multiple myeloma, any metastases thereof, or any combination thereof. In some embodiments, the cancer is a solid tumor, such as bladder cancer, colorectal cancer, sarcoma (e.g., fibrosarcoma), gastric cancer, lung cancer (e.g., non-small cell lung cancer (NSCLC)), or pancreatic cancer.

### Pharmaceutical Compositions and Administration Methods

Methods of preparing and administering a multimeric binding molecule, such as a dimeric or pentameric binding molecule as provided herein to a subject in need thereof are known or are readily determined in view of this disclosure. The route of administration of a multimeric binding molecule can be, for example, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration is a solution for injection, in particular, for intravenous or intraarterial injection or drip. A suitable pharmaceutical composition can comprise a buffer (e.g., acetate, phosphate, or citrate buffer), a surfactant (e.g., polysorbate), optionally a stabilizer agent (e.g., human albumin), etc.

As discussed herein, a multimeric binding molecule, such as a dimeric or pentameric binding molecule as provided herein can be administered in a pharmaceutically effective amount for the in vivo treatment of cancers expressing a target antigen, where one or more target antigen-binding domains of the multimeric binding molecule bind the target antigen, e.g., a tumor-specific or tumor-associated target antigen. In this regard, it will be appreciated that the disclosed binding molecules and compounds can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives, and the like. A pharmaceutically effective amount of a multimeric binding molecule, such as a dimeric or pentameric binding molecule as provided herein means an amount sufficient to achieve effective binding to a target and to achieve a therapeutic benefit. A pharmaceutically effective amount of a cancer therapy as provided herein means an amount sufficient to achieve a therapeutic benefit. Suitable formulations are described in Remington: The Science and Practice of Pharmacy (Elsevier Science) 23rd ed. (2020).

In keeping with the scope of the present disclosure, a multimeric binding molecule as provided herein can be administered to a subject in need of therapy in an amount sufficient to produce a therapeutic effect. A multimeric binding molecule as provided herein can be administered to the subject in a conventional dosage form prepared by combining the multimeric binding molecule of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

The terms “therapeutically effective dose or amount” or “effective amount” denote amount of a multimeric binding molecule, that when administered brings about a positive therapeutic response with respect to treatment of a patient with cancer.

The amount of a multimeric binding molecule to be administered is readily determined by one of ordinary skill in the art without undue experimentation given this disclosure. Factors influencing the mode of administration and the respective amount of a multimeric binding molecule include, but are not limited to, the severity of the disease, the history of the disease, and the age, height, weight, health, and physical condition of the individual undergoing therapy. Similarly, the amount of a multimeric binding molecule to be administered will be dependent upon the mode of administration and whether the subject will undergo a single dose or multiple doses of this agent.

### Further Embodiments

Embodiment 1. A multimeric binding molecule comprising five, four, or two bivalent binding units and a modified J chain, wherein each binding unit comprises two antibody heavy chains, each comprising an IgM or IgA heavy chain constant region or multimerizing variant or fragment thereof each associated with a target antigen-binding domain, and wherein the modified J chain comprises (a) a J chain or a functional fragment or variant thereof (“J”), (b) an anti-CD3 scFv (“C”), and (c) a heterologous polypeptide (“H”), wherein H comprises a polypeptide agonist of a T cell costimulatory molecule, wherein the J, C, and H are associated as a fusion protein.

Embodiment 2. The multimeric binding molecule of embodiment 1, wherein the T cell costimulatory molecule comprises CD28 or 4-1BB.

Embodiment 3. The multimeric binding molecule of embodiment 1 or embodiment 2, wherein H comprises an anti-CD28 antibody or antigen-binding fragment thereof or a 4-1BB ligand (“4-1BBL”) trimer.

Embodiment 4. The multimeric binding molecule of any one of embodiments 1 to 3, wherein the modified J chain comprises, in the N-terminal to C-terminal direction, C-J-H or H-J-C.

Embodiment 5. The multimeric binding molecule of embodiment 4, wherein C, J, and H are fused via amino acid linkers which can be the same or different.

Embodiment 6. The multimeric binding molecule of embodiment 5, wherein

each linker consists, independently, of 5 to 25 amino acids.

Embodiment 7. The multimeric binding molecule of embodiment 6, wherein each linker consists, independently, of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235).

Embodiment 8. The multimeric binding molecule of embodiment 7, wherein the linker consists of GGGGSGGGGS (SEQ ID NO: 10).

Embodiment 9. The multimeric binding molecule of embodiment 7, wherein the linker consists of GGGGSGGGGSGGGGS (SEQ ID NO: 11).

Embodiment 10. The multimeric binding molecule of embodiment 7, wherein the linker consists of GKPGSGKPGSGKPGSGKPGS (SEQ ID NO: 14).

Embodiment 11. The multimeric binding molecule of any one of embodiments 3 to 10, wherein H comprises an antigen-binding fragment of an anti-CD28 antibody.

Embodiment 12. The multimeric binding molecule of embodiment 11, wherein the antigen-binding fragment of the anti-CD28 antibody comprises a single-chain Fv (scFv) fragment.

Embodiment 13. The multimeric binding molecule of embodiment 12, wherein the anti-CD28 scFv fragment comprises a heavy chain variable region (VH) and a light chain variable region (VL), wherein the VH comprises VH complementarity-determining regions VHCDR1, VHCDR2, and VHCDR3 and the VL comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 100, SEQ ID NO: 120, SEQ ID NO: 127, SEQ ID NO: 146, SEQ ID NO: 153, and SEQ ID NO: 172; SEQ ID NO: 95, SEQ ID NO: 105, SEQ ID NO: 133, SEQ ID NO: 138, SEQ ID NO: 164, and SEQ ID NO: 165; SEQ ID NO: 98, SEQ ID NO: 106, SEQ ID NO: 130, SEQ ID NO: 151, SEQ ID NO: 157, and SEQ ID NO: 171; SEQ ID NO: 98, SEQ ID NO: 116, SEQ ID NO: 135, SEQ ID NO: 147, SEQ ID NO: 155, and SEQ ID NO: 175; SEQ ID NO: 103, SEQ ID NO: 115, SEQ ID NO: 135, SEQ ID NO: 147, SEQ ID NO: 155, and SEQ ID NO: 175; SEQ ID NO: 96, SEQ ID NO: 110, SEQ ID NO: 125, SEQ ID NO: 147, SEQ ID NO: 155, and SEQ ID NO: 174; SEQ ID NO: 96, SEQ ID NO: 110, SEQ ID NO: 125, SEQ ID NO: 150, SEQ ID NO: 159, and SEQ ID NO: 176; SEQ ID NO: 104, SEQ ID NO: 123, SEQ ID NO: 137, SEQ ID NO: 150, SEQ ID NO: 159, and SEQ ID NO: 176; SEQ ID NO: 91, SEQ ID NO: 118, SEQ ID NO: 126, SEQ ID NO: 146, SEQ ID NO: 154, and SEQ ID NO: 173; SEQ ID NO: 101, SEQ ID NO: 111, SEQ ID NO: 129, SEQ ID NO: 139, SEQ ID NO: 161, and SEQ ID NO: 169; SEQ ID NO: 97, SEQ ID NO: 112, SEQ ID NO: 124, SEQ ID NO: 142, SEQ ID NO: 163, and SEQ ID NO: 166; SEQ ID NO: 94, SEQ ID NO: 108, SEQ ID NO: 132, SEQ ID NO: 149, SEQ ID NO: 160, and SEQ ID NO: 177; SEQ ID NO: 93, SEQ ID NO: 109, SEQ ID NO: 131, SEQ ID NO: 148, SEQ ID NO: 158, and SEQ ID NO: 178; SEQ ID NO: 99, SEQ ID NO: 117, SEQ ID NO: 128, SEQ ID NO: 147, SEQ ID NO: 155, and SEQ ID NO: 174; SEQ ID NO: 99, SEQ ID NO: 117, SEQ ID NO: 128, SEQ ID NO: 150, SEQ ID NO: 159, and SEQ ID NO: 176; SEQ ID NO: 102, SEQ ID NO: 107, SEQ ID NO: 136, SEQ ID NO: 140, SEQ ID NO: 162, and SEQ ID NO: 170; SEQ ID NO: 102,
SEQ ID NO: 114, SEQ ID NO: 136, SEQ ID NO: 140, SEQ ID NO: 162, and SEQ ID NO: 170; SEQ ID NO: 102, SEQ ID NO: 113, SEQ ID NO: 136, SEQ ID NO: 143, SEQ ID NO: 162, and SEQ ID NO: 170; SEQ ID NO: 91, SEQ ID NO: 119, SEQ ID NO: 126, SEQ ID NO: 146, SEQ ID NO: 154, and SEQ ID NO: 173; SEQ ID NO: 92, SEQ ID NO: 121, SEQ ID NO: 134, SEQ ID NO: 152, SEQ ID NO: 156, and SEQ ID NO: 167; or SEQ ID NO: 92, SEQ ID NO: 122, SEQ ID NO: 134, SEQ ID NO: 141, SEQ ID NO: 156, and SEQ ID NO: 168.

Embodiment 14. The multimeric binding molecule of embodiment 13, wherein the VH and VL of the anti-CD28 scFv comprise amino acid sequences at least 80%, 85%, 90%, 95%, or 100% identical to the amino acid sequences SEQ ID NO: 61 and SEQ ID NO: 81, SEQ ID NO: 56 and SEQ ID NO: 90, SEQ ID NO: 57 and SEQ ID NO: 86, SEQ ID NO: 58 and SEQ ID NO: 83, SEQ ID NO: 59 and SEQ ID NO: 83, SEQ ID NO: 60 and SEQ ID NO: 82, SEQ ID NO: 60 and SEQ ID NO: 87, SEQ ID NO: 62 and SEQ ID NO: 87, SEQ ID NO: 63 and SEQ ID NO: 85, SEQ ID NO: 64 and SEQ ID NO: 76, SEQ ID NO: 65 and SEQ ID NO: 75, SEQ ID NO: 66 and SEQ ID NO: 89, SEQ ID NO: 67 and SEQ ID NO: 88, SEQ ID NO: 68 and SEQ ID NO: 82, SEQ ID NO: 68 and SEQ ID NO: 87, SEQ ID NO: 69 and SEQ ID NO: 78, SEQ ID NO: 70 and SEQ ID NO: 78, SEQ ID NO: 71 and SEQ ID NO: 80, SEQ ID NO: 72 and SEQ ID NO: 84, SEQ ID NO: 73 and SEQ ID NO: 77, or SEQ ID NO: 74 and SEQ ID NO: 79, respectively.

Embodiment 15. The multimeric binding molecule of embodiment 13 or embodiment 14, wherein the VH and VL are fused via an amino acid linker.

Embodiment 16. The multimeric binding molecule of embodiment 15, wherein the linker consists of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGGGGG (SEQ ID NO: 235).

Embodiment 17. The multimeric binding molecule of embodiment 15, wherein the linker consists of GGGGSGGGGS (SEQ ID NO: 10).

Embodiment 18. The multimeric binding molecule of embodiment 15, wherein

the linker consists of GGGGSGGGGSGGGGS (SEQ ID NO: 11).

Embodiment 19. The multimeric binding molecule of embodiment 15, wherein the linker consists of GKPGSGKPGSGKPGSGKPGS (SEQ ID NO: 14).

Embodiment 20. The multimeric binding molecule of embodiment 11, wherein the antigen-binding fragment of an antibody is an anti-CD28 single domain heavy chain variable region (sdVH) or an anti-CD28 single domain light chain variable region (sdVL).

Embodiment 21. The multimeric binding molecule of embodiment 20, wherein the anti-CD28 sdVH comprises complementarity-determining regions CDR1, CDR2, and CDR3, wherein the CDR1, CDR2, and CDR3 comprise, respectively, the amino acid sequences SEQ ID NO: 183, SEQ ID NO: 187, and SEQ ID NO: 191; SEQ ID NO: 184, SEQ ID NO: 188, and SEQ ID NO: 192; or SEQ ID NO: 185, SEQ ID NO: 189, and SEQ ID NO: 193; or wherein the anti-CD28 sdVL comprises complementarity-determining regions CDR1, CDR2, and CDR3, wherein the CDR1, CDR2, and CDR3 comprise, respectively, the amino acid sequences SEQ ID NO: 186, SEQ ID NO: 190, and SEQ ID NO: 194.

Embodiment 22. The multimeric binding molecule of embodiment 21, wherein the anti-CD28 sdVH comprises an amino acid sequence at least 80%, 85%, 90%, 95%, or 100% identical to the amino acid sequence SEQ ID NO: 179, SEQ ID NO: 180, or SEQ ID NO: 181, or wherein the anti-CD28 sdVL comprises an amino acid sequence at least 80%, 85%, 90%, 95%, or 100% identical to the amino acid sequence SEQ ID NO: 182.

Embodiment 23. The multimeric binding molecule of any one of embodiments 3 to 10, wherein H comprises a 4-1BBL trimer.

Embodiment 24. The multimeric binding molecule of embodiment 23, wherein the 4-1BBL trimer comprises three 4-1BBL monomers.

Embodiment 25. The multimeric binding molecule of embodiment 24, wherein each 4-1BBL monomer is a soluble fragment of human 4-1BBL comprising amino acids X to 254 of SEQ ID NO: 15, wherein X is an integer from 50 to 99.

Embodiment 26. The multimeric binding molecule of embodiment 25, wherein the soluble fragments of 4-1BBL each comprise amino acids 71 to 254 of SEQ ID NO: 15 (SEQ ID NO: 197).

Embodiment 27. The multimeric binding molecule of embodiment 25 or embodiment 26, wherein the soluble fragments of 4-1BBL each comprise amino acids 58 to 254 of SEQ ID NO: 15 (SEQ ID NO: 196).

Embodiment 28. The multimeric binding molecule of any one of embodiments 24 to 27, wherein the 4-1BBL monomers are fused via amino acid linkers which can the same or different.

Embodiment 29. The multimeric binding molecule of embodiment 28, wherein each linker consists, independently, of 5 to 25 amino acids.

Embodiment 30. The multimeric binding molecule of embodiment 29, wherein each linker consists, independently, of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235).

Embodiment 31. The multimeric binding molecule of embodiment 30, wherein the linker consists of GGGGSGGGGS (SEQ ID NO: 10)

Embodiment 32. The multimeric binding molecule of embodiment 30, wherein the linker consists of GGGGSGGGGSGGGGS (SEQ ID NO: 11).

Embodiment 33. The multimeric binding molecule of embodiment 30, wherein the linker consists of GKPGSGKPGSGKPGSGKPGS (SEQ ID NO: 14).

Embodiment 34. The multimeric binding molecule of any one of embodiments 23 to 33, wherein H comprises the amino acid sequence SEQ ID NO: 198, SEQ ID NO: 199, SEQ ID NO: 200, or SEQ ID NO: 201.

Embodiment 35. The multimeric binding molecule of any one of embodiments 1 to 34, wherein the C comprises a heavy chain variable region (VH) and a light chain variable region (VL), wherein the VH of C comprises VH complementarity-determining regions VHCDR1, VHCDR2, and VHCDR3 and the VL of C comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 25, SEQ ID NO: 26, SEQ ID NO: 27, SEQ ID NO: 29, SEQ ID NO: 30, and SEQ ID NO: 31; SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; SEQ ID NO: 35, SEQ ID NO: 43, SEQ ID NO: 47, SEQ ID NO: 39, SEQ ID NO: 40, and SEQ ID NO: 49, or SEQ ID NO: 25, SEQ ID NO: 26, SEQ ID NO: 51, SEQ ID NO: 53, SEQ ID NO: 54, and SEQ ID NO: 55.

Embodiment 36. The multimeric binding molecule of embodiment 35, wherein the VHCDR1, VHCDR2, VHCDR3, VLCDR1, VLCDR2, and VLCDR3 of C comprise, respectively, SEQ ID NO: 25, SEQ ID NO: 26, SEQ ID NO: 27, SEQ ID NO: 29, SEQ ID NO: 30, and SEQ ID NO: 31.

Embodiment 37. The multimeric binding molecule of embodiment 35, wherein the VH and VL of C comprise an amino acid sequence at least 80%, 85%, 90%, 95%, or 100% identical to the amino acid sequence of SEQ ID NO: 24 and SEQ ID NO: 28; SEQ ID NO: 16 and SEQ ID NO: 20; SEQ ID NO: 24 and SEQ ID NO: 32; SEQ ID NO: 34 and SEQ ID NO: 38; SEQ ID NO: 42 and SEQ ID NO: 44; or SEQ ID NO: 46 and SEQ ID NO: 48, respectively.

Embodiment 38. The multimeric binding molecule of embodiment 37, wherein the C comprises the VH and VL amino acid sequences SEQ ID NO: 24 and SEQ ID NO: 28, respectively.

Embodiment 39. The multimeric binding molecule of any one of embodiments to 38, wherein the VH and VL are fused via an amino acid linker.

Embodiment 40. The multimeric binding molecule of embodiment 39, wherein the linker consists of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235).

Embodiment 41. The multimeric binding molecule of embodiment 40, wherein the linker consists of GGGGSGGGGS (SEQ ID NO: 10).

Embodiment 42. The multimeric binding molecule of embodiment 40, wherein the linker consists of GGGGSGGGGSGGGGS (SEQ ID NO: 11).

Embodiment 43. The multimeric binding molecule of embodiment 40, wherein the linker consists of GKPGSGKPGSGKPGSGKPGS (SEQ ID NO: 14).

Embodiment 44. The multimeric binding molecule of any one of embodiments 1 to 43, wherein J comprises SEQ ID NO: 7 or a functional fragment or variant thereof.

Embodiment 45. The multimeric binding molecule of any one of embodiments 1 to 44, wherein J comprises a variant J chain with an alanine substitution at the amino acid position corresponding to amino acid Y102 of the mature wild-type human J chain (SEQ ID NO: 7).

Embodiment 46. The multimeric binding molecule of any one of embodiments 1 to 45, wherein J comprises the amino acid sequence SEQ ID NO: 8 (“J*”).

Embodiment 47. The multimeric binding molecule of any one of embodiments 1 to 22 or 35 to 46, wherein the modified J chain comprises the amino acid sequence SEQ ID NO: 233, SEQ ID NO: 231, SEQ ID NO: 232, or SEQ ID NO: 234.

Embodiment 48. The multimeric binding molecule of any one of embodiments 1 to 10 or 23 to 46, wherein the modified J chain comprises the amino acid sequence SEQ ID NO: 213 or SEQ ID NO: 214.

Embodiment 49. A multimeric binding molecule comprising five, four, or two bivalent binding units and a modified J chain, wherein each binding unit comprises two antibody heavy chains, each comprising an IgM or IgA heavy chain constant region or multimerizing variant or fragment thereof each associated with a target antigen-binding domain, and wherein the modified J chain comprises a J chain or a functional fragment or variant thereof and a 4-1BBL trimer associated as a fusion protein, wherein the 4-1BBL trimer comprises three 4-1BBL monomers each comprising a soluble fragment of 4-1BBL.

Embodiment 50. The multimeric binding molecule of embodiment 49, wherein each 4-1BBL monomer is a soluble fragment of human 4-1BBL comprising amino acids X to 254 of SEQ ID NO: 15, wherein X is an integer from 50 to 99.

Embodiment 51. The multimeric binding molecule of embodiment 50, wherein the soluble fragments of 4-1BBL each comprise amino acids 71 to 254 of SEQ ID NO: 15 (SEQ ID NO: 197).

Embodiment 52. The multimeric binding molecule of embodiment 50 or embodiment 51, wherein the soluble fragments of 4-1BBL each comprise amino acids 58 to 254 of SEQ ID NO: 15 (SEQ ID NO: 196).

Embodiment 53. The multimeric binding molecule of any one of embodiments 49 to 52, wherein the 4-1BBL monomers are fused via amino acid linkers which can the same or different.

Embodiment 54. The multimeric binding molecule of embodiment 53, wherein each linker consists, independently, of 5 to 25 amino acids.

Embodiment 55. The multimeric binding molecule of embodiment 54, wherein each linker consists, independently, of the amino acid sequence (GGGGS)n (SEQ ID NO: 279), wherein n is an integer between 2 and 5, GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGSGGGG (SEQ ID NO: 235).

Embodiment 56. The multimeric binding molecule of embodiment 55, wherein the linker consists of GGGGSGGGGS (SEQ ID NO: 10).

Embodiment 57. The multimeric binding molecule of embodiment 55, wherein the linker consists of GGGGSGGGGSGGGGS (SEQ ID NO: 11).

Embodiment 58. The multimeric binding molecule of embodiment 55, wherein the linker consists of GKPGSGKPGSGKPGSGKPGS (SEQ ID NO: 14).

Embodiment 59. The multimeric binding molecule of any one of embodiments 49 to 58, wherein the 4-1BB trimer comprises the amino acid sequence SEQ ID NO: 198, SEQ ID NO: 199, SEQ ID NO: 200, or SEQ ID NO: 201.

Embodiment 60. The multimeric binding molecule of any one of embodiments 49 to 58, wherein the 4-1BBL trimer is fused to the N terminus of the J chain or fragment or variant thereof, or fused to the C terminus of the J chain or fragment or variant thereof, or wherein a copy of the 4-1BB trimer is fused to both the N terminus and the C terminus of the J chain or fragment or variant thereof.

Embodiment 61. The multimeric binding molecule of any one of embodiments 49 to 60 wherein the J chain or a functional fragment or variant thereof comprises the mature human J chain SEQ ID NO: 7 or a functional fragment or variant thereof.

Embodiment 62. The multimeric binding molecule of any one of embodiments 49 to 61, wherein the J comprises a variant J chain with an alanine substitution at the amino acid position corresponding to amino acid Y102 of the mature wild-type human J chain (SEQ ID NO: 7).

Embodiment 63. The multimeric binding molecule of any one of embodiments 49 to 62, wherein the J comprises the amino acid sequence SEQ ID NO: 8 (“J*”).

Embodiment 64. The multimeric binding molecule of any one of embodiments 49 to 63, wherein the modified J chain comprises the amino acid sequence SEQ ID NO: 202, SEQ ID NO: 203, SEQ ID NO: 204, SEQ ID NO: 205, SEQ ID NO: 206, SEQ ID NO: 207, SEQ ID NO: 208, SEQ ID NO: 209, SEQ ID NO: 210, SEQ ID NO: 211.

Embodiment 65. The multimeric binding molecule of any one of embodiments 1 to 64, which is an IgM antibody or IgM-like antibody comprising five bivalent binding units, wherein each binding unit comprises two IgM heavy chain constant regions or multimerizing fragments or variants thereof, each comprising an IgM Cμ4 domain and an IgM tailpiece domain.

Embodiment 66. The multimeric binding molecule of embodiment 65, wherein each IgM heavy chain constant region or multimerizing fragment or variant thereof further comprise a Cμ1 domain, a Cμ2 domain, a Cμ3 domain, or any combination thereof.

Embodiment 67. The multimeric binding molecule of embodiment 65 or embodiment 66, wherein the IgM heavy chain constant regions or multimerizing fragments or variants thereof are human IgM constant regions.

Embodiment 68. The multimeric binding molecule of embodiment 67, wherein each IgM heavy chain constant region comprises the amino acid sequence SEQ ID NO: 1, SEQ ID NO: 2, or a multimerizing variant or fragment thereof.

Embodiment 69. The multimeric binding molecule of embodiment 67 or embodiment 68, wherein each IgM heavy chain constant region is a variant human IgM constant region comprising one or more single amino acid substitutions, deletions, or insertions relative to SEQ ID NO: 1 or SEQ ID NO: 2, and wherein the multimeric binding molecule has reduced CDC activity relative to a multimeric binding molecule where each IgM heavy chain constant region comprises the amino acid sequence SEQ ID NO: 1 or SEQ ID NO: 2.

Embodiment 70. The multimeric binding molecule of embodiment 69, wherein each variant human IgM constant region comprises an amino acid substitution corresponding to position L310 of SEQ ID NO: 1 or SEQ ID NO: 2, an amino acid substitution corresponding to position P311 of SEQ ID NO: 1 or SEQ ID NO: 2, an amino acid substitution corresponding to position P313 of SEQ ID NO: 1 or SEQ ID NO: 2, an amino acid substitution corresponding to position K315 of SEQ ID NO: 1 or SEQ ID NO: 2, or any combination thereof.

Embodiment 71. The multimeric binding molecule of any one of embodiments 67 to 70, wherein each IgM heavy chain constant region is a variant human IgM constant region comprising one or more single amino acid substitutions, deletions, or insertions relative to SEQ ID NO: 1 or SEQ ID NO: 2, and wherein the multimeric binding molecule exhibits increased serum half-life upon administration to a subject animal relative to a multimeric binding molecule where each IgM heavy chain constant region comprises the amino acid sequence SEQ ID NO: 1 or SEQ ID NO: 2, which is administered in the same way to the same animal species.

Embodiment 72. The multimeric binding molecule of embodiment 71, wherein each variant IgM heavy chain constant region comprises half-life altering amino acid substitutions at one or more amino acid positions corresponding to amino acid E345, S401, E402, or E403 of SEQ ID NO: 1 or SEQ ID NO: 2.

Embodiment 73. The multimeric binding molecule of any one of embodiments 1 to 64, which is dimeric or tetrameric IgA antibody or IgA-like antibody comprising two or four bivalent binding units, wherein each binding unit comprises two IgA heavy chain constant regions or multimerizing fragments or variants thereof each comprising a Cα3 domain and an IgA tailpiece (αtp) domain.

Embodiment 74. The multimeric binding molecule of embodiment 73, wherein the IgA heavy chain constant regions or multimerizing fragments or variants thereof each further comprise a Cα1 domain, a Cα2 domain, an IgA hinge region, or any combination thereof.

Embodiment 75. The multimeric binding molecule of embodiment 73 or embodiment 74, wherein each IgA heavy chain constant region or multimerizing fragment or variant thereof is a human IgA constant region.

Embodiment 76. The multimeric binding molecule of any one of embodiments 1 to 75, wherein each target antigen-binding domain comprises a heavy chain variable region (VH) and a light chain variable region (VL), or a single-domain heavy chain variable region (sdVH).

Embodiment 77. The multimeric binding molecule of embodiment 76, wherein each heavy chain comprises, from N-terminus to C-terminus, a sdVH fused to the heavy chain constant region or multimerizing fragment or variant thereof.

Embodiment 78. The multimeric binding molecule of embodiment 76, wherein each heavy chain comprises, from N-terminus to C-terminus, a VH fused to the heavy chain constant region or multimerizing fragment or variant thereof.

Embodiment 79. The multimeric binding molecule of embodiment 78, wherein each binding unit further comprises two antibody light chains, each comprising, from N-terminus to C-terminus, a VL fused to a light chain constant region.

Embodiment 80. The multimeric binding molecule of any one of embodiments 1 to 79, wherein at least three, four, five, six, seven, eight, nine, or ten of the target antigen-binding domains are identical.

Embodiment 81. The multimeric binding molecule of embodiment 80, wherein the at least three, four, five, six, seven, eight, nine, or ten identical target antigen-binding domains specifically bind to a tumor target antigen.

Embodiment 82. The multimeric binding molecule of embodiment 81, wherein the tumor target antigen is CD38, CD20, CD19, CD22, CD28, B cell maturation antigen (BCMA), CD123, PD-L1, tumor-associated calcium signal transducer 2 (TROP-2), mesothelin, Muc16, prostate-specific membrane antigen (PSMA), or Six-Transmembrane Epithelial Antigen of Prostate-1·(STEAP-1).

Embodiment 83. The multimeric binding molecule of embodiment 82, wherein the tumor target antigen is CD20.

Embodiment 84. The multimeric binding molecule of embodiment 82, wherein the tumor target antigen is CD38.

Embodiment 85. The multimeric binding molecule of embodiment 82, wherein the tumor target antigen is mesothelin.

Embodiment 86. The multimeric binding molecule of embodiment 82, wherein the tumor target antigen is TROP-2.

Embodiment 87. The multimeric binding molecule of any one of embodiments 81 to 86, wherein the multimeric binding molecule conditionally activates T cells in the presence of the target antigen.

Embodiment 88. The multimeric binding molecule of any one of embodiments 81 to 87, wherein the multimeric binding molecule activates T cells after 7 days in the presence of the target antigen.

Embodiment 89. The multimeric binding molecule of embodiment 88, wherein the multimeric binding molecule activates T cells after 10 days in the presence of the target antigen.

Embodiment 90. The multimeric binding molecule of embodiment 89, wherein the multimeric binding molecule activates T cells after 14 days in the presence of the target antigen.

Embodiment 91. The multimeric binding molecule of any one of embodiments 81 to 90, wherein the multimeric binding molecule directs T cell-mediated killing of cells expressing the target antigen after 7 days in the presence of the target antigen.

Embodiment 92. The multimeric binding molecule of embodiment 91, wherein the multimeric binding molecule directs T cell-mediated killing of cells expressing the target antigen after 10 days in the presence of the target antigen.

Embodiment 93. The multimeric binding molecule of embodiment 92, wherein the multimeric binding molecule directs T cell-mediated killing of cells expressing the target antigen after 14 days in the presence of the target antigen.

Embodiment 94. The multimeric binding molecule of any one of embodiments 87 to 93, wherein the T cells are CD4 positive T cells.

Embodiment 95. The multimeric binding molecule of any one of embodiments 87 to 93, wherein the T cells are CD8 positive T cells.

Embodiment 96. A composition comprising the multimeric binding molecule of any one of embodiments 1 to 95 and a pharmaceutically acceptable carrier.

Embodiment 97. A polynucleotide comprising a nucleic acid sequence that encodes modified J chain of the multimeric binding molecule of any one of embodiments 1 to 95.

Embodiment 98. A vector comprising the polynucleotide of embodiment 97.

Embodiment 99. A composition comprising the polynucleotide of embodiment 97, a polynucleotide encoding a heavy chain of the multimeric binding molecule of any one of embodiments 1 to 95.

Embodiment 100. The composition of embodiment 99, further comprising a polynucleotide encoding a light chain of the multimeric binding molecule of any one of embodiments 1 to 95.

Embodiment 101. A vector comprising the composition of embodiment 99 or embodiment 100.

Embodiment 102. A host cell comprising the composition of embodiment 99 or embodiment 100, or the vector of embodiment 101, wherein the host cell can express the multimeric binding molecule of any one of embodiments 1 to 95.

Embodiment 103. A method of producing the multimeric binding molecule of any one of embodiments 1 to 95, comprising culturing the host cell of embodiment 102, and recovering the multimeric binding molecule.

Embodiment 104. A method of treating cancer, comprising administering to a subject in need of treatment the multimeric binding molecule of any one of embodiments 1 to 95.

Embodiment 105. The multimeric binding molecule of any one of embodiments 1 to 95, for use in treating cancer.

Embodiment 106. Use of the multimeric binding molecule of any one of embodiments 1 to 95 in the preparation of a medicament for the treatment of cancer.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All the references cited above, as well as all references cited herein, are incorporated herein by reference in their entireties.

The following examples are offered by way of illustration and not by way of limitation.

### EXAMPLES

### Example 1: Construction of Antibodies

To produce various IgM antibodies, modified J chains comprising a 4-1BBL trimer with three copies of either the “58” fragment (SEQ ID NO: 196) or “71” fragment (SEQ ID NO: 197) of 4-1BBL on the N and/or C terminus of a J chain comprising a Y to A amino acid substitution at position 102 (“Y102A” or “J*,” amino acid sequence of the variant presented as SEQ ID NO: 8) that enhances serum half-life of IgM pentamers that comprise the J chain variant were prepared. See U.S. Pat. No. 10,899,835. The 4-1BBL 15 monomers were connected by a ten or fifteen-amino acid long linker (SEQ ID NOS: 10 and 11, respectively) and the trimers were also connected to the J chain by these linkers. Some molecules also comprise an anti-CD3 scFv (SEQ ID NO: 219). The IgM antibodies produced comprise target antigen-binding domains that bind to exemplary tumor-specific or tumor-associated target antigens CD20 or mesothelin. A list of molecules made 20 comprising 4-1BBL are shown in Table 11 (“MSLN” is mesothelin).

TABLE 11
Antibodies with 4-1BBL on J chain

| | | Target | | | | 4- | J and | Modified |
|---|---|---|---|---|---|---|---|---|
| | | VH/VL | Anti- | 4- | 4- | 1BBL | 4- | J chain |
| Ab | IgM | SEQ ID | CD3 | 1BBL | 1BBL | internal | 1BBL | SEQ ID |
| Name | target | NOs | location | Fragment | location | linkers | linker | NO |
| A1 | CD20 | 144, 145 | N/A | 71 | N term | 10 | 10 | 206 |
| A2 | CD20 | 144, 145 | N/A | 71 | C term | 10 | 10 | 207 |
| A3 | CD20 | 144, 145 | N/A | 71 | N term | 10 | 15 | 209 |
| A4 | CD20 | 144, 145 | N/A | 71 | C term | 10 | 15 | 208 |
| A5 | CD20 | 144, 145 | N/A | 71 | N term | 15 | 15 | 205 |
| A6 | CD20 | 144, 145 | N/A | 71 | C term | 15 | 15 | 204 |
| A7 | CD20 | 144, 145 | N/A | 71 | N and | 15 | 15 | 211 |
| | | | | | C term | | | |
| A8 | CD20 | 144, 145 | N/A | 58 | N term | 15 | 15 | 203 |
| A9 | CD20 | 144, 145 | N/A | 58 | C term | 15 | 15 | 202 |
| A10 | CD20 | 144, 145 | N/A | 58 | N and | 15 | 15 | 210 |
| | | | | | C term | | | |
| A11 | CD20 | 144, 145 | C term | 71 | N term | 15 | 15 | 214 |
| A12 | CD20 | 144, 145 | N term | 71 | C term | 15 | 15 | 213 |
| B1 | MSLN | 262, 263 | N/A | 71 | N term | 10 | 10 | 206 |
| B2 | MSLN | 262, 263 | N/A | 71 | C term | 10 | 10 | 207 |
| B3 | MSLN | 262, 263 | N/A | 71 | N term | 15 | 15 | 205 |
| B4 | MSLN | 262, 263 | N/A | 71 | C term | 15 | 15 | 204 |

Additional IgM antibodies were generated with modified J chains comprising one of four anti-CD28 scFvs: W (VH: SEQ ID NO: 69, VL: SEQ ID NO: 78, scFv: SEQ ID NO: 284), X (VH: SEQ ID NO: 58, VL: SEQ ID NO: 83, scFv: SEQ ID NO: 225), Y (VH: SEQ ID NO: 59, VL: SEQ ID NO: 83, scFv: SEQ ID NO: 226), or Z (VH: SEQ ID NO: 61, VL: SEQ ID NO: 81, scFv: SEQ ID NO: 227), and optionally an anti-CD3 scFv (SEQ ID NO: 219). A list of molecules made comprising anti-CD28 scFvs is provided in Table 12.

TABLE 12
Antibodies with anti-CD28 on J* chain

| | | Target | | | | Modified |
|---|---|---|---|---|---|---|
| | | VH/VL | Anti- | Anti- | Anti- | J chain |
| Antibody | IgM | SEQ ID | CD3 | CD28 | CD28 | SEQ ID |
| Name | target | NOs | location | scFv | location | NO |
| C1 | CD20 | 144, 145 | N/A | X | C term | 228 |
| C2 | CD20 | 144, 145 | N/A | Y | C term | 229 |
| C3 | CD20 | 144, 145 | N/A | Z | C term | 230 |
| C4 | CD20 | 144, 145 | N term | X | C term | 231 |
| C5 | CD20 | 144, 145 | N term | Y | C term | 232 |
| C6 | CD20 | 144, 145 | N term | Z | C term | 233 |
| C7 | CD20 | 144, 145 | N term | Z | C term | 234 |

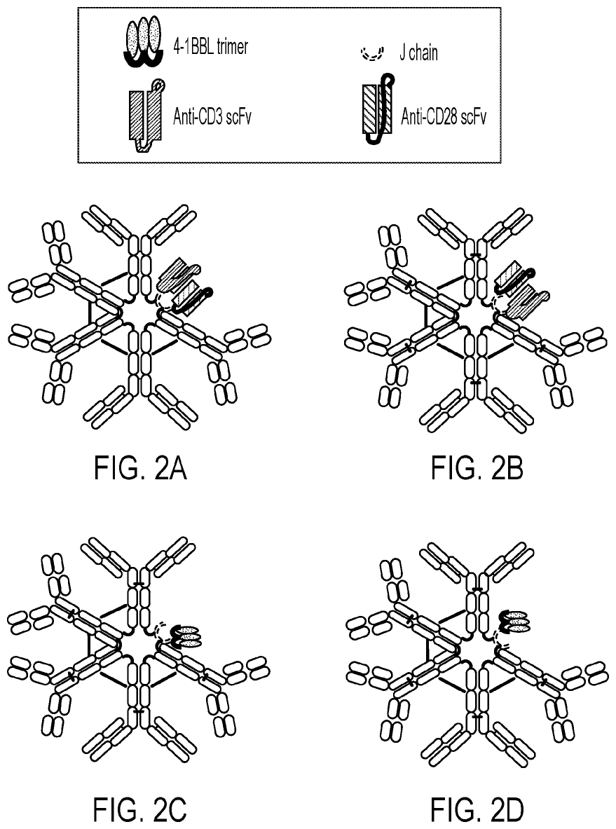

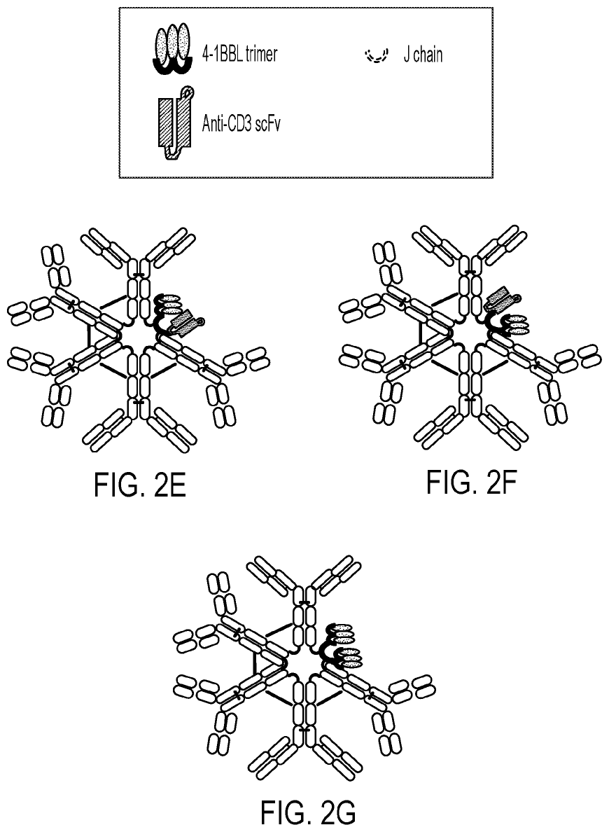

Schematics of exemplary IgM antibodies made and contemplated are shown in FIGS. 2A-2G.

Constructs were made according to standard cloning protocols. In brief, heavy chain variable regions (VH) and light chain variable regions (VL) of exemplary target antigen binding domains were cloned into an IgM format containing a modified J chain. The resulting constructs were expressed in Expi293 cells and purified according to the methods described in Keyt et al., Antibodies: 9:53, doi: 10.3390/antib9040053 (2020).

Additional control molecules were also made as described above or purchased commercially and are shown in Table 13.

TABLE 13
Control Molecules

| Antibody | | VH/VL and J |
|---|---|---|
| Name | Description | chain SEQ ID NOs |
| D1 | Anti-CD20 IgM with J* chain | 144, 145, 8 |
| D2 | 4-1BBL trimer fused to an Fc | Acrobiosystems |
| | | # 41L-H5269 |
| D3 | Anti-4-1BB IgM antibody with | 270, 271 |
| | unmodified J chain | |
| D4 | Anti-4-1BB IgG antibody | 270, 271 |
| D5 | Anti-mesothelin IgM with | 262, 263, 7 |
| | unmodified J chain | |
| D6 | Anti-mesothelin IgM with J chain | 262, 263, 269 |
| | with anti-CD3 scFv | |
| D7 | Anti-CD20 IgM with J* chain with | 144, 145, 220 |
| | anti-CD3 scFv | |
| D8 | Anti-CD20 × anti-CD3 IgG #1 | 237, 238, 239 |
| D9 | Anti-CD20 × anti-CD3 IgG #2 | 240, 241, 242 |
| D10 | Anti-CD20 IgM with J chain with | 144, 145, 243 |
| | HSA and anti-CD3 scFv | |
| D11 | Anti-TROP-2 IgM with J* chain | 248, 249, 8 |
| D12 | Anti-TROP-2 IgM with J* chain | 248, 249, 220 |
| | with anti-CD3 scFv | |
| D13 | Anti-mesothelin IgM with | 262, 263, 8 |
| | unmodified J* chain | |
| D14 | Anti-mesothelin IgM with J* chain | 262, 263, 220 |
| | with anti-CD3 scFv | |
| D15 | Anti-virus isotype control with | 266, 267, 234 |
| | modified J* chain with anti-CD3- | |
| | AA and anti-CD28-Z | |
| D16 | TROP-2 × CD3 bispecific IgG | 274, 275, 276 |
| D17 | MSLN × CD3 bispecific IgG | 277, 278, 276 |

### Example 2: Binding 4-1BB+ Cells

To determine the binding capabilities of IgM antibodies containing 4-1BBL-modified J chain constructs, a flow cytometry-based binding assay was performed.

Human 4-1BB+293T cells (Crown Bioscience #C2012) were dissociated using enzyme-free cell dissociation buffer (Gibco #13150-016) and washed twice with stain buffer (BD Pharmingen™ #554656). Binding assays were carried out in 96-well V-bottom plates, at a cell density of 30,000 cells/well.

IgM antibodies containing 4-1BBL-modified J chains A1-A6 and A8-A9, described in Example 1, were tested, along with the negative control D1. Antibody concentrations were adjusted to 10 nM in stain buffer and added to cells to obtain a final volume of 100 μL/well. Assay plates were then incubated on ice for 1 hour, and cells were washed three times with stain buffer. 1,000-fold diluted mouse anti-human Kappa-AF647 (SouthernBiotech #9230-31) and goat anti-human Fc-AF647 (SouthernBiotech #2014-31) secondary antibodies were added at 100 μL/well to all antibody wells. Assay plates were then incubated on ice for 45 minutes, and cells were washed three times using stain buffer. 1,000-fold diluted 7-aminoactinomycin D (7-AAD) viability dye was then added to the cells, and fluorescence was analyzed using an INTELLICYT® IQUE® flow cytometer. Data was plotted as antibody concentration (x-axis) and relative fluorescence as a percentage of live cells (y-axis), using GraphPad Prism 8 software.

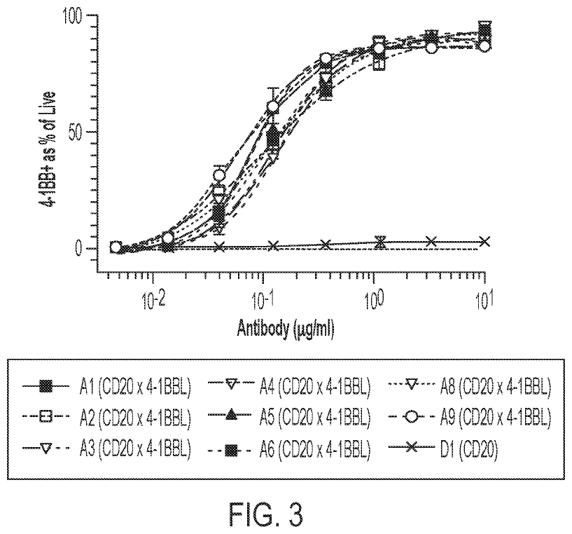

FIG. 3 shows IgM antibodies having a 4-1BBL-modified J chain in various formats bound to 4-1BB+ cells, while the negative control antibody D1 (no 4-1BBL) did not bind to 4-1BB+ cells.

### Example 3: Biolayer Interferometry Affinity Measurement Assay

The binding affinities and avidity of 4-1BBL comprising antibodies, A5, A6, or A10, or 4-1BBL trimer fused to an Fc, D2 (Acrobiosystems #41L-H5269), were determined by biolayer interferometry (BLI) on an Octet-384 (Sartorius/ForteBio, NY, USA) using Anti-Penta-His biosensors (Sartorius/Fortebio Cat #18-5120). PBST (1×PBS+1% BSA+0.05% Tween-20) buffer was used as reagent dilution and sensor hydration buffer. The experiment followed a six-step sequential assay at 24 °C. First, biosensors were hydrated for 10 minutes. Samples and buffer were applied in a 384-well plate. After the initial baseline of 30 s, sensors were loaded with 10 nM 4-1BB-Fc with AVITAG™ for 240 s, then moved into buffer for a baseline for 30 s. Next, various concentrations of antibody or 4-1BBL-Fc fusion were associated for 240 s and dissociated for 500 s in PBST. Results were analyzed by ForteBio Data Analysis software 9.0 using global fit model. The K_{D} values are shown in Table 14. Binding over time of various concentrations of A5, A6, A9, and D2 is shown in FIGS. 4A-4D, respectively.

TABLE 14
Binding Kinetics

| | KD [nM] | Kon(1/Ms) | Kdis(1/s) |
|---|---|---|---|
| A5 | <0.01 | 1.56E+05 | <1.0E−07 |
| A6 | <0.01 | 4.34E+05 | <1.0E−07 |
| A9 | <0.01 | 3.81E+05 | <1.0E−07 |
| D2 | 0.02 | 2.40E+06 | 5.35E−05 |

### Example 4: Luciferase Reporter Assay Using CD20 Coating

To determine the ability of 4-1BBL trimers on the J chain of IgM antibodies to agonize 4-1BB in the presence or absence of the IgM antibody target, a series of luciferase reporter assays were run in the presence or absence of CD20. Antibodies A5, A6, A8, and A9 described in Example 1 were tested, along with control molecules D1 (anti-CD20 IgM with no 4-1BBL on a J* chain), D2 (4-1BBL trimer fused to Fc) with or without anti-Fc crosslinking, D3 (anti-4-1BB IgM antibody) and D4 (anti-4-1BB IgG antibody) with or without anti-Fc crosslinking.

For assays performed in the presence of CD20, a 96-well opaque white plate was coated with 100 μl/well 50 nM human CD20 with a TrxA tag (Acrobio #CDO-H5143, LOT 2113-7BNR2-GC) phosphate buffered saline (PBS) and incubated at 37 °C for 1.5 hours and then removed with shaking.

Adherent cell line 293T/huCD137+ cells (Crown Bio #C2012 lot #160331-IGM042020) was cultured in DMEM+10% FBS+1 μg/ml Puromycin and dissociated using Trypsin-free Cell Dissociation buffer. The viable cell density was adjusted to 6.0 x 10⁵ cells/ml (30,000 cells/well). Then, 50 μl cells were added per well to both CD20-coated and non-coated plates.

50 μL per well of a serial dilution of A5, A6, A8, A9, D1, D2, and D3 in DMEM+10% FBS+1 μg/ml Puromycin was added. For conditions with crosslinking, and 10 μg/mL of anti-human Fc antibody was added to the cells.

Jurkat human 4-1BB positive Luciferase reporter cells (Promega #JA2351) cells were thawed at 37 °C for 2 mins, washed once in complete RPMI-1640+ 10% heat inactivated FBS buffer. Jurkat cell density was adjusted to 6.25×10⁵ viable cells/mL. 20 ul of 4-1BB+ Reporter cells were added to the wells.

The plates were incubated at 37 °C for 16 hours in a 5% CO₂ incubator. 100 μL of BIO-GLO™ (Promega #G7940) were added to cells directly and incubated for 10 min at room temperature. Luminescence (RLU) was measured using a Perkin Elmer ENVISION® reader. Graphs were plotted using antibody concentration on the x-axis and RLU on the y-axis using GraphPad Prism 8 software.

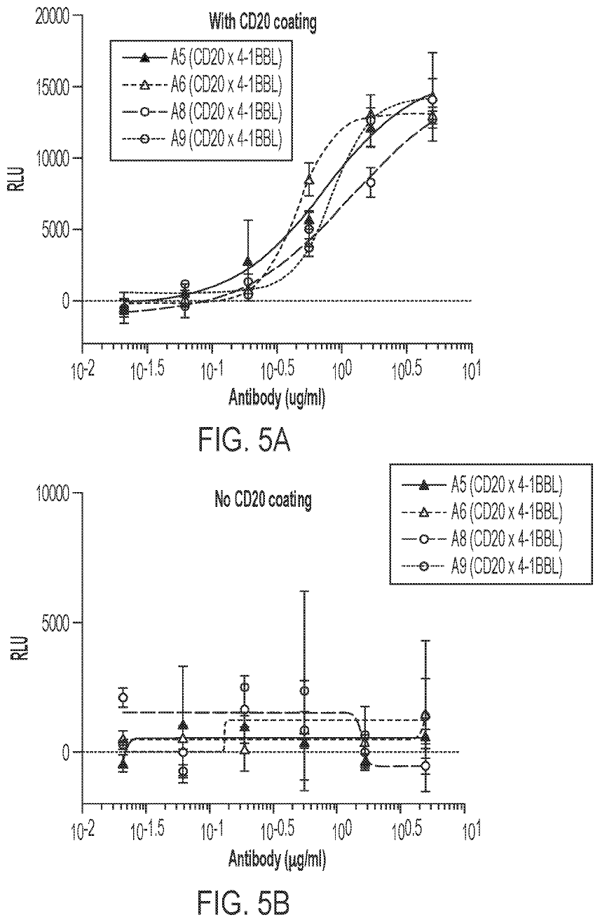

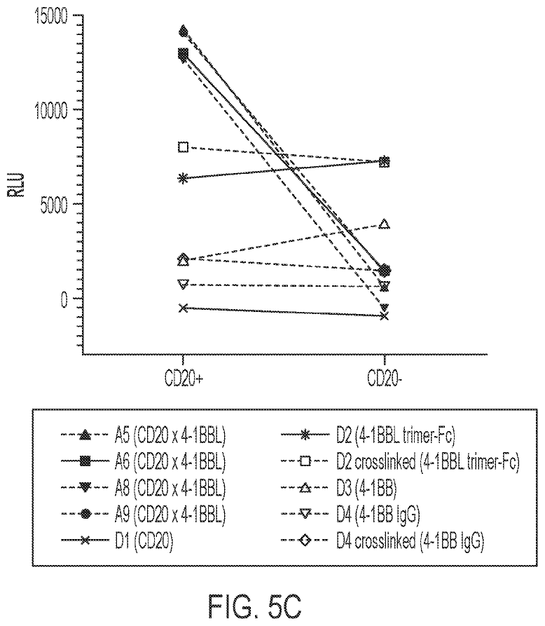

The luminescence measured at each concentration for A5, A6, A8, and A9 with CD20 coating or without CD20 coating is shown in FIGS. 5A and 5B, respectively. FIG. 5C shows the luminescence measured at the highest concentration tested for A5, A6, A8, A9, D1, D2 (with and without crosslinking), D3, and D4 (with and without crosslinking) in the presence or absence of CD20. In the presence of CD20, the anti-CD20 IgM antibodies comprising 4-1BBL trimer (A5, A6, A8, and A9) on the J chain showed greater agonist activity than the anti-4-1BB IgM antibody (D3), the anti-4-1BB IgG antibody (D4) with or without crosslinking, the 4-1BBL trimer fused to an Fc (D2) with or without crosslinking, or the anti-CD20 IgM without the 4-1BBL trimer (D1). However, in the absence of CD20, the anti-CD20 IgM antibodies comprising 4-1BBL trimer on the J chain exhibited less agonist activity than 4-1BBL trimer fused to an Fc (D2), with or without crosslinking, or the anti-4-1BB IgM antibody (D3).

### Example 5: Luciferase Reporter Assay Using CD20-Expressing Cells

Jurkat human 4-1BB positive Luciferase reporter cells (Promega #JA2351) cells were thawed at 37 °C for 2 mins and washed once in complete assay buffer (RPMI-1640+10% heat inactivated FBS buffer). Jurkat cell density was adjusted to 6.25×10⁵ viable cells/mL. CD20+ target cells (Ramos cells) and CD20-target cells (OPM-2) cell density was adjusted to 7.5×10⁵ viable cell/mL in the assay buffer.

Test molecules A1, A2, A3, A4, A5, A6, A8, A9, D1, or D2 were diluted to 20 nM and then serially diluted in assay buffer. 10 μl of test molecule was added into 384-well opaque plates followed by 10 μL of CD20+ and CD20-cells. The wells were incubated at room temperature for 15 minutes. Finally, 20 μL of 4-1BB+ reporter cells were added to the wells and the wells were incubated at 37 °C for 16 hours. 20 μL per well of BIO-GLO™ Luciferase reagent (Promega #G7941) was added to each well and luminescence (RLU) was read on ENVISION® (Perkin Elmer 2103). Graphs were plotted using antibody concentration on the x-axis and RLU on the y-axis using GraphPad Prism 8 software.

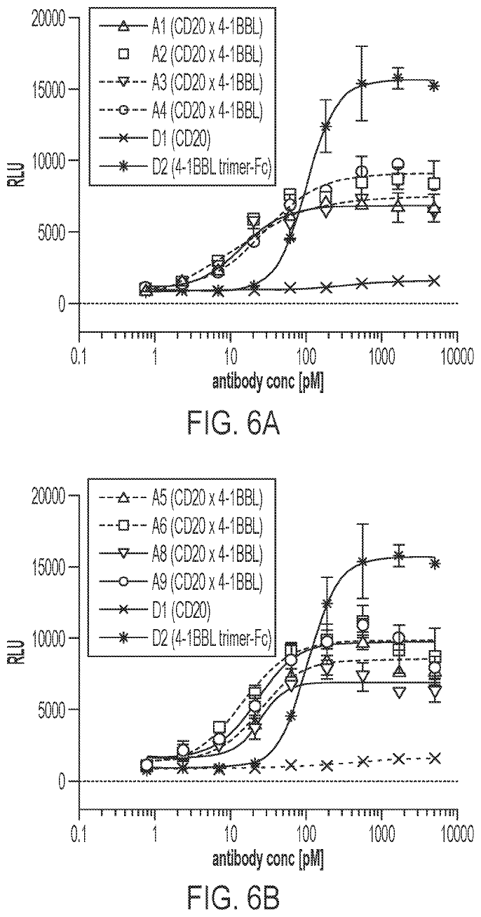

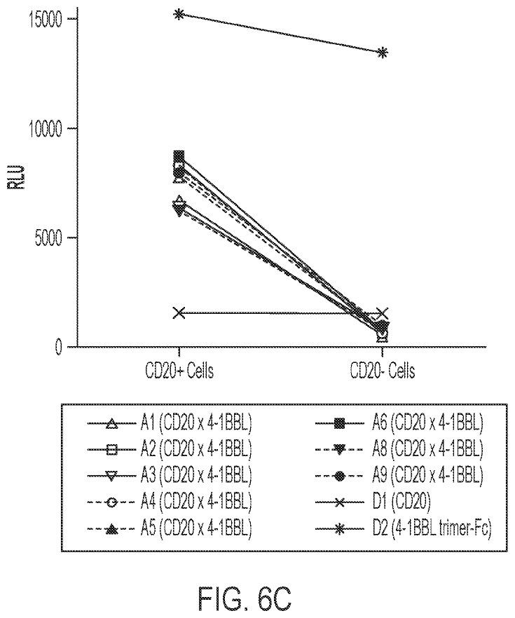

The luminescence measured at each concentration for A1, A2, A3, A4, A5, A6, A8, A9, D1, and D2 with CD20+ (Ramos) cells is shown in FIGS. 6A (A1, A2, A3, A4, D1, and D2) and 6B (A5, A6, A8, A9, D1, and D2). FIG. 6C shows the luminescence measured at the highest concentration tested for A1, A2, A3, A4, A5, A6, A8, A9, D1, and D2 in the presence of CD20+ cells (Ramos) or CD20-cells (OPM-2).

In the presence of CD20-expressing cells, the anti-CD20 IgM antibodies comprising 4-1BBL trimer on the J chain showed agonist activity. However, in the presence of cells that do not express CD20, the anti-CD20 IgM antibodies comprising 4-1BBL trimer on the J chain did not show agonist activity. D2, the 4-1BBL trimer-Fc fusion, showed agonist activity regardless of the expression of CD20.

### Example 6: Luciferase Reporter Assay Using CD20-Expressing Cells

The assay described in Example 5 was repeated with cells expressing high levels of CD20 (DOHH2), low levels of CD20 (CA46), and no CD20 (OPM-2). In addition to the antibodies tested in Example 5, A1, A2, A3, A4, A5, A6, A8, A9, D1, and D2, antibodies A7 and A10 (4-1BBL trimer on both the N and C terminus, respectively) and A11 and A12 (4-1BBL trimer on one terminus and anti-CD3 scFv on the other terminus, respectively) were assessed.

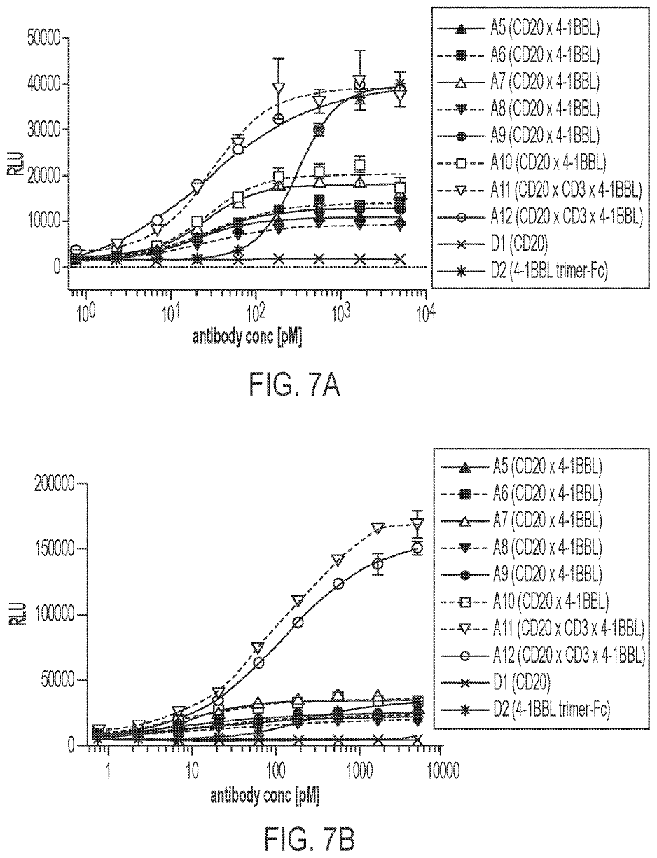

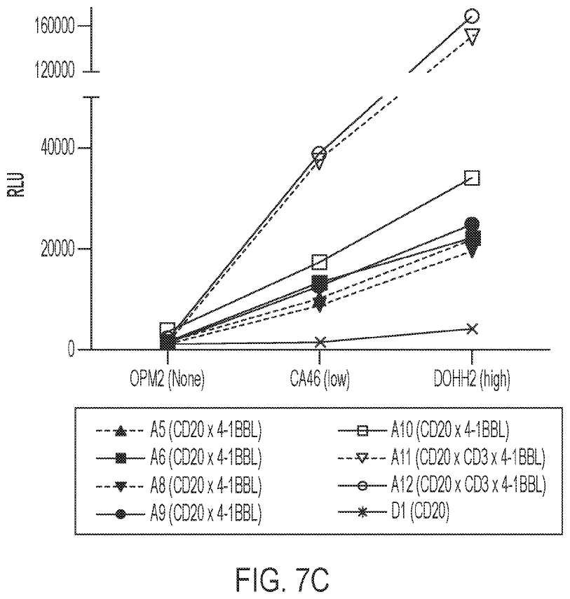

The luminescence measured at each concentration for A5, A6, A7, A8, A9, 10, A11, A12, D1, and D2 with low levels of CD20 (CA46) cells is shown in FIG. 7A;
luminescence measured with high levels of CD20 (DOHH2) cells is shown in FIG. 7B. FIG. 7C shows the luminescence measured at the highest concentration tested for A5, A6, A8, A9, A10, A11, A12, and D1 in the presence of cells expressing high levels of CD20 (DOHH2), cells expressing low levels of CD20 (CA46), or CD20-cells (OPM-2).

All anti-CD20 IgM antibodies comprising 4-1BBL trimer on the J chain showed CD20-level dependent agonist activity. Antibodies with two copies of the 4-1BBL trimer showed higher levels of agonist activity; antibodies with an anti-CD3 scFv and a 4-1BBL trimer showed the highest levels of agonist activity.

### Example 7: Antibody Binding Measured by ELISA

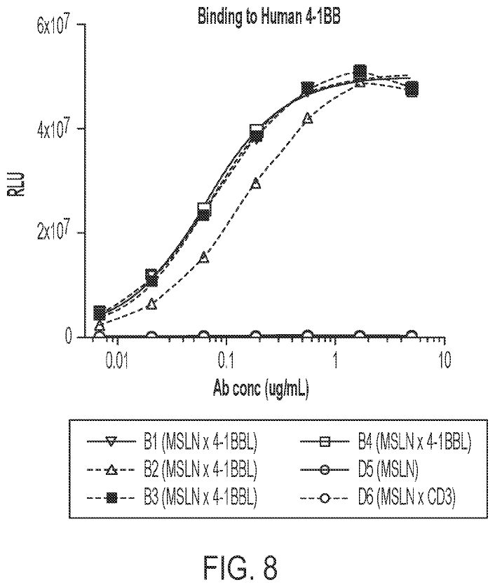

As described in Example 1, 6 IgM antibodies, B1, B2, B3, B4, D5, and D6, were made to bind mesothelin. B1-B4 comprise 4-1BBL trimer on the J chain, D5 comprises an unmodified J chain, and D6 comprises an anti-CD3 scFv on the J chain. Binding to 4-1BB was then measured in ELISA assays as follows. 96-well white polystyrene ELISA plates (Pierce 15042) were coated with 100 μL per well of 1.0 μg/mL recombinant target 4-1BB overnight at 4 °C. Plates were then washed 5 times with 0.05% PBS-Tween and blocked with 2% BSA-PBS. After blocking, 100 μL of serial dilutions of B1, B2, B3, B4, D5, or D6 were added to the wells and incubated at room temperature for 2 hours. The plates were then washed 10 times and incubated with HRP conjugated mouse anti-human kappa (Southern Biotech, 9230-05; 1:6000 diluted in 2% BSA-PBS) for 30 min. After 10 final washes using 0.05% PBS-Tween, the plates were read out using SuperSignal chemiluminescent substrate (ThermoFisher, 37070). Luminescent data were collected on an ENVISION® plate reader (PerkinElmer) and analyzed with GraphPad Prism using a 4-parameter logistic model. Binding to 4-1BB is shown in FIG. 8. Antibodies B1-B4 bound 4-1BB while D5 and D6 did not.

### Example 8: Luciferase Reporter Assay Using Mesothelin-Expressing Cells

HEK-293T cells or mesothelin expressing HEK-293T cells were adjusted to 1.5×10⁵ viable cell/mL in complete assay buffer (RPMI-1640+ 10% heat inactivated FBS). 20 μL mesothelin+ and mesothelin-cells were added into each well of 384-well opaque plates and incubated overnight at 37 °C with 5% CO₂ to allow cells to attach to the plates.

The next day, test molecules B1, B2, B3, B4, D5, or D6 were diluted to 20 nM and then serially diluted in assay buffer. Jurkat human 4-1BB positive Luciferase reporter cells (Promega #JA2351) were thawed at 37 °C for 2 mins, washed once in assay buffer. Jurkat cell density was adjusted to 1.5×10⁶ viable cells/mL. 10 μl of test molecule was added into 384-well opaque plates. The plates were incubated at 37 °C for 15 minutes. Finally, 10 μL of 4-1BB+ reporter cells were added to the wells and the wells were incubated at 37 °C for 24 hours. 20 μL per well of BIO-GLO™ Luciferase reagent (Promega #G7941) was added to each well and luminescence (RLU) was read on ENVISION® (Perkin Elmer 2103). Graphs were plotted using antibody concentration on the x-axis and RLU on the y-axis using GraphPad Prism 8 software.

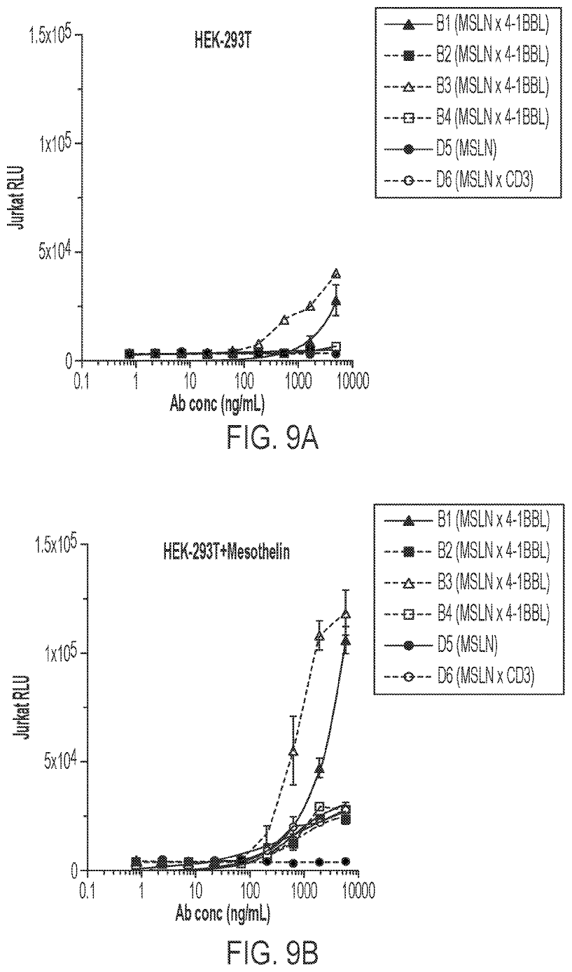

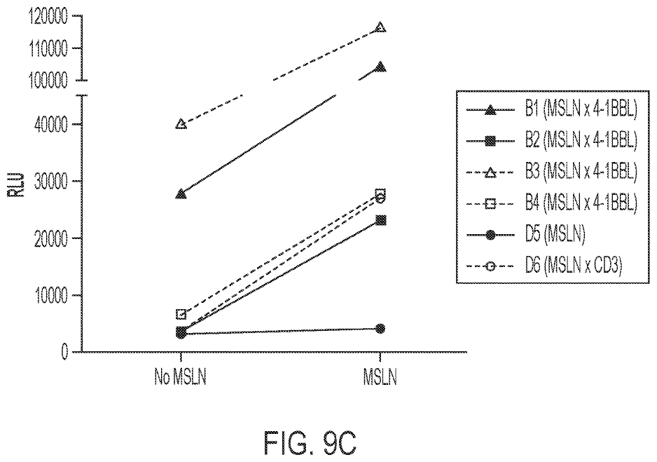

The luminescence measured at each concentration for B1, B2, B3, B4, D5, or D6 with mesothelin-cells is shown in FIG. 9A and with mesothelin+ cells in FIG. 9B. FIG. 9C shows the luminescence measured at the highest concentration tested for B1, B2, B3, B4, D5, and D6 in the presence of mesothelin+ cells or mesothelin-cells.

In the presence of mesothelin-expressing cells, the anti-mesothelin IgM antibodies comprising 4-1BBL trimer on the J chain showed agonist activity, with IgM antibodies having 4-1BBL on the N terminus showing greater agonist activity than IgM antibodies having 4-1BBL on the C terminus. However, in the presence of cells that do not express mesothelin, the anti-mesothelin IgM antibodies comprising 4-1BBL trimer on the C terminus of the J chain did not show agonist activity. Anti-mesothelin IgM antibodies comprising 4-1BBL trimer on the N terminus of the J chain did show agonist activity in the presence of cells that do not express mesothelin, although the agonist activity was significantly less than that in the presence of mesothelin.

### Example 9: T Cell Activation

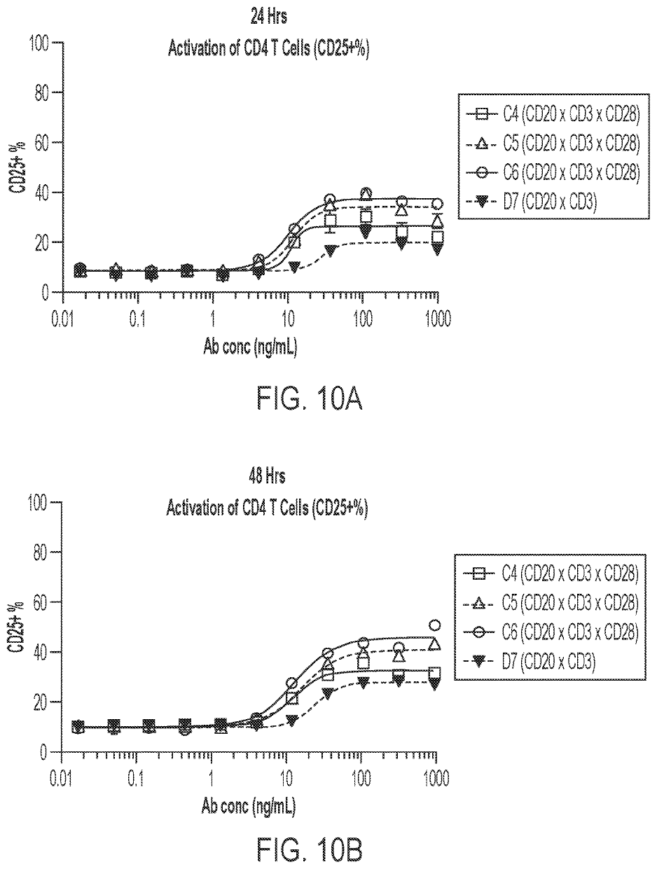

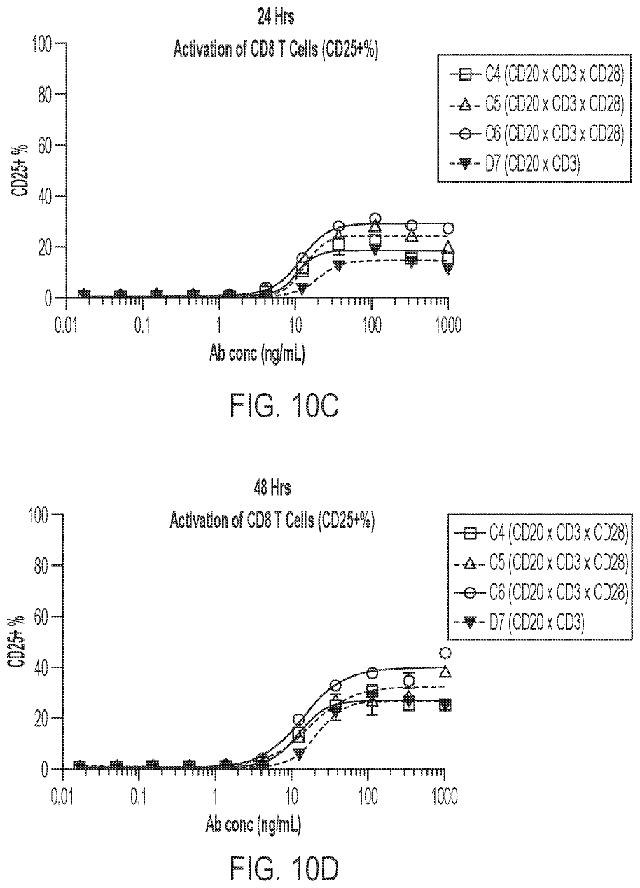

The ability of the antibodies comprising a J chain comprising an anti-CD28 scFv and an anti-CD3 scFv to enhance T cell activation was assessed as follows. 100×10³ human PBMCs were cultured in the presence of serial dilutions of C4, C5, C6, and D7 (as described in Example 1) in 200 μL total volume of culture media (RPMI-1640+ 10% heat inactivated FBS) for 24 or 48 hours. Cells were stained for FACS analysis with the following staining panel: anti-CD19 FITC, anti-CD3 PerCP-Cy5.5, anti-CD8 BV785, anti-CD25 APC, and anti-CD4 BV605 from Biolegend, and the Fixable viability dye EFLUOR® 780 (ThermoFisher, 65-0865-14). Activation of CD4+ T cells at various concentrations after 24 and 48 hours is shown in FIGS. 10A-10B, respectively. Activation of CD8+ T cells at various concentrations after 24 and 48 hours is shown in FIGS. 10C-10D, respectively. The combination of CD28 and CD3 stimulation (Antibodies C4, C5, and C6) lead to greater T cell activation than CD3 stimulation alone (Antibody D7).

Generally, exemplary IgM antibodies with anti-CD3 x anti-CD28-comprising J chains exhibited increased binding to T cells when compared to IgM antibodies with only anti-CD3 or only anti-CD28 on the J chain, suggesting dual engagement on T cells. Illustrative IgM antibodies with dual signal engagement possessed improved T cell activation compared to illustrative IgM antibodies with signal 1 (anti-CD3) only, while no or minimal T cell activation was observed in the absence of signal 1 engagement or in the absence of target cells.

### Example 10: T Cell-Directed Cytotoxicity

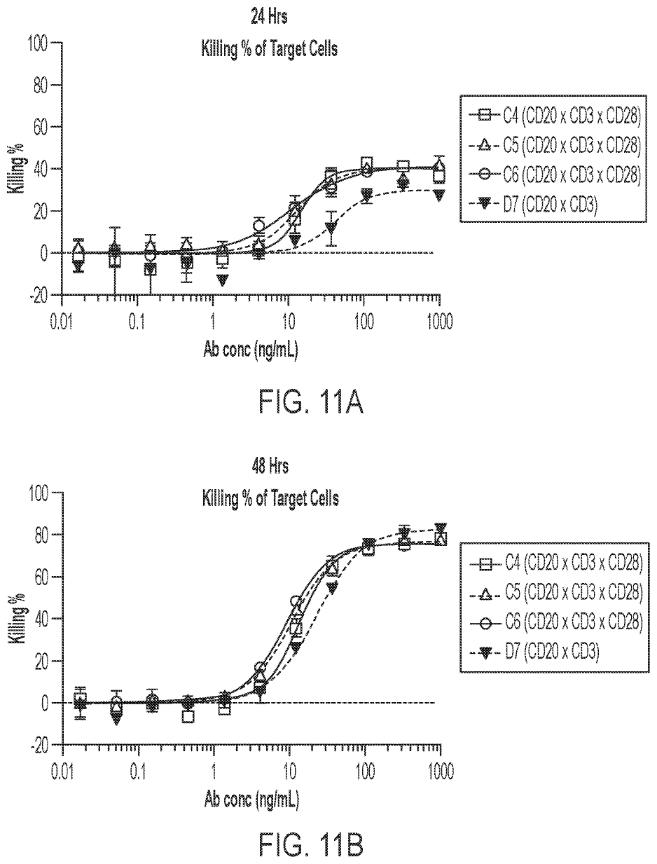

The ability of the antibodies comprising a J chain comprising an anti-CD28 scFv and an anti-CD3 scFv to enhance T cell-directed cytotoxicity (TDCC) was assessed as follows. 100×10³ human PBMCs were co-cultured with Ramos cells in the presence of serial dilutions of C4, C5, C6, and D7 (as described in Example 1) in 200 μL total volume of culture media (RPMI-1640+ 10% heat inactivated FBS) for 24 or 48 hours. 50 μl of supernatant was removed and frozen at −80 °C for later cytokine release analysis. Cells were stained for FACS analysis with the following staining panel: anti-CD19 FITC, anti-CD3 PerCP-Cy5.5, anti-CD8 BV785, anti-CD25 APC, and anti-CD4 BV605 from Biolegend, and the Fixable viability dye EFLUOR® 780 (ThermoFisher, 65-0865-14). The number of viable CD19+B cells in each well were compared to spiked-in counting beads to calculate percentage killing. The data was then analyzed and plotted with GraphPad Prism. Dose response curves after 24 and 48 are shown in FIGS. 11A-11B, respectively. The combination of CD28 and CD3 stimulation (Antibodies C4, C5, and C6) lead to greater TDCC activity than CD3 stimulation alone (Antibody D7).

### Example 11: Cytokine Release

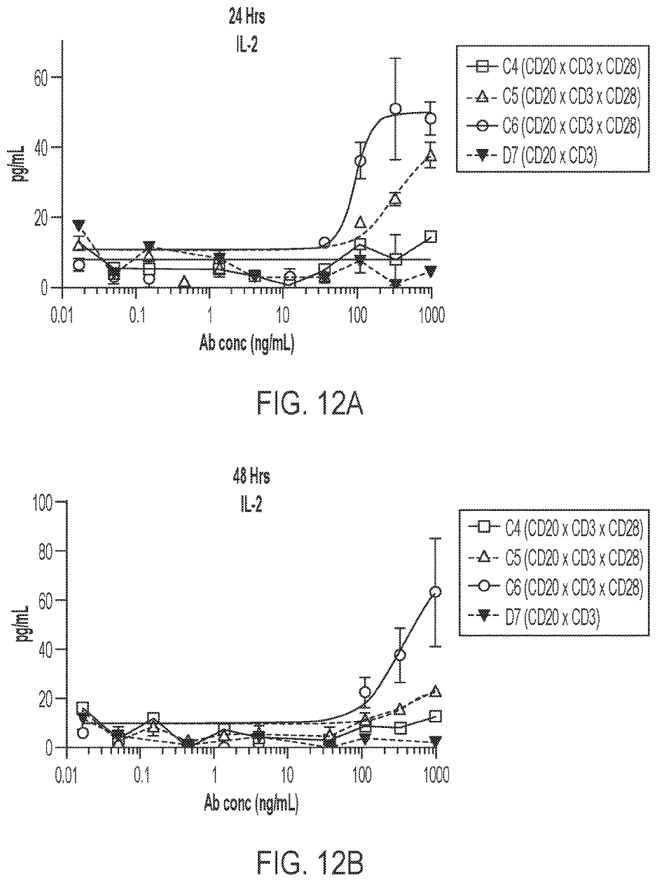

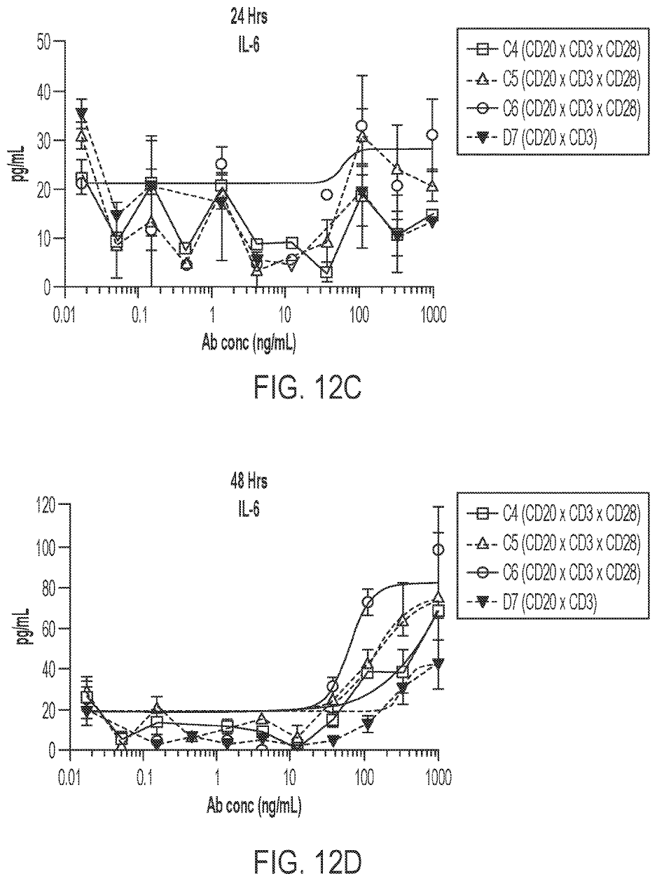

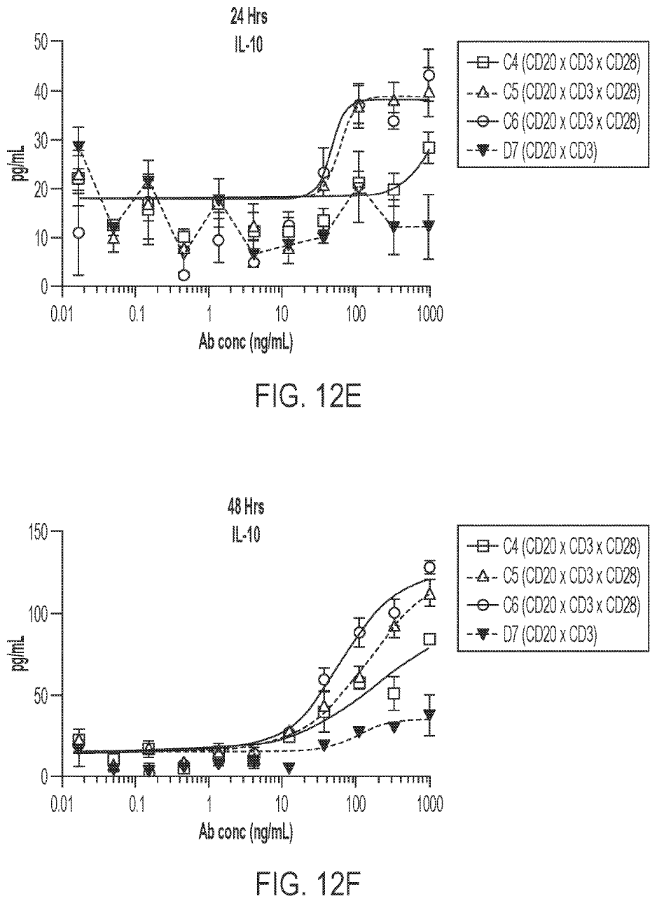

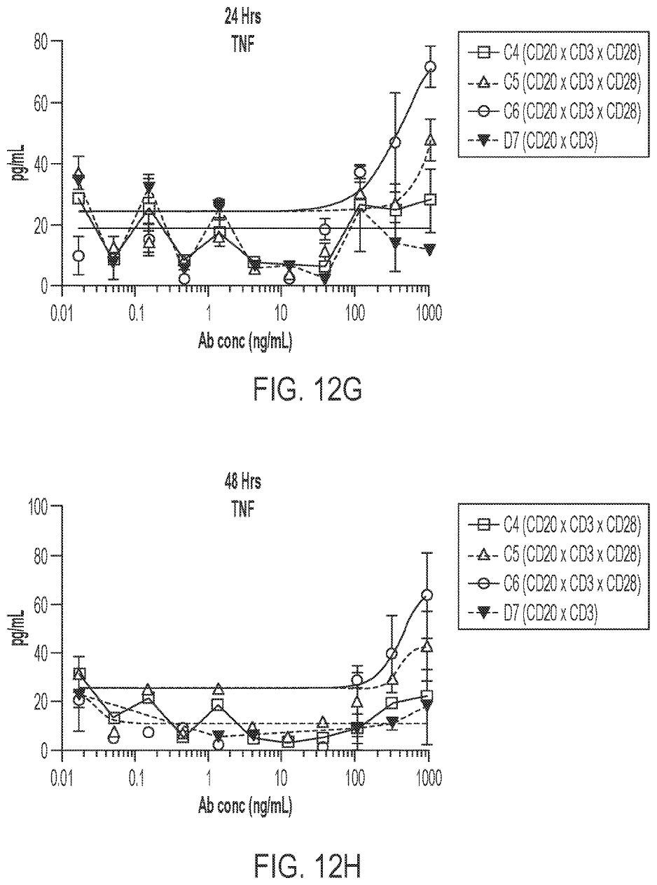

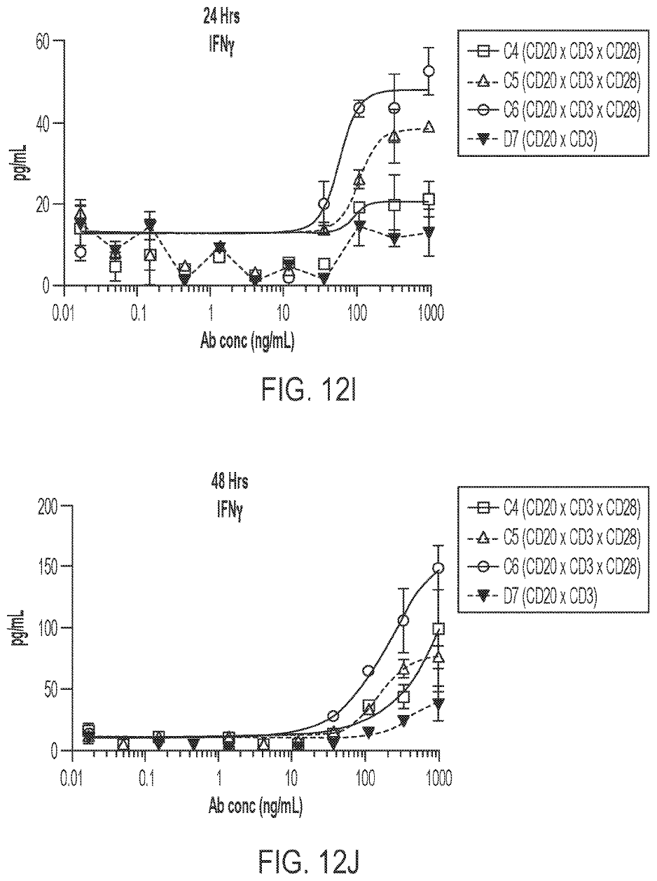

Supernatants from TDCC assays obtained in Example 10 were assayed for a panel of cytokines including IL-2, IL-6, IL-10, TNFα, and IFNγ using a cytometric bead array human Th1-2 cytokine kit II (BD, 551809) according to manufacturer's protocol except using smaller volumes to accommodate plate-based assays. The results were then analyzed with GraphPad Prism. Cytokine concentrations in supernatants from PBMC treated with various concentrations of antibodies after 24 hours and 48 hours are shown in FIGS. 12A and 12B, respectively for IL-2, FIGS. 12C and 12D, respectively for IL-6, FIGS. 12E and 12F, respectively for IL-10, FIGS. 12G and 12H, respectively for TNFα, and FIGS. 12I and 12J, respectively for IFNγ. Enhanced TDCC and T cell activation correlated with cytokine response.

### Example 12: Construction of Additional Antibodies

Additional IgM pentameric antibodies, which are described in Table 15, were produced according to the methods described in Example 1. IgM-based Anti-CD38 antibodies are disclosed in PCT Publication No. WO2023150677A2, which is incorporated herein by reference in its entirety. The VH and VL sequences are provided in Table 26.

TABLE 15
Additional Antibodies

| | | | | | VH, VL, |
|---|---|---|---|---|---|
| | | Anti- | Anti- | Anti- | Modified |
| Antibody | IgM | CD3 AA | CD28 | CD28 | J*chain |
| Name | target | location | scFv | location | SEQ ID NO |
| E1 | CD38 | N term | Z | C term | 272, 273, 233 |
| D18 | CD38 | N term | N/A | N/A | 272, 273, 220 |

### Example 13: T Cell Activation

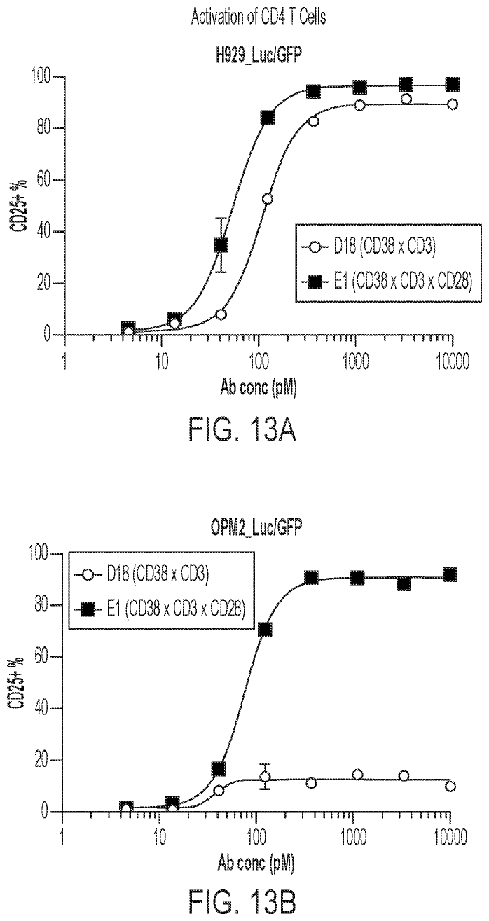

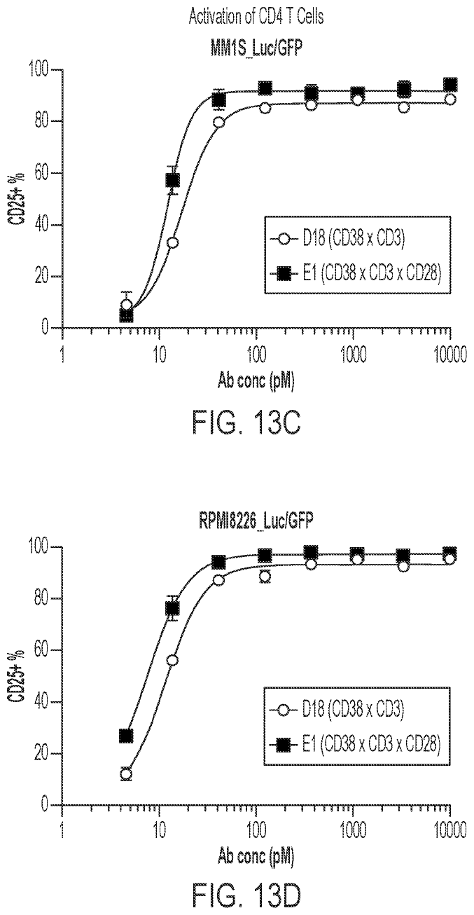

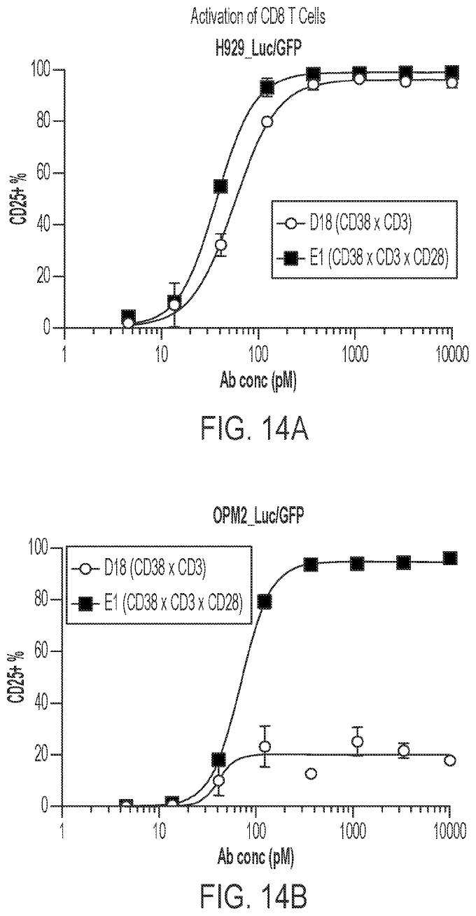

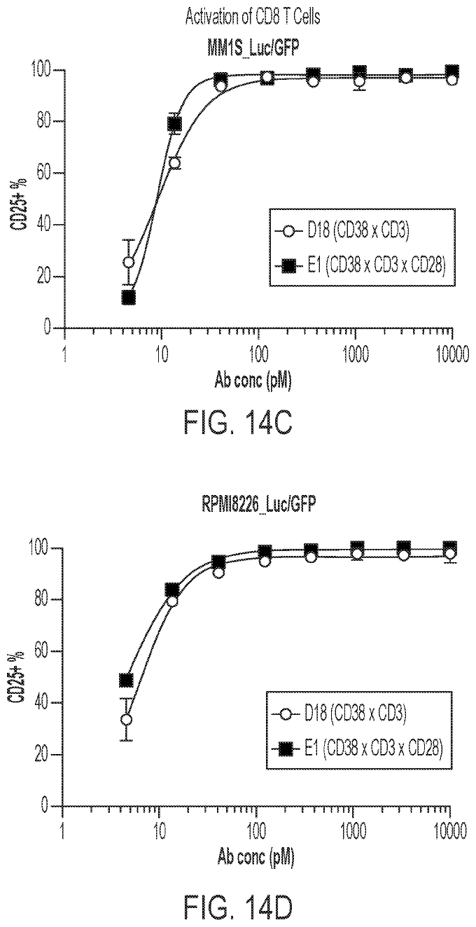

The ability of the exemplary anti-CD38 IgM antibody E1, as described in Example 12, to enhance T cell activation compared to the anti-CD38 IgM antibody D18, as described in Example 12, was assessed as follows. 100×10³ human PBMCs were co-H929_Luc/GFP, cultured with OPM2_Luc/GFP, MMIS_Luc/GFP, or RPMI8226_Luc/GFP cells in the presence of serial dilutions of E1 and D18 in 200 μL total volume of culture media (RPMI-1640+ 10% heat inactivated FBS) for 48 hours. Cells were stained for FACS analysis with the following staining panel: anti-CD19 FITC, anti-CD3 PerCP-Cy5.5, anti-CD8 BV785, anti-CD25 APC, and anti-CD4 BV605 from Biolegend, using the fixable viability dye EFLUOR® 780 (ThermoFisher, 65-0865-14). Activation of CD4+ T cells co-cultured with H929_Luc/GFP, OPM2_Luc/GFP, MMIS_Luc/GFP, or RPMI8226_Luc/GFP cells at various concentrations of E1 and D18 after 48 hours is shown in FIGS. 13A-13D, respectively. Activation of CD8+ T cells co-cultured with H929 Luc/GFP, OPM2 Luc/GFP, MMIS_Luc/GFP, or RPMI8226_Luc/GFP cells at various concentrations of E1 and D18 after 72 hours is shown in FIGS. 14A-14D, respectively. The combination of CD28 and CD3 stimulation (E1) lead to greater T cell activation than CD3 stimulation alone (D18).

### Example 14: T Cell-Directed Cytotoxicity

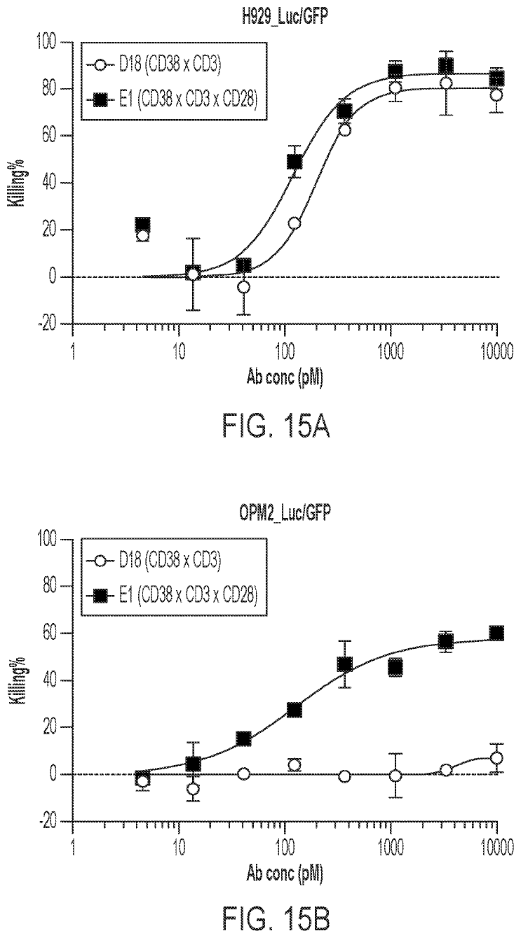

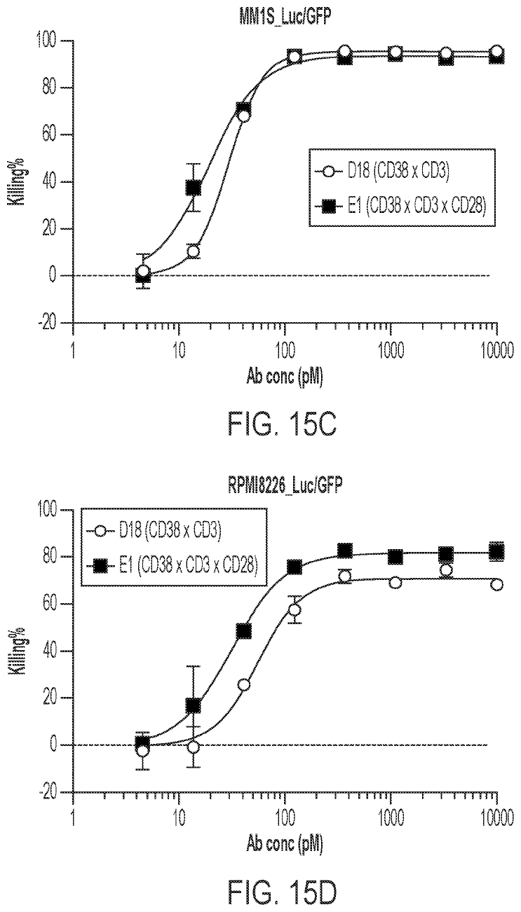

The ability of the exemplary anti-CD38 IgM antibody E1, as described in Example 12, to enhance T cell-directed cytotoxicity (TDCC) compared to the anti-CD38 IgM antibody D18, as described in Example 12, was assessed as follows. 10×10³ human PBMCs were co-cultured with 10×10³ H929_Luc/GFP, OPM2_Luc/GFP, MMIS_Luc/GFP, or RPMI8226_Luc/GFP cells in the presence of serial dilutions of E1 and D18 in 200 μL total volume of culture media (RPMI-1640+ 10% heat inactivated FBS) for 72 hours. 50 μl of supernatant was removed and frozen at −80 °C for later cytokine release analysis. Cells were stained for FACS analysis with the following staining panel: anti-CD3 PerCP-Cy5.5, anti-CD8 BV785, anti-CD25 APC, and anti-CD4 BV605 from Biolegend, using the fixable viability dye EFLUOR® 780 (ThermoFisher, 65-0865-14). The number of viable GFP+ target cells in each well were compared to spiked-in counting beads to calculate percentage killing. The data was then analyzed and plotted with GraphPad Prism. Percentage killing of H929_Luc/GFP, OPM2_Luc/GFP, MMIS_Luc/GFP, or RPMI8226_Luc/GFP cells at various doses after 72 hours are shown in FIGS. 15A-15D, respectively. The combination of CD28 and CD3 stimulation (E1) lead to greater TDCC activity than CD3 stimulation alone (D18).

### Example 15: Cytokine Release

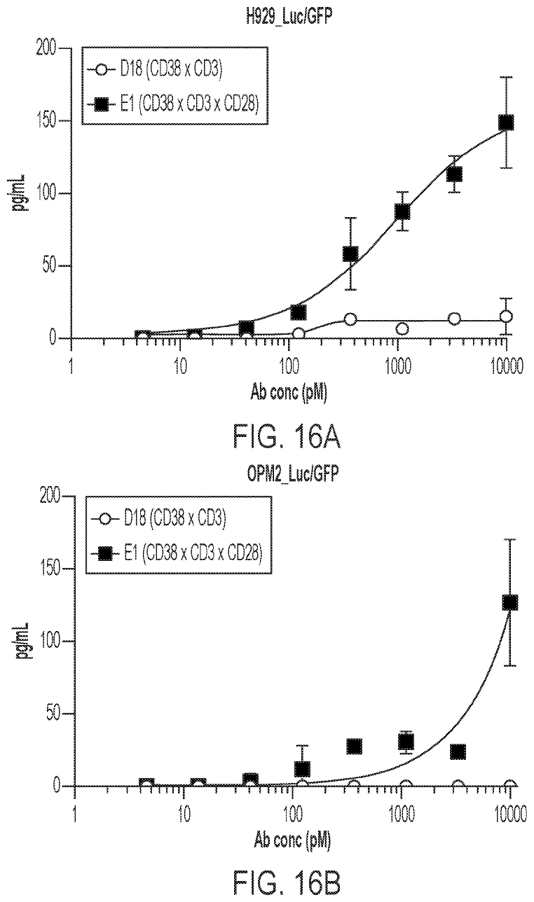

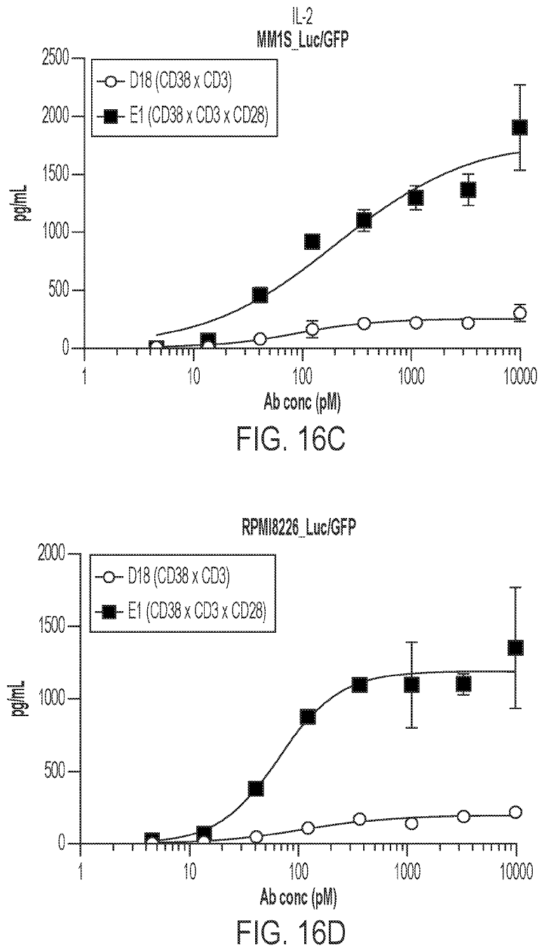

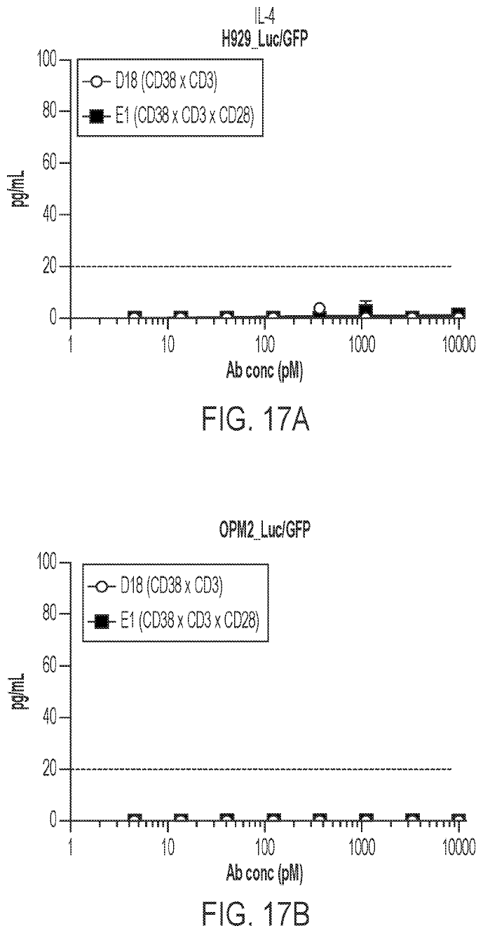

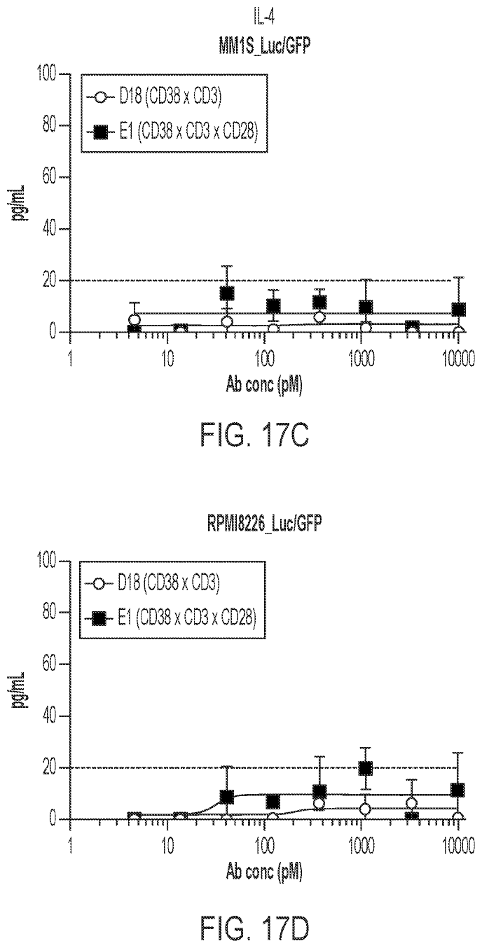

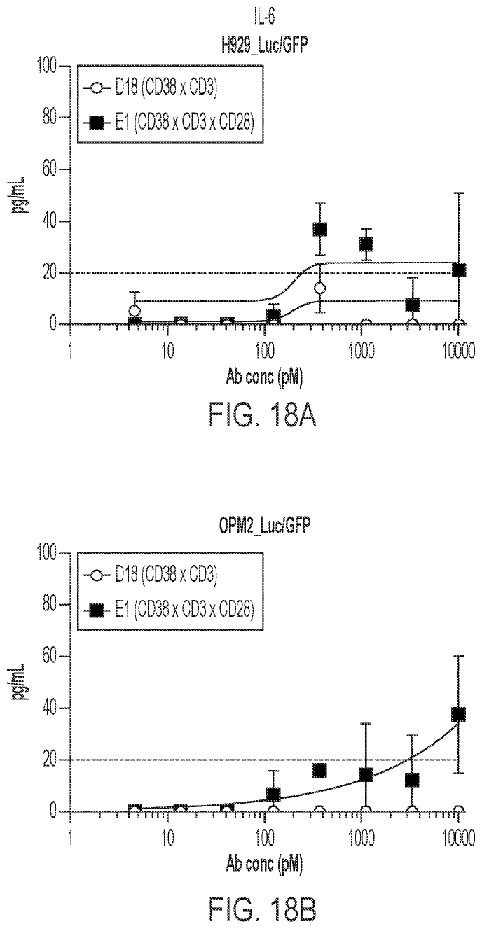

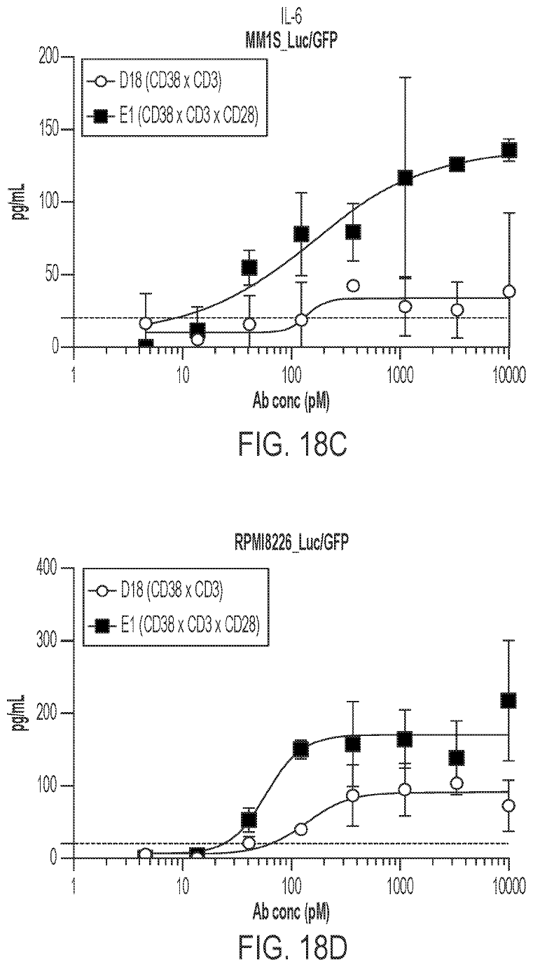

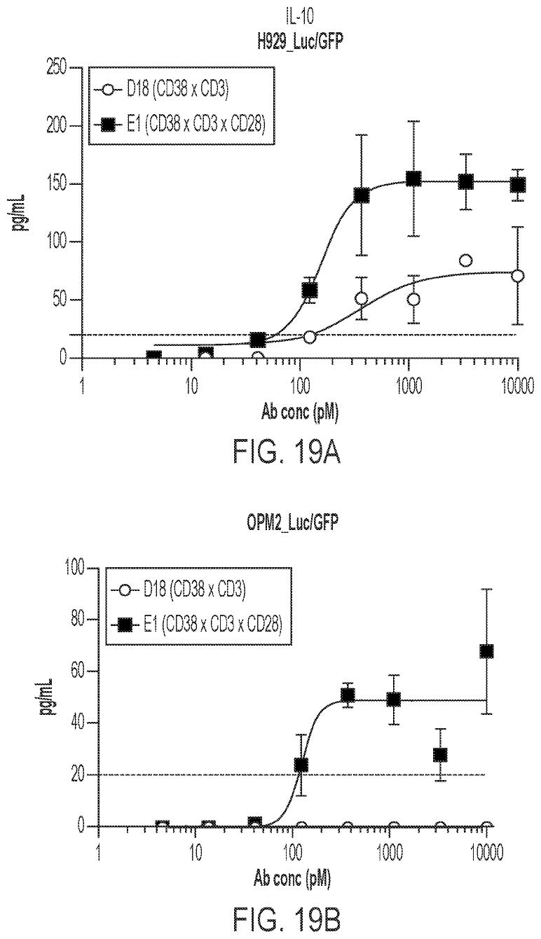

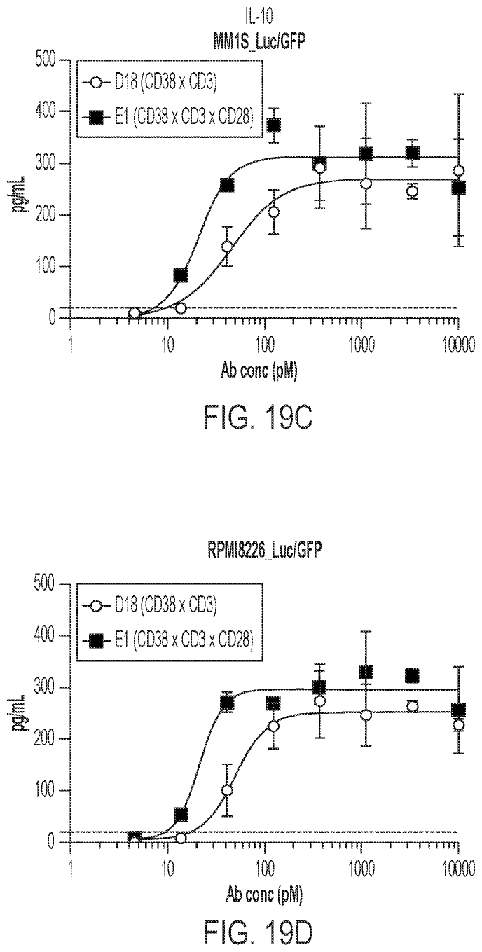

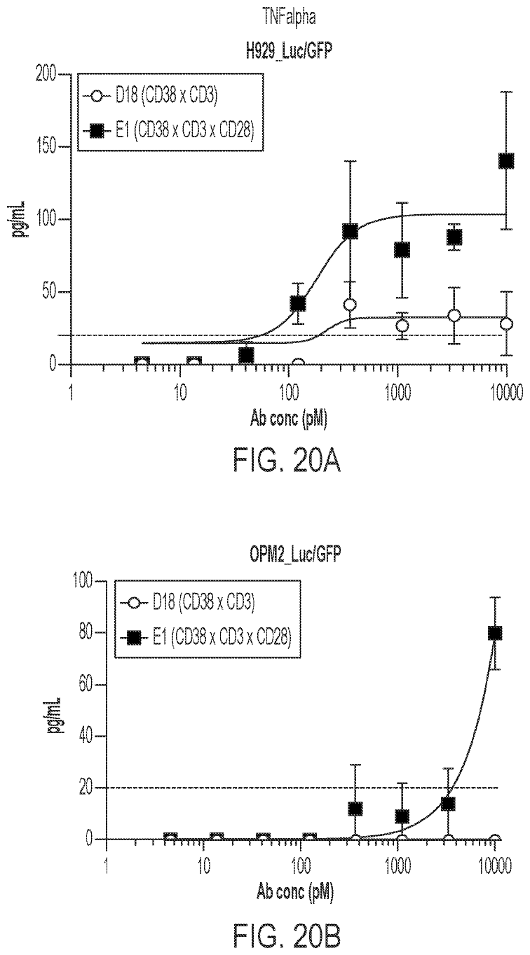

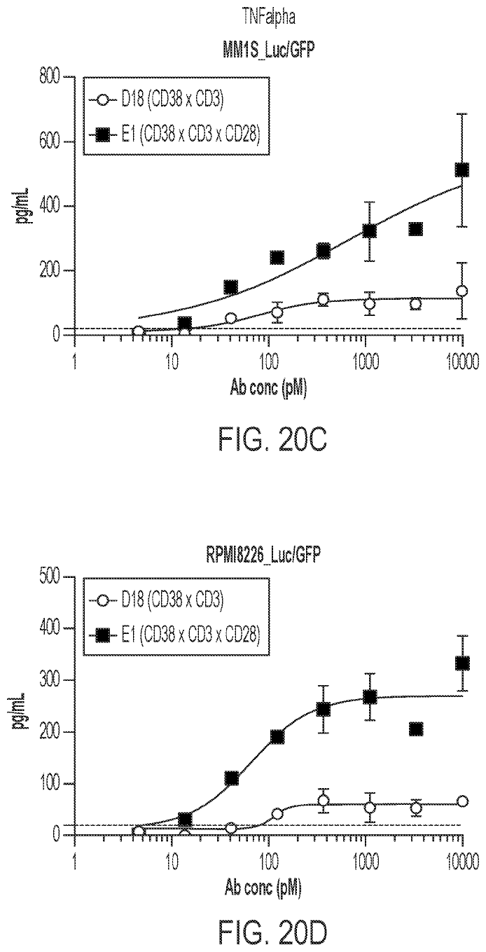

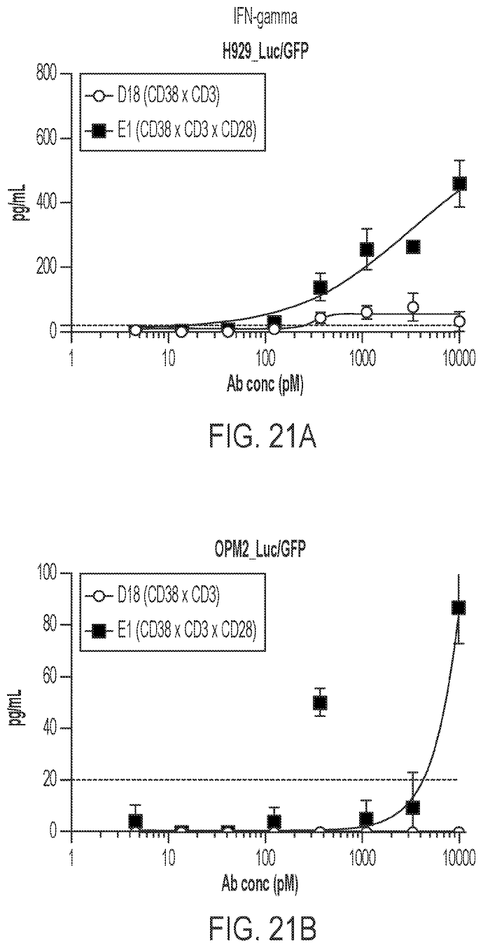

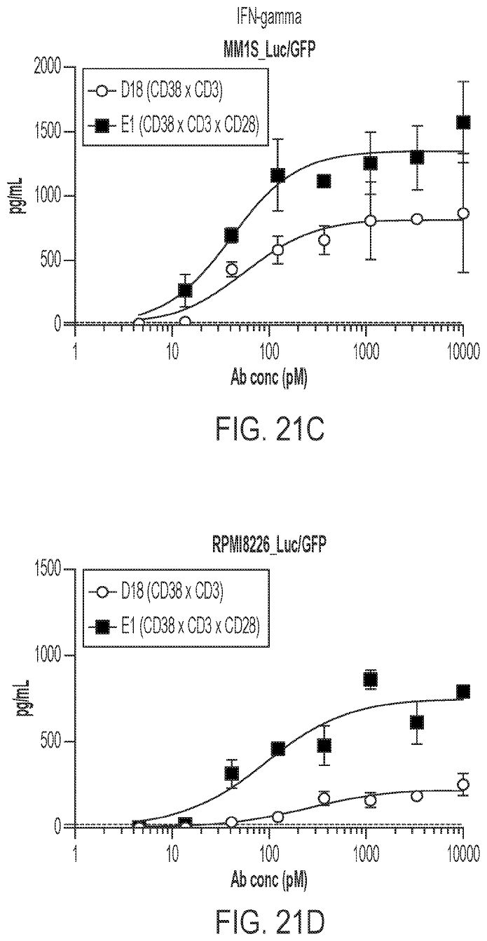

Supernatants from TDCC assays obtained in Example 14 were assayed for a panel of cytokines including IL-2, IL-4, IL-6, IL-10, TNFα, and IFNγ with cytometric bead array_human Th1-2 cytokine kit II (BD, 551809) according to manufacturer's protocol except using smaller volumes to accommodate plate-based assays. The results were then analyzed with GraphPad Prism. Cytokine concentrations in supernatants from a co-culture of PBMC cells and H929_Luc/GFP, OPM2_Luc/GFP, MMIS_Luc/GFP, and RPMI8226_Luc/GFP cells after 48 hours treated with D18 or E1 are shown in FIGS. 16A-16D, respectively for IL-2, FIGS. 17A-17D, respectively for IL-4, FIGS. 18A-18D, respectively for IL-6, FIGS. 19A-19D, respectively for IL-10, FIGS. 20A-20D, respectively for TNFα, and FIGS. 21A-21D, respectively for IFNγ. Enhanced TDCC and T cell activation correlated with cytokine response.

### Example 16: T Cell Activation

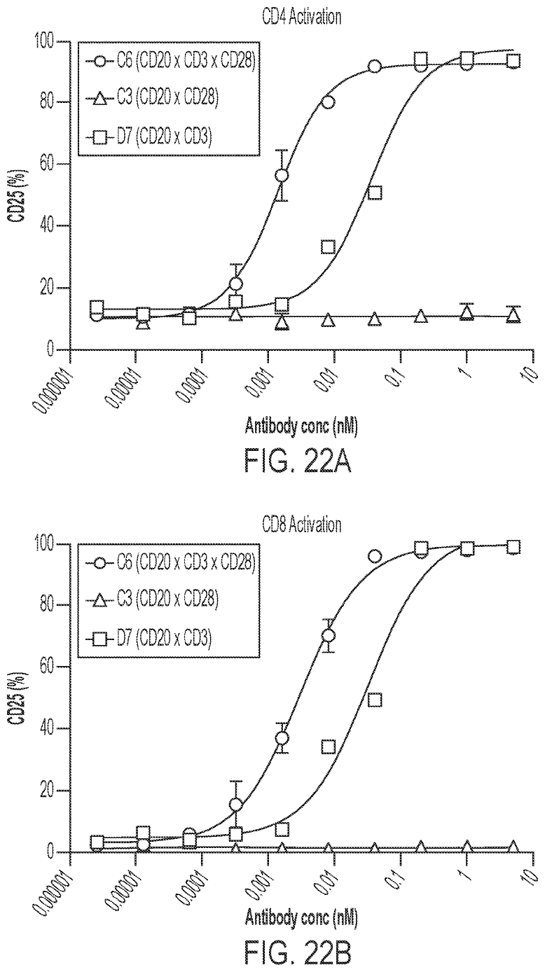

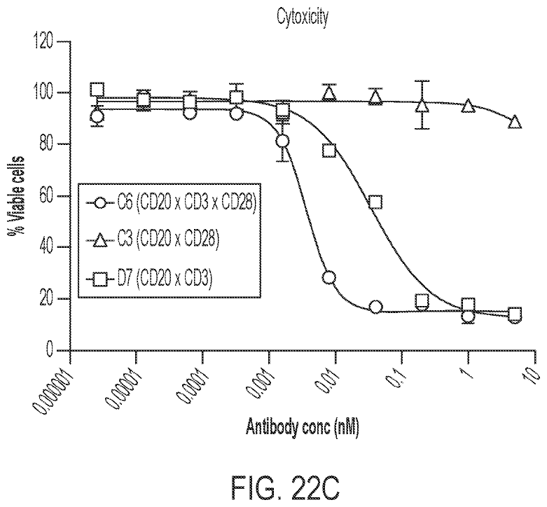

The ability of the exemplary anti-CD20 x anti-CD3 x anti-CD28 IgM antibody C6, as described in Example 1, to enhance T cell activation compared to the anti-CD20 x anti-CD3 IgM antibody D7, as described in Example 1, and the anti-CD20 x anti-CD28 IgM antibody C3, as described in Example 1, was assessed as follows. 50×10³ human PBMCs were co-cultured with Raji-Luc-GFP cells in the presence of serial dilutions of C6, D7, or C3 in 200 μL total volume of culture media (RPMI-1640+ 10% heat inactivated FBS) for 96 hours. Cells were stained for FACS analysis with the following antibody panel: anti-CD3-BV421, anti-CD8-BV650, anti-CD25-BV785, and anti-CD4-BV605 from Biolegend, using the fixable viability dye Aqua (Biolegend #423102). Activation of CD4+ or CD8+ T cells co-cultured with Raji-Luc-GFP cells at various concentrations of C6, D7, or C3 after 96 hours is shown in FIGS. 22A-22B, respectively. The combination of CD28 and CD3 stimulation (C6) lead to greater T cell activation than CD3 (D7) or CD28 (C3) stimulation alone.

### Example 17: T Cell-Directed Cytotoxicity

The ability of the exemplary anti-CD20 x anti-CD3 x anti-CD28 IgM antibody C6, as described in Example 1, compared to the anti-CD20 x anti-CD3 IgM antibody D7, as described in Example 1, and the anti-CD20 x anti-CD28 IgM antibody C3, as described in Example 1, to enhance T cell-directed cytotoxicity (TDCC) was assessed as follows. 50 x 103 human PBMCs were co-cultured with Raji-Luc-GFP cells in the presence of serial dilutions of C6, D7, or C3 in 200 μL total volume of culture media (RPMI-1640+ 10% heat inactivated FBS) for 96 hours. 100 μL of supernatant was removed and frozen at −80 °C for later cytokine release analysis. Cells were stained with fixable viability dye Aqua (Biolegend #423102). The number of viable Raji-luc-GFP cells (gated GFP+Aqua−) in each well was compared to the antibody untreated group to calculate percentage killing. The data were then plotted with GraphPad Prism. Percentage killing of Raji-Luc-GFP cells at various concentrations after 96 hours is shown in FIG. 22C. The combination of CD28 and CD3 stimulation (C6) lead to greater TDCC activity than CD3 (D7) or CD28 (C3) stimulation alone.

### Example 18: Cytokine Release

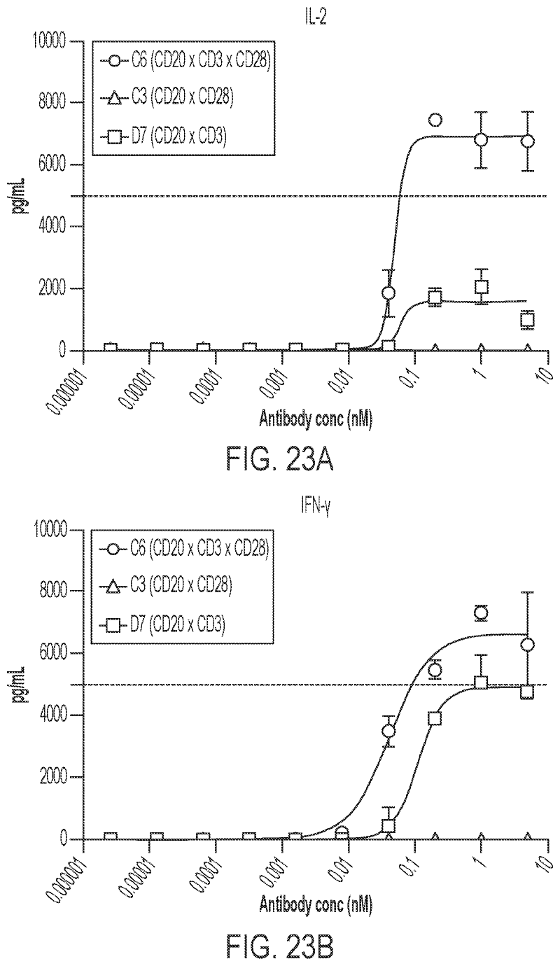

Supernatants from TDCC assays obtained in Example 17 were assayed for cytokines including IL-2 and IFN-γ using the cytometric bead array human Th1-2 cytokine kit II (BD, 551809) according to manufacturer's protocol except using smaller volumes to accommodate plate-based assays. The results were then plotted with GraphPad Prism. Cytokine IL-2 and IFNγ concentrations in supernatants from a co-culture of PBMC cells and Raji-Luc-GFP cells after 96 hours treated with C6, D7, or C3 are shown in FIGS. 23A and 23B, respectively. Enhanced TDCC and T cell activation correlated with cytokine response.

### Example 19: In Vivo Activity

Antitumor activity of the exemplary IgM antibodies: C3 (the anti-CD20 x anti-CD28 IgM antibody described in Example 1), C7 (the anti-CD20 x anti-CD3 x anti-CD28 IgM antibody described in Example 1), and D7 (the anti-CD20 x anti-CD3 IgM antibody described in Example 1) was evaluated in a Ramos human B lymphocyte cancer xenograft model. NSG-MHC I/II DKO mice were engrafted with 10⁷ human PBMCs on day −14. The mice were implanted subcutaneously with 2×10⁶ Ramos cells on day 0. Two days later, animals were dosed intravenously every other day for 6 doses (q2dx6) or every other day for 12 doses (q2dx12) as shown in Table 16. Tumor size was measured 2-3 times per week. Samples of whole blood were taken 4 days post PBMC engraftment, 4 hours post-first dose, 4 hours after dose 6, and between doses 6 and 12. Additionally, at the study end point, tumors, spleen, and blood were harvested.

TABLE 16
Dosing Schedule

| | | | | Test | Amount | |
|---|---|---|---|---|---|---|
| Group | n | Ramos | hPBMC | Article | (mg/kg) | Regimen |
| 1 | 6 | 2 × 10\hat{ }6 | — | — | — | q2dx12 |
| 2 | 8 | 2 × 10\hat{ }6 | 10 × 10\hat{ }6 | Vehicle | — | q2dx6 |
| 3 | 8 | 2 × 10\hat{ }6 | 10 × 10\hat{ }6 | C3 | 10 | q2dx6 |
| 4 | 8 | 2 × 10\hat{ }6 | 10 × 10\hat{ }6 | C7 | 10 | q2dx6 |
| 5 | 8 | 2 × 10\hat{ }6 | 10 × 10\hat{ }6 | C7 | 1 | q2dx12 |
| 6 | 8 | 2 × 10\hat{ }6 | 10 × 10\hat{ }6 | C7 | 0.1 | q2dx6 |
| 7 | 8 | 2 × 10\hat{ }6 | 10 × 10\hat{ }6 | D7 | 10 | q2dx6 |
| 8 | 8 | 2 × 10\hat{ }6 | 10 × 10\hat{ }6 | D7 | 1 | q2dx12 |
| 9 | 8 | 2 × 10\hat{ }6 | 10 × 10\hat{ }6 | D7 | 0.1 | q2dx6 |

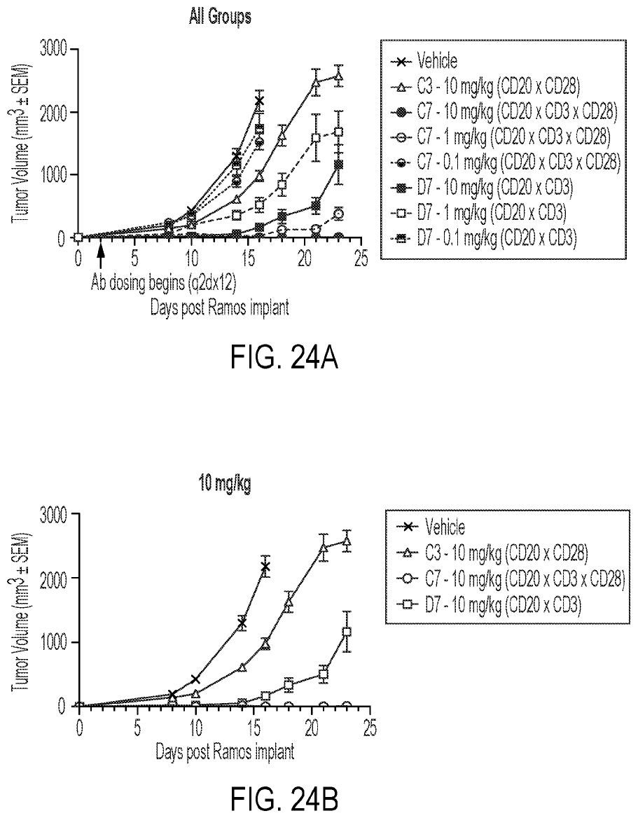

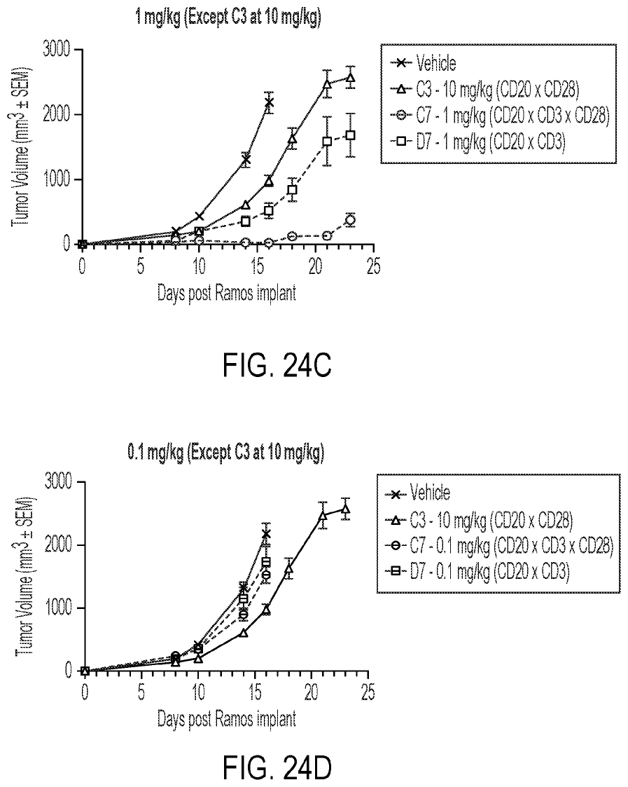

Graphs of tumor volumes versus days post-Ramos implant (i.e., time, in days) up to day 23 for the various treatment groups are provided in FIGS. 24A-D. Tumor volumes over time for each group are provided in FIG. 24A, tumor volumes over time for the 10 mg/kg groups (groups 3, 4, and 7) are shown in FIG. 24B, tumor volumes for the 1 mg/kg C7 and D7 groups, and for the 10 mg/kg C3 group are shown in FIG. 24C. Tumor volumes for the 0.1 mg/kg C7 and D7 groups and the 10 mg/kg C3 group are shown in FIG. 24D. Tumor volume was lower for the C7 (anti-CD20 x anti-CD3 x anti-CD28) treatment groups at 1 mg/kg and 10 mg/kg than for any other treatment group as shown in FIG. 24A. As shown in FIG. 24B, for group 4 (C7 dosed at 10 mg/kg), tumor volume was notably 0 mm³ at day 23.

Table 17 provides a summary of tumor growth inhibition (TGI) and the number of tumor-free (TF) mice for the various treatment groups determined at day 16 (the last day of the study where all mice in the vehicle group were non-expired).

TABLE 17
Tumor Growth Inhibition (TGI)

| | | % TGI | No. of |
|---|---|---|---|
| Group | Treatment Group Description | (day 16) | TF Mice |
| 4 | C7 - CD20 × CD3 × CD28 - 10 mg/kg | 101.1 | 8 |
| 5 | C7 - CD20 × CD3 × CD28 - 1 mg/kg | 100.4 | 1 |
| 6 | C7 - CD20 × CD3 × CD28 - 0.1 mg/kg | 35.3 | 0 |
| 7 | D7 - CD20 × CD3 - 10 mg/kg | 92.2 | 5 |
| 8 | D7 - CD20 × CD3 - 1 mg/kg | 76.5 | 0 |
| 9 | D7 - CD20 × CD3 - 0.1 mg/kg | 22.4 | 0 |

As shown in the data in Table 17, for the treatment groups evaluated, percent tumor growth inhibition was the greatest for the 10 mg/kg C7 and the 1 mg/kg C7 treatment groups (groups 4 and 5, respectively) with the anti-CD20 x anti-CD3 x anti-CD28 IgM antibody. The next highest percent tumor growth inhibition was observed for group 7 (D7 (anti-CD20 x anti-CD3 IgM antibody dosed at 10 mg/kg).

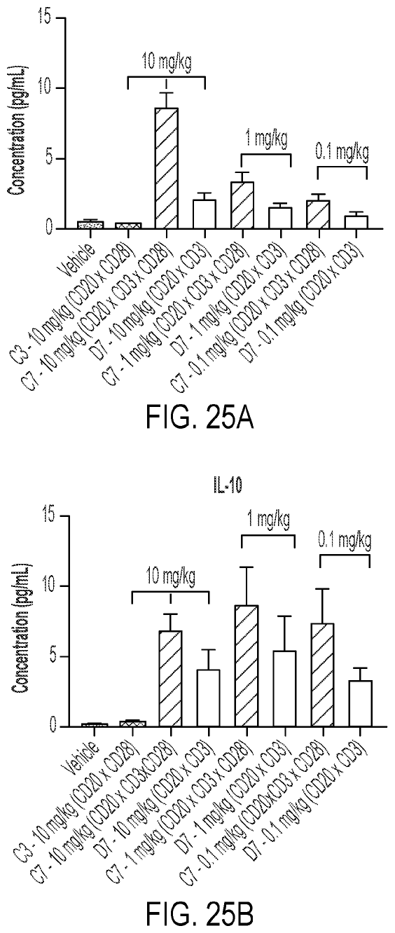

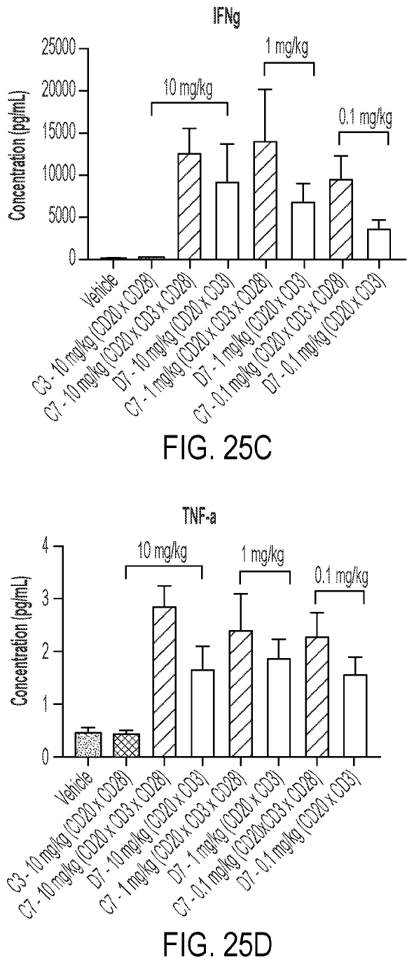

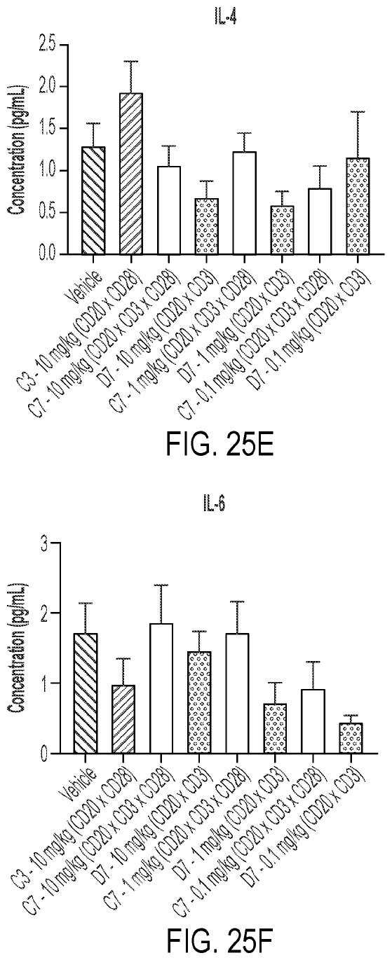

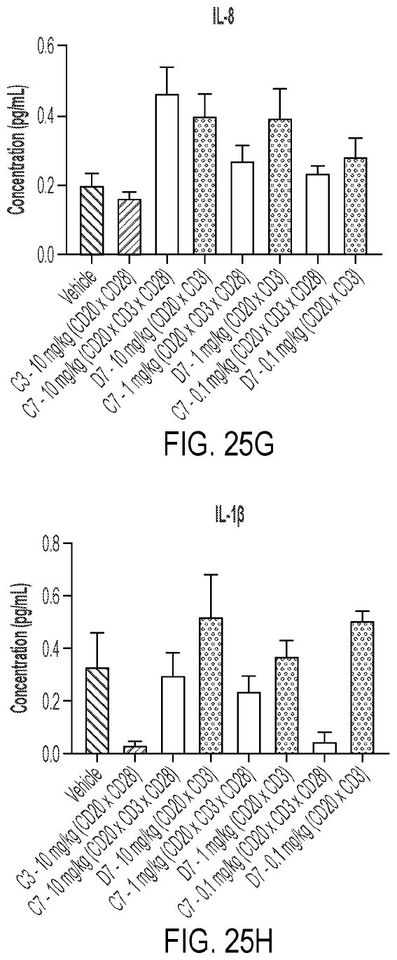

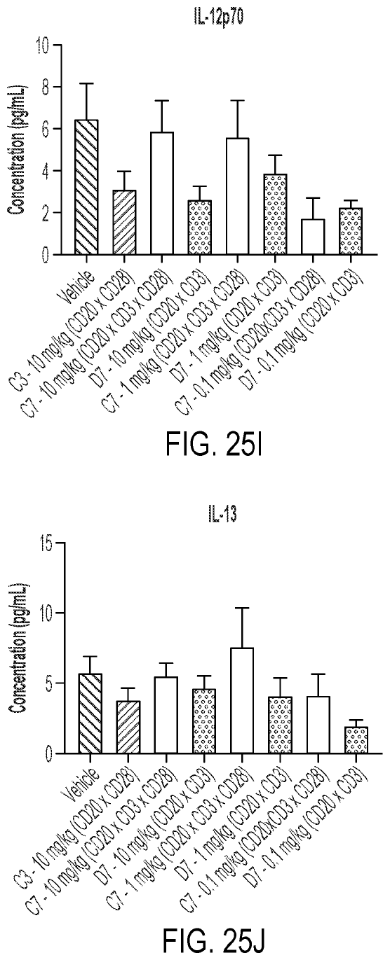

Harvested serum samples from each treatment group were analyzed 4 hours post first dose for cytokines IL-2, IL-10, IFN-γ, TNF-α, IL-4, IL-6, IL-8, IL-1β, IL-12p70, and IL-13 concentrations using a MSD multiplex cytokine assay kit (Meso Scale Discovery) according to manufacturer's protocol. The results were then plotted with GraphPad Prism. IL-2, IL-10, IFN-γ, TNF-α, IL-4, IL-6, IL-8, IL-1β, IL-12p70, and IL-13 serum concentrations are shown in FIGS. 25A-25J, respectively. At each dose level, C7 (anti-CD20 x anti-CD3 x anti-CD28) stimulated greater IL-2, IL-10, IFN-γ, TNF-α, IL-6 and IL-13 release than D7 (anti-CD20 x anti-CD3) at the same dose, and greater release than C3 (anti-CD20 x anti-CD28) at 10 mg/kg. Turning to serum levels of IL-4 shown in FIG. 25E, at 10 mg/kg, C3 stimulated greater IL-4 release than did C7 or D7 at the same dose; at a dose of 1 mg/kg, C7 stimulated greater IL-4 release than did D7; at a dose of 0.1 mg/kg, D7 stimulated greater IL-4 release than did C7. Turning to serum levels of IL-8 shown in FIG. 25G, at a dose of 10 mg/kg, C7 stimulated IL-8 to a greater extent than either D7 or C3; at doses of 1 mg/kg and 0.1 mg/kg, D7 stimulated IL-8 to a greater extent than C7. As shown in FIG. 25H, D7 stimulated IL-1β to a greater extent than did C7 or C3 at a dose of 10 mg/kg, and stimulated IL-1ß to a greater extent than did C7 at doses of both 1 mg/kg and 0.1 mg/kg. Turning to FIG. 25I, at a dose of 10 mg/kg, C7 stimulated IL-12p70 to a greater extent than either D7 or C3; similarly, at a dose of 1 mg/kg, C7 stimulated IL-12p70 to a greater extent than D7; in contrast, at a dose of 0.1 mg/kg, D7 stimulated IL-12p70 to a somewhat greater extent than C7.

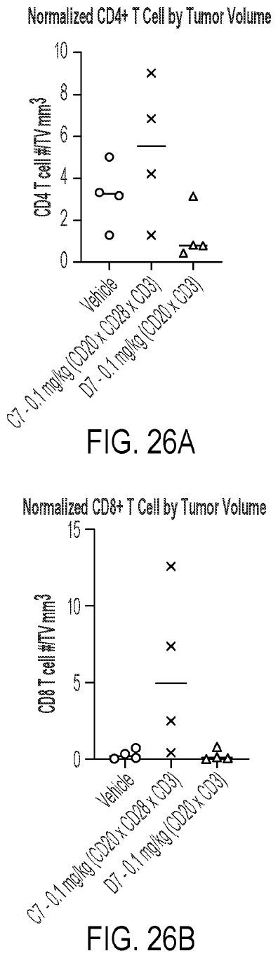

The number of T cells at the tumor site for each treatment was assessed as follows. Tumors were harvested and cells were stained for flow cytometry analysis with the following antibody staining panel: anti-mouseCD45-BV785, anti-humanCD45-APC/Cy7, anti-humanCD8-PerCP-Cy5.5, anti-humanCD4-AF700 using the fixable viability dye BV510 (Biolegend). The number of infiltrated CD4+ T cells or CD8+ T cells normalized to tumor volume at the 0.1 mg/kg doses are shown in FIGS. 26A and 26B, respectively. C7 (anti-CD20 x anti-CD3 x anti-CD28) increased both CD4+ and CD8+ T cells at the tumor site to a greater extent than both vehicle and D7 (anti-CD20 x anti-CD3).

### Example 20: Long Term T Cell Dependent Cellular Cytotoxicity (TDCC), T Cell Proliferation and Cytokine Release

Long term T cell dependent cellular cytotoxicity (TDCC), T cell proliferation and cytokine release was assessed in an in vitro model with human PBMC and Ramos tumor cells.

PBMCs and CD20-expressing target tumor cells (Ramos) were washed and resuspended in culture media (RPMI1640+ 10% heat inactivated FBS). Co-culture assays were set up in 96-well U-bottom tissue culture plates with 200 μL culture media per well at different PBMC to Ramos cell ratios with no antibody, or in the presence of antibodies D8, D9, D10, D7, or C6 (as described in Example 1), all at a concentration of 10 nM. Three different effector (PBMC)-to-target (Ramos cells) ratios were tested: 3×10⁴ PBMC and 1×10⁴ Ramos cells per well were seeded for the 3:1 ratio, 1×10⁴ PBMC and 1×10⁴ Ramos cells per well were seeded for the 1:1 ratio, 1×10⁴ PBMC and 3×10⁴ Ramos cells per well were seeded for the 1:3 ratio. The plates were then placed in a humidified 37 °C incubator with 5% CO₂. At 3-day, 7-day, 10-day, and 14-day time points, the plates were taken out, and the cell mixtures were resuspended in each well by pipetting. 100 μL resuspended cell culture mix were taken from each well and transferred to new tissue culture plates and replenished with 100 μL fresh media containing the same concentrations of antibodies for continued culture at 37 °C and 5% CO₂. The cell culture mixes in the original tissue culture plates were used to assay for TDCC and for CD4+ and CD8+ T cell proliferation at each time point, and for cytokine analysis at the 3-day (72-hour) time point.

The cell culture mixes to be assayed were centrifuged at 300 g for 5 min, and 50 μL of supernatant samples were collected from each well and frozen at −80 °C for cytokine analysis later. 5000 counting beads (Life Technologies, catalog #C36950) were then added to each well and mixed with the cells by pipetting. The cells and counting beads mixtures were again centrifuged, and the cells and counting beads mixtures were resuspended in the multi-color flow antibody cocktail to stain human CD3, CD4, CD8, CD25 and CD19. The fixable viability dye (EFLUOR® 780, Thermo Fisher Scientific, catalog #65-0865-14) was also included in the staining cocktail for viability staining. After 30 min incubation at 4 °C, the samples were washed with 200 μL FACS stain buffer (BD Biosciences, catalog #554656) and then resuspended in 25 μL FACS stain buffer for flow cytometry analysis using INTELLICYT® IQUE® Screener PLUS (Sartorius) and the INTELLICYT® FORECYT® software (Sartorius).

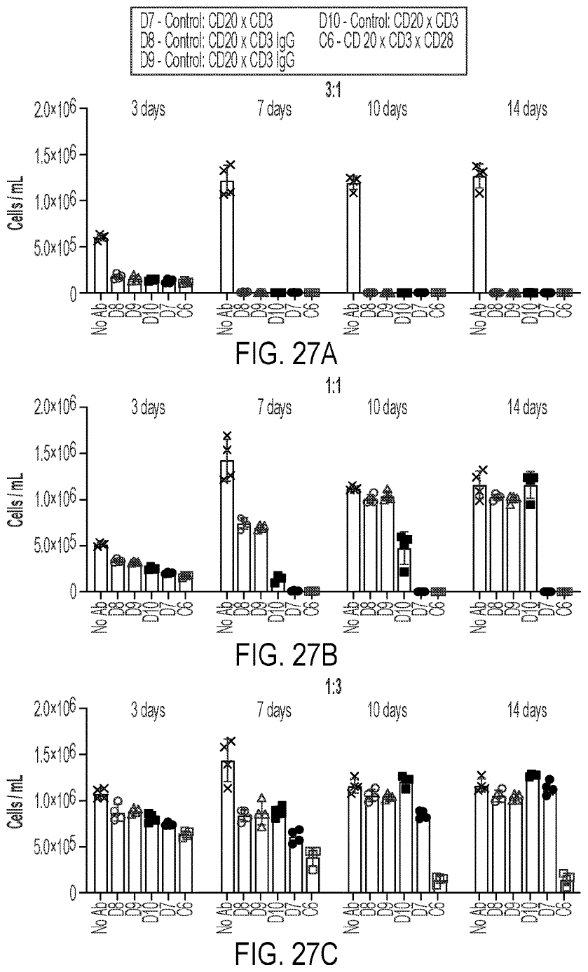

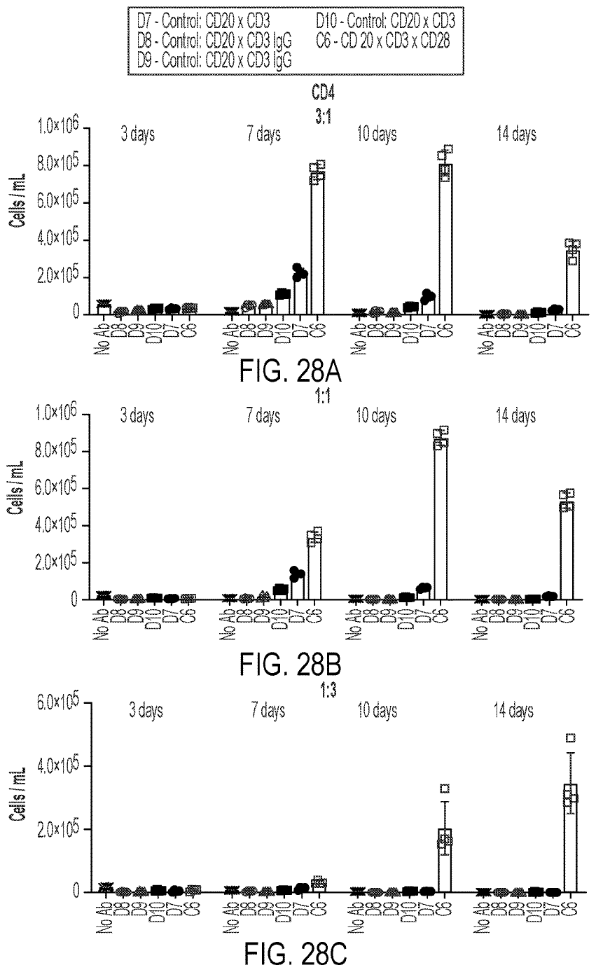

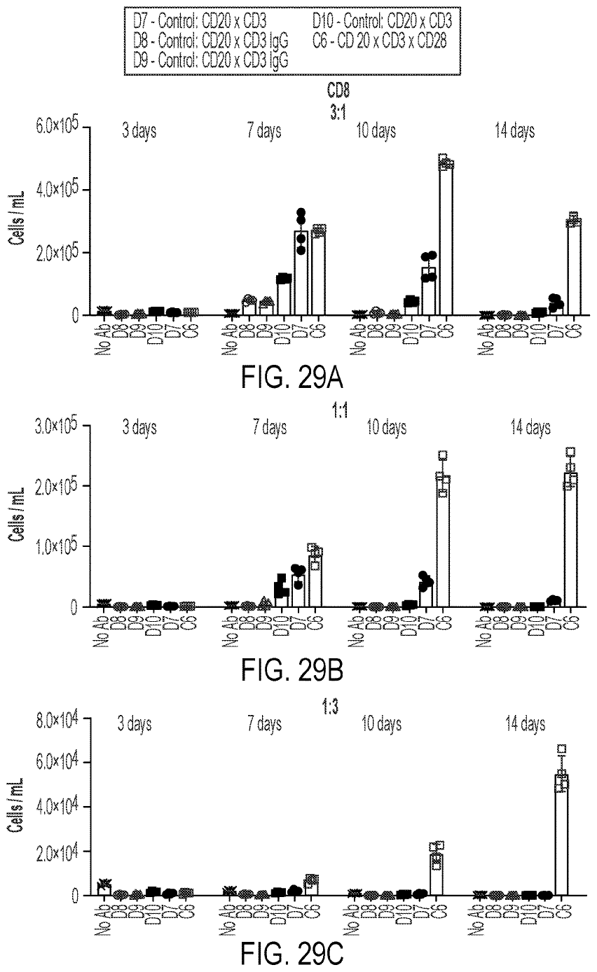

For the TDCC and T cell proliferation analyses, target cells (Ramos and primary B cells) were identified by CD19 staining and T cell subsets were identified by CD3, CD4, CD8 and CD25 staining results from the FACS analyses. The number of live target cells and T cell subsets in each well were calculated by comparing the number of target cell events to the number of counting bead events. Cell numbers were then divided by the volume of the cell culture sample to get the cell density in the culture and monitored over multiple time points to evaluate the long-term cytotoxicity and T cell proliferation effects of different test articles. The TDCC results for the three different effector-to-target ratios are shown in FIGS. 27A-C, respectively. The CD4 T cell proliferation results at the three different effector-to-target ratios are shown in FIGS. 28A-C, respectively. The CD8 T cell proliferation results at the three different effector-to-target ratios are shown in FIGS. 29A-C, respectively.

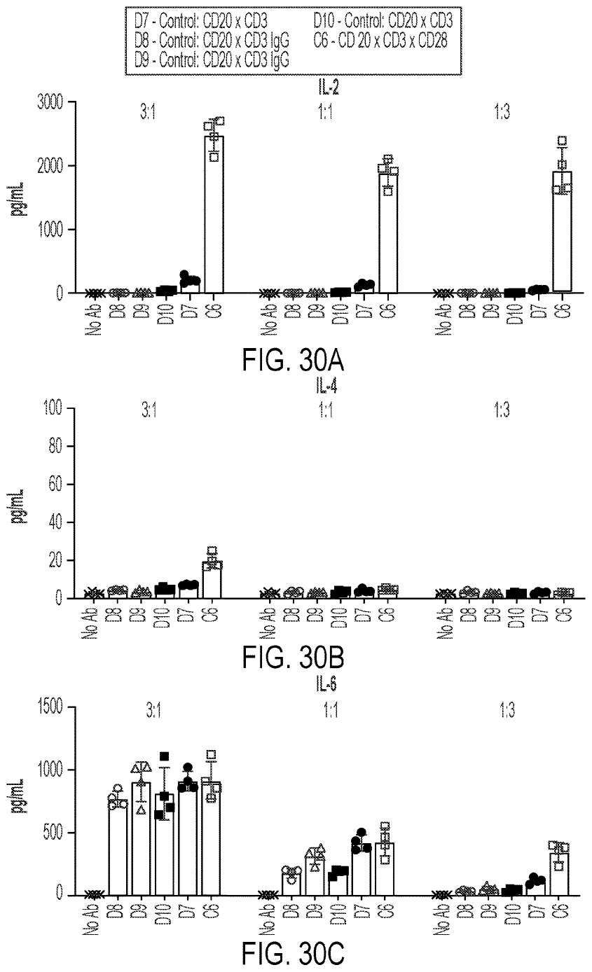

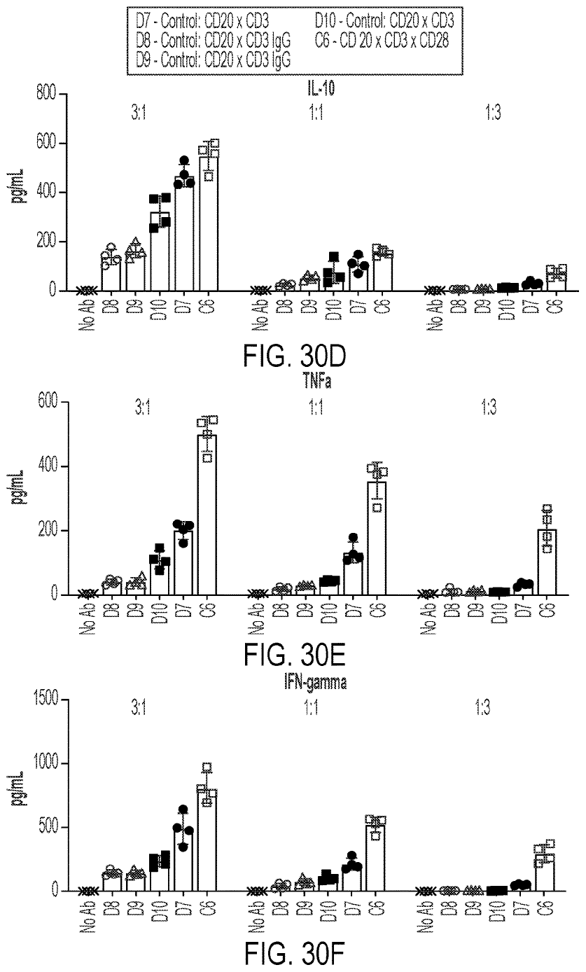

To measure the cytokine release mediated by the test articles, the supernatant samples from the 3-day (72-hour) time point were thawed and analyzed by flow cytometry, using the Human Th1/Th2 Cytokine Cytometric Bead Array (CBA) Kit II from BD Biosciences (catalog #551809) based on manufacture's recommendation, except using smaller culture volume to accommodate a plate-based format. The flow cytometry analysis was performed on the INTELLICYT® IQUE® Screener PLUS and data was analyzed using the INTELLICYT® FORECYT® software, including standard curve fitting and cytokine concentration calculations. The levels of IL-2, IL-4, IL-6, IL-10, TNFα, and IFN-γ at each of the three different effector-to-target ratios are shown in FIGS. 30A-F, respectively.

### Example 21: In Vivo Efficacy-Survival Study

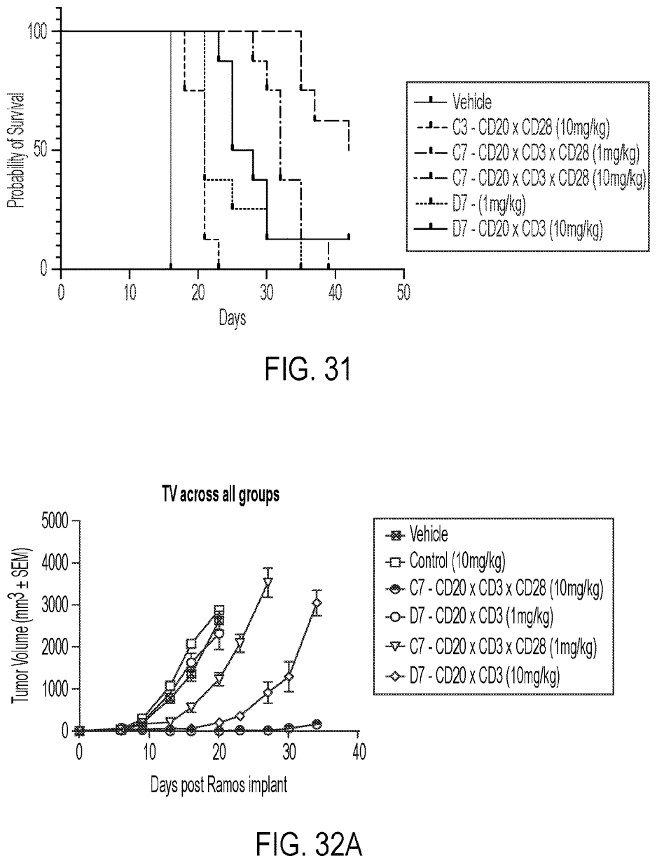

Antitumor activity of the exemplary IgM antibodies: C3 (the anti-CD20 x anti-CD28 IgM antibody described in Example 1), C7 (the anti-CD20 x anti-CD3 x anti-CD28 IgM antibody described in Example 1), and D7 (the anti-CD20 x anti-CD3 IgM antibody described in Example 1) was evaluated in a Ramos human B lymphocyte cancer xenograft model by examining overall survival. NSG-MHC I/II DKO mice were engrafted with 10⁷ human PBMCs on day −14. The mice were implanted subcutaneously with 2×10⁶ Ramos cells on day 0. Two days later, animals were dosed intravenously every other day for 6 doses (q2dx6) or every other day for 12 doses (q2dx12) as shown in Table 18. The low dose groups received a total of 12 doses while other groups received 6 doses. Mice were euthanized when tumor sizes reached 2000 mm³. Median overall survival in days for each treatment group is provided in Table 18. Survival probabilities for each of the treatment groups over time were determined using the Kaplan-Meier method; the resulting survival curve is shown in FIG. 31.

As shown in Table 18, at both the 10 mg/kg and 1 mg/kg doses, mice treated with C7 showed significantly longer median overall survival than did mice treated with C3 or D7, or with D7, respectively. The median overall survival of mice treated with C7 at the 10 mg/kg dose was approximately twice that of mice treated with C3, and approximately 1.6 times that of mice treated with D7, whereas the median overall survival of mice treated with C7 at the 1 mg/kg dose was approximately 1.5 times that of mice treated with D7.

TABLE 18
Dosing Regimen

| | | | | | Median |
|---|---|---|---|---|---|
| | | | | | Overall |
| | | | Dose | Dosing | Survival |
| Group | N | Test article | (mg/kg) | Schedule | (days) |
| 2 | 8 | Vehicle | — | q2d × 6 | 16 |
| 3 | 8 | C3 (CD20 × CD28) | 10 | q2d × 6 | 21 |
| 5 | 8 | C7 CD20 × CD3 × CD28) | 1 | q2d × 12 | 32 |
| 4 | 8 | C7 (CD20 × CD3 × CD28) | 10 | q2d × 6 | 42 |
| 8 | 8 | D7 (CD20 × CD3) | 1 | q2d × 12 | 21 |
| 7 | 8 | D7 CD20 × CD3 | 10 | q2d × 6 | 26.5 |

### Example 22: In Vivo Efficacy

In an additional preclinical efficacy study, the antitumor activity of the exemplary IgM antibodies, C7 (the anti-CD20 x anti-CD3 x anti-CD28 IgM antibody described in Example 1), D7 (the anti-CD20 x anti-CD3 IgM antibody described in Example 1), and an isotype control antibody D15 was evaluated in a Ramos human B lymphocyte cancer xenograft model. NSG-MHC I/II DKO mice were engrafted with 10⁷ human PBMCs on day −14. The mice were implanted subcutaneously with 2×10⁶ Ramos cells on day 0. Two days later, animals were dosed intravenously biweekly for a total of 12 doses (biwx12) as shown in Table 19. Tumor size was measured 2-3 times per week. Samples of whole blood were taken 4 days post PBMC engraftment, 4 hours post-first dose, 4 hours after dose 6, and between doses 6 and 12. Additionally, at the study end point, tumors, spleen, and blood were harvested.

TABLE 19
Dosing Schedule

| | | | hPBMC | Test | Amount | | |
|---|---|---|---|---|---|---|---|
| Group | N | Ramos | (#5662) | Article | (mg/kg) | Route | Regimen |
| 1 | 8 | 2 × 10\hat{ }6 | 10 × 10\hat{ }6 | Vehicle | — | i.v | biwx12 |
| 2 | 8 | 2 × 10\hat{ }6 | 10 × 10\hat{ }6 | Control D15 | 10 | i.v | biwx12 |
| 3 | 8 | 2 × 10\hat{ }6 | 10 × 10\hat{ }6 | C7 | 10 | i.v | biwx12 |
| | | | | (CD20 × CD3 × CD28) | | | |
| 4 | 8 | 2 × 10\hat{ }6 | 10 × 10\hat{ }6 | C7 | 1 | i.v | biwx12 |
| | | | | (CD20 × CD3 × CD28) | | | |
| 5 | 8 | 2 × 10\hat{ }6 | 10 × 10\hat{ }6 | D7 (CD20 × CD3) | 10 | i.v | biwx12 |
| 6 | 8 | 2 × 10\hat{ }6 | 10 × 10\hat{ }6 | D7 (CD20 × CD3) | 1 | i.v | biwx12 |

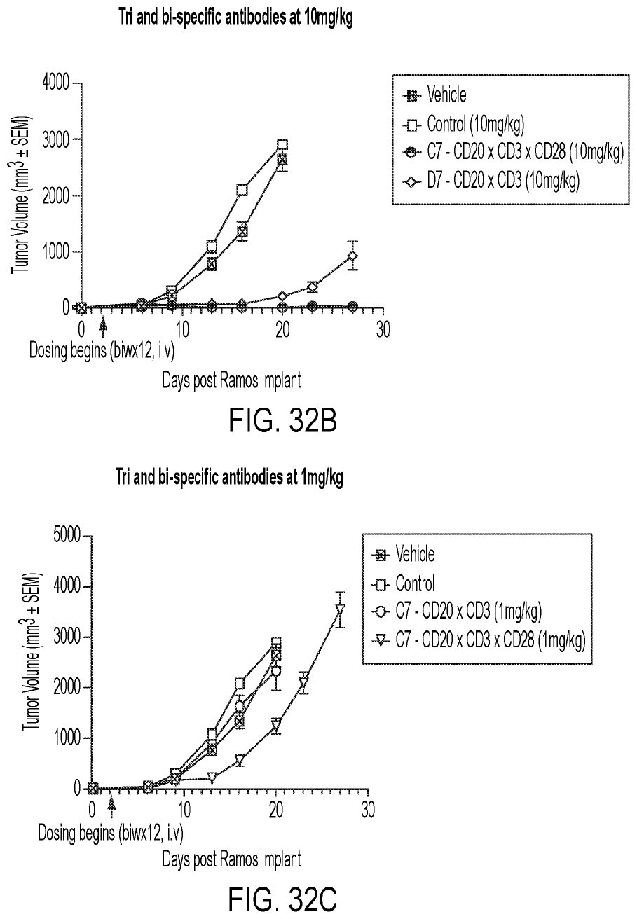

Graphs of tumor volumes versus days post-Ramos implant (i.e., time, in days) for each of the treatment groups are provided in FIGS. 32A-C. Tumor volumes over time for each group are provided in FIG. 32A; tumor volumes over time for the 10 mg/kg treatment groups (groups 2, 3, and 5) are shown in FIG. 32B; tumor volumes for the 1 mg/kg C7 and D7 treatment groups, and for the 10 mg/kg control group are shown in FIG. 32C. Tumor volume over time was lower for the C7 (anti-CD20 x anti-CD3 x anti-CD28) treatment groups at dosages of both 1 mg/kg and 10 mg/kg than for any other treatment group.

Table 20 provides a summary of tumor growth inhibition (TGI) and the number of tumor-free (TF) mice in the various treatment groups determined at day 16 (the last day of the study where all mice in the vehicle group were non-expired). As shown in Table 20, in this preclinical model and experiment, antibody C7 provided a superior antitumor effect when compared to antibody D7 at an equivalent dose.

TABLE 20
Tumor Growth Inhibition (TGI)

| | | | # of |
|---|---|---|---|
| Group | Treatment groups | % TGI | TF mice |
| 3 | C7 - CD20 × CD3 × CD28 @10 mg/kg | 102.6 | 8 |
| 4 | C7 - CD20 × CD3 × CD28 @1 mg/kg | 66.4 | 0 |
| 5 | D7 - CD20 × CD3 @10 mg/kg | 98.8 | 3 |
| 6 | D7 - CD20 × CD3 @1 mg/kg | −23.9 | 0 |

*% % TGI and # of TF mice are from day 16 of study, the last day where all mice in vehicle group were non-expired.

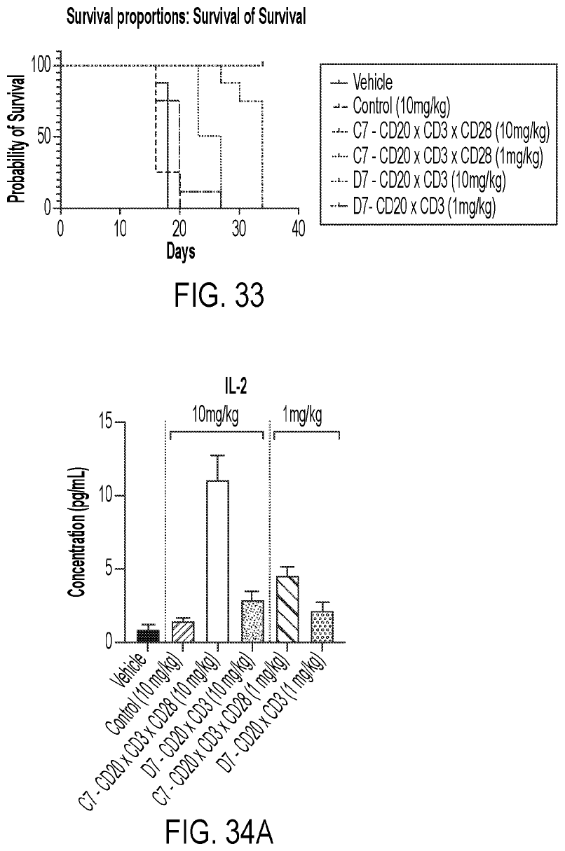

Median overall survival (OS) in days for each treatment group is provided in Table 21. Survival probabilities for each of the treatment groups over time were determined using the Kaplan-Meier method; the resulting survival curve is shown in FIG. 33.

TABLE 21
Median Overall Survival

| | | Dose | Median OS |
|---|---|---|---|
| Group | Test article | (mg/kg) | (day) |
| 1 | Vehicle | — | 18 |
| 2 | Control | 10 | 16 |
| 3 | C7 - CD20 × | 10 | Undefined* |
| | CD3 × CD28 | | |
| 4 | C7 - CD20 × | 1 | 25 |
| | CD3 × CD28 | | |
| 5 | D7 - CD20 × CD3 | 10 | 34 |
| 6 | D7 - CD20 × CD3 | 1 | 20 |

*All 8 mice in the CD20 × CD3 × CD28 10 mpk group were alive at study termination on D 34. 5/8 mice were TF.

As shown in Table 21, at both the 10 mg/kg and 1 mg/kg doses, mice treated with C7 showed longer median overall survival than did mice treated with D7, and mice treated with C7 or D7 demonstrated prolonged survival over both the vehicle and the control groups. At the termination of the study, all mice in the C7 (10 mg/kg) treatment group were alive, such that a value of median overall survival could not be calculated, with five of the eight mice being tumor-free at day 34.

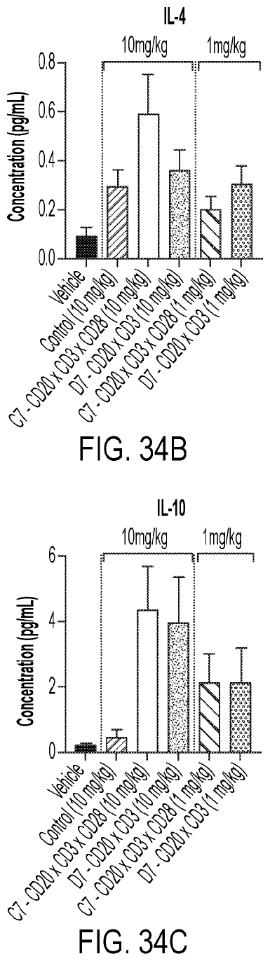

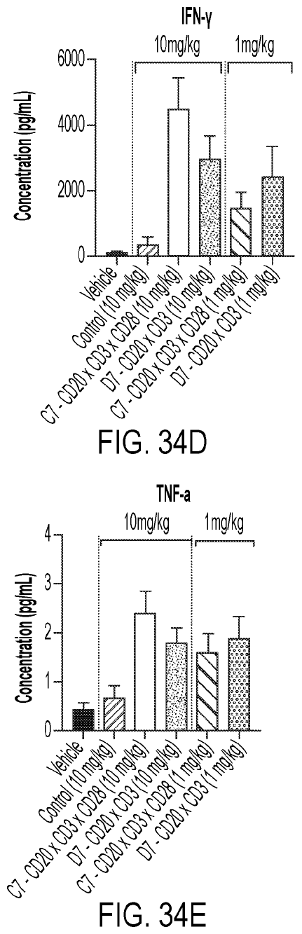

Harvested serum samples from each treatment group were analyzed 4 hours post first dose for cytokines IL-2, IL-4, IL-10, IFN-γ, and TNF-α concentrations using an MSD multiplex cytokine assay kit (Meso Scale Discovery) according to manufacturer's protocol. The results were then plotted with GraphPad Prism. IL-2, IL-4, IL-10, IFN-γ, TNF-α serum concentrations (pg/mL) are shown in FIGS. 34A-34E, respectively. In this experiment, C7 (anti-CD20 x anti-CD3 x anti-CD28) stimulated greater IL-2 release than D7 (anti-CD20 x anti-CD3) at both the 10 mg/kg and 1 mg/kg dosages. For the other cytokines examined, IL-4, IL-10, IFN-γ, and TNF-α, C7 stimulated greater cytokine release at the 10 mg/kg dosage than D7, while at the lower dosage amount, D7 stimulated greater or about equal (IL-10) cytokine release than C7.

### Example 23: Pharmacodynamic (PD) Study

A pharmacodynamic study was carried out using a Ramos human B lymphocyte cancer xenograft model. NSG-MHC I/II DKO mice were engrafted with 10⁷ human PBMCs on day −14. The mice were implanted subcutaneously with 2×10⁶ Ramos cells on day 0. On day 3, samples of whole blood were withdrawn for CD3 engraftment check. Antibody dosing commenced on day 5 or day 6 when the tumors had reached a volume of from 100 to 150 mm³ (typically closer to 100 mm³). Mice were treated with vehicle (group 1), a control antibody from Table 13 in which the IgM pentamer binds to an irrelevant non-mammalian target, D15 (group 2), exemplary antibody C7 (the anti-CD20 x anti-CD3 x anti-CD28 IgM antibody described in Example 1, group 3), or exemplary antibody D7 (the anti-CD20 x anti-CD3 IgM antibody described in Example 1, group 4). Mice were dosed biweekly for administration of a total of three doses (biwx3). Over the course of the study, tumor size and body weight were measured 3 times per week. Samples of whole blood were collected twenty-four hours following administration of the third dose. At the study end point, tumors, spleen, and peripheral blood were harvested for flow cytometry and immunohistochemistry analysis.

TABLE 22
Dosing Schedule

| | | | | Test | Amount | | |
|---|---|---|---|---|---|---|---|
| Group | N | Ramos | hPBMC | Article | (mg/kg) | Route | Regimen |
| 1 | 8 | 2 × 10\hat{ }6 | 10 × 10\hat{ }6 | Vehicle | — | i.v | biwx3 |
| 2 | 8 | 2 × 10\hat{ }6 | 10 × 10\hat{ }6 | Control | 10 | i.v | biwx3 |
| 3 | 8 | 2 × 10\hat{ }6 | 10 × 10\hat{ }6 | C7- | 10 | i.v | biwx3 |
| | | | | CD20 × CD3 × CD28 | | | |
| 4 | 8 | 2 × 10\hat{ }6 | 10 × 10\hat{ }6 | D7- | 10 | i.v | biwx3 |
| | | | | CD20 × CD3 | | | |

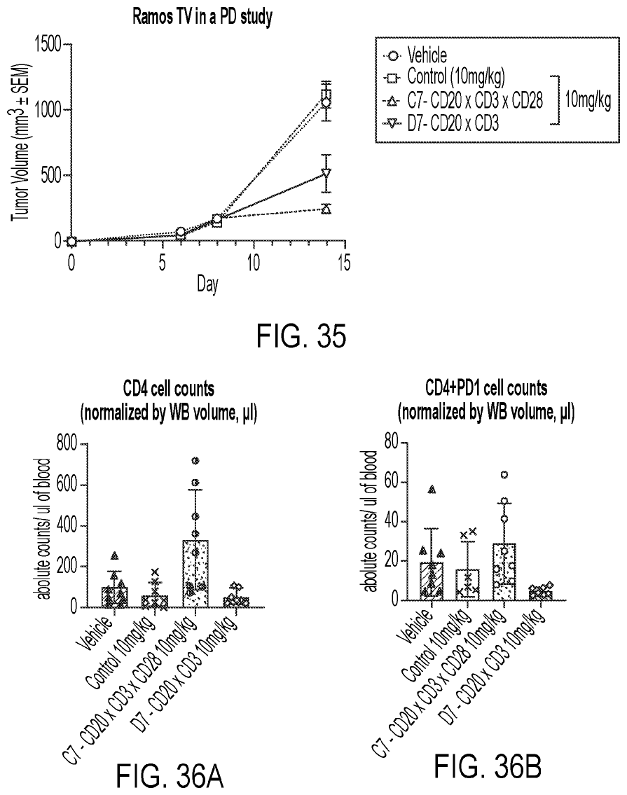

Tumor volume over time for the various treatment groups (groups 1, 2, 3, and 4) is shown in FIG. 35. Tumor volume over time was lower for the C7 (anti-CD20 x anti-CD3 x anti-CD28) and D7 (CD20×CD3) treatment groups in comparison to the vehicle and control groups. At day 14, the tumor volume of the group of mice treated with antibody C7 (group 3) was lower than that of the group of mice treated with antibody D7 (group 4).

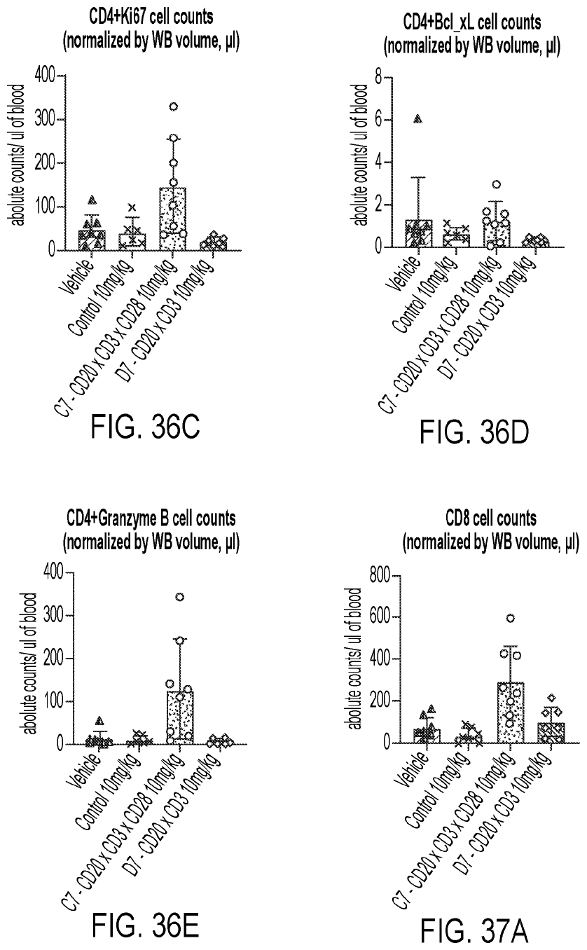

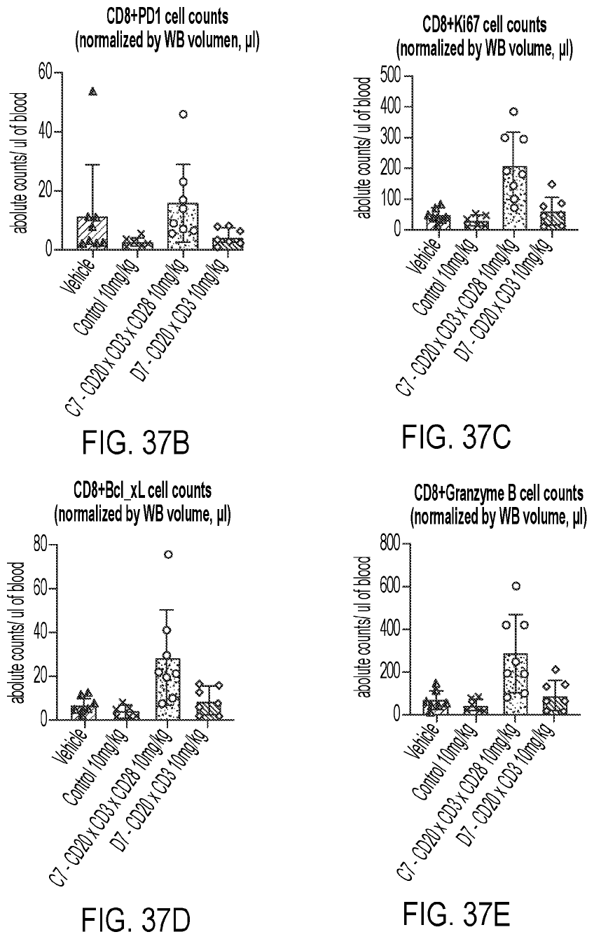

Harvested peripheral blood was incubated with RBC lysis buffer (Thermo Fisher Scientific) and washed by Pharmingen Stain™ Buffer (BD Biosciences). Cells were stained for FACS analysis with the following antibody staining panel: anti-Bcl_xL-APC (Cell Signaling), anti-humanCD45-APC/Cy7, anti-mouseCD45-BV785, anti-CCR7 FITC, anti-CD4-AF700, anti-CD8-PerCp/Cy5.5, anti-PD1-BV605, anti-CD45PRO-PE/Cy7, anti-K167-BV421, and anti-granzyme b-PE from Biolegend. Live/dead cells were stained using ZOMBIE AQUA™ fixable viability kit (Biolegend). Absolute counts of CD4+ and CD8+ T cell subsets in peripheral blood normalized to blood volume are shown in FIGS. 36A-E and FIGS. 37A-E, respectively.

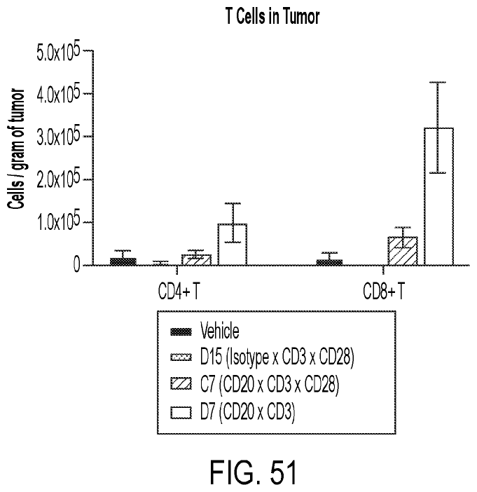

Mice were sacrificed after 3 doses and tumor samples were collected and analyzed by flow cytometry analysis and IHC staining in which immunohistochemistry was used to evaluate the presence of tumor-infiltrating T cells. The results of the flow cytometry analysis are shown in FIG. 51, in which cells per gram of tumor are shown for the treatment groups described above. As shown, T cell infiltration into the tumor was notably enhanced for the exemplary co-stimulatory CD20xCD3xCD28 IgM antibody, C7.

For the immunohistochemistry analysis, formalin-fixed paraffin embedded (FFPE) sections from the mice were stained with anti-CD3 and anti-CD8 to identify tumor-infiltrating T cells. The exemplary costimulatory IgM, C7 (CD20xCD3xCD28)) resulted in increased tumor-infiltrating T cells compared to a bispecific IgM, D7 (CD20xCD3), while the non-targeting control IgM with CD3xCD28 J chain, D15, and the vehicle control had no effect (data not shown).

### Example 24: Construction and Characterization of Antibodies Targeting Solid Tumors

Additional IgM pentameric antibodies, which are described in Table 23, were produced according to the methods described in Example 1.

TABLE 23
Solid Tumor Targeting Antibodies

| | | | | | Anti- | |
|---|---|---|---|---|---|---|
| | | | | | Tumor | |
| | | | | | Target | Modified |
| | | Anti- | Anti- | Anti- | VH, VL | J chain |
| Antibody | IgM | CD3 AA | CD28 | CD28 | SEQ ID | SEQ ID |
| Name | target | location | scFv | location | NOs | NO |
| T1 | TROP-2 | N-Term | Z | C term | 248, 249 | 233 |
| T2 | TROP-2 | N/A | Z | C term | 248, 249 | 230 |
| T3 | TROP-2 | N-term | N/A | N/A | 248, 249 | 220 |
| M1 | Mesothelin | N term | Z | C term | 262, 263 | 233 |
| M2 | Mesothelin | N/A | Z | C term | 262, 263 | 230 |
| M3 | Mesothelin | N term | N/A | N/A | 262, 263 | 220 |

TABLE 24
Anti-TROP-2 Binding Domains

| | VH | | VL | |
|---|---|---|---|---|
| Source | SEQ | | SEQ | |
| (Nickname) | ID NO | VH | ID NO | VL |
| WO199801222 | 244 | QVQLQESGAELVMPGASVKL | 245 | EFMDILMTQSPSSLSASLGGKVT |
| 7 (TROP-2- | | SCKASGYTFINYWMHWVKQR | | ISCKASQDINNYIAWYQHKPGKG |
| 1) | | PGQGLEWIGEISPSDSYTDY | | PRLLIHFTSTLQPGIPSRFSGSG |
| | | NQKFKGKATLTVDTSSNAAY | | SGRDYSFSISNLEPEDIGTYFCL |
| | | MQLSGLTFEDSAVYYCTRGG | | QYNYLWTFGGGTKLEIK |
| | | KSLGFFDYWGQGTTLTVSS | | |
| U.S. Pat. No. | 246 | QVQLQQSGSELKKPGASVKV | 247 | DIQLTQSPSSLSASVGDRVSITC |
| 9,849,176B2 | | SCKASGYTFTNYGMNWVKQA | | KASQDVSIAVAWYQQKPGKAPKL |
| (TROP-2-2) | | PGQGLKWMGWINTYTGEPTY | | LIYSASYRYTGVPDRFSGSGSGT |
| | | TDDFKGRFAFSLDTSVSTAY | | DETLTISSLQPEDFAVYYCQQHY |
| | | LQISSLKADDTAVYFCARGG | | ITPLTFGAGTKVEIKR |
| | | FGSSYWYFDVWGQGSLVTVS | | |
| | | S | | |
| US201223751 | 248 | QVQLVQSGPEVKKPGASVKV | 249 | DIVMTQSPDSLAVSLGERATINC |
| 8A1 (TROP- | | SCKASGYTFTIYWLGWVRQA | | KSSQSLLNSGNQQNYLAWYQQKP |
| 2-3) | | PGQGLEWMGNIFPGSAYINY | | GQPPKLLIYGASTRESGVPDRES |
| | | NEKFKGKVTITADTSTSTAY | | GSGSGTDETLTISSLQAEDVAVY |
| | | MELSSLRSEDTAVYFCAREG | | YCQSDHIYPYTFGQGTKLEIK |
| | | SNSGYWGQGTLVTVSS | | |
| U.S. Pat. No. | 250 | QVQLVQSGAEVKKPGASVKV | 251 | DIQMTQSPSSLSASVGDRVTITC |
| 9,850,312B2 | | SCKASGYTFTTAGMQWVRQA | | KASQDVSTAVAWYQQKPGKAPKL |
| (TROP-2-4) | | PGQGLEWMGWINTHSGVPKY | | LIYSASYRYTGVPSRFSGSGSGT |
| | | AEDFKGRVTISADTSTSTAY | | DETLTISSLQPEDFAVYYCQQHY |
| | | LQLSSLKSEDTAVYYCARSG | | ITPLTFGQGTKLEIKRT |
| | | FGSSYWYFDVWGQGTLVTVS | | |
| | | S | | |
| US200802132 | 252 | QIQLVQSGHEVKKPGASVKV | 253 | DIQMTQSPSSLSASVGDRVTITC |
| 67A1 (TROP- | | SCKASGYTFTNYGMNWVRQA | | KASQDVSIAVAWYQQKPGKAPKV |
| 2-5) | | PGQGLEWMGWINTKTGEPTY | | LIYSASYRYTGVPDRFSGSGSGT |
| | | AEEFKGRFVFSLETSASTAY | | DFTFTISSLQPEDIAVYYCQQHY |
| | | LQISSLKAEDMAMYFCGRGG | | ITPLTFGGGTKVEIK |
| | | YGSSYWYFDVWGQGTTVTVS | | |
| | | S | | |
| U.S. Pat. No. | 254 | QVQLQESGPGLVKPSETLSL | 255 | DIVMTQSPDSLAVSLGERATINC |
| 9,399,074B2 | | TCTVSGGSISSYGVHWIRQP | | RASKSVSTSGYSYMHWYQQKPGQ |
| (TROP-2-6) | | PGKGLEWIGVIWTSGVTDYN | | PPKLLIYLASNLESGVPDRESGS |
| | | SALMGRVTISVDTSKNQFSL | | GSGTDETLTISSLQAEDVAVYYC |
| | | KLSSVTAADTAVYYCARDGD | | QHSRELPYTFGQGTKLEIK |
| | | YDRYTMDYWGQGTLVTVSS | | |

TABLE 25
Anti-Mesothelin Binding Domains

| | VH | | VL | |
|---|---|---|---|---|
| Source | SEQ | | SEQ | |
| (Nickname) | ID NO | VH | ID NO | VL |
| U.S. Pat. No. | 256 | QVKLKQSGGGLVQPGGSLKV | 257 | DVVLTQSPAIMSASPGEKVTMTC |
| 5,489,525A | | SCAASGEDFSRYWMSWVRQA | | SASSSVSYMYWHQQKPGSSPRLL |
| (MSLN-1) | | PGKGLEWIGEINPDSSTIVY | | IYDTSNLASGVPVRFSGSGSGTS |
| | | TPSLKDKFIMSRDNAKNTLY | | YSLTISRMEAEDAATYYCQQWSS |
| | | LQTSKVRSADTALYYCARRG | | YPPTFGGGTKLEIKR |
| | | SHYYGYRTGYFDVWGAGTTV | | |
| | | TVSS | | |
| US201700295 | 258 | QVQLQESGPGLVKPSETLSL | 259 | QSVLTQPPSVSGAPGQRVTISCT |
| 02A1 (MSLN- | | TCTVSGGSISSSSYYWGWIR | | GSSSNIGAGYDVHWYQQVPGTAP |
| 2 ) | | QPPGKGLEWIGSIYYSGITN | | KLLIYGNSKRPSGVPDRESGSKS |
| | | YNPSLKSRVTISVDTSKNQF | | GTSASLAITGLQAEDEADYYCQS |
| | | SLKLSSVTAADTAVYYCARP | | YDSSLGGWVFGGGTKLTVL |
| | | SNYDAFDIWGQGTMVTVSS | | |
| US201700295 | 260 | QVQLVESGGGLVKPGGSLRL | 261 | DIQMTQSPSSVSASVGDRVTITC |
| 02A1 (MSLN- | | SCAASGFTFSDYYMTWIRQA | | RASQGITRWLAWYQQKPGKAPKL |
| 3) | | PGKGLEWISYISSSGSTIYY | | LIYAASVLQSGVPSRFSGSGSGT |
| | | ADSVKGRFTISRDNAKNSLY | | DFTLTISSLQPEDFATYYCQQSN |
| | | LQMNSLRAEDTAVYYCARDR | | SFPRTFGQGTKVEIK |
| | | NSHFDYWGQGTLVTVSS | | |
| US201700295 | 262 | QVQLVESGGGLVKPGGSLRL | 263 | DIQMTQSPSSVSASVGDRVTITC |
| 02A1 (MSLN- | | SCAASGFTFSDHYMSWIRQA | | RASQDISRWLAWYQQKPGKAPKL |
| 4) | | PGKGLEWFSYISSSGGIIYY | | LISAASRLQSGVPSRFSGSGSGT |
| | | ADSVKGRFTISRDNAKNSLY | | DFTLTISSLQPEDFAIYYCQQAK |
| | | LQMNSLRAEDTAVYYCARDV | | SFPRTFGQGTKVEIK |
| | | GSHFDYWGQGTLVTVSS | | |
| US201700295 | 264 | EVQLVESGGGLIQPGGSLRL | 265 | DIVMTQSPDSLAVSLGERATINC |
| 02A1 (MSLN- | | SCAVSGFTVSSKFMTWVRQA | | KSSQSVLYSSNNKNYLAWYQQKP |
| 5) | | PGKGLEWVSVIYSGGKTYYA | | GQPPKLLIYWASTRESGVPDRES |
| | | DSVKGRFTISRDNSKNTLYL | | GSGSGTDETLTISSLQAEDVAVY |
| | | QMNSLRAEDTAVYYCARDSG | | YCQQYYSTPPTFGQGTKVEIK |
| | | GWGYFDYWGQGTLVTVSS | | |

T cell dependent cytotoxicity: The T cell dependent cytotoxicity (TDCC) activity of the various solid tumor targeting constructs as shown in Table 23 on the ovarian cancer cell line OVCAR3 (ATCC Cat. No. HTB-161) (and on additional cell lines as described below including HEK293T/TROP2, A431 and SKOV-3) was assessed by Real Time Cell Analysis or luminescence as follows.

TDCC activities were evaluated using XCELLIGENCE™ real time cell analyzer (RTCA), which utilizes microelectrodes fused to the bottom surface of special 96-well E-plates to measure the impedance to electron flow caused by the presence of adherent cells (represented by the normalized cell index calculated by the XCELLIGENCE™ software). Adherent target tumor cells were added to the 96-well E-plates at the specified seeding densities. The E-plates were loaded to RTCA inside a humidified cell incubator with 5% CO₂ at 37 °C for 24 hours to allow target tumor cells to settle and attach to the bottom of the plates. Effector cells (PBMC or T cells) and test articles were then added to the wells before resuming the culture and real time cell index monitoring. Cell index data were exported and analyzed with GraphPad Prism. In some cases, supernatant samples were collected at specified time points for cytokine analysis using the Human Th1/Th2 Cytokine Cytometric Bead Array (CBA) Kit II from BD Biosciences (catalog #551809). At the end of the co-culture assay, T cell activation can be evaluated by collecting cell samples from the E-plates for flow cytometry analysis of various biomarkers (CD3, CD4, CD8 and CD25).

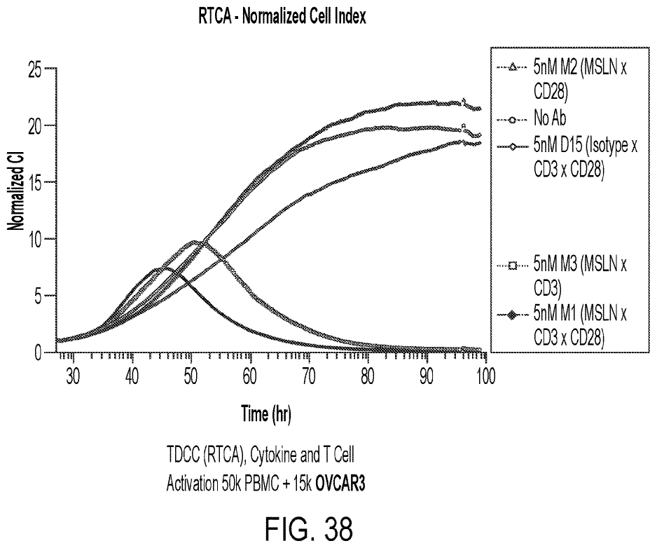

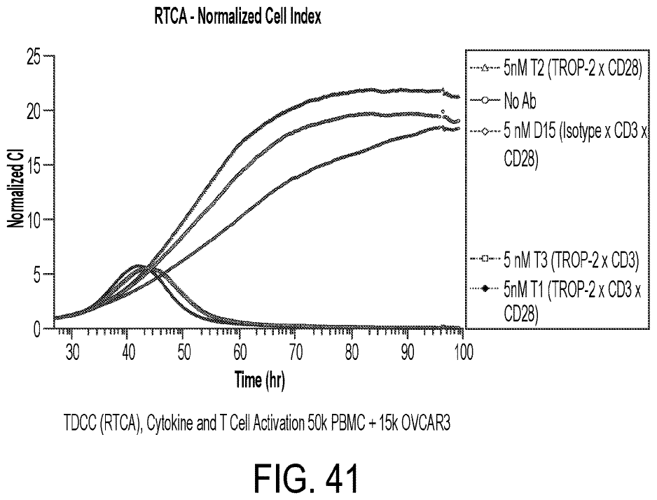

The results for the mesothelin-targeting constructs are shown in FIG. 38, and the results for the TROP-2-targeting constructs are shown in FIG. 41. The constructs with costimulatory activity (binding to CD2 and CD28), M1, and T1, showed slightly improved activity over the constructs binding just to CD3, M3 and T3, while the constructs binding just to the tumor target and CD28, M2 and T2, showed activity comparable to the no antibody and isotype control constructs.

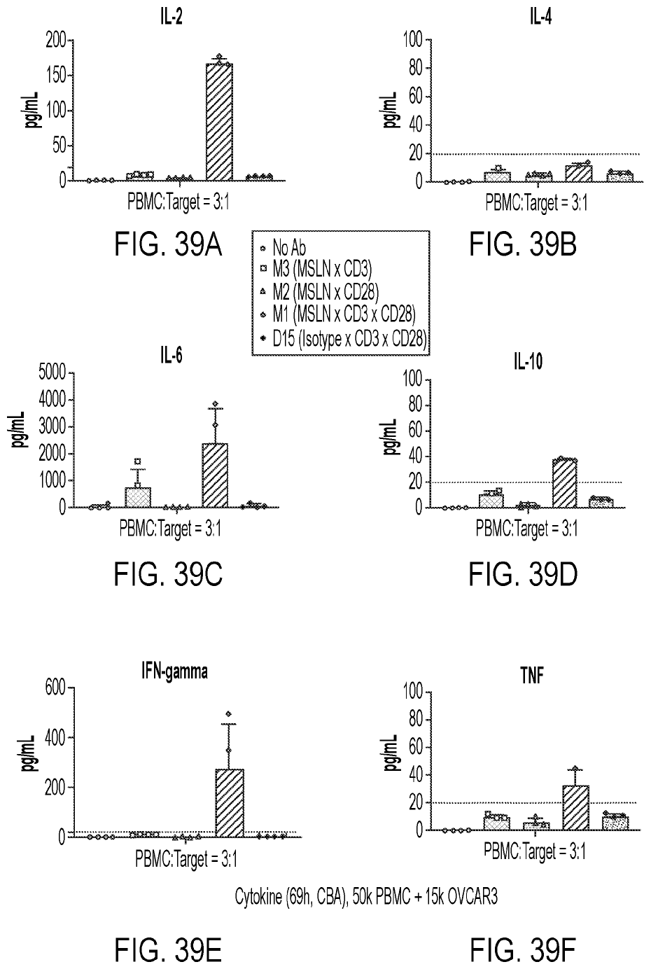

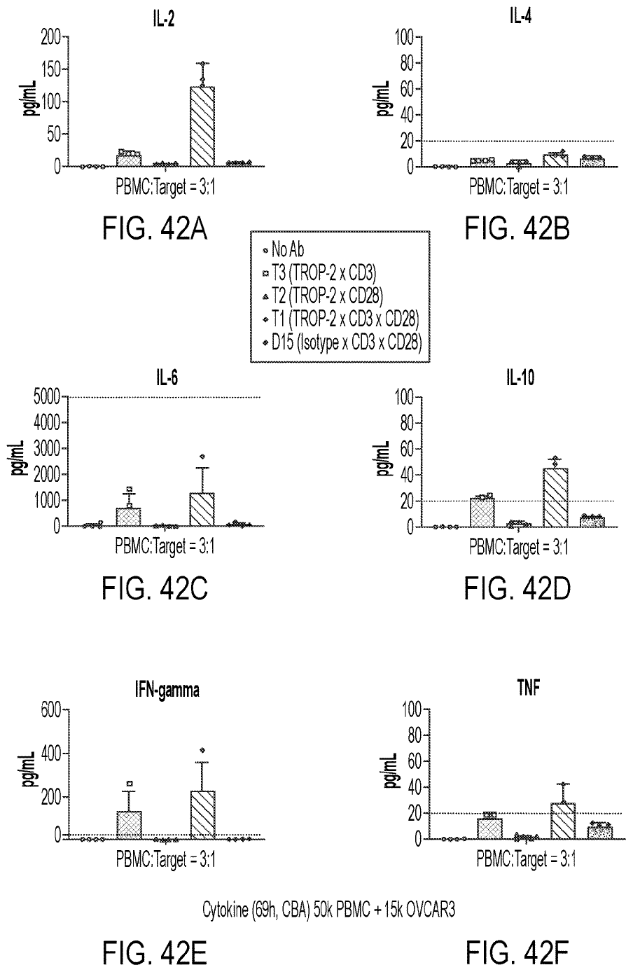

Cytokine Release: To measure the cytokine release mediated by the test articles, supernatant samples from the 72-hour time point of the RTCA assays shown in FIGS. 38 and 41 were thawed and analyzed by flow cytometry, using the Human Th1/Th2 Cytokine Cytometric Bead Array (CBA) Kit II from BD Biosciences (catalog #551809) based on manufacture's recommendation, except using smaller culture volume to accommodate a plate-based format. The flow cytometry analysis was performed on the INTELLICYT® IQUE® Screener PLUS and data was analyzed using the INTELLICYT® IQUE FORECYT® software, including standard curve fitting and cytokine concentration calculations. The levels of IL-2, IL-4, IL-6, IL-10, IFN-γ, and TNFα are shown for the anti-mesothelin constructs in FIGS. 39A-39F, respectively, and for the anti-TROP-2 constructs in FIGS. 42A-42F, respectively.

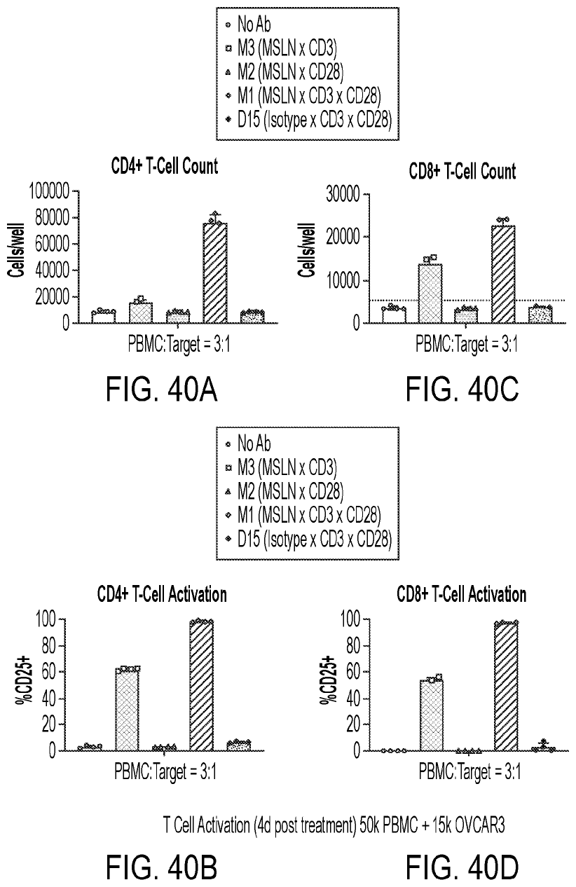

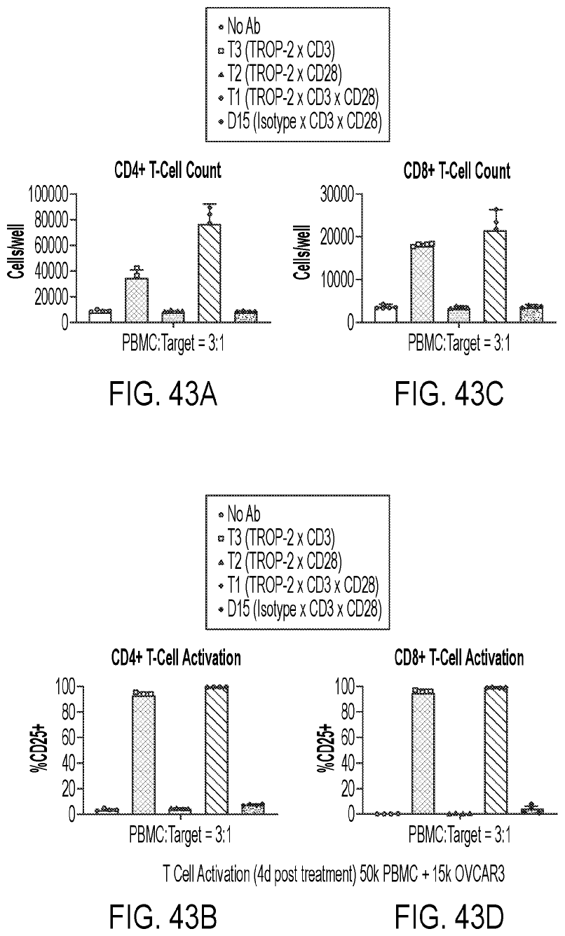

T-cell Activation: T cell numbers and the extent of T cell activation for both CD4+ and CD8+ T cell subsets at the 96-hour time point of the RTCA assays shown in FIGS. 38 and 41 were measured as follows. Cells from each well were collected by pipetting up and down multiple times and transferred to a new U-bottom 96-well plate. Cells were stained for FACS analysis with the following staining panel: anti-CD3 PerCP-Cy5.5, anti-CD8 BV785, anti-CD25 APC, and anti-CD4 BV605 from Biolegend, and the Fixable viability dye EFLUOR® 780 (ThermoFisher, 65-0865-14). The number of different T cell subsets in each well were compared to spiked-in counting beads to calculate T cell number and T cell activation status were evaluated based on CD25 staining result. The data was then analyzed and plotted with GraphPad Prism. The results for the mesothelin-targeting constructs are shown in FIGS. 40A-40D, and the results for the TROP-2-targeting constructs are shown in FIGS. 43A-43D. The constructs with costimulatory activity (binding to CD3 and CD28), M1, and T1, showed slightly improved T cell counts and T cell activation over the constructs binding just to CD3, M3 and T3, while the constructs binding just to the tumor target and CD28, M2 and T2, showed activity comparable to the no antibody and isotype control constructs.

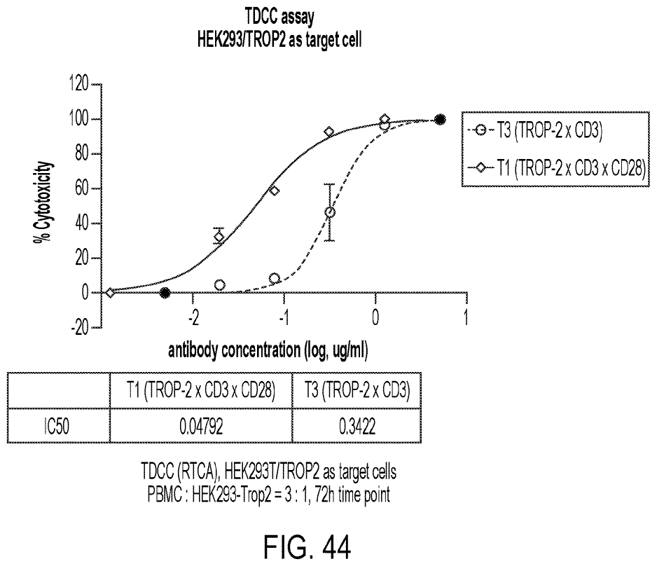

T cell Dependent Cytotoxicity: Antibody-mediated redirected T cell killing of HEK293/TROP-2 cells (generated in-house) was measured in a TDCC assay in which human PBMCs as effector cells (E) and TROP2 transduced HEK293 cells as target cells (T) at an E:T ratio 3:1 were incubated with serial dilutions of indicated antibodies in 96-well plates for 72 hours at 37 °C in a humidified 5% CO₂ incubator. After incubation, the impedance was used to evaluate the cell viability by RTCA as described above. The percent of cytotoxicity following antibody treatment was calculated by normalization of cell index from no antibody treatment group; results are provided in FIG. 44. The IC50 values determined for antibodies T1 and T3 were 0.04792 and 0.3422, respectively. Data are representative of 2 independent donors.

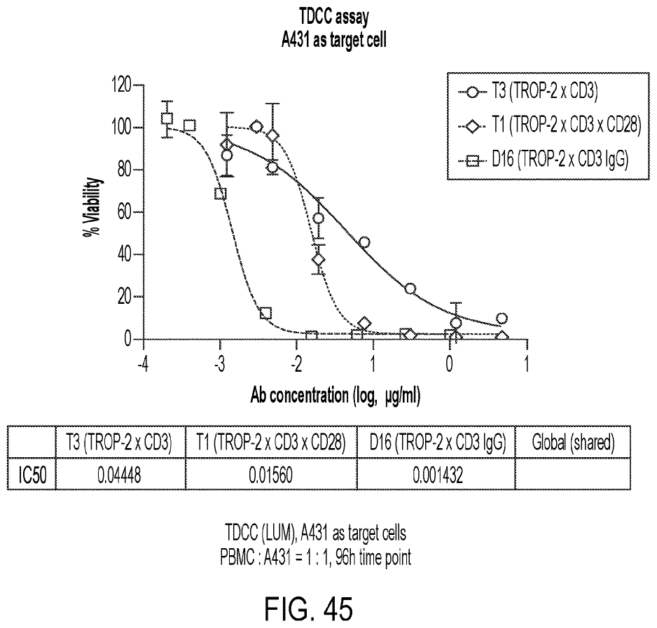

The T cell dependent cytotoxicity (TDCC) activity of various solid tumor targeting constructs as shown in Table 23 on the epidermoid cancer cell line A431 (American Type Culture Collection, ATCC catalogue No. CRL-1555-LUC2) was assessed by luminescence as follows. Antibody-mediated redirected T cell killing of A431-Luc cells was measured in a TDCC assay in which human PBMCs as effector cells (E) and A431-Luc cells as target cells (T) at an ET ratio 1:1 were incubated with serial dilutions of indicated antibodies in 96-well plates for 96 hours at 37 °C in a humidified 5% CO₂ incubator. After incubation, a luminescence-based viability assay was used to determine the viable cells as described below. The % of cell viability following antibody treatment with T3, T1, or D16 was calculated by normalization of viable cells from no antibody treatment group. Data are representative of 3 independent donors. Results are provided in FIG. 45. The IC50 values determined for antibodies T3, T1, and D16 were 0.04448, 0.01560, and 0.001432, respectively.

Luciferase tagged adherent target tumor cells were added to the 96-well cell culture plates at the specified seeding densities and cultured in a humidified cell incubator with 5% CO₂ at 37 °C for 24 hours to allow target tumor cells to settle and attach to the bottom. Effector cells (PBMC or T cells) and test articles were then added to the wells before resuming the culture. After incubation, the viability of the luciferase tagged tumor cells was determined by luminescent readout using the BIO-GLO™ luciferase assay system from Promega (catalogue number G7940), and the data was analyzed using GraphPad Prism.

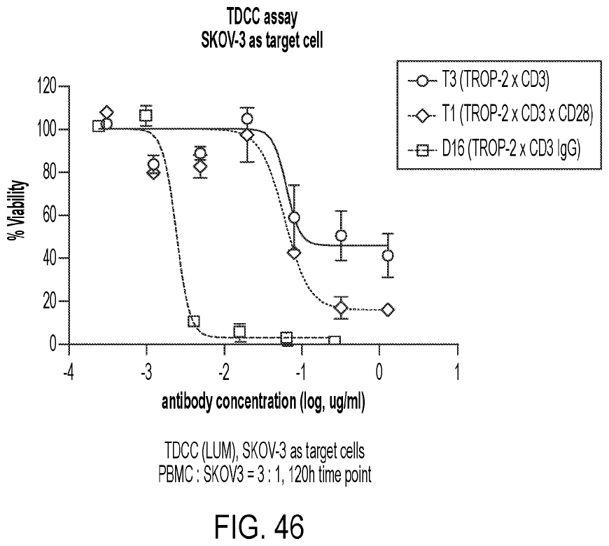

The T cell dependent cytotoxicity (TDCC) activity of various solid tumor targeting constructs as shown in Table 23 on the ovarian adenocarcinoma cell line SKOV-3 (BPS Bioscience, catalogue No. 7842) was assessed by luminescence as follows. Antibody-mediated redirected T cell killing of SKOV3-Luc cells was measured in a TDCC assay in which human PBMCs as effector cells (E) and SKOV3-Luc cells as target cells (T) at an E:T ratio 3:1 were incubated with serial dilutions of indicated antibodies in 96-well plates for 120 hours at 37 °C in a humidified 5% CO₂ incubator. After incubation, a luminescence-based viability assay as described above was used to determine the viable cells. The percent of cell viability following treatment with antibodies T3, T1, or D16 was calculated by normalization of viable cells from no antibody treatment group. Data are representative of 3 independent donors. Results are provided in FIG. 46.

Additional T cell dependent cytotoxicity (TDCC) activity evaluations and analyses were carried out of various exemplary solid tumor targeting constructs as shown in Table 23 (D16, T3, T1, D17, M3 and M1) on the ovarian cancer cell line OVCAR3 (ATCC Cat. No. HTB-161); additional cytokine release measurements were also carried out.

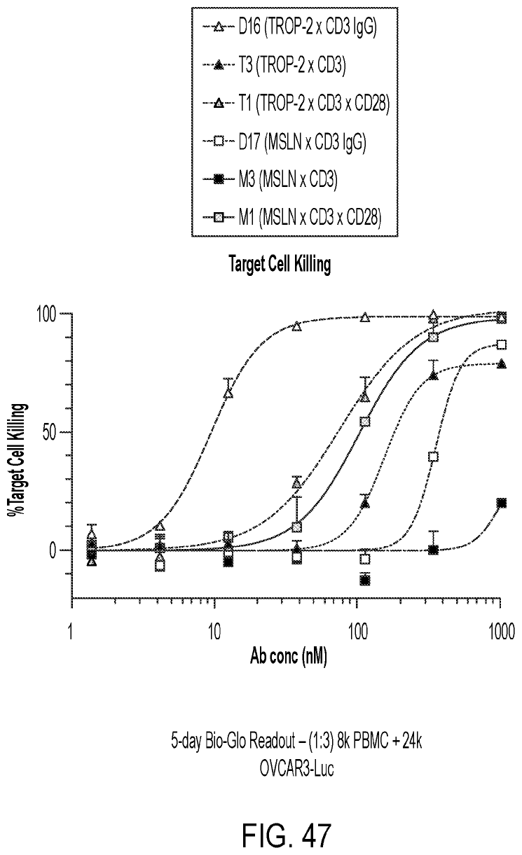

Antibody-mediated redirected T cell killing of OVCAR3 cells was measured in a TDCC assay in which human PBMCs as effector cells (E) and OVCAR3-Luc cells as target cells (T) at an E:T ratio of 1:3 were incubated with serial dilutions of antibodies D16, T3, T1, D17, M3 and M1 in 96-well plates for 120 hours (5 days) at 37 °C in a humidified 5% CO₂ incubator. After incubation, the viability of the luciferase-tagged tumor cells was determined by luminescent readout using the BIO-GLO™ luciferase assay system from Promega (catalogue number G7940), and the data was analyzed using GraphPad Prism. Results are provided graphically in FIG. 47 which shows percent target cell killing versus antibody concentration for antibodies D16, T3, T1, D17, M3 and M1.

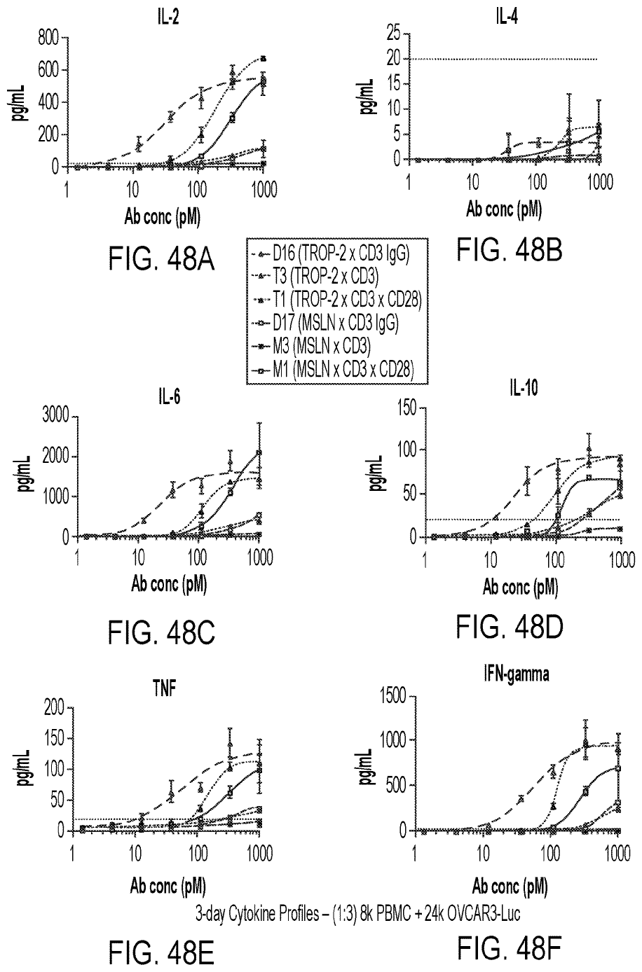

Cytokine Release: Cytokine release mediated by the test articles was determined as described above using a Human Th1/Th2 Cytokine Cytometric Bead Array (CBA) Kit II from BD Biosciences using supernatant samples from the 72-hour (3-day) time point. Levels of IL-2, IL-4, IL-6, IL-10, TNFα (also referred to herein as TNF) and IFN-γ mediated by the test articles are shown for in FIGS. 48A-48F, respectively.

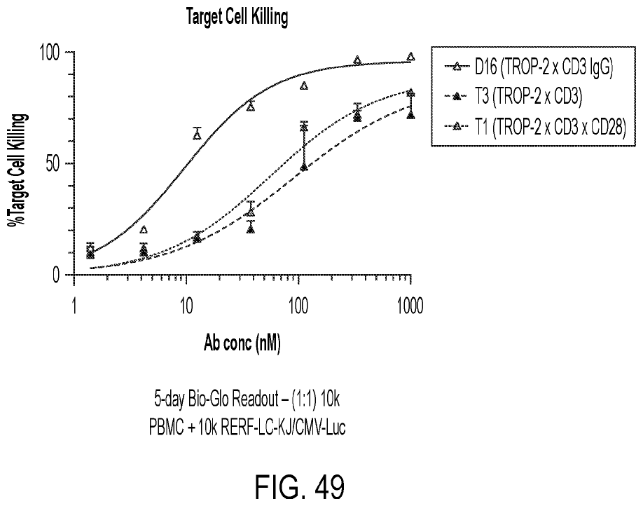

T cell Dependent Cytotoxicity: Additionally, antibody-mediated redirected T cell killing of REFR cells RERF-LC-KJ/CMV-Luc cells, or RERF-Luc, (JCRB cell bank, catalogue No. JCRB1558) was measured in a TDCC assay in which human PBMCs as effector cells (E) and RERF cells as target cells (T) at an E:T ratio of 1:1 were incubated with serial dilutions of antibodies D16, T3, and T1 in 96-well plates for 120 hours (5 days) at 37 °C in a humidified 5% CO₂ incubator. After incubation, the viability of the luciferase-tagged tumor cells was determined by luminescent readout using the BIO-GLO™ luciferase assay system from Promega (catalogue number G7940), and the data was analyzed using GraphPad Prism. Results are provided graphically in FIG. 49.

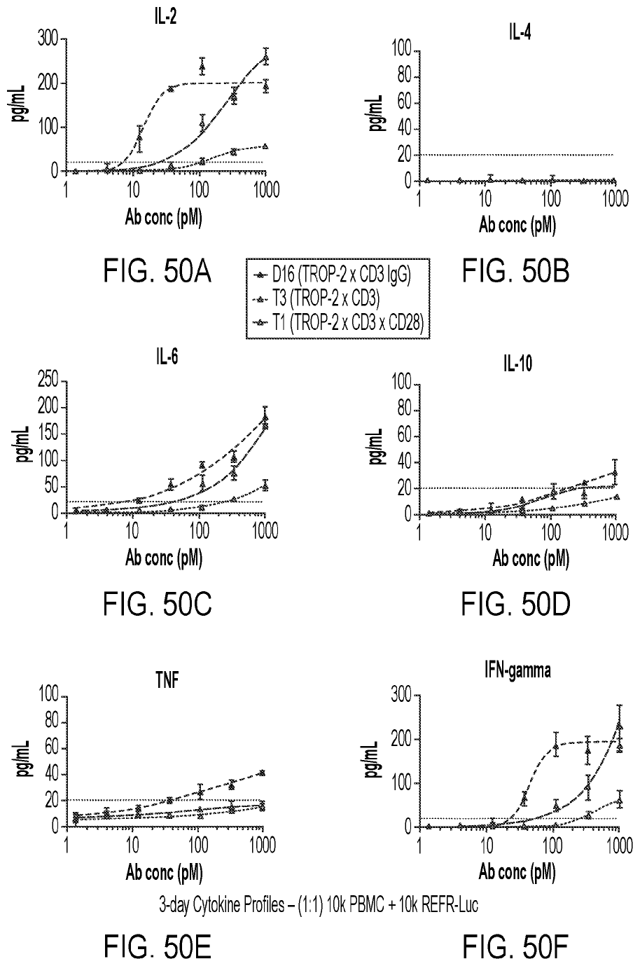

Cytokine Release: Cytokine release mediated by the test articles was determined as described above using supernatant samples from the 72-hour (3-day) time point. Levels of IL-2, IL-4, IL-6, IL-10, TNF, and IFN-γ mediated by the test articles are shown for in FIGS. 50A-50F, respectively.

### Example 25: T-Cell Dependent Cellular Toxicity Assay Using High, Medium and Low-TROP-2 Expressing Cell Lines

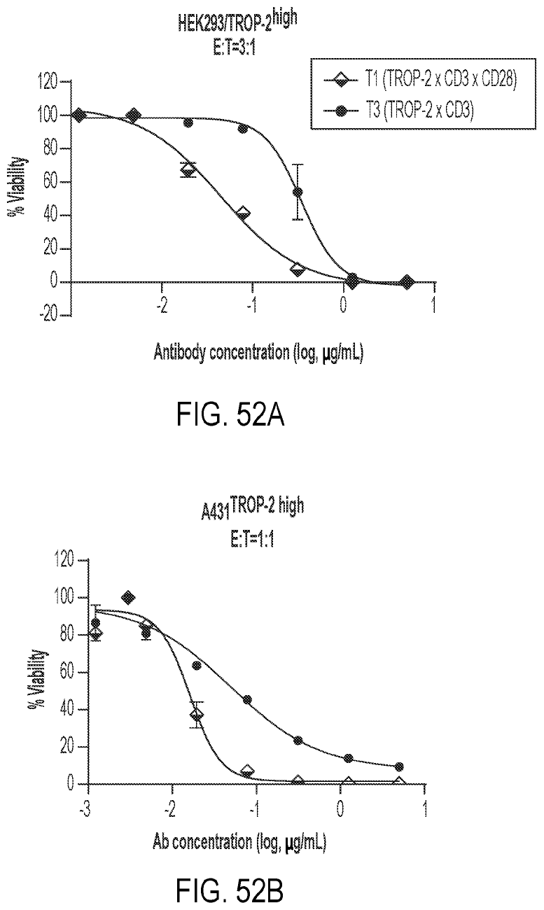

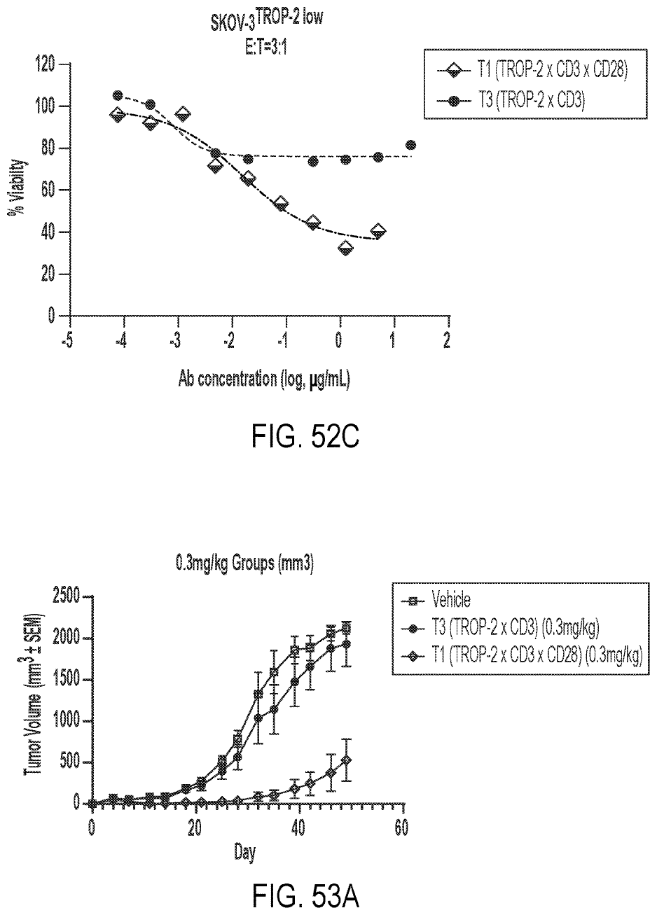

Antibody-mediated redirected T cell killing by exemplary IgM antibodies with or without CD-28 co-stimulation, T1 (anti-TROP-2 x anti-CD3 x anti-CD28 IgM antibody) and T3 (anti-TROP-2 x anti-CD3 IgM), respectively, was evaluated in a T cell-dependent cellular cytotoxicity (TDCC) assay. Human PBMCs as effector cells (E) were co-cultured with the following high, medium, or low TROP2-expressing cells: TROP-2 transduced HEK293T cells with TROP-2 high expression, A431 melanoma cells with TROP-2 high expression, and SKOV-3 ovarian cancer cells with TROP-2 medium/low expression, as target cells (T), at E:T ratios of 3:1, 1:1, and 3:1, respectively, in the presence of serial dilutions of T1 or T3 antibody. Incubation was carried out for 3-4 days at 37 °C in a humidified 5% CO₂ incubator. After incubation, the impedance was used to evaluate the cell viability by RTCA. The percent viability following antibody treatment was calculated by normalization of viable cells from a “no antibody” treatment group. Plots of percent cell viability versus antibody concentration (log, μg/mL) are shown in FIG. 52A (HEK293/TROP-2^{high} target cells, E:T=3.1), FIG. 52B (A431^{TROP-2 high} target cells, E:T=1:1) and FIG. 52C (SCOV-3TROP-2low target cells, E:T=3:1). As shown in each of the three plots, enhanced cytotoxicity by an exemplary IgM-antibody, (T1, anti-TROP-2 x anti-CD3 x anti-CD28) with CD28-co-stimulation was observed on TROP-2 transduced HEK293T cells with TROP-2^{high} expression, A431 melanoma cells with TROP-2 high expression, and on SKOV-3 ovarian cancer cells with TROP-2 medium/low expression when compared to an illustrative IGM antibody without CD28 co-stimulation (T3, anti-TROP-2 x anti-CD3 IgM).

### Example 26: In Vivo Study: Antitumor Activity, Cytokine Release and Anti-Apoptotic T Cell Counts

In a preclinical efficacy study, the antitumor activity of exemplary IgM antibodies, T1 (anti-TROP-2 x anti-CD3 x anti-CD28 IgM antibody) and T3 (anti-TROP-2 x anti-CD3 IgM), was evaluated in a HEK293T/TROP-2 xenograft model. NSG-MHCI/II DKO mice were engrafted with 10⁷ human PBMCs on day −14. On day 0, mice were implanted subcutaneously with 10⁷ TROP-2 transduced HEK293T cells. Animals were dosed intravenously biweekly with antibody T1 or antibody T3, at doses of 0.3 mg/kg, 1 mg/kg, or 3 mg/kg, or vehicle (1×PBS) (see Table 26), starting on day 3 for a total of 12 doses (biwx12). For the 3 mg/kg treatment group, antibody dosing was stopped after 7 doses to compare the prolonged effect of treatment with the illustrative anti-TROP-2 x anti-CD3 x anti-CD28 IgM antibody versus the illustrative anti-TROP-2 x anti-CD3 IgM antibody (that lacks a CD28 co-stimulatory signal).

TABLE 26
Treatment Groups

| Treatment Group | No. of Mice | Dose | |
|---|---|---|---|
| T1 (TROP-2 × | 7 | 0.3 | mg/kg |
| CD3 × CD28 IgM) | | | |
| T3 (TROP-2 × CD3 IgM) | 7 | 0.3 | mg/kg |
| T1 (TROP-2 × | 7 | 1 | mg/kg |
| CD3 × CD28 IgM) | | | |
| T3 (TROP-2 × CD3 IgM) | 7 | 1 | mg/kg |
| T1 (TROP-2 × | 7 | 3 | mg/kg |
| CD3 × CD28 IgM) | | | |
| T3 (TROP-2 × CD3 IgM) | 7 | 3 | mg/kg |

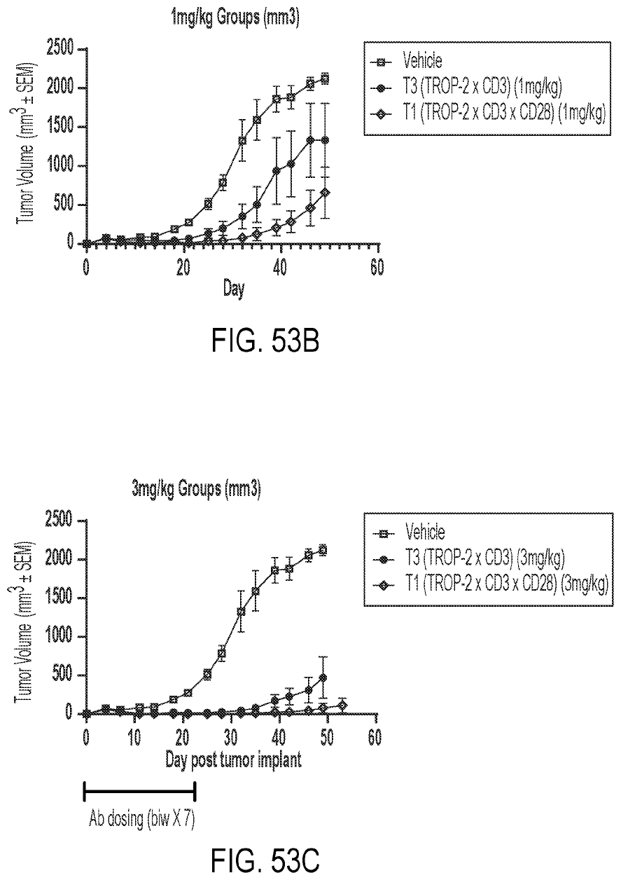

Graphs of tumor volume over time for the various treatment groups is provided in FIGS. 53A (0.3 mg/kg groups), 53B (1 mg/kg groups) and 53C (3 mg/kg groups). As shown in all three graphs, treatment with an IgM antibody such as T1 (with CD28 co-stimulation) resulted in enhanced in vivo antitumor activity over exemplary IgM antibody T3 (lacking CD28 co-stimulation), e.g., when evaluated in a humanized HEK293/TROP-2 xenograft model, particularly at low doses.

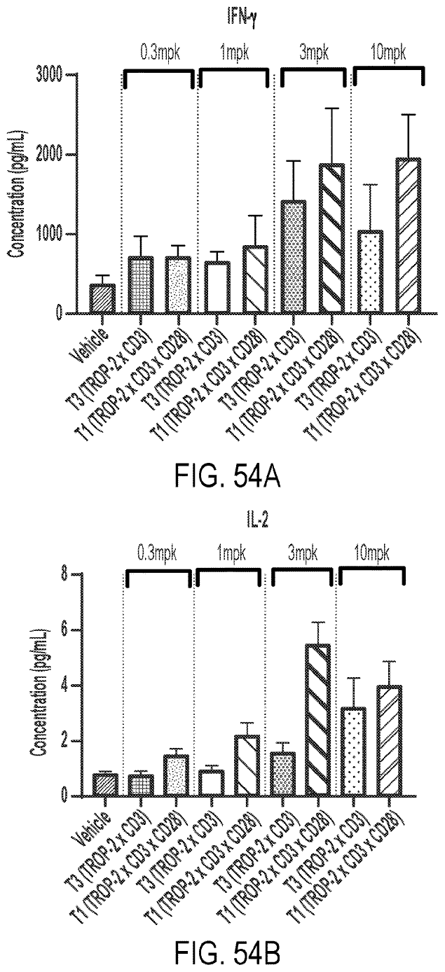

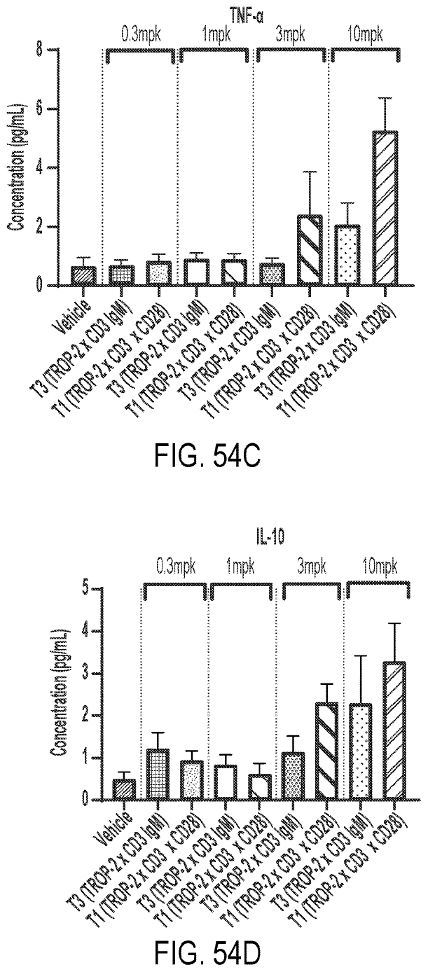

Harvested serum samples from each of the treatment groups were collected 4 hours post first dose and analyzed for cytokines IFN-γ, IL-2, TNF-α, and IL-10 using an MSD multiplex cytokine assay kit (Meso Scale Discovery) according to manufacturer's protocol. The results were then plotted with GraphPad Prism. Serum concentrations of IFN-γ, IL-2, TNF-α, and IL-10 are shown in FIGS. 54A-54D, respectively. A dose-dependent increase in the levels of released cytokines interleukin-2, interferon-gamma, tumor necrosis factor-alpha, and interleukin-10 was observed in the T1 IgM antibody treatment groups, i.e., following treatment with an exemplary TROP-2×CD3 x CD28 IgM antibody (i.e., with CD28 co-stimulation).

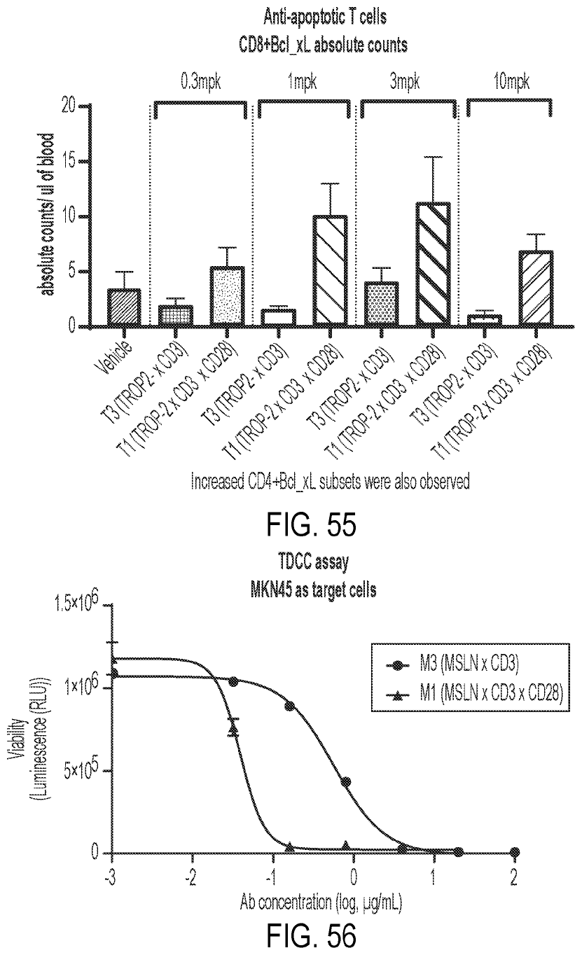

Mouse peripheral blood samples were harvested on day 12 (24 hours pre-4th dose). Blood samples were processed and stained for flow cytometry analysis with an antibody cocktail containing ZOMBIE AQUA™ BV510 viability dye, anti-human CD45 (clone 2D1, APC/Cy7), anti-mouse CD45 (clone 30F-11, AF700), anti-CD4 (clone OKT4, BV785), anti-CD8 (clone SK1, PerCp/Cy5.5), anti-Bcl_xL (clone 54H6, APC), purchased from either Biolegend or Cell Signaling Technology. Samples were then analyzed by flow cytometry analysis. An increase in the absolute cell counts of anti-apoptotic Bcl_xL positive CD8 (FIG. 55) and CD4 T (data not shown) cells was observed in peripheral blood samples from treatment groups treated with the exemplary IgM antibody, T1: TROP-2×CD3 x CD28 IgM (with CD28 co-stimulation), in comparison to T3 (TROP-2 x CD3 IgM) and vehicle treatment groups.

### Example 27: T-Cell Dependent Cellular Toxicity Assay

The T cell dependent cytotoxicity (TDCC) activity of the following exemplary IgM antibodies, both with and without costimulatory activity, M1 (anti-mesothelin (MSLN) x anti-CD3 x anti-CD28 IgM antibody) and M3 (anti-mesothelin (MSLN) x anti-CD3 IgM antibody), respectively, as shown in Table 23, on the gastric cancer cell line, MKN45, was assessed by luminescence as follows. Antibody-mediated, redirected T-cell killing of MKN45-Luc cells was measured in a TDCC assay in which human PBMCs as effector cells (E) and MKN45-Luc cells as target cells (T) at an E:T ratio of 3:1 were incubated with serial dilutions of exemplary antibodies M1 and M3 in 96-well plates for 4 days at 37 °C in a humidified 5% CO₂ incubator. Following incubation, a luminescence-based viability assay as previously described was used to determine the viable cells. The percent of cell viability following antibody treatment was calculated by normalization of viable cells from the “no antibody” treatment group. Data are representative of 3 independent donors. Results are shown graphically in FIG. 56. As shown in the figure, improved cytotoxicity was observed upon treatment of MKN45 cells with the exemplary antibody, M1 (with CD28 co-stimulation), over antibody M3 (lacking CD28 co-stimulation) in this illustrative TDCC assay.

### Example 28: In Vivo Study: Antitumor Activity and Cytokine Release

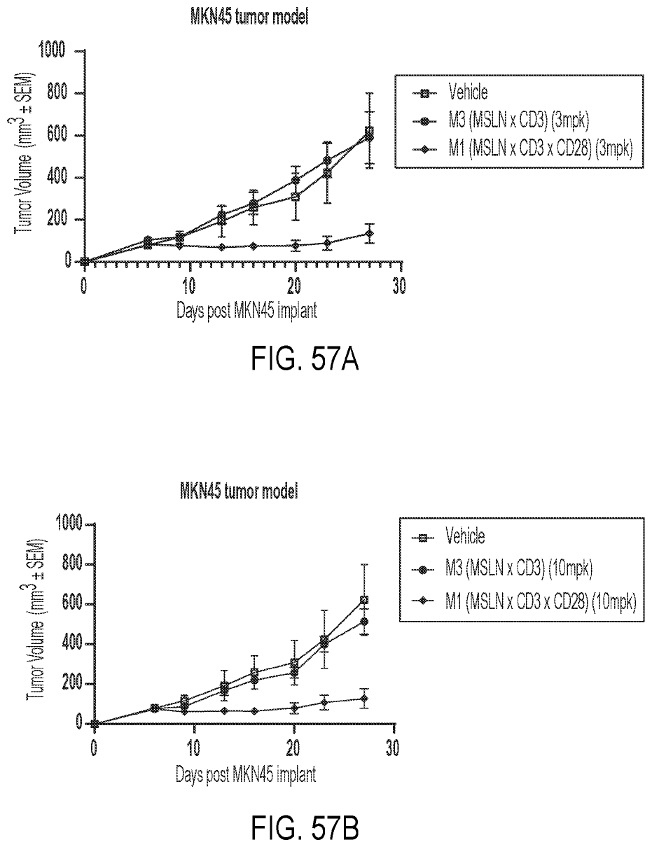

In a preclinical study, the antitumor activity of exemplary IgM antibodies, both with and without costimulatory activity, M1 (anti-mesothelin (MSLN) x anti-CD3 x anti-CD28 IgM antibody) and M3 (anti-mesothelin (MSLN) x anti-CD3 IgM antibody), respectively, as shown in Table 23, was evaluated in a humanized MKN45 gastric cancer xenograft model. NSG-MHCI/II DKO mice were engrafted with 10⁷ human PBMCs on day −14. On day 0, mice were implanted subcutaneously with 10⁷ MKN45 cells. Animals (n=7 per group) were dosed intravenously biweekly with antibody M1, antibody M3, or vehicle (1×PBS) at doses of antibody of 3 mg/kg or 10 mg/kg, starting on day 3 for a total of 12 doses (biwx12). Results are shown graphically as average tumor volume over time (days post MKN45 cell implant) in FIGS. 57A (3 mg/kg dose) and 57B (10 mg/kg dose). As shown in FIGS. 57A and 57B, treatment with an IgM antibody such as M1 (anti-mesothelin (MSLN) x anti-CD3 x anti-CD28, i.e., with CD28 co-stimulation) resulted in enhanced in vivo antitumor activity in comparison to exemplary IgM antibody M3 (anti-mesothelin (MSLN) x anti-CD3, i.e., lacking CD28 co-stimulation), e.g., when evaluated in a humanized MKN45 gastric cancer xenograft model.

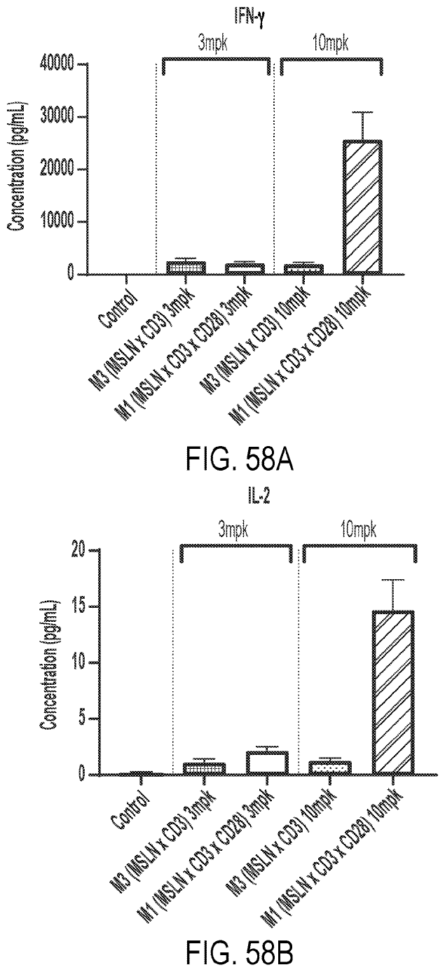

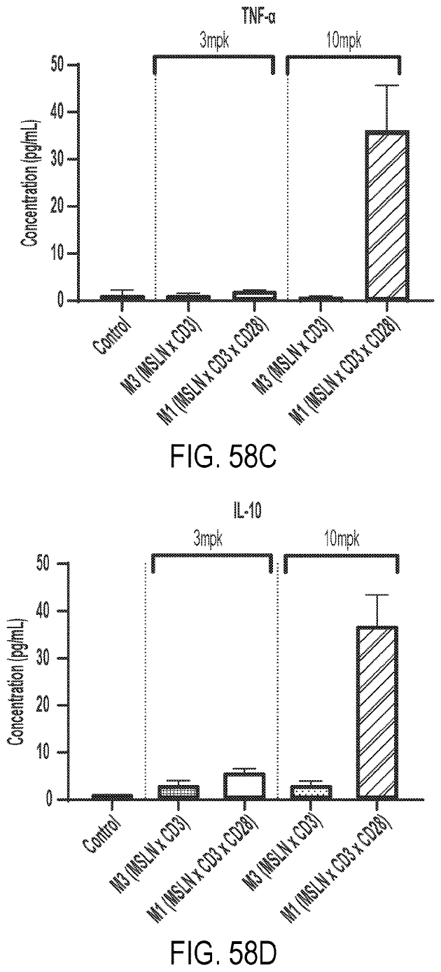

Cytokine Release: Harvested serum samples from each of the treatment groups were collected 4 hours post first dose and analyzed for cytokines IFN-γ, IL-2, TNF-α, and IL-using an MSD multiplex cytokine assay kit (Meso Scale Discovery) according to manufacturer's protocol. The results were then plotted with GraphPad Prism. Serum concentrations of IFN-γ, IL-2, TNF-α, and IL-10 are shown in FIGS. 58A, B, C, and D, respectively. A dose-dependent increase in the levels of released cytokines interleukin-2, interferon-gamma, tumor necrosis factor-alpha, and interleukin-10 was observed in the M1 IgM antibody treatment groups, i.e., following treatment with an exemplary MSLN xCD3xCD28 IgM antibody (i.e., with an IgM antibody with CD28 co-stimulation).

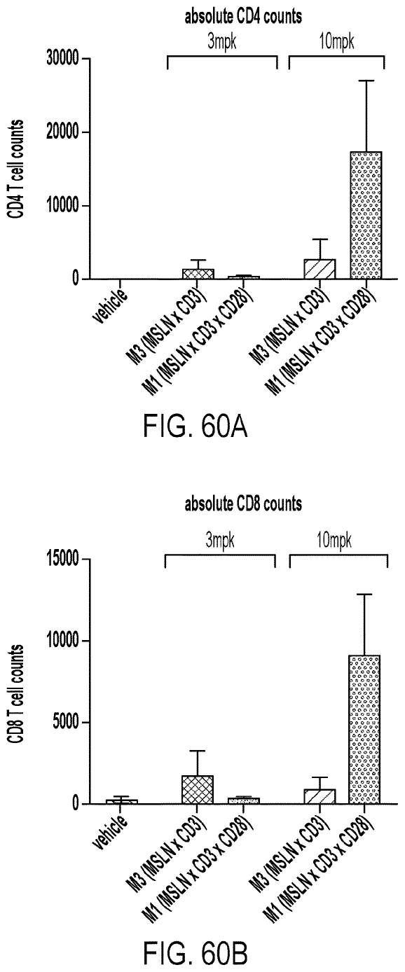

Intra-tumoral T cell counts: Mouse tumor samples were harvested on day 43 (end point). Tumor samples were processed and stained with an antibody cocktail containing viability dye (ZOMBIE AQUA™ BV510), anti-human CD45 (clone 2D1, APC/Cy7), anti-mouse CD45 (clone 30F-11, AF700), anti-CD4 (clone OKT4, BV785), and anti-CD8 (clone SK1, PerCp/Cy5.5), all reagents purchased from Biolegend. Samples were then subject to flow cytometry and gated for absolute CD4+ T cell counts (FIG. 60A) and absolute CD8+ T cell counts (FIG. 60B). An increase in the absolute cell counts for both CD4 and CD8 T cells were observed in tumor following CD28 co-stimulation at the higher dose.

### Example 29: Comparison of Various IgG and IgM Anti-CD28 Anti-CD3 Antibody Combinations in Non-Targeted T Cell Activation and Cytokine Release

The ability of various combinations of anti-CD28 and/or anti CD3 IgM or IgG4 antibodies to activate T cells and induce cytokine release was assessed using a plate-based assay by the following method. See, e.g., Stebbings, R., et al. J Immunol 179:3325-3331 (2007). This experiment compares two anti-CD28 binding domains, anti-CD28 Z (VH and VL amino acid sequences SEQ ID NO: 61 and SEQ ID NO: 81, respectively) and an anti-CD28 binding domain with known strong superagonist activity, W (VH and VL amino acid sequences SEQ ID NO: 69 and SEQ ID NO: 78, respectively). The constructs listed in Table 27 either alone or in combination were bound to the solid phase in a plate-based in vitro assay and were contacted with human PBMCs from two different donors. The PBMCs were assayed for T cell activation and the resulting culture supernatants were assayed for cytokine release.

TABLE 27
Isotype Control Antibodies

| Antibody | | VH/VL and J |
|---|---|---|
| Name | Description | chain SEQ ID NOs |
| D15 | Anti-virus isotype control with | 266, 267, 234 |
| | modified J* chain with anti-CD3- | |
| | AA and anti-CD28-Z | |
| D20 | Anti-virus isotype control with | 266, 267, 220 |
| | modified J* chain with anti-CD3- | |
| | AA | |
| D21 | Anti-virus isotype control with | 266, 267, 230 |
| | modified J* chain with anti-CD28 Z | |
| D22 | Anti-virus isotype control with | 266, 267, 282 |
| | modified J* chain with anti-CD28 | |
| | W | |
| D23 | Anti-virus isotype control with | 266, 267, 283 |
| | modified J* chain with anti-CD3- | |
| | AA and anti-CD28-W | |
| D24 | Anti-virus isotype control with | 266, 267, 8 |
| | unmodified J* | |
| D25 | Anti-CD3-AA human IgG4 | 24, 28, NA |
| D26 | Anti-CD28-Z human IgG4 | 61, 81, NA |
| D27 | Anti-CD28-W human IgG4 | 69, 78, NA |
| D28 | Anti-virus human IgG4 control | 266, 267, NA |

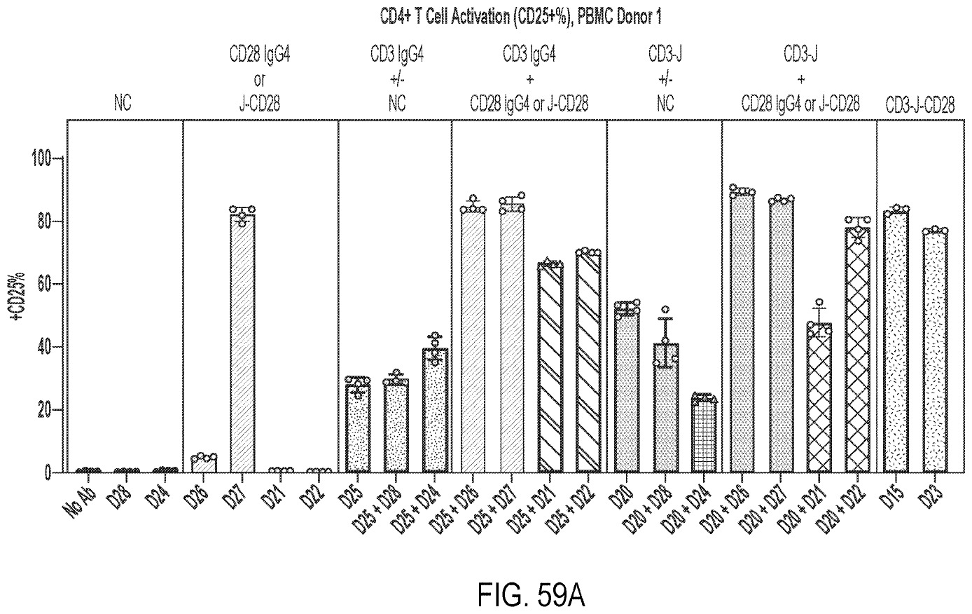

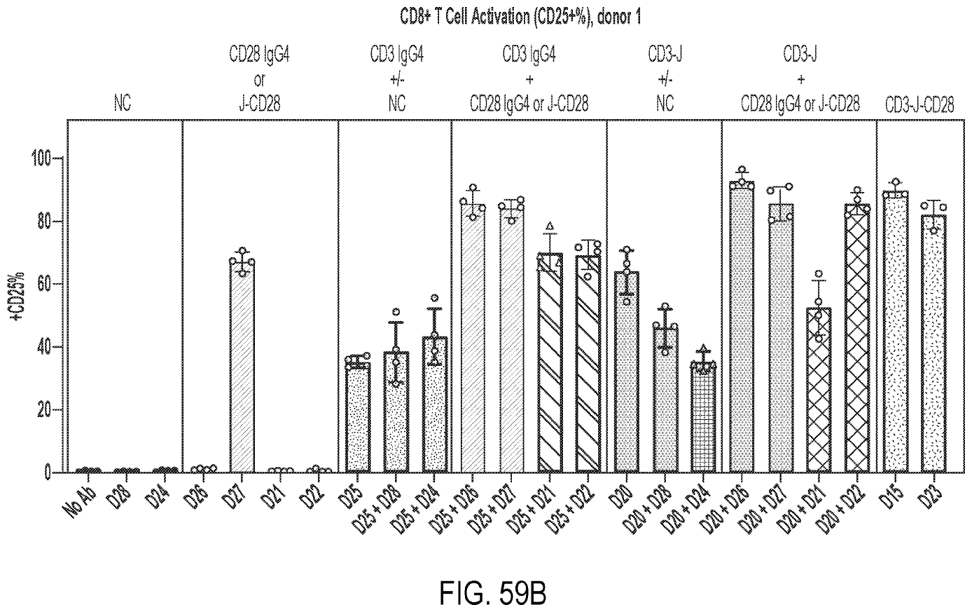

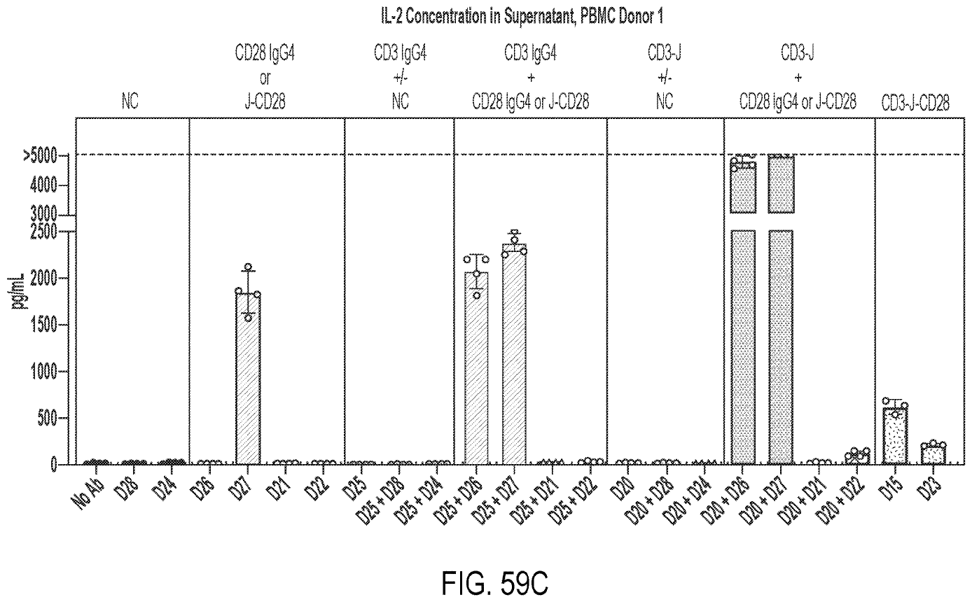

The various antibody constructs (100 μg/mL each in 25 μL phosphate-buffered saline (PBS)) were dispensed either alone or in combination into wells of 96-well U-bottom plates. Each condition was set up in triplicate or quadruplicate. The plates were left inside a ventilated biosafety cabinet without lids to be air dried overnight, in order to immobilize the antibodies in the wells. The plates were then washed once with 200 μL PBS and another time with 200 μL culture media (RPMI1640+ 10% heat inactivated FBS). Frozen human PBMCs from two different donors were thawed in a 37 °C water bath, washed with culture media, and then resuspended in culture media at 0.25×10⁶ cells/mL. The resuspended human PBMCs (200 μL, containing 5×10⁴ cells) were dispensed into each well of the washed plates coated with antibodies. The plates were then incubated for 72 hours at 37 °C with 5% CO₂ and 95% humidity. The cell pellets and culture supernatants were recovered. IL-2 levels in the cell culture supernatant samples were analyzed using the Human Th1/Th2 Cytokine Cytometric Bead Array (CBA) Kit II (BD, cat #551809). The IL-2 levels measured for one exemplary PBMC donor are shown in FIG. 59C. The cultured PBMC pellets were stained with fluorescent labeled antibodies against CD3, CD4, CD8 and CD25 for flow analysis to evaluate T cell activation status (CD25 positive percentage) of CD4 and CD8 T cell populations. T cell activation of PBMCs of the same exemplary PBMC donor are shown in FIG. 59A (CD4+ T cells) and FIG. 59B (CD8+ T cells).

Anti-CD28 W, as an IgG4 antibody (D28), showed strong T cell activation and strong IL-2 release even in the absence of CD3 stimulation, while anti-CD28 Z as an IgG4 antibody (D27) showed only weak T cell activation and no significant IL-2 release in the absence of CD3 stimulation. Both the anti-CD28 W IgG4 antibody (D28) and the anti-CD28 Z IgG4 antibody (D27) in combination with anti-CD3 AA as an IgG4 antibody (D25+D28 or D25+D27) or in combination with an anti-virus IgM isotype control with a J chain expressing an anti-CD3 AA ScFv (D20+D28 or D20+D27) showed strong T cell activation and high IL-2 release, in some cases going beyond the quantitation limit of the cytokine readout assay (5000 μg IL-2/mL). Neither anti-CD28 W nor anti-CD28 Z, expressed on the J chain of an anti-virus isotype control IgM (D22 and D21, respectively) showed significant T cell activation of cytokine release in the absence of CD3 stimulation. Moreover, cytokine release from D22 and D21 remained low even in the presence of separate anti-CD3 IgG4 molecules (D25+D21 or D25+D22) or separate anti-viral IgM isotype control molecules expressing anti-CD3 on the J chain (D20+D21 or D20+D22). Finally, efficient T cell activation and moderate IL-2 release was observed for the trispecific anti-viral IgM isotype control molecules in which both anti-CD28 Z or anti-CD28 W and anti-CD3 AA were co-expressed on the J chain (D15 and D23, respectively).

TABLE 28
Anti-CD38 Binding Domain

| | VH | | VL. | |
|---|---|---|---|---|
| Source | SEQ | | SEQ | |
| (Nickname) | ID NO | VH | ID NO | VL |
| WO202315067 | 272 | QAYLQQSGAEVVRSGASVKM | 273 | NIVLTQSPASLAVSLGQRATISC |
| 7A2 (E1) | | SCKAAGYTFTSYNMHWVKQT | | RASESLDTYGNSFMYWYQQKPGQ |
| | | PGQGLEWIGYIYPGNGGTNY | | PPKLLIYLASSLESGVPARFSGS |
| | | NQKFKGKATLTADTSSTTAY | | GSRTDFTLTIDPVEADDAATYYC |
| | | MQISSLTSEDSAVYFCARGA | | HQNHEDPWTFGGGTKLEIK |
| | | SMITTGAWFAYWGQGTLVTV | | |
| | | SA | | |

TABLE 29
Other Sequences in the Disclosure

| SEQ | | |
|---|---|---|
| ID | Nickname (source) | Sequence |
| 1 | Human IgM Constant | GSASAPTLFPLVSCENSPSDTSSVAVGCLAQDF |
| | region IMGT allele | LPDSITFSWKYKNNSDISSTRGFPSVLRGGKYA |
| | IGHM*03 (GenBank: | ATSQVLLPSKDVMQGTDEHVVCKVQHPNGN |
| | pir\|S37768\|) | KEKNVPLPVIAELPPKVSVFVPPRDGFFGNPRK |
| | | SKLICQATGFSPRQIQVSWLREGKQVGSGVTT |
| | | DQVQAEAKESGPTTYKVTSTLTIKESDWLSQS |
| | | MFTCRVDHRGLTFQQNASSMCVPDQDTAIRV |
| | | FAIPPSFASIFLTKSTKLTCLVTDLTTYDSVTIS |
| | | WTRQNGEAVKTHTNISESHPNATFSAVGEASI |
| | | CEDDWNSGERFTCTVTHTDLPSPLKQTISRPK |
| | | GVALHRPDVYLLPPAREQLNLRESATITCLVT |
| | | GFSPADVFVQWMQRGQPLSPEKYVTSAPMPE |
| | | PQAPGRYFAHSILTVSEEEWNTGETYTCVVAH |
| | | EALPNRVTERTVDKSTGKPTLYNVSLVMSDTA |
| | | GTCY |
| 2 | Human IgM Constant | GSASAPTLFPLVSCENSPSDTSSVAVGCLAQDF |
| | region IMGT allele | LPDSITFSWKYKNNSDISSTRGFPSVLRGGKYA |
| | IGHM*04 (GenBank: | ATSQVLLPSKDVMQGTDEHVVCKVQHPNGN |
| | sp\|P01871.4\|) | KEKNVPLPVIAELPPKVSVFVPPRDGFFGNPRK |
| | | SKLICQATGFSPRQIQVSWLREGKQVGSGVTT |
| | | DQVQAEAKESGPTTYKVTSTLTIKESDWLGQS |
| | | MFTCRVDHRGLTFQQNASSMCVPDQDTAIRV |
| | | FAIPPSFASIFLTKSTKLTCLVTDLTTYDSVTIS |
| | | WTRQNGEAVKTHTNISESHPNATFSAVGEASI |
| | | CEDDWNSGERFTCTVTHTDLPSPLKQTISRPK |
| | | GVALHRPDVYLLPPAREQLNLRESATITCLVT |
| | | GFSPADVFVQWMQRGQPLSPEKYVTSAPMPE |
| | | PQAPGRYFAHSILTVSEEEWNTGETYTCVVAH |
| | | EALPNRVTERTVDKSTGKPTLYNVSLVMSDTA |
| | | GTCY |
| 3 | Human IgA1 heavy chain | ASPTSPKVFPLSLCSTQPDGNVVIACLVQGFFP |
| | constant region,e.g., amino | QEPLSVTWSESGQGVTARNFPPSQDASGDLYT |
| | acids 144 to 496 of | TSSQLTLPATQCLAGKSVTCHVKHYTNPSQDV |
| | GenBank AIC59035.1 | TVPCPVPSTPPTPSPSTPPTPSPSCCHPRLSLHRP |
| | | ALEDLLLGSEANLTCTLTGLRDASGVTFTWTP |
| | | SSGKSAVQGPPERDLCGCYSVSSVLPGCAEPW |
| | | NHGKTFTCTAAYPESKTPLTATLSKSGNTFRPE |
| | | VHLLPPPSEELALNELVTLTCLARGFSPKDVLV |
| | | RWLQGSQELPREKYLTWASRQEPSQGTTTFA |
| | | VTSILRVAAEDWKKGDTFSCMVGHEALPLAF |
| | | TQKTIDRLAGKPTHVNVSVVMAEVDGTCY |
| 4 | Human IgA2 heavy chain | ASPTSPKVFPLSLDSTPQDGNVVVACLVQGFFP |
| | constant region,e.g., amino | QEPLSVTWSESGQNVTARNFPPSQDASGDLYT |
| | acids 1 to 340 of GenBank | TSSQLTLPATQCPDGKSVTCHVKHYTNSSQDV |
| | P01877.4 | TVPCRVPPPPPCCHPRLSLHRPALEDLLLGSEA |
| | | NLTCTLTGLRDASGATFTWTPSSGKSAVQGPP |
| | | ERDLCGCYSVSSVLPGCAQPWNHGETFTCTA |
| | | AHPELKTPLTANITKSGNTFRPEVHLLPPPSEEL |
| | | ALNELVTLTCLARGFSPKDVLVRWLQGSQELP |
| | | REKYLTWASRQEPSQGTTTYAVTSILRVAAED |
| | | WKKGETFSCMVGHEALPLAFTQKTIDRMAGK |
| | | PTHINVSVVMAEADGTCY |
| 5 | Precursor Human Secretory | MLLFVLTCLLAVFPAISTKSPIFGPEEVNSVEG |
| | Component | NSVSITCYYPPTSVNRHTRKYWCRQGARGGCI |
| | | TLISSEGYVSSKYAGRANLTNFPENGTFVVNIA |
| | | QLSQDDSGRYKCGLGINSRGLSFDVSLEVSQG |
| | | PGLLNDTKVYTVDLGRTVTINCPFKTENAQKR |
| | | KSLYKQIGLYPVLVIDSSGYVNPNYTGRIRLDI |
| | | QGTGQLLFSVVINQLRLSDAGQYLCQAGDDS |
| | | NSNKKNADLQVLKPEPELVYEDLRGSVTFHC |
| | | ALGPEVANVAKFLCRQSSGENCDVVVNTLGK |
| | | RAPAFEGRILLNPQDKDGSFSVVITGLRKEDAG |
| | | RYLCGAHSDGQLQEGSPIQAWQLFVNEESTIP |
| | | RSPTVVKGVAGGSVAVLCPYNRKESKSIKYW |
| | | CLWEGAQNGRCPLLVDSEGWVKAQYEGRLSL |
| | | LEEPGNGTFTVILNQLTSRDAGFYWCLTNGDT |
| | | LWRTTVEIKIIEGEPNLKVPGNVTAVLGETLKV |
| | | PCHFPCKFSSYEKYWCKWNNTGCQALPSQDE |
| | | GPSKAFVNCDENSRLVSLTLNLVTRADEGWY |
| | | WCGVKQGHFYGETAAVYVAVEERKAAGSRD |
| | | VSLAKADAAPDEKVLDSGFREIENKAIQDPRL |
| | | FAEEKAVADTRDQADGSRASVDSGSSEEQGG |
| | | SSRALVSTLVPLGLVLAVGAVAVGVARARHR |
| | | KNVDRVSIRSYRTDISMSDFENSREFGANDNM |
| | | GASSITQETSLGGKEEFVATTESTTETKEPKKA |
| | | KRSSKEEAEMAYKDFLLQSSTVAAEAQDGPQ |
| | | EA |
| 6 | Precursor Human J Chain | MKNHLLFWGVLAVFIKAVHVKAQEDERIVLV |
| | | DNKCKCARITSRIIRSSEDPNEDIVERNIRIIVPL |
| | | NNRENISDPTSPLRTRFVYHLSDLCKKCDPTEV |
| | | ELDNQIVTATQSNICDEDSATETCYTYDRNKC |
| | | YTAVVPLVYGGETKMVETALTPDACYPD |
| 7 | Mature Human J Chain | QEDERIVLVDNKCKCARITSRIIRSSEDPNEDIV |
| | | ERNIRIIVPLNNRENISDPTSPLRTRFVYHLSDL |
| | | CKKCDPTEVELDNQIVTATQSNICDEDSATETC |
| | | YTYDRNKCYTAVVPLVYGGETKMVETALTPD |
| | | ACYPD |
| 8 | J Chain Y102A mutation | QEDERIVLVDNKCKCARITSRIIRSSEDPNEDIV |
| | | ERNIRIIVPLNNRENISDPTSPLRTRFVYHLSDL |
| | | CKKCDPTEVELDNQIVTATQSNICDEDSATETC |
| | | ATYDRNKCYTAVVPLVYGGETKMVETALTPD |
| | | ACYPD |
| 9 | “5” Peptide linker | GGGGS |
| 10 | “10” Peptide linker | GGGGSGGGGS |
| 11 | “15” Peptide linker | GGGGSGGGGSGGGGS |
| 12 | “20” Peptide linker | GGGGSGGGGSGGGGSGGGGS |
| 13 | “25” Peptide Linker | GGGGSGGGGSGGGGSGGGGSGGGGS |
| 14 | “15KP” Peptide Linker | GKPGSGKPGSGKPGSGKPGS |
| 235 | 14-mer Linker (in SEQ | GGGGSGGGGSGGGG |
| | ID NO: 233) | |
| 279 | (GGGGS)ₙ multiple | (GGGGS)ₙ, where n is an integer between 2 and 5 |
| | linker | |
| 280 | (GGGGS)ₙ multiple | (GGGGS)ₙ, where n is an integer between 1 and 5 |
| | linker | |
| 281 | (GGGGS)ₙ multiple | (GGGGS)ₙ, where n is an integer between 2 and 3 |
| | linker | |
| 15 | 4-1BBL human | MEYASDASLDPEAPWPPAPRARACRVLPWAL |
| | | VAGLLLLLLLAAACAVFLACPWAVSGARASP |
| | | GSAASPRLREGPELSPDDPAGLLDLRQGMFAQ |
| | | LVAQNVLLIDGPLSWYSDPGLAGVSLTGGLSY |
| | | KEDTKELVVAKAGVYYVFFQLELRRVVAGEG |
| | | SGSVSLALHLQPLRSAAGAAALALTVDLPPAS |
| | | SEARNSAFGFQGRLLHLSAGQRLGVHLHTEAR |
| | | ARHAWQLTQGATVLGLFRVTPEIPAGLPSPRS |
| | | E |
| 215 | V scFv | QVQLVQSGAEVKKPGASVKVSCKASGYTFISY |
| | | TMHWVRQAPGQGLEWMGYINPRSGYTHYNQ |
| | | KLKDKATLTADKSASTAYMELSSLRSEDTAV |
| | | YYCARSAYYDYDGFAYWGQGTLVTVSSGGG |
| | | GSGGGGSGGGGSDIQMTQSPSSLSASVGDRVT |
| | | ITCSASSSVSYMNWYQQKPGKAPKRLIYDTSK |
| | | LASGVPSRFSGSGSGTDFTLTISSLQPEDFATYY |
| | | CQQWSSNPPTFGGGTKVEIK |
| 216 | V15J | QVQLVQSGAEVKKPGASVKVSCKASGYTFISY |
| | | TMHWVRQAPGQGLEWMGYINPRSGYTHYNQ |
| | | KLKDKATLTADKSASTAYMELSSLRSEDTAV |
| | | YYCARSAYYDYDGFAYWGQGTLVTVSSGGG |
| | | GSGGGGSGGGGSDIQMTQSPSSLSASVGDRVT |
| | | ITCSASSSVSYMNWYQQKPGKAPKRLIYDTSK |
| | | LASGVPSRFSGSGSGTDFTLTISSLQPEDFATYY |
| | | CQQWSSNPPTFGGGTKVEIKGGGGSGGGGSG |
| | | GGGSQEDERIVLVDNKCKCARITSRIIRSSEDP |
| | | NEDIVERNIRIIVPLNNRENISDPTSPLRTRFVY |
| | | HLSDLCKKCDPTEVELDNQIVTATQSNICDEDS |
| | | ATETCYTYDRNKCYTAVVPLVYGGETKMVET |
| | | ALTPDACYPD |
| 217 | V15J* | QVQLVQSGAEVKKPGASVKVSCKASGYTFISY |
| | | TMHWVRQAPGQGLEWMGYINPRSGYTHYNQ |
| | | KLKDKATLTADKSASTAYMELSSLRSEDTAV |
| | | YYCARSAYYDYDGFAYWGQGTLVTVSSGGG |
| | | GSGGGGSGGGGSDIQMTQSPSSLSASVGDRVT |
| | | ITCSASSSVSYMNWYQQKPGKAPKRLIYDTSK |
| | | LASGVPSRFSGSGSGTDFTLTISSLQPEDFATYY |
| | | CQQWSSNPPTFGGGTKVEIKGGGGSGGGGSG |
| | | GGGSQEDERIVLVDNKCKCARITSRIIRSSEDP |
| | | NEDIVERNIRIIVPLNNRENISDPTSPLRTRFVY |
| | | HLSDLCKKCDPTEVELDNQIVTATQSNICDEDS |
| | | ATETCATYDRNKCYTAVVPLVYGGETKMVET |
| | | ALTPDACYPD |
| 218 | SP34 scFv | EVQLVESGGGLVQPKGSLKLSCAASGFTFNTY |
| | | AMNWVRQAPGKGLEWVARIRSKYNNYATYY |
| | | ADSVKDRFTISRDDSQSILYLQMNNLKTEDTA |
| | | MYYCVRHGNFGNSYVSWFAYWGQGTLVTVS |
| | | SGGGGSGGGGSGGGGSQAVVTQESALTTSPG |
| | | ETVTLTCRSSTGAVTTSNYANWVQEKPDHLFT |
| | | GLIGGTNKRAPGVPARFSGSLIGDKAALTITGA |
| | | QTEDEAIYFCALWYSNLWVFGGGTKLTVL |
| 219 | AA scFv | EVQLLESGGGLVQPGGSLRLSCAASGFTFDTY |
| | | AMNWVRQAPGKGLEWVARIRSKYNNYATYY |
| | | ADSVKDRFTISRDDSKSTLYLQMESLRAEDTA |
| | | VYYCVRHANFGAGYVSWFAHWGQGTLVTVS |
| | | SGGGGSGGGGSGGGGSQTVVTQEPSLSVSPGG |
| | | TVTLTCGSSTGAVTTSNYANWVQQTPGQAPR |
| | | GLIGGTDKRAPGVPDRFSGSLLGDKAALTITG |
| | | AQAEDEADYYCALWYSNHWVFGGGTKLTVL |
| 220 | AA15J* | EVQLLESGGGLVQPGGSLRLSCAASGFTFDTY |
| | | AMNWVRQAPGKGLEWVARIRSKYNNYATYY |
| | | ADSVKDRFTISRDDSKSTLYLQMESLRAEDTA |
| | | VYYCVRHANFGAGYVSWFAHWGQGTLVTVS |
| | | SGGGGSGGGGSGGGGSQTVVTQEPSLSVSPGG |
| | | TVTLTCGSSTGAVTTSNYANWVQQTPGQAPR |
| | | GLIGGTDKRAPGVPDRFSGSLLGDKAALTITG |
| | | AQAEDEADYYCALWYSNHWVFGGGTKLTVL |
| | | GGGGSGGGGSGGGGSQEDERIVLVDNKCKCA |
| | | RITSRIIRSSEDPNEDIVERNIRIIVPLNNRENISD |
| | | PTSPLRTRFVYHLSDLCKKCDPTEVELDNQIVT |
| | | ATQSNICDEDSATETCATYDRNKCYTAVVPLV |
| | | YGGETKMVETALTPDACYPD |
| 221 | AB scFv | EVQLLESGGGLVQPGGSLRLSCAASGFTFDTY |
| | | AMNWVRQAPGKGLEWVARIRSKYNNYATYY |
| | | ADSVKDRFTISRDDSKSTLYLQMESLRAEDTA |
| | | VYYCVRHANFGAGYVSWFAHWGQGTLVTVS |
| | | SGGGGSGGGGSGGGGSQTVVTQEPSLSVSPGG |
| | | TVTLTCGSSTGAVTTSNYANWVQQTPGQAPR |
| | | GLIGGTDKRAPGVPDRFSGSLLGDKAALTITG |
| | | AQAEDEADYYCALWYSDLWVFGGGTKLTVL |
| 222 | AC scFv | QVQLVQSGAEVKKPGASVKVSCKASGFNIKD |
| | | YYMHWVRQAPGQRLEWMGWIDLENANTIYD |
| | | AKFQGRVTITRDTSASTAYMELSSLRSEDTAV |
| | | YYCARDAYGRYFYDVWGQGTLVTVSSGGGG |
| | | SGGGGSGGGGSDIVMTQSPDSLAVSLGERATI |
| | | NCKSSQSLLNARTGKNYLAWYQQKPGQPPKL |
| | | LIYWASTRESGVPDRFSGSGSGTDFTLTISSLQ |
| | | AEDVAVYYCKQSYSRRTFGGGTK VEIK |
| 223 | AD scFv | QVQLVQSGAEVKKPGASVKVSCKASGFNIKD |
| | | YYMHWVRQAPGQRLEWIGWIDLENANTVYD |
| | | AKFQGRVTITRDTSASTAYMELSSLRSEDTAV |
| | | YYCARDAYGRYFYDVWGQGTLVTVSSGGGG |
| | | SGGGGSGGGGSDIVMTQSPDSLAVSLGERATI |
| | | NCKSSQSLLNARTGKNYLAWYQQKPGQPPKL |
| | | LIYWASTRESGVPDRFSGSGSGTDFTLTISSLQ |
| | | AEDVAVYYCKQSYFRRTFGGGTKVEIK |
| 224 | AE scFv | QVQLVQSGAEVKKPGASVKVSCKASGFNIKD |
| | | YYMHWVRQAPGQRLEWIGWIDLENANTVYD |
| | | AKFQGRVTITRDTSASTAYMELSSLRSEDTAV |
| | | YYCARDAYGQYFYDVWGQGTLVTVSSGGGG |
| | | SGGGGSGGGGSDIVMTQSPDSLAVSLGERATI |
| | | NCKSSQSLLNARTGKNYLAWYQQKPGQPPKL |
| | | LIYWASTRESGVPDRFSGSGSGTDFTLTISSLQ |
| | | AEDVAVYYCTQSYFRRTFGGGTK VEIK |
| 225 | anti-CD28 X scFv | EVQLLESGGGLVQPGGSLRLSCAASGFTFSSY |
| | (US20220119530A1) | AMSWVRQAPGKGLEWVSTISGSGDSTYYADS |
| | | VKGRFTISRDNSKNTLYLQMNSLRAEDTAVY |
| | | YCAKSGPGLRQVGFDYWGQGTLVTVSSGKPG |
| | | SGKPGSGKPGSGKPGSDIQMTQSPSSLSASVGD |
| | | RVTITCRASQSISSYLNWYQQKPGKAPKLLIYA |
| | | ASSLQSGVPSRFSGSGSGTDFTLTISSLQPEDFA |
| | | TYYCQQVYSTPFTFGQGTKLEIK |
| 226 | anti-CD28 Y scFv | EVQLLESGGGLVQPGGSLRLSCAASGFTFSSY |
| | (US20220119530A1) | YMSWVRQAPGKGLEWVSTISESGDSTYYADS |
| | | VKGRFTISRDNSKNTLYLQMNSLRAEDTAVY |
| | | YCAKSGPGLRQVGFDYWGQGTLVTVSSGKPG |
| | | SGKPGSGKPGSGKPGSDIQMTQSPSSLSASVGD |
| | | RVTITCRASQSISSYLNWYQQKPGKAPKLLIYA |
| | | ASSLQSGVPSRFSGSGSGTDFTLTISSLQPEDFA |
| | | TYYCQQVYSTPFTFGQGTKLEIK |
| 227 | anti-CD28 Z scFv | QVQLQESGPGLVKPSETLSLTCAVSGFSLTSYG |
| | (US20040116675) | VHWIRQPPGKGLEWLGVIWPGGGTNFNSALM |
| | | SRLTISEDTSKNQVSLKLSSVTAADTAVYYCA |
| | | RDRAYGNYLYAMDYWGQGTLVTVSSGKPGS |
| | | GKPGSGKPGSGKPGSDIQMTQSPSSLSASVGD |
| | | RVTITCRASESVEYYVTSLMQWYQQKPGKAP |
| | | KLLIYAASNVDSGVPSRFSGSGSGTDFTLTISSL |
| | | QPEDIATYYCQQSRKVPFTFGGGTK VEIK |
| 228 | J*-anti-CD28 X | QEDERIVLVDNKCKCARITSRIIRSSEDPNEDIV |
| | | ERNIRIIVPLNNRENISDPTSPLRTRFVYHLSDL |
| | | CKKCDPTEVELDNQIVTATQSNICDEDSATETC |
| | | ATYDRNKCYTAVVPLVYGGETKMVETALTPD |
| | | ACYPDGGGGSGGGGSGGGGSEVQLLESGGGL |
| | | VQPGGSLRLSCAASGFTFSSYAMSWVRQAPG |
| | | KGLEWVSTISGSGDSTYYADSVKGRFTISRDN |
| | | SKNTLYLQMNSLRAEDTAVYYCAKSGPGLRQ |
| | | VGFDYWGQGTLVTVSSGKPGSGKPGSGKPGS |
| | | GKPGSDIQMTQSPSSLSASVGDRVTITCRASQS |
| | | ISSYLNWYQQKPGKAPKLLIYAASSLQSGVPS |
| | | RFSGSGSGTDFTLTISSLQPEDFATYYCQQVYS |
| | | TPFTFGQGTKLEIK |
| 229 | J*-anti-CD28 Y | QEDERIVLVDNKCKCARITSRIIRSSEDPNEDIV |
| | | ERNIRIIVPLNNRENISDPTSPLRTRFVYHLSDL |
| | | CKKCDPTEVELDNQIVTATQSNICDEDSATETC |
| | | ATYDRNKCYTAVVPLVYGGETKMVETALTPD |
| | | ACYPDGGGGSGGGGSGGGGSEVQLLESGGGL |
| | | VQPGGSLRLSCAASGFTFSSYYMSWVRQAPG |
| | | KGLEWVSTISESGDSTYYADSVKGRFTISRDNS |
| | | KNTLYLQMNSLRAEDTAVYYCAKSGPGLRQV |
| | | GFDYWGQGTLVTVSSGKPGSGKPGSGKPGSG |
| | | KPGSDIQMTQSPSSLSASVGDRVTITCRASQSIS |
| | | SYLNWYQQKPGKAPKLLIYAASSLQSGVPSRF |
| | | SGSGSGTDFTLTISSLQPEDFATYYCQQVYSTP |
| | | FTFGQGTKLEIK |
| 230 | J*-anti-CD28 Z | QEDERIVLVDNKCKCARITSRIIRSSEDPNEDIV |
| | | ERNIRIIVPLNNRENISDPTSPLRTRFVYHLSDL |
| | | CKKCDPTEVELDNQIVTATQSNICDEDSATETC |
| | | ATYDRNKCYTAVVPLVYGGETKMVETALTPD |
| | | ACYPDGGGGSGGGGSGGGGSQVQLQESGPGL |
| | | VKPSETLSLTCAVSGFSLTSYGVHWIRQPPGK |
| | | GLEWLGVIWPGGGTNFNSALMSRLTISEDTSK |
| | | NQVSLKLSSVTAADTAVYYCARDRAYGNYLY |
| | | AMDYWGQGTLVTVSSGKPGSGKPGSGKPGSG |
| | | KPGSDIQMTQSPSSLSASVGDRVTITCRASESV |
| | | EYYVTSLMQWYQQKPGKAPKLLIYAASNVDS |
| | | GVPSRFSGSGSGTDFTLTISSLQPEDIATYYCQQ |
| | | SRKVPFTFGGGTKVEIK |
| 231 | AA-J*-anti-CD28 X | EVQLLESGGGLVQPGGSLRLSCAASGFTFDTY |
| | | AMNWVRQAPGKGLEWVARIRSKYNNYATYY |
| | | ADSVKDRFTISRDDSKSTLYLQMESLRAEDTA |
| | | VYYCVRHANFGAGYVSWFAHWGQGTLVTVS |
| | | SGGGGSGGGGSGGGGSQTVVTQEPSLSVSPGG |
| | | TVTLTCGSSTGAVTTSNYANWVQQTPGQAPR |
| | | GLIGGTDKRAPGVPDRFSGSLLGDKAALTITG |
| | | AQAEDEADYYCALWYSNHWVFGGGTKLTVL |
| | | GGGGSGGGGSGGGGSQEDERIVLVDNKCKCA |
| | | RITSRIIRSSEDPNEDIVERNIRIIVPLNNRENISD |
| | | PTSPLRTRFVYHLSDLCKKCDPTEVELDNQIVT |
| | | ATQSNICDEDSATETCATYDRNKCYTAVVPLV |
| | | YGGETKMVETALTPDACYPDGGGGSGGGGSG |
| | | GGGSEVQLLESGGGLVQPGGSLRLSCAASGFT |
| | | FSSYAMSWVRQAPGKGLEWVSTISGSGDSTY |
| | | YADSVKGRFTISRDNSKNTLYLQMNSLRAEDT |
| | | AVYYCAKSGPGLRQVGFDYWGQGTLVTVSSG |
| | | KPGSGKPGSGKPGSGKPGSDIQMTQSPSSLSAS |
| | | VGDRVTITCRASQSISSYLNWYQQKPGKAPKL |
| | | LIYAASSLQSGVPSRFSGSGSGTDFTLTISSLQP |
| | | EDFATYYCQQVYSTPFTFGQGTKLEIK |
| 232 | AA-J*-anti-CD28 Y | EVQLLESGGGLVQPGGSLRLSCAASGFTFDTY |
| | | AMNWVRQAPGKGLEWVARIRSKYNNYATYY |
| | | ADSVKDRFTISRDDSKSTLYLQMESLRAEDTA |
| | | VYYCVRHANFGAGYVSWFAHWGQGTLVTVS |
| | | SGGGGSGGGGSGGGGSQTVVTQEPSLSVSPGG |
| | | TVTLTCGSSTGAVTTSNYANWVQQTPGQAPR |
| | | GLIGGTDKRAPGVPDRFSGSLLGDKAALTITG |
| | | AQAEDEADYYCALWYSNHWVFGGGTKLTVL |
| | | GGGGSGGGGSGGGGSQEDERIVLVDNKCKCA |
| | | RITSRIIRSSEDPNEDIVERNIRIIVPLNNRENISD |
| | | PTSPLRTRFVYHLSDLCKKCDPTEVELDNQIVT |
| | | ATQSNICDEDSATETCATYDRNKCYTAVVPLV |
| | | YGGETKMVETALTPDACYPDGGGGSGGGGSG |
| | | GGGSEVQLLESGGGLVQPGGSLRLSCAASGFT |
| | | FSSYYMSWVRQAPGKGLEWVSTISESGDSTYY |
| | | ADSVKGRFTISRDNSKNTLYLQMNSLRAEDTA |
| | | VYYCAKSGPGLRQVGFDYWGQGTLVTVSSGK |
| | | PGSGKPGSGKPGSGKPGSDIQMTQSPSSLSASV |
| | | GDRVTITCRASQSISSYLNWYQQKPGKAPKLLI |
| | | YAASSLQSGVPSRFSGSGSGTDFTLTISSLQPED |
| | | FATYYCQQVYSTPFTFGQGTKLEIK |
| 233 | AA-J*-anti-CD28 Z | EVQLLESGGGLVQPGGSLRLSCAASGFTFDTY |
| | | AMNWVRQAPGKGLEWVARIRSKYNNYATYY |
| | | ADSVKDRFTISRDDSKSTLYLQMESLRAEDTA |
| | | VYYCVRHANFGAGYVSWFAHWGQGTLVTVS |
| | | SGGGGSGGGGSGGGGSQTVVTQEPSLSVSPGG |
| | | TVTLTCGSSTGAVTTSNYANWVQQTPGQAPR |
| | | GLIGGTDKRAPGVPDRFSGSLLGDKAALTITG |
| | | AQAEDEADYYCALWYSNHWVFGGGTKLTVL |
| | | GGGGGGGGSGGGGSQEDERIVLVDNKCKCA |
| | | RITSRIIRSSEDPNEDIVERNIRIIVPLNNRENISD |
| | | PTSPLRTRFVYHLSDLCKKCDPTEVELDNQIVT |
| | | ATQSNICDEDSATETCATYDRNKCYTAVVPLV |
| | | YGGETKMVETALTPDACYPDGGGGSGGGGSG |
| | | GGGQVQLQESGPGLVKPSETLSLTCAVSGFSL |
| | | TSYGVHWIRQPPGKGLEWLGVIWPGGGTNFN |
| | | SALMSRLTISEDTSKNQVSLKLSSVTAADTAV |
| | | YYCARDRAYGNYLYAMDYWGQGTLVTVSSG |
| | | KPGSGKPGSGKPGSGKPGSDIQMTQSPSSLSAS |
| | | VGDRVTITCRASESVEYYVTSLMQWYQQKPG |
| | | KAPKLLIYAASNVDSGVPSRFSGSGSGTDFTLT |
| | | ISSLQPEDIATYYCQQSRKVPFTFGGGTKVEIK |
| 234 | AA-J*-anti-CD28 Z- | EVQLLESGGGLVQPGGSLRLSCAASGFTFDTY |
| | 2 | AMNWVRQAPGKGLEWVARIRSKYNNYATYY |
| | | ADSVKDRFTISRDDSKSTLYLQMESLRAEDTA |
| | | VYYCVRHANFGAGYVSWFAHWGQGTLVTVS |
| | | SGGGGSGGGGSGGGGSQTVVTQEPSLSVSPGG |
| | | TVTLTCGSSTGAVTTSNYANWVQQTPGQAPR |
| | | GLIGGTDKRAPGVPDRFSGSLLGDKAALTITG |
| | | AQAEDEADYYCALWYSNHWVFGGGTKLTVL |
| | | GGGGSGGGGSGGGGSQEDERIVLVDNKCKCA |
| | | RITSRIIRSSEDPNEDIVERNIRIIVPLNNRENISD |
| | | PTSPLRTRFVYHLSDLCKKCDPTEVELDNQIVT |
| | | ATQSNICDEDSATETCATYDRNKCYTAVVPLV |
| | | YGGETKMVETALTPDACYPDGGGGSGGGGSG |
| | | GGGSQVQLQESGPGLVKPSETLSLTCAVSGFS |
| | | LTSYGVHWIRQPPGKGLEWLGVIWPGGGTNF |
| | | NSALMSRLTISEDTSKNQVSLKLSSVTAADTA |
| | | VYYCARDRAYGNYLYAMDYWGQGTLVTVSS |
| | | GKPGSGKPGSGKPGSGKPGSDIQMTQSPSSLSA |
| | | SVGDRVTITCRASESVEYYVTSLMQWYQQKP |
| | | GKAPKLLIYAASNVDSGVPSRFSGSGSGTDFTL |
| | | TISSLQPEDIATYYCQQSRKVPFTFGGGTKVEI |
| | | K |
| 195 | Human 4-1BB | MGNSCYNIVATLLLVLNFERTRSLQDPCSNCP |
| | (GenBank: | AGTFCDNNRNQICSPCPPNSFSSAGGQRTCDIC |
| | AAA53133.1) | RQCKGVFRTRKECSSTSNAECDCTPGFHCLGA |
| | | GCSMCEQDCKQGQELTKKGCKDCCFGTFNDQ |
| | | KRGICRPWTNCSLDGKSVLVNGTKERDVVCG |
| | | PSPADLSPGASSVTPPAPAREPGHSPQIISFFLAL |
| | | TSTALLFLLFFLTLRFSVVKRGRKKLLYIFKQP |
| | | FMRPVQTTQEEDGCSCRFPEEEEGGCEL |
| 236 | Human CD28 (GenBank: | MLRLLLALNLFPSIQVTGNKILVKQSPMLVAY |
| | AAH93698.1) | DNAVNLSCKYSYNLFSREFRASLHKGLDSAVE |
| | | VCVVYGNYSQQLQVYSKTGFNCDGKLGNESV |
| | | TFYLQNLYVNQTDIYFCKIEVMYPPPYLDNEK |
| | | SNGTIIHVKGKHLCPSPLFPGPSKPFWVLVVVG |
| | | GVLACYSLLVTVAFIIFWVRSKRSRLLHSDYM |
| | | NMTPRRPGPTRKHYQPYAPPRDFAAYRS |
| 144 | anti-CD20 VH, | EVQLVQSGAEVKKPGESLKISCKGSGYSFTSYWIGWVRQMPG |
| | U.S. Pat. No. 1,0787,520 | KGLEWMGIIYPGDSDTRYSPSFQGQVTISADKSITTAYLQWS |
| | | SLKASDTAMYYCARHPSYGSGSPNFDYWGQGTLVTVSS |
| 145 | anti-CD20 VL, | DIVMTQTPLSSPVTLGQPASISCRSSQSLVYSDGNTYLSWLQ |
| | U.S. Pat. No. 1,0787,520 | QRPGQPPRLLIYKISNRFSGVPDRESGSGAGTDETLKISRVE |
| | | AEDVGVYYCVQATQFPLTFGGGTKVEIK |
| 237 | anti-CD20 IgG | EVQLVESGGGLVQPGGSLRLSCAASGYTFTSYNMHWVRQAPG |
| | subunit 1,FDA UNII | KGLEWVGAIYPGNGDTSYNQKFKGRFTISVDKSKNTLYLQMN |
| | LDJ89SSOYG | SLRAEDTAVYYCARVVYYSNSYWYFDVWGQGTLVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHK |
| | | PSNTKVDKKVEPKSCDKTHTCPPCPAPELLGGPSVFLFPPKP |
| | | KDTLMISRTPEVTCVVVDVSHEDPEVKENWYVDGVEVHNAKT |
| | | KPREEQYGSTYRVVSVLTVLHQDWLNGKEYKCKVSNKALPAP |
| | | IEKTISKAKGQPREPQVYTLPPSREEMTKNQVSLWCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| 238 | anti-CD20 IgG | EVQLVQSGAEVKKPGASVKVSCKASGYTFTNYYIHWVRQAPG |
| | subunit 2,FDA UNII | QGLEWIGWIYPGDGNTKYNEKFKGRATLTADTSTSTAYLELS |
| | LDJ89SSOYG | SLRSEDTAVYYCARDSYSNYYFDYWGQGTLVTVSSASTKGPS |
| | | VFPLAPSSKSTSGGTAALGCLVKDYFPEPVTVSWNSGALTSG |
| | | VHTFPAVLQSSGLYSLSSVVTVPSSSLGTQTYICNVNHKPSN |
| | | TKVDKKVEPKSCDKTHTCPPCPAPELLGGPSVFLFPPKPKDT |
| | | LMISRTPEVTCVVVDVSHEDPEVKFNWYVDGVEVHNAKTKPR |
| | | EEQYGSTYRVVSVLTVLHQDWINGKEYKCKVSNKALPAPIEK |
| | | TISKAKGQPREPQVYTLPPSREEMTKNQVSLSCAVKGFYPSD |
| | | IAVEWESNGQPENNYKTTPPVLDSDGSFFLVSKLTVDKSRWQ |
| | | QGNVFSCSVMHEALHNHYTQKSLSLSPGK |
| 239 | anti-CD20 IgG | DIVMTQSPDSLAVSLGERATINCKSSQSLLNSRTRKNYLAWY |
| | subunit 3,FDA UNII | QQKPGQPPKLLIYWASTRESGVPDRESGSGSGTDETLTISSL |
| | LDJ89SSOYG | QAEDVAVYYCTQSFILRTFGQGTKVEIKRTVAAPSVFIFPPS |
| | | DEQLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| 240 | anti-CD20 IgG | EVQLVESGGGLVQPGRSLRLSCVASGFTENDYAMHWVRQAPG |
| | subunit 1,FDA UNII | KGLEWVSVISWNSDSIGYADSVKGRFTISRDNAKNSLYLQMH |
| | 8R5CM46UIO | SLRAEDTALLYCAKDNHYGSGSYYYYQYGMDVWGQGTTVTVS |
| | | SASTKGPSVFPLAPCSRSTSESTAALGCLVKDYFPEPVTVSW |
| | | NSGALTSGVHTFPAVLQSSGLYSLSSVVTVPSSSLGTKTYTC |
| | | NVDHKPSNTKVDKRVESKYGPPCPPCPAPPVAGPSVFLEPPK |
| | | PKDTLMISRTPEVTCVVVDVSQEDPEVQFNWYVDGVEVHNAK |
| | | TKPREEQFNSTYRVVSVLTVLHQDWLNGKEYKCKVSNKGLPS |
| | | SIEKTISKAKGQPREPQVYTLPPSQEEMTKNQVSLTCLVKGF |
| | | YPSDIAVEWESNGQPENNYKTTPPVLDSDGSFFLYSRLTVDK |
| | | SRWQEGNVFSCSVMHEALHNHYTQKSLSLSLGK |
| 241 | anti-CD20 IgG | EVQLVESGGGLVQPGRSLRLSCAASGFTEDDYTMHWVRQAPG |
| | subunit,FDA UNII | KGLEWVSGISWNSGSIGYADSVKGRFTISRDNAKKSLYLQMN |
| | 28R5CM46UIO | SLRAEDTALLYCAKDNSGYGHYYYGMDVWGQGTTVTVASAST |
| | | KGPSVFPLAPCSRSTSESTAALGCLVKDYFPEPVTVSWNSGA |
| | | LTSGVHTFPAVLQSSGLYSLSSVVTVPSSSLGTKTYTCNVDH |
| | | KPSNTKVDKRVESKYGPPCPPCPAPPVAGPSVFLFPPKPKDT |
| | | LMISRTPEVTCVVVDVSQEDPEVQFNWYVDGVEVHNAKTKPR |
| | | EEQFNSTYRVVSVLTVLHQDWLNGKEYKCKVSNKGLPSSIEK |
| | | TISKAKGQPREPQVYTLPPSQEEMTKNQVSLTCLVKGFYPSD |
| | | IAVEWESNGQPENNYKTTPPVLDSDGSFFLYSRLTVDKSRWQ |
| | | EGNVFSCSVMHEALHNRFTQKSLSLSLGK |
| 242 | anti-CD20 IgG | EIVMTQSPATLSVSPGERATLSCRASQSVSSNLAWYQQKPGQ |
| | subunit 3-4,FDA | APRLLIYGASTRATGIPARFSGSGSGTEFTLTISSLQSEDFA |
| | UNII 8R5CM46UIO | VYYCQHYINWPLTFGGGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSEN |
| | | RGEC |
| 243 | V15J15HSA | QVQLVQSGAEVKKPGASVKVSCKASGYTFISYTMHWVRQAPG |
| | | QGLEWMGYINPRSGYTHYNQKLKDKATLTADKSASTAYMELS |
| | | SLRSEDTAVYYCARSAYYDYDGFAYWGQGTLVTVSSGGGGSG |
| | | GGGSGGGGSDIQMTQSPSSLSASVGDRVTITCSASSSVSYMN |
| | | WYQQKPGKAPKRLIYDTSKLASGVPSRFSGSGSGTDFTLTIS |
| | | SLQPEDFATYYCQQWSSNPPTFGGGTKVEIKGGGGSGGGGSG |
| | | GGGSQEDERIVLVDNKCKCARITSRIIRSSEDPNEDIVERNI |
| | | RIIVPLNNRENISDPTSPLRTRFVYHLSDLCKKCDPTEVELD |
| | | NQIVTATQSNICDEDSATETCYTYDRNKCYTAVVPLVYGGET |
| | | KMVETALTPDACYPDGGGGSGGGGSGGGGSDAHKSEVAHRFK |
| | | DLGEENFKALVLIAFAQYLQQCPFEDHVKLVNEVTEFAKTCV |
| | | ADESAENCDKSLHTLFGDKLCTVATLRETYGEMADCCAKQEP |
| | | ERNECFLQHKDDNPNLPRLVRPEVDVMCTAFHDNEETELKKY |
| | | LYEIARRHPYFYAPELLFFAKRYKAAFTECCQAADKAACLLP |
| | | KLDELRDEGKASSAKQRLKCASLQKFGERAFKAWAVARLSQR |
| | | FPKAEFAEVSKLVTDLTKVHTECCHGDLLECADDRADLAKYI |
| | | CENQDSISSKLKECCEKPLLEKSHCIAEVENDEMPADLPSLA |
| | | ADFVESKDVCKNYAEAKDVFLGMFLYEYARRHPDYSVVLLLR |
| | | LAKTYETTLEKCCAAADPHECYAKVEDEFKPLVEEPQNLIKQ |
| | | NCELFKQLGEYKFQNALLVRYTKKVPQVSTPTLVEVSRNLGK |
| | | VGSKCCKHPEAKRMPCAEDYLSVVLNQLCVLHEKTPVSDRVT |
| | | KCCTESLVNRRPCFSALEVDETYVPKEFNAETFTFHADICTL |
| | | SEKERQIKKQTALVELVKHKPKATKEQLKAVMDDFAAFVEKC |
| | | CKADDKETCFAEEGKKLVAASQAALGL |
| 266 | Anti-Viral Isotype | QVTLRESGPALVKPTQTLTLTCTFSGESLSTSGMSVGWIRQP |
| | control VH | PGKALEWLADIWWDDKKDYNPSLKSRLTISKDTSKNQVVLKV |
| | (U.S. Pat. No. 7,083,784B2) | TNMDPADTATYYCARSMITNWYFDVWGAGTTVTVSS |
| 267 | Anti-Viral Isotype | DIQMTQSPSTLSASVGDRVTITCKCQLSVGYMHWYQQKPGKA |
| | control VL | PKLLIYDTSKLASGVPSRFSGSGSGTEFTLTISSLQPDDEAT |
| | (U.S. Pat. No. 7,083,784B2) | YYCFQGSGYPFTFGGGTKLEIK |
| 269 | unmodified J chain | EVQLLESGGGLVQPGGSLRLSCAASGETEDTYAMNWVRQAPG |
| | with anti-CD3 scFv | KGLEWVARIRSKYNNYATYYADSVKDRFTISRDDSKSTLYLQ |
| | | MESLRAEDTAVYYCVRHANFGAGYVSWFAHWGQGTLVTVSSG |
| | | GGGSGGGGSGGGGSQTVVTQEPSLSVSPGGTVTLTCGSSTGA |
| | | VTTSNYANWVQQTPGQAPRGLIGGTDKRAPGVPDRESGSLLG |
| | | DKAALTITGAQAEDEADYYCALWYSNHWVFGGGTKLTVLGGG |
| | | GSGGGGSGGGGSQEDERIVLVDNKCKCARITSRIIRSSEDPN |
| | | EDIVERNIRIIVPLNNRENISDPTSPLRTRFVYHLSDLCKKC |
| | | DPTEVELDNQIVTATQSNICDEDSATETCYTYDRNKCYTAVV |
| | | PLVYGGETKMVETALTPDACYPD |
| 270 | Anti-4-1BB VH | EVQLLESGGGLVQPGGSLRLSCAASGFTFSSYAMSWVRQAPG |
| | U.S. Pat. No. | KGLEWVSAISGSGGSTYYADSVKGRFTISRDNSKNTLYLQMN |
| | 10,279,040B1 | SLRAEDTAVYYCAKDSPFLLDDYYYYYYMDVWGKGTTVTVSS |
| 271 | Anti-4-1BB VL | DIQMTQSPSSVSASVGDRVTITCRASQGISSWLAWYQQKPGK |
| | U.S. Pat. No. | APKLLIYAASSLQSGVPSRFSGSGSGTDFTLTISSLQPEDFA |
| | 10,279,040B1 | TYYCQQGHLFPITFGGGTKVEIK |
| 274 | TROP-2 bispecific | QVQLVQSGPEVKKPGASVKVSCKASGYTFTIYWLGWVRQAPG |
| | IgG TROP-2 binding | QGLEWMGNIFPGSAYINYNEKFKGKVTITADTSTSTAYMELS |
| | heavy chain | SLRSEDTAVYFCAREGSNSGYWGQGTLVTVSSASTKGPSVFP |
| | | LAPSSKSTSGGTAALGCLVKDYFPEPVTVSWNSGALTSGVHT |
| | | FPAVLQSSGLYSLSSVVTVPSSSLGTQTYICNVNHKPSDTKV |
| | | DKKVEPKSCDKTHTCPPCPAPPVAGPSVFLFPPKPKDTLMIS |
| | | RTPEVTCVVVDVKHEDPEVKENWYVDGVEVHNAKTKPREEEY |
| | | NSTYRVVSVLTVLHQDWLNGKEYKCKVSNKALPAPIEKTISK |
| | | AKGQPREPQVYTLPPSREEMTKNQVSLTCDVSGFYPSDIAVE |
| | | WESDGQPENNYKTTPPVLDSDGSFFLYSKLTVDKSRWEQGDV |
| | | FSCSVMHEALHNHYTQKSLSLSPGK |
| 275 | TROP-2 bispecific | DIVMTQSPDSLAVSLGERATINCKSSQSLLNSGNQQNYLAWY |
| | IgG TROP-2 binding | QQKPGQPPKLLIYGASTRESGVPDRESGSGSGTDETLTISSL |
| | light chain | QAEDVAVYYCQSDHIYPYTFGQGTKLEIKRTVAAPSVFIFPP |
| | | SDEQLKSGTASVVCLLNNFYPREAKVQWKVDNALQSGNSQES |
| | | VTEQDSKDSTYSLSSTLTLSKADYEKHKVYACEVTHQGLSSP |
| | | VTKSENRGEC |
| 276 | Anti-CD3 binding arm | EVQLLESGGGLVQPGGSLRLSCAASGFTFDTYAMNWVRQAPG |
| | for bispecific IgGs | KGLEWVARIRSKYNNYATYYADSVKDRFTISRDDSKSTLYLQ |
| | (TROP-2 AND MSLN) | MESLRAEDTAVYYCVRHANFGAGYVSWFAHWGQGTLVTVSSG |
| | | GGGSGGGGSGGGGSQTVVTQEPSLSVSPGGTVTLTCGSSTGA |
| | | VTTSNYANWVQQTPGQAPRGLIGGTDKRAPGVPDRESGSLLG |
| | | DKAALTITGAQAEDEADYYCALWYSNHWVFGGGTKLTVLEPK |
| | | SSDKTHTCPPCPAPPVAGPSVFLFPPKPKDTLMISRTPEVTC |
| | | VVVDVKHEDPEVKENWYVDGVEVHNAKTKPREEQYNSTYRVV |
| | | SVLTVLHQDWLNGKEYKCKVSNKALPAPIEKTISKAKGQPRE |
| | | PQVYTLPPSREQMTKNQVKLTCLVKGFYPSDIAVEWESNGQP |
| | | ENNYKTTPPVLDSDGSFFLYSKLTVDKSRWQQGNVFSCSVMH |
| | | EALHNHYTQKSLSLSPGK |
| 277 | MSLN bispecific IgG | QVQLVESGGGLVKPGGSLRLSCAASGFTFSDHYMSWIRQAPG |
| | MSLN binding heavy | KGLEWESYISSSGGIIYYADSVKGRFTISRDNAKNSLYLQMN |
| | chain | SLRAEDTAVYYCARDVGSHFDYWGQGTLVTVSSASTKGPSVE |
| | | PLAPSSKSTSGGTAALGCLVKDYFPEPVTVSWNSGALTSGVH |
| | | TFPAVLQSSGLYSLSSVVTVPSSSLGTQTYICNVNHKPSDTK |
| | | VDKKVEPKSCDKTHTCPPCPAPPVAGPSVFLFPPKPKDTLMI |
| | | SRTPEVTCVVVDVKHEDPEVKENWYVDGVEVHNAKTKPREEE |
| | | YNSTYRVVSVLTVLHQDWLNGKEYKCKVSNKALPAPIEKTIS |
| | | KAKGQPREPQVYTLPPSREEMTKNQVSLTCDVSGFYPSDIAV |
| | | EWESDGQPENNYKTTPPVLDSDGSFFLYSKLTVDKSRWEQGD |
| | | VESCSVMHEALHNHYTQKSLSLSPGK |
| 278 | MSLN bispecific IgG | DIQMTQSPSSVSASVGDRVTITCRASQDISRWLAWYQQKPGK |
| | MSLN binding light | APKLLISAASRLQSGVPSRFSGSGSGTDFTLTISSLQPEDFA |
| | chain | IYYCQQAKSFPRTFGQGTKVEIKRTVAAPSVFIFPPSDEQLK |
| | | SGTASVVCLLNNFYPREAKVQWKVDNALQSGNSQESVTEQDS |
| | | KDSTYSLSSTLTLSKADYEKHKVYACEVTHQGLSSPVTKSEN |
| | | RGEC |
| 282 | J*-anti-CD28 W | QEDERIVLVDNKCKCARITSRIIRSSEDPNEDIVERNIRIIV |
| | | PLNNRENISDPTSPLRTRFVYHLSDLCKKCDPTEVELDNQIV |
| | | TATQSNICDEDSATETCATYDRNKCYTAVVPLVYGGETKMVE |
| | | TALTPDACYPDGGGGSGGGGSGGGGSQVQLVQSGAEVKKPGA |
| | | SVKVSCKASGYTFTSYYIHWVRQAPGQGLEWIGCIYPGNVNT |
| | | NYNEKFKDRATLTVDTSISTAYMELSRLRSDDTAVYFCTRSH |
| | | YGLDWNFDVWGQGTTVTVSSGKPGSGKPGSGKPGSGKPGSDI |
| | | QMTQSPSSLSASVGDRVTITCHASQNIYVWLNWYQQKPGKAP |
| | | KLLIYKASNLHTGVPSRFSGSGSGTDETLTISSLQPEDFATY |
| | | YCQQGQTYPYTFGGGTKVEIK |
| 283 | AAJ *-anti-CD28 W | EVQLLESGGGLVQPGGSLRLSCAASGFTFDTYAMNWVRQAPG |
| | | KGLEWVARIRSKYNNYATYYADSVKDRFTISRDDSKSTLYLQ |
| | | MESLRAEDTAVYYCVRHANFGAGYVSWFAHWGQGTLVTVSSG |
| | | GGGSGGGGSGGGGSQTVVTQEPSLSVSPGGTVTLTCGSSTGA |
| | | VTTSNYANWVQQTPGQAPRGLIGGTDKRAPGVPDRESGSLLG |
| | | DKAALTITGAQAEDEADYYCALWYSNHWVFGGGTKLTVLGGG |
| | | GSGGGGSGGGGSQEDERIVLVDNKCKCARITSRIIRSSEDPN |
| | | EDIVERNIRIIVPLNNRENISDPTSPLRTRFVYHLSDLCKKC |
| | | DPTEVELDNQIVTATQSNICDEDSATETCATYDRNKCYTAVV |
| | | PLVYGGETKMVETALTPDACYPDGGGGSGGGGSGGGGQVQLV |
| | | QSGAEVKKPGASVKVSCKASGYTFTSYYIHWVRQAPGQGLEW |
| | | IGCIYPGNVNTNYNEKFKDRATLTVDTSISTAYMELSRLRSD |
| | | DTAVYFCTRSHYGLDWNFDVWGQGTTVTVSSGKPGSGKPGSG |
| | | KPGSGKPGSDIQMTQSPSSLSASVGDRVTITCHASQNIYVWL |
| | | NWYQQKPGKAPKLLIYKASNLHTGVPSRFSGSGSGTDETLTI |
| | | SSLQPEDFATYYCQQGQTYPYTFGGGTKVEIK |
| 284 | anti-CD28 W scFv | QVQLVQSGAEVKKPGASVKVSCKASGYTFTSYYIHWVRQAPG |
| | (U.S. Pat. No. 8,586,386) | QGLEWIGCIYPGNVNTNYNEKFKDRATLTVDTSISTAYMELS |
| | | RLRSDDTAVYFCTRSHYGLDWNFDVWGQGTTVTVSSGKPGSG |
| | | KPGSGKPGSGKPGSDIQMTQSPSSLSASVGDRVTITCHASQN |
| | | IYVWLNWYQQKPGKAPKLLIYKASNLHTGVPSRFSGSGSGTD |
| | | FTLTISSLQPEDFATYYCQQGQTYPYTFGGGTKVEIK |

What is claimed is:

1. A multimeric binding molecule comprising five, four, or two bivalent binding units and a modified J chain,
wherein each binding unit comprises two antibody heavy chains, each comprising an IgM or IgA heavy chain constant region or multimerizing variant or fragment thereof each associated with a target antigen-binding domain, and
wherein the modified J chain comprises (a) a J chain or a functional fragment or variant thereof (“J”), (b) an anti-CD3 scFv (“C”), and (c) a heterologous polypeptide (“H”), wherein H comprises a polypeptide agonist of a T cell costimulatory molecule, wherein the J, C, and H are associated as a fusion protein.

2. The multimeric binding molecule of claim 1, wherein the T cell costimulatory molecule comprises CD28 or 4-1BB.

3. The multimeric binding molecule of claim 2, wherein H comprises an anti-CD28 antibody or antigen-binding fragment thereof or a 4-1BB ligand (“4-1BBL”) trimer.

4. The multimeric binding molecule of claim 1, wherein the modified J chain comprises, in the N-terminal to C-terminal direction, C-J-H or H-J-C.

5. The multimeric binding molecule of claim 4, wherein C, J, and H are fused via amino acid linkers, which can be the same or different.

6. The multimeric binding molecule of claim 3, wherein H comprises an antigen-binding fragment of an anti-CD28 antibody.

7. The multimeric binding molecule of claim 6, wherein the antigen-binding fragment of the anti-CD28 antibody is a single-chain Fv (scFv) fragment comprising a heavy chain variable region (VH) and a light chain variable region (VL), wherein the VH comprises VH complementarity-determining regions VHCDR1, VHCDR2, and VHCDR3 and the VL comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 100, SEQ ID NO: 120, SEQ ID NO: 127, SEQ ID NO: 146, SEQ ID NO: 153, and SEQ ID NO: 172; SEQ ID NO: 95, SEQ ID NO: 105, SEQ ID NO: 133, SEQ ID NO: 138, SEQ ID NO: 164, and SEQ ID NO: 165; SEQ ID NO: 98, SEQ ID NO: 106, SEQ ID NO: 130, SEQ ID NO: 151, SEQ ID NO: 157, and SEQ ID NO: 171; SEQ ID NO: 98, SEQ ID NO: 116, SEQ ID NO: 135, SEQ ID NO: 147, SEQ ID NO: 155, and SEQ ID NO: 175; SEQ ID NO: 103, SEQ ID NO: 115, SEQ ID NO: 135, SEQ ID NO: 147, SEQ ID NO: 155, and SEQ ID NO: 175; SEQ ID NO: 96, SEQ ID NO: 110, SEQ ID NO: 125, SEQ ID NO: 147, SEQ ID NO: 155, and SEQ ID NO: 174; SEQ ID NO: 96, SEQ ID NO: 110, SEQ ID NO: 125, SEQ ID NO: 150, SEQ ID NO: 159, and SEQ ID NO: 176; SEQ ID NO: 104, SEQ ID NO: 123, SEQ ID NO: 137, SEQ ID NO: 150, SEQ ID NO: 159, and SEQ ID NO: 176; SEQ ID NO: 91, SEQ ID NO: 118, SEQ ID NO: 126, SEQ ID NO: 146, SEQ ID NO: 154, and SEQ ID NO: 173; SEQ ID NO: 101, SEQ ID NO: 111, SEQ ID NO: 129, SEQ ID NO: 139, SEQ ID NO: 161, and SEQ ID NO: 169; SEQ ID NO: 97, SEQ ID NO: 112, SEQ ID NO: 124, SEQ ID NO: 142, SEQ ID NO: 163, and SEQ ID NO: 166; SEQ ID NO: 94, SEQ ID NO: 108, SEQ ID NO: 132, SEQ ID NO: 149, SEQ ID NO: 160, and SEQ ID NO: 177; SEQ ID NO: 93, SEQ ID NO: 109, SEQ ID NO: 131, SEQ ID NO: 148, SEQ ID NO: 158, and SEQ ID NO: 178; SEQ ID NO: 99, SEQ ID NO: 117, SEQ ID NO: 128, SEQ ID NO: 147, SEQ ID NO: 155, and SEQ ID NO: 174; SEQ ID NO: 99, SEQ ID NO: 117, SEQ ID NO: 128, SEQ ID NO: 150, SEQ ID NO: 159, and SEQ ID NO: 176; SEQ ID NO: 102, SEQ ID NO: 107, SEQ ID NO: 136, SEQ ID NO: 140, SEQ ID NO: 162, and SEQ ID NO: 170; SEQ ID NO: 102, SEQ ID NO: 114, SEQ ID NO: 136, SEQ ID NO: 140, SEQ ID NO: 162, and SEQ ID NO: 170; SEQ ID NO: 102, SEQ ID NO: 113, SEQ ID NO: 136, SEQ ID NO: 143, SEQ ID NO: 162, and SEQ ID NO: 170; SEQ ID NO: 91, SEQ ID NO: 119, SEQ ID NO: 126, SEQ ID NO: 146, SEQ ID NO: 154, and SEQ ID NO: 173; SEQ ID NO: 92, SEQ ID NO: 121, SEQ ID NO: 134, SEQ ID NO: 152, SEQ ID NO: 156, and SEQ ID NO: 167; or SEQ ID NO: 92, SEQ ID NO: 122, SEQ ID NO: 134, SEQ ID NO: 141, SEQ ID NO: 156, and SEQ ID NO: 168.

8. The multimeric binding molecule of claim 7, wherein the VH and VL of the anti-CD28 scFv comprise amino acid sequences at least 80%, 85%, 90%, 95%, or 100% identical to the amino acid sequences SEQ ID NO: 61 and SEQ ID NO: 81, SEQ ID NO: 56 and SEQ ID NO: 90, SEQ ID NO: 57 and SEQ ID NO: 86, SEQ ID NO: 58 and SEQ ID NO: 83, SEQ ID NO: 59 and SEQ ID NO: 83, SEQ ID NO: 60 and SEQ ID NO: 82, SEQ ID NO: 60 and SEQ ID NO: 87, SEQ ID NO: 62 and SEQ ID NO: 87, SEQ ID NO: 63 and SEQ ID NO: 85, SEQ ID NO: 64 and SEQ ID NO: 76, SEQ ID NO: 65 and SEQ ID NO: 75, SEQ ID NO: 66 and SEQ ID NO: 89, SEQ ID NO: 67 and SEQ ID NO: 88, SEQ ID NO: 68 and SEQ ID NO: 82, SEQ ID NO: 68 and SEQ ID NO: 87, SEQ ID NO: 69 and SEQ ID NO: 78, SEQ ID NO: 70 and SEQ ID NO: 78, SEQ ID NO: 71 and SEQ ID NO: 80, SEQ ID NO: 72 and SEQ ID NO: 84, SEQ ID NO: 73 and SEQ ID NO: 77, or SEQ ID NO: 74 and SEQ ID NO: 79, respectively.

9. The multimeric binding molecule of claim 7, wherein the VH and VL are fused via an amino acid linker.

10. The multimeric binding molecule of claim 1, wherein the C comprises a heavy chain variable region (VH) and a light chain variable region (VL), wherein the VH of C comprises VH complementarity-determining regions VHCDR1, VHCDR2, and VHCDR3 and the VL of C comprises VL complementarity-determining regions VLCDR1, VLCDR2, and VLCDR3, wherein the VHCDR1, VHCDR2, VHCDR3, VLCDR1, VLCDR2, and VLCDR3 comprise, respectively, the amino acid sequences SEQ ID NO: 25, SEQ ID NO: 26, SEQ ID NO: 27, SEQ ID NO: 29, SEQ ID NO: 30, and SEQ ID NO: 31; SEQ ID NO: 17, SEQ ID NO: 18, SEQ ID NO: 19, SEQ ID NO: 21, SEQ ID NO: 22, and SEQ ID NO: 23; SEQ ID NO: 25, SEQ ID NO: 26, SEQ ID NO: 27, SEQ ID NO: 29, SEQ ID NO: 30, and SEQ ID NO: 33; SEQ ID NO: 35, SEQ ID NO: 36, SEQ ID NO: 37, SEQ ID NO: 39, SEQ ID NO: 40, and SEQ ID NO: 41; SEQ ID NO: 35, SEQ ID NO: 43, SEQ ID NO: 37, SEQ ID NO: 39, SEQ ID NO: 40, and SEQ ID NO: 45; SEQ ID NO: 35, SEQ ID NO: 43, SEQ ID NO: 47, SEQ ID NO: 39, SEQ ID NO: 40, and SEQ ID NO: 49, or SEQ ID NO: 25, SEQ ID NO: 26, SEQ ID NO: 51, SEQ ID NO: 53, SEQ ID NO: 54, and SEQ ID NO: 55.

11. The multimeric binding molecule of claim 10, wherein the VH and VL of C comprise an amino acid sequence at least 80%, 85%, 90%, 95%, or 100% identical to the amino acid sequence of SEQ ID NO: 24 and SEQ ID NO: 28; SEQ ID NO: 16 and SEQ ID NO: 20; SEQ ID NO: 24 and SEQ ID NO: 32; SEQ ID NO: 34 and SEQ ID NO: 38; SEQ ID NO: 42 and SEQ ID NO: 44; or SEQ ID NO: 46 and SEQ ID NO: 48, respectively.

12. The multimeric binding molecule of claim 10, wherein the VH and VL are fused via an amino acid linker.

13. The multimeric binding molecule of any one of claim 5, 9, or 12, wherein the linker consists of the amino acid sequence (GGGGS)n, wherein n is an integer between 2 and (SEQ ID NO:279), GGGGSGGGGS (SEQ ID NO: 10), GGGGSGGGGSGGGGS (SEQ ID NO: 11), GKPGSGKPGSGKPGSGKPGS (SEQ ID NO: 14), or GGGGSGGGGGGGG (SEQ ID NO: 235).

14. The multimeric binding molecule of claim 1, wherein J comprises SEQ ID NO: 7 or a functional fragment or variant thereof.

15. The multimeric binding molecule of claim 14, wherein J comprises a variant J chain with an alanine substitution at the amino acid position corresponding to amino acid Y102 of the mature wild-type human J chain (SEQ ID NO: 7).

16. The multimeric binding molecule of claim 15, wherein J comprises the amino acid sequence SEQ ID NO: 8 (“J*”).

17. The multimeric binding molecule of claim 16, wherein the modified J chain comprises the amino acid sequence SEQ ID NO: 233, SEQ ID NO: 231, SEQ ID NO: 232, or SEQ ID NO: 234.

18. The multimeric binding molecule of claim 1, wherein the multimeric binding molecule is an IgM antibody comprising five bivalent binding units, each comprising two IgM heavy chain constant regions or multimerizing fragments or variants thereof each comprising an IgM Cμ4 domain and an IgM tailpiece domain.

19. The multimeric binding molecule of claim 18, wherein each IgM heavy chain constant region or multimerizing fragment or variant thereof further comprises a Cμ1 domain, a Cμ2 domain, a Cμ3 domain, or any combination thereof.

20. The multimeric binding molecule of claim 18, wherein each IgM heavy chain constant region or multimerizing fragment or variant thereof is a human IgM constant region comprising the amino acid sequence SEQ ID NO: 1, SEQ ID NO: 2, or a multimerizing variant or fragment thereof.

21. The multimeric binding molecule of any one of claims 1 to 20, wherein each target antigen-binding domain is identical and comprises a heavy chain variable region (VH) and a light chain variable region (VL), or a single-domain heavy chain variable region (sdVH).

22. The multimeric binding molecule of claim 21, wherein each heavy chain comprises, from N-terminus to C-terminus, the VH fused to the heavy chain constant region or multimerizing fragment or variant thereof.

23. The multimeric binding molecule of claim 22, wherein each binding unit further comprises two antibody light chains, each comprising, from N-terminus to C-terminus, the VL fused to a light chain constant region.

24. The multimeric binding molecule of any one of claims 1 to 20, wherein the target antigen is CD38, CD20, CD19, CD22, CD28, B cell maturation antigen (BCMA), CD123, PD-L1, tumor-associated calcium signal transducer 2 (TROP-2), mesothelin, Muc16, prostate-specific membrane antigen (PSMA), or Six-Transmembrane Epithelial Antigen of Prostate-1·(STEAP-1).

25. The multimeric binding molecule of any one of claims 1 to 20, wherein the multimeric binding molecule conditionally activates T cells in the presence of the target antigen.

26. The multimeric binding molecule of claim 25, wherein the T cells are CD4 positive T cells.

27. The multimeric binding molecule of claim 25, wherein the T cells are CD8 positive T cells.

28. A composition comprising the multimeric binding molecule of any one of claims 1 to 20 and a pharmaceutically acceptable carrier.

29. A polynucleotide comprising a nucleic acid sequence that encodes the modified J chain and a nucleic acid sequence that encodes the antibody heavy chain of the multimeric binding molecule of any one of claims 1 to 20.

30. A vector comprising the polynucleotide of claim 29.

31. A host cell comprising the vector of claim 30, wherein the host cell can express the multimeric binding molecule.

32. A method of producing the multimeric binding molecule of any one of claims 1 to 27, comprising culturing the host cell of claim 31, and recovering the multimeric binding molecule.

33. A method of treating cancer, comprising administering to a subject in need of treatment the multimeric binding molecule of any one of claims 1 to 20.
